SEQUENCE LISTING (229 sequences)

SEQ ID NO: 1 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQRDSYLWTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 227 aa, Artificial Sequence
  Description of Artificial Sequence Synthetic polypeptide
EISEVQLVESGGGLVQPGGSLRLSCAASGFTFSTYYIHWVRQAPGKGLEWVAVIYPYTGFTYYADSVKGRFTISADTSKNTAYLQMNSLRAEDTAVYYCARGEYTVLDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 3 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQVDSTPVTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 225 aa, Artificial Sequence
  Description of Artificial Sequence Synthetic polypeptide
EVQLVESGGGLVQPGGSLRLSCAASGFTFSSYYIHWVRQAPGKGLEWVAYIGSQGGFTDYADSVKGRFTISADTSKNTAYLQMNSLRAEDTAVYYCARQGYGYALDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 5 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYDYSPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 222 aa, Artificial Sequence
  Description of Artificial Sequence Synthetic polypeptide
EVQLVESGGGLVQPGGSLRLSCAASGFTFWSYYIHWVRQAPGKGLEWVAAITPYDGYTYYADSVKGRFTISADTSKNTAYLQMNSLRAEDTAVYYCARGSVYTGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 7 - protein, 665 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYIYGPLTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYIYGPLTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 224 aa, Artificial Sequence
  Description of Artificial Sequence Synthetic polypeptide
EVQLVESGGGLVQPGGSLRLSCAASGFTFDGYYIHWVRQAPGKGLEWVAAIESSSGYTYYADSVKGRFTISADTSKNTAYLQMNSLRAEDTAVYYCARAYYTGMDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 9 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYIYGPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 221 aa, Artificial Sequence
  Description of Artificial Sequence Synthetic polypeptide
EVQLVESGGGLVQPGGSLRLSCAASGFTFDGYYIHWVRQAPGKGLEWVAAIESSSGYTYYADSVKGRFTISADTSKNTAYLQMNSLRAEDTAVYYCARAYYTGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 11 - protein, 224 aa, Artificial Sequence; identical to SEQ ID NO: 8
  Description of Artificial Sequence Synthetic polypeptide

SEQ ID NO: 12 - protein, 447 aa, Artificial Sequence
  Description of Artificial Sequence Synthetic polypeptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, Artificial Sequence
  Description of Artificial Sequence Synthetic polypeptide
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 451 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, Artificial Sequence
  Description of Artificial Sequence Synthetic polypeptide
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 449 aa, Artificial Sequence
  Description of Artificial Sequence Synthetic polypeptide
EVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 213 aa, Artificial Sequence
  Description of Artificial Sequence Synthetic polypeptide
DIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 10 aa, Artificial Sequence
  Description of Artificial Sequence Synthetic peptide
DKTHTCPPCP

SEQ ID NO: 19 - protein, 455 aa, Artificial Sequence
  Description of Artificial Sequence Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, Artificial Sequence
  Description of Artificial Sequence Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 109 aa, Artificial Sequence
  Description of Artificial Sequence Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRT

SEQ ID NO: 22 - protein, 127 aa, Artificial Sequence
  Description of Artificial Sequence Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSAS

SEQ ID NO: 23 - protein, 441 aa, Artificial Sequence
  Description of Artificial Sequence Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 232 aa, Artificial Sequence
  Description of Artificial Sequence Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 25 - protein, 441 aa, Artificial Sequence
  Description of Artificial Sequence Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 26 - protein, 229 aa, Artificial Sequence
  Description of Artificial Sequence Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 27 - protein, 665 aa, Artificial Sequence
  Description of Artificial Sequence Synthetic polypeptide
  MOD_RES (91)..(96): Any amino acid
  MOD_RES (315)..(320): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 496 aa, Artificial Sequence
  Description of Artificial Sequence Synthetic polypeptide
MGWSLILLFLVAVATRVLSCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRASSGLNDIFEAQKIEWHEGTENLYFQSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSHHHHHHHH

SEQ ID NO: 29 - protein, 19 aa, Artificial Sequence
  Description of Artificial Sequence Synthetic peptide
MGWSLILLFLVAVATRVLS

SEQ ID NO: 30 - protein, 15 aa, Artificial Sequence
  Description of Artificial Sequence Synthetic peptide
GLNDIFEAQKIEWHE

SEQ ID NO: 31 - protein, 6 aa, Artificial Sequence
  Description of Artificial Sequence Synthetic peptide
ENLYFQ

SEQ ID NO: 32 - protein, 8 aa, Artificial Sequence
  Description of Artificial Sequence Synthetic 6xHis tag
HHHHHHHH

SEQ ID NO: 33 - protein, 432 aa, Artificial Sequence
  Description of Artificial Sequence Synthetic polypeptide
MGWSLILLFLVAVATRVLSPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRASSGLNDIFEAQKIEWHEGTENLYFQSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSHHHHHHHH

SEQ ID NO: 34 - protein, 390 aa, Artificial Sequence
  Description of Artificial Sequence Synthetic polypeptide
MGWSLILLFLVAVATRVLSRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRASSGLNDIFEAQKIEWHEGTENLYFQSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSHHHHHHHH

SEQ ID NO: 35 - protein, 372 aa, Artificial Sequence
  Description of Artificial Sequence Synthetic polypeptide
MGWSLILLFLVAVATRVLSAPCPPGHFSPGDNQACKPWTNCTLAGKHTLQPASNSSDAICEDRDPPATQPQETQGPPARPITVQPTEAWPRTSQGPSTRPVEVPGGRASSGLNDIFEAQKIEWHEGTENLYFQSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSHHHHHHHH

SEQ ID NO: 36 - protein, 8 aa, Artificial Sequence
  Description of Artificial Sequence Synthetic peptide
IKRTPREP

SEQ ID NO: 37 - protein, 8 aa, Artificial Sequence
  Description of Artificial Sequence Synthetic peptide
IKRTVREP

SEQ ID NO: 38 - protein, 7 aa, Artificial Sequence
  Description of Artificial Sequence Synthetic peptide
IKRTREP

SEQ ID NO: 39 - protein, 9 aa, Artificial Sequence
  Description of Artificial Sequence Synthetic peptide
IKRTVPREP

SEQ ID NO: 40 - protein, 8 aa, Artificial Sequence
  Description of Artificial Sequence Synthetic peptide
IKRTVAEP

SEQ ID NO: 41 - protein, 10 aa, Artificial Sequence
  Description of Artificial Sequence Synthetic peptide
IKRTVAPREP

SEQ ID NO: 42 - protein, 8 aa, Artificial Sequence
  Description of Artificial Sequence Synthetic peptide
SSASPREP

SEQ ID NO: 43 - protein, 8 aa, Artificial Sequence
  Description of Artificial Sequence Synthetic peptide
SSASTREP

SEQ ID NO: 44 - protein, 9 aa, Artificial Sequence
  Description of Artificial Sequence Synthetic peptide
SSASTPREP

SEQ ID NO: 45 - protein, 9 aa, Artificial Sequence
  Description of Artificial Sequence Synthetic peptide
SSASTKGEP

SEQ ID NO: 46 - protein, 10 aa, Artificial Sequence
  Description of Artificial Sequence Synthetic peptide
SSASTKGREP

SEQ ID NO: 47 - protein, 665 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWDDSPYTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQWDDSPYTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 230 aa, Artificial Sequence
  Description of Artificial Sequence Synthetic polypeptide
EVQLVESGGGLVQPGGSLRLSCAASGFTFLSYYIHWVRQAPGKGLEWVAYIDPYSGGTDYADSVKGRFTISADTSKNTAYLQMNSLRAEDTAVYYCARVGLSFYAQEPVLDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 49 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYTSHPGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 50 - protein, 225 aa, Artificial Sequence
  Description of Artificial Sequence Synthetic polypeptide
EVQLVESGGGLVQPGGSLRLSCAASGFTFSSYVIHWVRQAPGKGLEWVAYIFPYGGTTYYADSVKGRFTISADTSKNTAYLQMNSLRAEDTAVYYCARGYYYVSDRVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 51 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQVDSTPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 52 - protein, 222 aa, Artificial Sequence
  Description of Artificial Sequence Synthetic polypeptide
EVQLVESGGGLVQPGGSLRLSCAASGFTFSSYYIHWVRQAPGKGLEWVAYIGSQGGFTDYADSVKGRFTISADTSKNTAYLQMNSLRAEDTAVYYCARQGYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 53 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYARPPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - protein, 218 aa, Artificial Sequence
  Description of Artificial Sequence Synthetic polypeptide
EVQLVESGGGLVQPGGSLRLSCAASGFTFTDYHIHWVRQAPGKGLEWVAGISSYTGQTDYADSVKGRFTISADTSKNTAYLQMNSLRAEDTAVYYCARGISGGFGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 55 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWYSDPETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 56 - protein, 222 aa, Artificial Sequence
  Description of Artificial Sequence Synthetic polypeptide
EVQLVESGGGLVQPGGSLRLSCAASGFTFDGYYIHWVRQAPGKGLEWVAYIHPYGGYTRYADSVKGRFTISADTSKNTAYLQMNSLRAEDTAVYYCARTRYDTGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 57 - protein, 665 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWDDSPYTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDGSGSGSGSGSDIQMTQSPSSLSASVGDRVTITCRASQSVSSAVAWYQQKPGKAPKLLIYSASSLYSGVPSRFSGSRSGTDFTLTISSLQPEDFATYYCQQYTSHPGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 665 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWEESPYTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDGSGSGSGSGSDIQMTQSPSSLSASVGDRVTITCRASQSVSSAVAWYQQKPGKAPKLLIYSASSLYSGVPSRFSGSRSGTDFTLTISSLQPEDFATYYCQQYTSHPGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 665 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWEESPYTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQWEESPYTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 4 aa, Artificial Sequence
  Description of Artificial Sequence Synthetic peptide
SSYY

SEQ ID NO: 61 - protein, 4 aa, Artificial Sequence
  Description of Artificial Sequence Synthetic peptide
DGYY

SEQ ID NO: 62 - protein, 4 aa, Artificial Sequence
  Description of Artificial Sequence Synthetic peptide
SDYY

SEQ ID NO: 63 - protein, 4 aa, Artificial Sequence
  Description of Artificial Sequence Synthetic peptide
DGYY

SEQ ID NO: 64 - protein, 4 aa, Artificial Sequence
  Description of Artificial Sequence Synthetic peptide
SVYY

SEQ ID NO: 65 - protein, 4 aa, Artificial Sequence
  Description of Artificial Sequence Synthetic peptide
TDYH

SEQ ID NO: 66 - protein, 4 aa, Artificial Sequence
  Description of Artificial Sequence Synthetic peptide
TSYY

SEQ ID NO: 67 - protein, 4 aa, Artificial Sequence
  Description of Artificial Sequence Synthetic peptide
PTYY

SEQ ID NO: 68 - protein, 4 aa, Artificial Sequence
  Description of Artificial Sequence Synthetic peptide
WSYY

SEQ ID NO: 69 - protein, 4 aa, Artificial Sequence
  Description of Artificial Sequence Synthetic peptide
SSYY

SEQ ID NO: 70 - protein, 4 aa, Artificial Sequence
  Description of Artificial Sequence Synthetic peptide
SSYV

SEQ ID NO: 71 - protein, 4 aa, Artificial Sequence
  Description of Artificial Sequence Synthetic peptide
TRYY

SEQ ID NO: 72 - protein, 4 aa, Artificial Sequence
  Description of Artificial Sequence Synthetic peptide
SSYI

SEQ ID NO: 73 - protein, 4 aa, Artificial Sequence
  Description of Artificial Sequence Synthetic peptide
SWYP

SEQ ID NO: 74 - protein, 4 aa, Artificial Sequence
  Description of Artificial Sequence Synthetic peptide
FSYY

SEQ ID NO: 75 - protein, 4 aa, Artificial Sequence
  Description of Artificial Sequence Synthetic peptide
SSYY

SEQ ID NO: 76 - protein, 4 aa, Artificial Sequence
  Description of Artificial Sequence Synthetic peptide
STYY

SEQ ID NO: 77 - protein, 4 aa, Artificial Sequence
  Description of Artificial Sequence Synthetic peptide
SRYP

SEQ ID NO: 78 - protein, 4 aa, Artificial Sequence
  Description of Artificial Sequence Synthetic peptide
PSYL

SEQ ID NO: 79 - protein, 4 aa, Artificial Sequence
  Description of Artificial Sequence Synthetic peptide
SDYY

SEQ ID NO: 80 - protein, 4 aa, Artificial Sequence
  Description of Artificial Sequence Synthetic peptide
SSYY

SEQ ID NO: 81 - protein, 4 aa, Artificial Sequence
  Description of Artificial Sequence Synthetic peptide
DDYY

SEQ ID NO: 82 - protein, 4 aa, Artificial Sequence
  Description of Artificial Sequence Synthetic peptide
TSYI

SEQ ID NO: 83 - protein, 4 aa, Artificial Sequence
  Description of Artificial Sequence Synthetic peptide
LSYY

SEQ ID NO: 84 - protein, 4 aa, Artificial Sequence
  Description of Artificial Sequence Synthetic peptide
SSYL

SEQ ID NO: 85 - protein, 4 aa, Artificial Sequence
  Description of Artificial Sequence Synthetic peptide
SSYY

SEQ ID NO: 86 - protein, 4 aa, Artificial Sequence
  Description of Artificial Sequence Synthetic peptide
STYY

SEQ ID NO: 87 - protein, 4 aa, Artificial Sequence
  Description of Artificial Sequence Synthetic peptide
STYY

SEQ ID NO: 88 - protein, 4 aa, Artificial Sequence
  Description of Artificial Sequence Synthetic peptide
SDYW

SEQ ID NO: 89 - protein, 4 aa, Artificial Sequence
  Description of Artificial Sequence Synthetic peptide
SSYV

SEQ ID NO: 90 - protein, 4 aa, Artificial Sequence
  Description of Artificial Sequence Synthetic peptide
SFYY

SEQ ID NO: 91 - protein, 4 aa, Artificial Sequence
  Description of Artificial Sequence Synthetic peptide
ASYH

SEQ ID NO: 92 - protein, 4 aa, Artificial Sequence
  Description of Artificial Sequence Synthetic peptide
SHYY

SEQ ID NO: 93 - protein, 4 aa, Artificial Sequence
  Description of Artificial Sequence Synthetic peptide
SRYF

SEQ ID NO: 94 - protein, 4 aa, Artificial Sequence
  Description of Artificial Sequence Synthetic peptide
SSYA

SEQ ID NO: 95 - protein, 4 aa, Artificial Sequence
  Description of Artificial Sequence Synthetic peptide
SSYT

SEQ ID NO: 96 - protein, 4 aa, Artificial Sequence
  Description of Artificial Sequence Synthetic peptide
SDYI

SEQ ID NO: 97 - protein, 4 aa, Artificial Sequence
  Description of Artificial Sequence Synthetic peptide
SSYY

SEQ ID NO: 98 - protein, 4 aa, Artificial Sequence
  Description of Artificial Sequence Synthetic peptide
ASYE

SEQ ID NO: 99 - protein, 4 aa, Artificial Sequence
  Description of Artificial Sequence Synthetic peptide
YSYF

SEQ ID NO: 100 - protein, 10 aa, Artificial Sequence
  Description of Artificial Sequence Synthetic peptide
GIHPYSTETR

SEQ ID NO: 101 - protein, 10 aa, Artificial Sequence
  Description of Artificial Sequence Synthetic peptide
AIESSSGYTY

SEQ ID NO: 102 - protein, 10 aa, Artificial Sequence
  Description of Artificial Sequence Synthetic peptide
AITSTGGSTY

SEQ ID NO: 103 - protein, 10 aa, Artificial Sequence
  Description of Artificial Sequence Synthetic peptide
YIHPYGGYTR

SEQ ID NO: 104 - protein, 10 aa, Artificial Sequence
  Description of Artificial Sequence Synthetic peptide
AIDPGSSYTY

SEQ ID NO: 105 - protein, 10 aa, Artificial Sequence
  Description of Artificial Sequence Synthetic peptide
GISSYTGQTD

SEQ ID NO: 106 - protein, 10 aa, Artificial Sequence
  Description of Artificial Sequence Synthetic peptide
LIDPDSSITD

SEQ ID NO: 107 - protein, 10 aa, Artificial Sequence
  Description of Artificial Sequence Synthetic peptide
YIYSGGGSTR

SEQ ID NO: 108 - protein, 10 aa, Artificial Sequence
  Description of Artificial Sequence Synthetic peptide
AITPYDGYTY

SEQ ID NO: 109 - protein, 10 aa, Artificial Sequence
  Description of Artificial Sequence Synthetic peptide
YIGSQGGFTD

SEQ ID NO: 110 - protein, 10 aa, Artificial Sequence
  Description of Artificial Sequence Synthetic peptide
YIFPYGGTTY

SEQ ID NO: 111 - protein, 10 aa, Artificial Sequence
  Description of Artificial Sequence Synthetic peptide
YIAPQGRSTH

SEQ ID NO: 112 - protein, 10 aa, Artificial Sequence
  Description of Artificial Sequence Synthetic peptide
YIFPYSGETY

SEQ ID NO: 113 - protein, 10 aa, Artificial Sequence
  Description of Artificial Sequence Synthetic peptide
WIYPISGYTD

SEQ ID NO: 114 - protein, 10 aa, Artificial Sequence
  Description of Artificial Sequence Synthetic peptide
WISPYGKRTH

SEQ ID NO: 115 - protein, 10 aa, Artificial Sequence
  Description of Artificial Sequence Synthetic peptide
AIRPYGSDTS

SEQ ID NO: 116 - protein, 10 aa, Artificial Sequence
  Description of Artificial Sequence Synthetic peptide
QIDPTDWWTD

SEQ ID NO: 117 - protein, 10 aa, Artificial Sequence
  Description of Artificial Sequence Synthetic peptide
SIYPWGGYTY

SEQ ID NO: 118 - protein, 10 aa, Artificial Sequence
  Description of Artificial Sequence Synthetic peptide
YIHPYSGYTD

SEQ ID NO: 119 - protein, 10 aa, Artificial Sequence
  Description of Artificial Sequence Synthetic peptide
AIEPSDGYTY

SEQ ID NO: 120 - protein, 10 aa, Artificial Sequence
  Description of Artificial Sequence Synthetic peptide
YISPYGSYTK

SEQ ID NO: 121 - protein, 10 aa, Artificial Sequence
  Description of Artificial Sequence Synthetic peptide
  MOD_RES (2)..(2): Any amino acid
TXSSSHAYTY

SEQ ID NO: 122 - protein, 10 aa, Artificial Sequence
  Description of Artificial Sequence Synthetic peptide
LIASYDSYTD

SEQ ID NO: 123 - protein, 10 aa, Artificial Sequence
  Description of Artificial Sequence Synthetic peptide
YIDPYSGGTD

SEQ ID NO: 124 - protein, 10 aa, Artificial Sequence
  Description of Artificial Sequence Synthetic peptide
YIDPWDDGTQ

SEQ ID NO: 125 - protein, 10 aa, Artificial Sequence
  Description of Artificial Sequence Synthetic peptide
VISPYAGSTK

SEQ ID NO: 126 - protein, 10 aa, Artificial Sequence
  Description of Artificial Sequence Synthetic peptide
AISPYHGDTS

SEQ ID NO: 127 - protein, 10 aa, Artificial Sequence
  Description of Artificial Sequence Synthetic peptide
GIYSSGGYTF

SEQ ID NO: 128 - protein, 10 aa, Artificial Sequence
  Description of Artificial Sequence Synthetic peptide
YITPYGDETD

SEQ ID NO: 129 - protein, 10 aa, Artificial Sequence
  Description of Artificial Sequence Synthetic peptide
FIHPLSDSTG

SEQ ID NO: 130 - protein, 10 aa, Artificial Sequence
  Description of Artificial Sequence Synthetic peptide
YIQSEGSVTY

SEQ ID NO: 131 - protein, 10 aa, Artificial Sequence
  Description of Artificial Sequence Synthetic peptide
WISPSGSTTR

SEQ ID NO: 132 - protein, 10 aa, Artificial Sequence
  Description of Artificial Sequence Synthetic peptide
VIDPQADRTD

SEQ ID NO: 133 - protein, 10 aa, Artificial Sequence
  Description of Artificial Sequence Synthetic peptide
WIYSYGSTTG

SEQ ID NO: 134 - protein, 10 aa, Artificial Sequence
  Description of Artificial Sequence Synthetic peptide
WIDSGDGDTF

SEQ ID NO: 135 - protein, 10 aa, Artificial Sequence
  Description of Artificial Sequence Synthetic peptide
YIDSKGGYTS

SEQ ID NO: 136 - protein, 10 aa, Artificial Sequence
  Description of Artificial Sequence Synthetic peptide
YISSYGGYTS

SEQ ID NO: 137 - protein, 10 aa, Artificial Sequence
  Description of Artificial Sequence Synthetic peptide
SIYSDTDYTY

SEQ ID NO: 138 - protein, 10 aa, Artificial Sequence
  Description of Artificial Sequence Synthetic peptide
AIDPYDGETY

SEQ ID NO: 139 - protein, 10 aa, Artificial Sequence
  Description of Artificial Sequence Synthetic peptide
AIDSYSGDTY

SEQ ID NO: 140 - protein, 10 aa, Artificial Sequence
  Description of Artificial Sequence Synthetic peptide
GYYYVADHVF

SEQ ID NO: 141 - protein, 6 aa, Artificial Sequence
  Description of Artificial Sequence Synthetic peptide
AYYTGM

SEQ ID NO: 142 - protein, 6 aa, Artificial Sequence
  Description of Artificial Sequence Synthetic peptide
GDYTGM

SEQ ID NO: 143 - protein, 7 aa, Artificial Sequence
  Description of Artificial Sequence Synthetic peptide
TRYDTGM

SEQ ID NO: 144 - protein, 6 aa, Artificial Sequence
  Description of Artificial Sequence Synthetic peptide
SSYTAM

SEQ ID NO: 145 - protein, 6 aa, Artificial Sequence
  Description of Artificial Sequence Synthetic peptide
GISGGF

SEQ ID NO: 146 - protein, 6 aa, Artificial Sequence
  Description of Artificial Sequence Synthetic peptide
MDTIVL

SEQ ID NO: 147 - protein, 7 aa, Artificial Sequence
  Description of Artificial Sequence Synthetic peptide
TDASSAL

SEQ ID NO: 148 - protein, 7 aa, Artificial Sequence
  Description of Artificial Sequence Synthetic peptide
GSVYTGM

SEQ ID NO: 149 - protein, 7 aa, Artificial Sequence
  Description of Artificial Sequence Synthetic peptide
QGYGYAL

SEQ ID NO: 150 - protein, 10 aa, Artificial Sequence
  Description of Artificial Sequence Synthetic peptide
GYYYVSDRVM

SEQ ID NO: 151 - protein, 7 aa, Artificial Sequence
  Description of Artificial Sequence Synthetic peptide
SLYYGGM

SEQ ID NO: 152 - protein, 10 aa, Artificial Sequence
  Description of Artificial Sequence Synthetic peptide
GAYYYTDLVF

SEQ ID NO: 153 - protein, 9 aa, Artificial Sequence
  Description of Artificial Sequence Synthetic peptide
QGFVVGGAF

SEQ ID NO: 154 - protein, 13 aa, Artificial Sequence
  Description of Artificial Sequence Synthetic peptide
RYGRFGYRSYVAM

SEQ ID NO: 155 - protein, 13 aa, Artificial Sequence
  Description of Artificial Sequence Synthetic peptide
GYYYSWDYPPWVF

SEQ ID NO: 156 - protein, 12 aa, Artificial Sequence
  Description of Artificial Sequence Synthetic peptide
GYSPVFDMEFGL

SEQ ID NO: 157 - protein, 7 aa, Artificial Sequence
  Description of Artificial Sequence Synthetic peptide
ESGPGAM

SEQ ID NO: 158 - protein, 10 aa, Artificial Sequence
  Description of Artificial Sequence Synthetic peptide
GFYGGSDLVL

SEQ ID NO: 159 - protein, 6 aa, Artificial Sequence
  Description of Artificial Sequence Synthetic peptide
GDYPGM

SEQ ID NO: 160 - protein, 7 aa, Artificial Sequence
  Description of Artificial Sequence Synthetic peptide
SDYYGAL

SEQ ID NO: 161 - protein, 9 aa, Artificial Sequence
  Description of Artificial Sequence Synthetic peptide
ESVYPMGAM

SEQ ID NO: 162 - protein, 15 aa, Artificial Sequence
  Description of Artificial Sequence Synthetic peptide
SYDGVGHYLYGLGGF

SEQ ID NO: 163 - protein, 12 aa, Artificial Sequence
  Description of Artificial Sequence Synthetic peptide
VGLSFYAQEPVL

SEQ ID NO: 164 - protein, 12 aa, Artificial Sequence
  Description of Artificial Sequence Synthetic peptide
SWGQYYDYYDVF

SEQ ID NO: 165 - protein, 12 aa, Artificial Sequence
  Description of Artificial Sequence Synthetic peptide
GFGYYAEFDSAL

SEQ ID NO: 166 - protein, 6 aa, Artificial Sequence
  Description of Artificial Sequence Synthetic peptide
VEGIGM

SEQ ID NO: 167 - protein, 7 aa, Artificial Sequence
  Description of Artificial Sequence Synthetic peptide
TYRYYGM

SEQ ID NO: 168 - protein, 7 aa, Artificial Sequence
  Description of Artificial Sequence Synthetic peptide
IIQLLGM

SEQ ID NO: 169 - protein, 10 aa, Artificial Sequence
  Description of Artificial Sequence Synthetic peptide
GYYYSSDYVM

SEQ ID NO: 170 - protein, 7 aa, Artificial Sequence
  Description of Artificial Sequence Synthetic peptide
FDAYVAM

SEQ ID NO: 171 - protein, 7 aa, Artificial Sequence
  Description of Artificial Sequence Synthetic peptide
VYHGTGL

SEQ ID NO: 172 - protein, 7 aa, Artificial Sequence
  Description of Artificial Sequence Synthetic peptide
DYMYFVM

SEQ ID NO: 173 - protein, 7 aa, Artificial Sequence
  Description of Artificial Sequence Synthetic peptide
RSQYSVM

SEQ ID NO: 174 - protein, 12 aa, Artificial Sequence
  Description of Artificial Sequence Synthetic peptide
SLGYYYYGHGVF

SEQ ID NO: 175 - protein, 14 aa, Artificial Sequence
  Description of Artificial Sequence Synthetic peptide
SWYDTGHFGYDAVF

SEQ ID NO: 176 - protein, 12 aa, Artificial Sequence
  Description of Artificial Sequence Synthetic peptide
AAYPFYDYDPAF

SEQ ID NO: 177 - protein, 11 aa, Artificial Sequence
  Description of Artificial Sequence Synthetic peptide
EGFVYPSSTAL

SEQ ID NO: 178 - protein, 12 aa, Artificial Sequence
  Description of Artificial Sequence Synthetic peptide
DFSSYYGLAMGF

SEQ ID NO: 179 - protein, 14 aa, Artificial Sequence
  Description of Artificial Sequence Synthetic peptide
GYGDAYYFYEYGAM

SEQ ID NO: 180 - protein, 6 aa, Artificial Sequence
  Description of Artificial Sequence Synthetic peptide
FQDSPV

SEQ ID NO: 181 - protein, 6 aa, Artificial Sequence
  Description of Artificial Sequence Synthetic peptide
YIYGPL

SEQ ID NO: 182 - protein, 6 aa, Artificial Sequence
  Description of Artificial Sequence Synthetic peptide
YIYSPA

SEQ ID NO: 183 - protein, 6 aa, Artificial Sequence
  Description of Artificial Sequence Synthetic peptide
WYSDPE

SEQ ID NO: 184 - protein, 6 aa, Artificial Sequence
  Description of Artificial Sequence Synthetic peptide
YIYDPS

SEQ ID NO: 185 - protein, 6 aa, Artificial Sequence
  Description of Artificial Sequence Synthetic peptide
YARPPR

SEQ ID NO: 186 - protein, 6 aa, Artificial Sequence
  Description of Artificial Sequence Synthetic peptide
YYFWPW

SEQ ID NO: 187 - protein, 6 aa, Artificial Sequence
  Description of Artificial Sequence Synthetic peptide
YVSSPE

SEQ ID NO: 188 - protein, 6 aa, Artificial Sequence
  Description of Artificial Sequence Synthetic peptide
YDYSPA

SEQ ID NO: 189 - protein, 6 aa, Artificial Sequence
  Description of Artificial Sequence Synthetic peptide
VDSTPV

SEQ ID NO: 190 - protein, 6 aa, Artificial Sequence
  Description of Artificial Sequence Synthetic peptide
YTSHPG

SEQ ID NO: 191 - protein, 6 aa, Artificial Sequence
  Description of Artificial Sequence Synthetic peptide
FYSSPE

SEQ ID NO: 192 - protein, 6 aa, Artificial Sequence
  Description of Artificial Sequence Synthetic peptide
YSSSPV

SEQ ID NO: 193 - protein, 6 aa, Artificial Sequence
  Description of Artificial Sequence Synthetic peptide
WSAKLY

SEQ ID NO: 194 - protein, 6 aa, Artificial Sequence
  Description of Artificial Sequence Synthetic peptide
YTSSPY

SEQ ID NO: 195 - protein, 6 aa, Artificial Sequence
  Description of Artificial Sequence Synthetic peptide
ADYSLT

SEQ ID NO: 196 - protein, 6 aa, Artificial Sequence
  Description of Artificial Sequence Synthetic peptide
ASWGLT

SEQ ID NO: 197 - protein, 6 aa, Artificial Sequence
  Description of Artificial Sequence Synthetic peptide
YERIPY

SEQ ID NO: 198 - protein, 6 aa, Artificial Sequence
  Description of Artificial Sequence Synthetic peptide
YYGSLY

SEQ ID NO: 199 - protein, 6 aa, Artificial Sequence
  Description of Artificial Sequence Synthetic peptide
VTYTPL

SEQ ID NO: 200 - protein, 6 aa, Artificial Sequence
  Description of Artificial Sequence Synthetic peptide
YYTSPE

SEQ ID NO: 201 - protein, 6 aa, Artificial Sequence
  Description of Artificial Sequence Synthetic peptide
LSSWPL

SEQ ID NO: 202 - protein, 6 aa, Artificial Sequence
  Description of Artificial Sequence Synthetic peptide
SDSSPW

SEQ ID NO: 203 - protein, 6 aa, Artificial Sequence
  Description of Artificial Sequence Synthetic peptide
WDDSPY

SEQ ID NO: 204 - protein, 6 aa, Artificial Sequence
  Description of Artificial Sequence Synthetic peptide
LFSHPY

SEQ ID NO: 205 - protein, 6 aa, Artificial Sequence
  Description of Artificial Sequence Synthetic peptide
WYSTPY

SEQ ID NO: 206 - protein, 6 aa, Artificial Sequence
  Description of Artificial Sequence Synthetic peptide
AYGDLR

SEQ ID NO: 207 - protein, 6 aa, Artificial Sequence
  Description of Artificial Sequence Synthetic peptide
GYSDPQ

SEQ ID NO: 208 - protein, 6 aa, Artificial Sequence
  Description of Artificial Sequence Synthetic peptide
GSSSPL

SEQ ID NO: 209 - protein, 6 aa, Artificial Sequence
  Description of Artificial Sequence Synthetic peptide
YSDWPY

SEQ ID NO: 210 - protein, 6 aa, Artificial Sequence
  Description of Artificial Sequence Synthetic peptide
HSSSLE

SEQ ID NO: 211 - protein, 6 aa, Artificial Sequence
  Description of Artificial Sequence Synthetic peptide
VDTSLG

SEQ ID NO: 212 - protein, 6 aa, Artificial Sequence
  Description of Artificial Sequence Synthetic peptide
ADTQPL

SEQ ID NO: 213 - protein, 6 aa, Artificial Sequence
  Description of Artificial Sequence Synthetic peptide
WSSSPE

SEQ ID NO: 214 - protein, 6 aa, Artificial Sequence
  Description of Artificial Sequence Synthetic peptide
YHTSLH

SEQ ID NO: 215 - protein, 6 aa, Artificial Sequence
  Description of Artificial Sequence Synthetic peptide
YYGGLP

SEQ ID NO: 216 - protein, 6 aa, Artificial Sequence
  Description of Artificial Sequence Synthetic peptide
SYSSPY

SEQ ID NO: 217 - protein, 6 aa, Artificial Sequence
  Description of Artificial Sequence Synthetic peptide
YYSEPV

SEQ ID NO: 218 - protein, 6 aa, Artificial Sequence
  Description of Artificial Sequence Synthetic peptide
VHSYPS

SEQ ID NO: 219 - protein, 6 aa, Artificial Sequence
  Description of Artificial Sequence Synthetic peptide
WTRSLT

SEQ ID NO: 220 - protein, 11 aa, Artificial Sequence
  Description of Artificial Sequence Synthetic peptide
RASQSVSSAVA

SEQ ID NO: 221 - protein, 7 aa, Artificial Sequence
  Description of Artificial Sequence Synthetic peptide
SASSLYS

SEQ ID NO: 222 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWDDSPYTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYTSHPGTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 228 aa, Artificial Sequence
  Description of Artificial Sequence Synthetic polypeptide
EVQLVESGGGLVQPGGSLRLSCAASGFTFSSYVIHWVRQAPGKGLEWVAYIFPYGGTTYYADSVKGRFTISADTSKNTAYLQMNSLRAEDTAVYYCARGYYYVSDRVMDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 225 - protein, 441 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWDDSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 226 - protein, 227 aa, Artificial Sequence
  Description of Artificial Sequence Synthetic polypeptide
EVQLVESGGGLVQPGGSLRLSCAASGFTFLSYYIHWVRQAPGKGLEWVAYIDPYSGGTDYADSVKGRFTISADTSKNTAYLQMNSLRAEDTAVYYCARVGLSFYAQEPVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 227 - protein, 6 aa, Artificial Sequence
  Description of Artificial Sequence Synthetic peptide
WEESPY

SEQ ID NO: 228 - protein, 6 aa, Artificial Sequence
  Description of Artificial Sequence Synthetic peptide
GSGSGS

SEQ ID NO: 229 - protein, 5 aa, Artificial Sequence
  Description of Artificial Sequence Synthetic peptide
DKTHT

(19) United States
(12) Patent Application Publication
(10) Pub. No.: US 2021/0363265 A1
(43) Pub. Date: Nov. 25, 2021

(54) MULTIVALENT RECEPTOR-CLUSTERING AGONIST ANTIBODY CONSTRUCTS

(71) Applicant: INVENRA INC., Madison, WI (US)

(72) Inventors: Qufei LI, Middleton, WI (US); Lucas BAILEY, Fort Worth, TX (US); Bryan GLASER, Fitchburg, WI (US)

(21) Appl. No.: 16/641,605

(22) PCT Filed: Aug. 23, 2018
(86) PCT No.: PCT/US2018/047819

§ 371 Date: Feb. 24, 2020

Related U.S. Application Data
(60) Provisional application No. 62/549,913, filed on Aug. 24, 2017.
(60) Provisional application No. 62/646,321, filed on Mar. 21, 2018.

(51) Int. Cl. C07K 16/28

(52) U.S. Cl. CPC C07K 16/2878; C07K 2317/31; C07K 2317/75; C07K 2317/35; C07K 2317/21

(57) ABSTRACT

Multivalent receptor-clustering agonist antibody constructs are provided. The constructs are capable of (i) binding a cell surface receptor target that requires clustering for agonist activity, and (ii) clustering the receptor target on the cell surface in the absence of an independent cross-linking agent. Each of the target receptor-binding antigen binding sites of the construct is contributed by antibody variable region binding domains. Also provided are pharmaceutical compositions comprising the antibody construct, and methods of treating diseases, notably cancer, by administering therapeutically effective amounts of the pharmaceutical composition.

### 1. CROSS-REFERENCE TO RELATED APPLICATIONS

This application claims priority to Provisional Application No. 62/646,321, filed Mar. 21, 2018, and to Provisional Application No. 62/549,913, filed Aug. 24, 2017, the disclosures of which are incorporated by reference in their entirety for all purposes.

### 2. SEQUENCE LISTING

The instant application contains a Sequence Listing which has been submitted via EFS-Web and is hereby incorporated herein by reference in its entirety. Said ASCII copy, created on Month XX, 2018, is named XXXXXUS_sequencelisting.txt, and is X,XXX,XXX bytes in size.

### 3. BACKGROUND

The tumor necrosis factor receptor family (TNFR) is a protein superfamily of cytokine receptors involved in virtually every biological system, ranging from immune system physiology to neurobiology. Antagonists of the TNF/TNFR signaling axis are among the most successful drugs ever commercialized, and include receptor:Fc fusion constructs, such as etanercept (Enbrel), anti-TNFα antibodies, such as infliximab (Remicade), adalimumab (Humira) and golimumab (Simponi), and pegylated Fab′ constructs, such as Certolizumab pegol (Cimzia).

Agonists of the TNF/TNFR signaling axis also have therapeutic potential. Croft et al., Nature Rev. Drug Discovery 12:147-168 (2013). However, agonists of the TNFR have been much less successful: effective TNFR activation is much more difficult to achieve than blocking of the TNF and TNFR interactions, because activation of TNFR generally requires specific oligomerization (clustering the receptor trimers) and immobilization.

Fusion proteins comprising two ligand trimers (a pseudo-hexamer) can be effective oligomerizing agonists, but the ligands are small cysteine-rich domains and their oligomers generally possess poor biophysical properties, making them inferior drugs compared to antibodies. Conventional TNFR mAbs—which are bivalent and monospecific for a single TNFR epitope—typically possess low-to moderate TNFR agonist activity. Accordingly, additional crosslinking of the TNFR mAbs is required to potentiate agonistic activity. Various approaches to higher order oligomerization of anti-TNFR mAb have been pursued. In vitro, secondary antibodies that crosslink the agonistic antibody have been used, but an approach that requires co-localization of two exogenously administered antibodies is not suitable for therapy. Other approaches have relied on adventitious Fc engagement by FcγR on the surface of cells encountered in vivo. Problems with this approach include the need for cells with FcγR to be in close proximity to the antibody-decorated cell, and inherent competition for FcγR binding between the therapeutic antibody and endogenous antibodies. A recent approach uses cross-linking to tumor cells to drive the higher level oligomerization. See US Pub. No. 2017/0114141. Various constructs using Fc variants that hexamerize have also been proposed to increase the valency of crosslinking. However, these engineered constructs require customized expression and purification approaches.

A recent approach has also described use of tetravalent antibodies to stimulate T cells in the absence of a crosslinking reagent (see US Pub. No. 2018/0057598). However, use of tetravalent constructs can impact expression and, given the symmetry required in the system described, limit further optimization using non-symmetrical formats. In addition, antibody architectures other than tetravalent formats, e.g., bivalent bispecific formats, were determined to lack agonist activity.

There is, therefore, a need for an antibody construct capable of (i) binding a cell surface receptor target that requires clustering for agonist activity, and (ii) clustering the receptor target on the cell surface in the absence of an independent cross-linking agent. There is a further need for antibody constructs having these characteristics and that are also capable of high level expression, such as bivalent and trivalent constructs, with high fidelity pairing of cognate heavy chain pairs and cognate heavy and light chain pairs and that can be readily purified.

### 4. SUMMARY

We have developed multivalent antibody constructs, including multispecific antibody constructs, that are readily expressed to high levels in standard transient transfection systems with high fidelity pairing of cognate heavy chain pairs and cognate heavy and light chain pairs, and that can be purified in a single step to purity levels sufficient to allow in vitro assay.

Following a standard library panning campaign to identify phage-displayed human Fabs that bind the TNFRSF member OX40, we identified antigen binding sites for monovalent binding to OX40. Because our constructs are suitable for high throughput expression and assay, we then recloned antigen binding sites having specificity for different OX40 epitopes into a wide variety of monospecific bivalent, bispecific bivalent, monospecific trivalent, bispecific trivalent and trispecific trivalent combinations. We expressed and tested these multivalent constructs in high throughput assays for OX40 agonist activity, both in the absence and the presence of an agent that further cross-links the antibody construct. Mechanisms for antibody mediated receptor clustering both in the presence (FIG. 5A) and the absence (FIG. 5B) of a crosslinking reagent are illustrated.

Our constructs demonstrated a wide range of agonist activity in the absence of a crosslinking agent; some have agonist activity in the absence of cross-linker greater than that of crosslinked OX40 ligand. The best constructs exhibited agonist activity in the absence of independent crosslinking agent superior to that observed with three known mAb clinical candidates. A number of our constructs also demonstrated a wide range of increased activity upon further crosslinking.

Accordingly, in a first aspect, multivalent antibody constructs are provided. The constructs are capable of (i) binding a cell surface receptor target that requires clustering for agonist activity, and (ii) clustering the receptor target on the cell surface in the absence of an independent cross-linking agent or one or more Fc mutations that drive hexamer formation. In these constructs, each of the target receptor-binding antigen binding sites of the construct is contributed by antibody variable region binding domains.

In some aspects, the multivalent construct is monospecific.

In some aspects, the multivalent construct is multispecific. In some aspects, the multispecific multivalent comprises a first antigen binding site specific for a first epitope of the target receptor, and a second antigen binding site specific for a second antigenic target. In some aspects, the second antigenic target is a second epitope of the target receptor. In some aspects, the first epitope and the second epitope are non-overlapping epitopes. In some aspects, the second antigenic target is an epitope of a second protein. In some aspects, the second protein is a second cell surface receptor. In some aspects, the target cell surface receptor and the second cell surface receptor are commonly expressed on the surface of at least some mammalian cells.

In some aspects, the target receptor is a TNF Receptor superfamily (TNFRSF) member. In some aspects, the target receptor is OX40 (TNFRSF4), CD40 (TNFRSF5), or 4-1BB (TNFRSF9). In some aspects, the target receptor is a human TNFRSF. In some aspects, the target receptor is human OX40, human CD40, or human 4-1BB. In some aspects, the target receptor is human OX40.

In some aspects, the multivalent construct is bivalent. In some aspects, the bivalent construct is a bivalent (1×1) construct. In some aspects, the construct is monospecific. In some aspects, the construct is bispecific. In some aspects, the second antigenic target is a second epitope of the target receptor. In some aspects, the first epitope and the second epitope are non-overlapping epitopes. In some aspects, the second antigenic target is an epitope of a second protein. In some aspects, the second protein is a second cell surface receptor in the bivalent bispecific construct. In some aspects, the antigen binding site specific for a first epitope of the target receptor is an A:F antigen binding site. In some aspects, the antigen binding site specific for a first epitope of the target receptor is an H:L antigen binding site.

In some aspects, the multivalent construct is trivalent. In some aspects, the construct is a trivalent (2×1) construct. In some aspects, the trivalent construct is monospecific. In some aspects, the trivalent is bispecific.

In some aspects, the bispecific trivalent construct contains one copy of the antigen binding site (ABS) specific for a first epitope of the target receptor. In some aspects, the antigen binding site specific for a first epitope of the target receptor is an A:F antigen binding site. In some aspects, the antigen binding site specific for a first epitope of the target receptor is an N:P antigen binding site. In some aspects, the antigen binding site specific for a first epitope of the target receptor is an H:L antigen binding site.

In some aspects, the bispecific trivalent construct contains two copies of the antigen binding site specific for a first epitope of the target receptor. In some aspects, a first antigen binding site specific for a first epitope of the target receptor is an A:F antigen binding site. In some aspects, a first antigen binding site specific for a first epitope of the target receptor is an N:P antigen binding site. In some aspects, a first antigen binding site specific for a first epitope of the target receptor is an H:L antigen binding site. In some aspects, a first antigen binding site specific for a first epitope of the target receptor is an A:F antigen binding site and a second antigen binding site specific for a first epitope of the target receptor is an N:P antigen binding site. In some aspects, a first antigen binding site specific for a first epitope of the target receptor is an A:F antigen binding site and a second antigen binding site specific for a first epitope of the target receptor is an H:L antigen binding site. In some aspects, a first antigen binding site specific for a first epitope of the target receptor is an N:P antigen binding site and a second antigen binding site specific for a first epitope of the target receptor is an H:L antigen binding site. In some aspects, the second antigenic target is a second epitope of the target receptor. In some aspects, the first epitope and the second epitope are non-overlapping epitopes. In some aspects, the second antigenic target is an epitope of a second protein. In some aspects, the second protein is a second cell surface receptor.

In some aspects, the trivalent construct is trispecific. In some aspects, the antigen binding site specific for a first epitope of the target receptor is an A:F antigen binding site in the trispecific construct. In some aspects, the antigen binding site specific for a first epitope of the target receptor is an N:P antigen binding site in the trispecific construct. In some aspects, the antigen binding site specific for a first epitope of the target receptor is an H:L antigen binding site in the trispecific construct. In some aspects, the second antigenic target is a second epitope of the target receptor. In some aspects, the first epitope and the second epitope are non-overlapping epitopes. In some aspects, the second antigenic target is a first epitope of a second protein. In some aspects, the third antigenic target is a third epitope of the target receptor. In some aspects, the third antigenic target is a second epitope of a second protein. In some aspects, the first epitope of the second protein and the second epitope of the second protein are non-overlapping epitopes. In some aspects, the third antigenic target is a first epitope of a third protein in the trispecific construct. In some aspects, the second protein or third protein is a second or third cell surface receptor.

In some aspects, the trivalent construct is a trivalent (1×2) construct. In some aspects, the trivalent construct is monospecific. In some aspects, the trivalent construct is bispecific. In some aspects, the bispecific trivalent contains one copy of the antigen binding site (ABS) specific for a first epitope of the target receptor. In some aspects, the antigen binding site specific for a first epitope of the target receptor is an A:F antigen binding site in the bispecific trivalent construct. In some aspects, the antigen binding site specific for a first epitope of the target receptor is an R:T antigen binding site in the bispecific trivalent construct. In some aspects, the antigen binding site specific for a first epitope of the target receptor is an H:L antigen binding site in the bispecific trivalent construct.

In some aspects, the bispecific trivalent construct contains two copies of the antigen binding site specific for a first epitope of the target receptor. In some aspects, a first antigen binding site specific for a first epitope of the target receptor is an A:F antigen binding site in the bispecific trivalent construct. In some aspects, a first antigen binding site specific for a first epitope of the target receptor is an R:T antigen binding site in the bispecific trivalent construct. In some aspects, a first antigen binding site specific for a first epitope of the target receptor is an H:L antigen binding site in the bispecific trivalent construct. In some aspects, a first antigen binding site specific for a first epitope of the target receptor is an A:F antigen binding site and a second antigen binding site specific for a first epitope of the target receptor is an R:T antigen binding site in the bispecific trivalent construct. In some aspects, a first antigen binding site specific for a first epitope of the target receptor is an A:F antigen binding site and a second antigen binding site specific for a first epitope of the target receptor is an H:L antigen binding site in the bispecific trivalent construct. In some aspects, a first antigen binding site specific for a first epitope of the target receptor is an R:T antigen binding site and a second antigen binding site specific for a first epitope of the target receptor is an H:L antigen binding site in the bispecific trivalent construct. In some aspects, the second antigenic target is a second epitope of the target receptor. In some aspects, the first epitope and the second epitope are non-overlapping epitopes. In some aspects, the second antigenic target is an epitope of a second protein. In some aspects, the second protein is a second cell surface.

In some aspects, the trivalent construct is trispecific. In some aspects, the antigen binding site specific for a first epitope of the target receptor is an A:F antigen binding site in the trispecific trivalent construct. In some aspects, the antigen binding site specific for a first epitope of the target receptor is an R:T antigen binding site in the trispecific trivalent construct. In some aspects, the antigen binding site specific for a first epitope of the target receptor is an H:L antigen binding site in the trispecific trivalent construct. In some aspects, the second antigenic target is a second epitope of the target receptor. In some aspects, the second antigenic target is a first epitope of a second protein. In some aspects, the third antigenic target is a third epitope of the target receptor. In some aspects, the third antigenic target is a second epitope of a second protein. In some aspects, the first epitope of the second protein and the second epitope of the second protein are non-overlapping epitopes. In some aspects, the third antigenic target is a first epitope of a third protein. In some aspects, the second protein or third protein is a second or third cell surface receptor.

In some aspects, the presence of an independent cross-linking agent does not increase agonist activity in vitro above that achieved in the absence of the independent cross-linking agent. In some aspects, the presence of an independent cross-linking agent increases agonist activity in vitro above that achieved in the absence of the independent cross-linking agent. In some aspects, the presence of an independent cross-linking agent increases agonist in vitro activity 50% above activity observed in the absence of the independent cross-linking agent.

In some aspects, the bivalent (1×1) antibody constructs comprises a first, second, third, and fourth polypeptide chain, wherein: (a) the first polypeptide chain comprises a domain A, a domain B, a domain D, and a domain E, wherein the domains are arranged, from N-terminus to C-terminus, in a A-B-D-E orientation, and domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region domain amino acid sequence, domain J has a CH2 amino acid sequence, and K has a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the binding molecule.

In some aspects, the amino acid sequences of the B and the G domains are identical, wherein the sequence is an endogenous CH3 sequence.

In some aspects, the amino acid sequences of the B and the G domains are different and separately comprise respectively orthogonal modifications in an endogenous CH3 sequence, wherein the B domain interacts with the G domain, and wherein neither the B domain nor the G domain interacts with a CH3 domain lacking the orthogonal modification. In some aspects, the orthogonal modifications comprise mutations that generate engineered disulfide bridges between domain B and G. In some aspects, the mutations that generate engineered disulfide bridges are a S354C mutation in one of the B domain and G domain, and a 349C in the other domain. In some aspects, the orthogonal modifications comprise knob-in-hole mutations. In some aspects, the knob-in hole mutations are a T366W mutation in one of the B domain and G domain, and a T366S, L368A, and a Y407V mutation in the other domain. In some aspects, the orthogonal modifications comprise charge-pair mutations. In some aspects, the charge-pair mutations are a T366K mutation in one of the B domain and G domain, and a L351D mutation in the other domain.

In some aspects, the domain E has a CH3 amino acid sequence. In some aspects, the amino acid sequences of the E and K domains are identical, wherein the sequence is an endogenous CH3 sequence.

In some aspects, the amino acid sequences of the E and K domains are different. In some aspects, the different sequences separately comprise respectively orthogonal modifications in an endogenous CH3 sequence, wherein the E domain interacts with the K domain, and wherein neither the E domain nor the K domain interacts with a CH3 domain lacking the orthogonal modification. In some aspects, the orthogonal modifications comprise mutations that generate engineered disulfide bridges between domain E and K. In some aspects, the mutations that generate engineered disulfide bridges are a S354C mutation in one of the E domain and K domain, and a 349C in the other domain. In some aspects, the orthogonal modifications in the E and K domains comprise knob-in-hole mutations. In some aspects, the knob-in hole mutations are a T366W mutation in one of the E domain or K domain and a T366S, L368A, and a Y407V mutation in the other domain. In some aspects, the orthogonal modifications comprise charge-pair mutations. In some aspects, the charge-pair mutations are a T366K mutation in one of the E domain or K domain and a corresponding L351D mutation in the other domain.

In some aspects, the amino acid sequences of the E domain and the K domain are endogenous sequences of two different antibody domains, the domains selected to have a specific interaction that promotes the specific association between the first and the third polypeptides. In some aspects, the two different amino acid sequences are a CH1 sequence and a CL sequence. In some aspects, the domain I has a CL sequence and domain M has a CH1 sequence.

In some aspects, the domain H has a VL sequence and domain L has a VH sequence.

In some aspects, domain H has a VL amino acid sequence; domain I has a CL amino acid sequence; domain K has a CH3 amino acid sequence; domain L has a VH amino acid sequence; and domain M has a CH1 amino acid sequence.

In some aspects, the multivalent antibody constructs further comprise a sixth polypeptide chain, wherein: (a) the third polypeptide chain further comprises a domain R and a domain S, wherein the domains are arranged, from N-terminus to C-terminus, in a R-S-H-I-J-K orientation, and wherein domain R has a VL amino acid sequence and domain S has a constant domain amino acid sequence; (b) the binding molecule further comprises a sixth polypeptide chain, comprising: a domain T and a domain U, wherein the domains are arranged, from N-terminus to C-terminus, in a T-U orientation, and wherein domain T has a VH amino acid sequence and domain U has a constant domain amino acid sequence; and (c) the third and the sixth polypeptides are associated through an interaction between the R and the T domains and an interaction between the S and the U domains to form the binding molecule.

In some aspects, (a) the amino acid sequences of domain R and domain A are identical, the amino acid sequences of domain H is different from domain R and A, the amino acid sequences of domain S and domain B are identical, the amino acid sequences of domain I is different from domain S and B, the amino acid sequences of domain T and domain F are identical, the amino acid sequences of domain L is different from domain T and F, the amino acid sequences of domain U and domain G are identical, the amino acid sequences of domain M is different from domain U and G and (b) the interaction between the A domain and the F domain form a first antigen binding site specific for a first antigen, the interaction between the H domain and the L domain form a second antigen binding site specific for a second antigen, and the domain R and domain T form a third antigen binding site specific for the first antigen.

In some aspects, (a) the amino acid sequences of domain R and domain H are identical, the amino acid sequences of domain A is different from domain R and H, the amino acid sequences of domain S and domain I are identical, the amino acid sequences of domain B is different from domain S and I, the amino acid sequences of domain T and domain L are identical, the amino acid sequences of domain F is different from domain T and L, the amino acid sequences of domain U and domain M are identical, the amino acid sequences of domain G is different from domain U and M and (b) the interaction between the A domain and the F domain form a first antigen binding site specific for a first antigen, the interaction between the H domain and the L domain form a second antigen binding site specific for a second antigen, and the domain R and domain T form a third antigen binding site specific for the second antigen.

In some aspects, (a) the amino acid sequences of domain R, domain A, and domain H are different, the amino acid sequences of domain S, domain B, and domain I are different, the amino acid sequences of domain T, domain F, and domain L are different, and the amino acid sequences of domain U, domain G, and domain M are different; and (b) the interaction between the A domain and the F domain form a first antigen binding site specific for a first antigen, the interaction between the H domain and the L domain form a second antigen binding site specific for a second antigen, and the domain R and domain T form a third antigen binding site specific for a third antigen.

In some aspects, the multivalent antibody constructs further comprise a fifth polypeptide chain, wherein: (a) the first polypeptide chain further comprises a domain N and a domain O, wherein the domains are arranged, from N-terminus to C-terminus, in a N-O-A-B-D-E orientation, and wherein domain N has a VL amino acid sequence, domain O has a CH3 amino acid sequence; (b) the binding molecule further comprises a fifth polypeptide chain, comprising: a domain P and a domain Q, wherein the domains are arranged, from N-terminus to C-terminus, in a P-Q orientation, and wherein domain P has a VH amino acid sequence and domain Q has a CH3 amino acid sequence; and (c) the first and the fifth polypeptides are associated through an interaction between the N and the P domains and an interaction between the O and the Q domains to form the binding molecule.

In some aspects, (a) the amino acid sequences of domain N and domain A are identical, the amino acid sequences of domain H is different from domains N and A, the amino acid sequences of domain O and domain B are identical, the amino acid sequences of domain I is different from domains O and B, the amino acid sequences of domain P and domain F are identical, the amino acid sequences of domain L is different from domains P and F, the amino acid sequences of domain Q and domain G are identical, the amino acid sequences of domain M is different from domains Q and G; and (b) wherein the interaction between the A domain and the F domain form a first antigen binding site specific for a first antigen, the interaction between the H domain and the L domain form a second antigen binding site specific for a second antigen, and the domain N and domain P form a third antigen binding site specific for the first antigen.

In some aspects, (a) the amino acid sequences of domain N, domain A, and domain H are different, the amino acid sequences of domain O, domain B, and domain I are different, the amino acid sequences of domain P, domain F, and domain L are different, and the amino acid sequences of domain Q, domain G, and domain M are different; and (b) the interaction between the A domain and the F domain form a first antigen binding site specific for a first antigen, the interaction between the H domain and the L domain form a second antigen binding site specific for a second antigen, and the domain N and domain P form a third antigen binding site specific for a third antigen.

In some aspects, the sequence that links the A domain and the B domain is IKRTPREP or IKRTVREP. In some aspects, the sequence that links the F domain and the G domain is SSASPREP. In some aspects, at least one CH3 amino acid sequence has a C-terminal tripeptide insertion linking the CH3 amino acid sequence to a hinge amino acid sequence, wherein the tripeptide insertion is selected from the group consisting of PGK, KSC, and GEC.

In some aspects, the sequences are human sequences. In some aspects, at least one CH3 amino acid sequence is an IgG sequence. In some aspects, the IgG sequences are IgG1 sequences.

In some aspects, at least one CH3 amino acid sequence has one or more isoallotype mutations. In some aspects, the isoallotype mutations are D356E and L358M. In some aspects, the CL amino acid sequence is a Ckappa sequence.

Also described herein are OX40 binding molecules, comprising a first antigen binding site specific for an OX40 antigen, wherein the first antigen binding site comprises: A) a CDR1, a CDR2, and a CDR3 amino acid sequences of a specific light chain variable region (VL), wherein the CDR1, CDR2, and CDR3 VL sequences are selected from Table 4 corresponding to a specific OX40 antigen binding site (ABS); and B) a CDR1, a CDR2, and a CDR3 amino acid sequences of a specific heavy chain variable region (VH), wherein the CDR1, CDR2, and CDR3 VH sequences are selected from Table 3 corresponding to the specific OX40 ABS.

In some aspects, the first antigen binding site is specific for a first epitope of the OX40 antigen. In some aspects, the OX40 antigen comprises an OX40 domain selected from the group consisting of: OX40 amino acids 2-214, OX40 amino acids 66-214, OX40 amino acids 108-214, and OX40 amino acids 127-214. In some aspects, the OX40 antigen comprises a human OX40 antigen.

In some aspects, the first antigen binding site comprises a VL CDR1 comprising SEQ ID NO:220, a VL CDR2 comprising SEQ ID NO:221, and a VL CDR3 comprising SEQ ID NO:203, and a VH CDR1 comprising SEQ ID NO:83, a VH CDR2 comprising SEQ ID NO:123, and a VH CDR3 comprising SEQ ID NO:163. In some aspects, the first antigen binding site comprises a VL CDR1 comprising SEQ ID NO:220, a VL CDR2 comprising SEQ ID NO:221, and a VL CDR3 comprising SEQ ID NO:227, and a VH CDR1 comprising SEQ ID NO:83, a VH CDR2 comprising SEQ ID NO:123, and a VH CDR3 comprising SEQ ID NO:163. In some aspects, the first antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150.

In some aspects, the OX40 antigen binding molecule further comprises a second antigen binding site. In some aspects, the second antigen binding site is specific for the OX40 antigen. In some aspects, the second antigen binding site is specific for the first epitope of the OX40 antigen. In some aspects, the second antigen binding site is specific for a second epitope of the OX40 antigen. In some aspects, the first epitope and the second epitope are non-overlapping epitopes. In some aspects, the first antigen binding site comprises a VL CDR1 comprising SEQ ID NO:220, a VL CDR2 comprising SEQ ID NO:221, and a VL CDR3 comprising SEQ ID NO:203, and a VH CDR1 comprising SEQ ID NO:83, a VH CDR2 comprising SEQ ID NO:123, and a VH CDR3 comprising SEQ ID NO:163; and the second antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150. In some aspects, the first antigen binding site comprises a VL CDR1 comprising SEQ ID NO:220, a VL CDR2 comprising SEQ ID NO:221, and a VL CDR3 comprising SEQ ID NO:227, and a VH CDR1 comprising SEQ ID NO:83, a VH CDR2 comprising SEQ ID NO:123, and a VH CDR3 comprising SEQ ID NO:163; and the second antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150.

In some aspects, the second antigen binding site is specific for a second antigen different from the OX40 antigen. In some aspects, the second antigen is a second cell surface receptor.

In some aspects, the OX40 antigen binding molecule comprises an antibody format selected from the group consisting of: full-length antibodies, Fab fragments, Fvs, scFvs, tandem scFvs, Diabodies, scDiabodies, DARTs, tandAbs, and minibodies.

In some aspects, the OX40 antigen binding molecule comprises: a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence c) the first and the second polypeptides are associated through an interaction between the A and the F domain and an interaction between the B domain and the G domain to form the OX40 antigen binding molecule, and wherein the interaction between the A domain and the F domain form a first antigen binding site.

In some aspects, the OX40 antigen binding molecule further comprises: a third and a fourth polypeptide chain, wherein: (a) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, and domains I, J, and K have a constant region domain amino acid sequence; (b) the fourth polypeptide chain comprises a domain L and a domain M, wherein the domains are arranged, from N-terminus to C-terminus, in a L-M orientation, and wherein domain L has a variable region domain amino acid sequence and domain M has a constant region amino acid sequence; (c) the third and the fourth polypeptides are associated through an interaction between the H and the L domains and an interaction between the I and the M domains; and (d) the first and the third polypeptides are associated through an interaction between the D domain and the J domain and an interaction between the E domain and the K domain to form the OX40 antigen binding molecule, and wherein the interaction between the H domain and the L domain form a second antigen binding site.

In some aspects, the first antigen binding site is specific for the OX40 antigen. In some aspects, the second antigen binding site is specific for the OX40 antigen. In some aspects, the first antigen binding site is specific for a first epitope of the OX40 antigen and the second antigen binding site is specific for a second epitope of the OX40 antigen. In some aspects, the first epitope and the second epitope are non-overlapping epitopes.

In some aspects, domain B and domain G have a CH3 amino acid sequence. In some aspects, the amino acid sequences of the B domain and the G domain are identical, wherein the sequence is an endogenous CH3 sequence. In some aspects, the amino acid sequences of the B domain and the G domain are different and separately comprise respectively orthogonal modifications in an endogenous CH3 sequence, wherein the B domain interacts with the G domain, and wherein neither the B domain nor the G domain significantly interacts with a CH3 domain lacking the orthogonal modification. In some aspects, the orthogonal modifications of the B domain and the G domain comprise mutations that generate engineered disulfide bridges between the B domain and the G domain. In some aspects, the mutations of the B domain and the G domain that generate engineered disulfide bridges are a S354C mutation in one of the B domain and G domain, and a 349C in the other domain.

In some aspects, the orthogonal modifications of the B domain and the G domain comprise knob-in-hole mutations. In some aspects, the knob-in hole mutations of the B domain and the G domain are a T366W mutation in one of the B domain and G domain, and a T366S, L368A, and a Y407V mutation in the other domain.

In some aspects, the orthogonal modifications of the B domain and the G domain comprise charge-pair mutations. In some aspects, the charge-pair mutations of the B domain and the G domain are a T366K mutation in one of the B domain and G domain, and a L351D mutation in the other domain.

In some aspects, domain B and domain G have an IgM CH2 amino acid sequence or an IgE CH2 amino acid sequence. In some aspects, the IgM CH2 amino acid sequence or the IgE CH2 amino acid sequence comprise orthogonal modifications.

In some aspects, domain I has a CL sequence and domain M has a CH1 sequence. In some aspects, domain I has a CH1 sequence and domain M has a CL sequence. In some aspects, the CH1 sequence and the CL sequence each comprise one or more orthogonal modifications, wherein a domain having the CH1 sequence does not significantly interact with a domain having a CL sequence lacking the orthogonal modification. In some aspects, the orthogonal modifications in the CH1 sequence and the CL sequence comprise mutations that generate engineered disulfide bridges between the at least one CH1 domain and a CL domain, the mutations selected from the group consisting of: an engineered cysteine at position 138 of the CH1 sequence and position 116 of the CL sequence; an engineered cysteine at position 128 of the CH1 sequence and position 119 of the CL sequence, and an engineered cysteine at position 129 of the CH1 sequence and position 210 of the CL sequence. In some aspects, the orthogonal modifications in the CH1 sequence and the CL sequence comprise mutations that generate engineered disulfide bridges between the at least one CH1 domain and a CL domain, wherein the mutations comprise and engineered cysteines at position 128 of the CH1 sequence and position 118 of a CL Kappa sequence. In some aspects, the orthogonal modifications in the CH1 sequence and the CL sequence comprise mutations that generate engineered disulfide bridges between the at least one CH1 domain and a CL domain, the mutations selected from the group consisting of: a F118C mutation in the CL sequence with a corresponding A141C in the CH1 sequence; a F118C mutation in the CL sequence with a corresponding L128C in the CH1 sequence; and a S162C mutations in the CL sequence with a corresponding P171C mutation in the CH1 sequence. In some aspects, the orthogonal modifications in the CH1 sequence and the CL sequence comprise charge-pair mutations between the at least one CH1 domain and a CL domain, the charge-pair mutations selected from the group consisting of: a F118S mutation in the CL sequence with a corresponding A141L in the CH1 sequence; a F118A mutation in the CL sequence with a corresponding A141L in the CH1 sequence; a F118V mutation in the CL sequence with a corresponding A141L in the CH1 sequence; and a T129R mutation in the CL sequence with a corresponding K147D in the CH1 sequence. In some aspects, the orthogonal modifications in the CH1 sequence and the CL sequence comprise charge-pair mutations between the at least one CH1 domain and a CL domain, the charge-pair mutations selected from the group consisting of: a N138K mutation in the CL sequence with a corresponding G166D in the CH1 sequence, and a N138D mutation in the CL sequence with a corresponding G166K in the CH1 sequence.

In some aspects, domain A has a VL amino acid sequence and domain F has a VH amino acid sequence. In some aspects, domain A has a VH amino acid sequence and domain F has a VL amino acid sequence. In some aspects, domain H has a VL amino acid sequence and domain L has a VH amino acid sequence. In some aspects, domain H has a VH amino acid sequence and domain L has a VL amino acid sequence.

In some aspects, domain D and domain J have a CH2 amino acid sequence.

In some aspects, the E domain has a CH3 amino acid sequence.

In some aspects, the amino acid sequences of the E domain and the K domain are identical, wherein the sequence is an endogenous CH3 sequence. In some aspects, the amino acid sequences of the E domain and the K domain are different. In some aspects, the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification. In some aspects, the orthogonal modifications comprise mutations that generate engineered disulfide bridges between the E domain and the K domain. In some aspects, the mutations that generate engineered disulfide bridges are a S354C mutation in one of the E domain and the K domain, and a 349C in the other domain. In some aspects, the orthogonal modifications in the E domain and the K domain comprise knob-in-hole mutations. In some aspects, the knob-in hole mutations are a T366W mutation in one of the E domain or the K domain and a T366S, L368A, and a Y407V mutation in the other domain.

In some aspects, the orthogonal modifications in the E domain and the K domain comprise charge-pair mutations. In some aspects, the charge-pair mutations are a T366K mutation in one of the E domain or the K domain and a corresponding L351D mutation in the other domain.

In some aspects, the amino acid sequences of the E domain and the K domain are endogenous sequences of two different antibody domains, the domains selected to have a specific interaction that promotes the specific association between the first and the third polypeptides. In some aspects, the two different amino acid sequences are a CH1 sequence and a CL sequence.

The In some aspects, the OX40 antigen binding molecule further comprises a third antigen binding site. In some aspects, the third antigen binding site is specific for an OX40 antigen. In some aspects, the first antigen binding site and the third antigen binding site are specific for the same OX40 antigen. In some aspects, the first antigen binding site comprises a VL CDR1 comprising SEQ ID NO:220, a VL CDR2 comprising SEQ ID NO:221, and a VL CDR3 comprising SEQ ID NO:203, and a VH CDR1 comprising SEQ ID NO:83, a VH CDR2 comprising SEQ ID NO:123, and a VH CDR3 comprising SEQ ID NO:163. In some aspects, the first antigen binding site comprises a VL CDR1 comprising SEQ ID NO:220, a VL CDR2 comprising SEQ ID NO:221, and a VL CDR3 comprising SEQ ID NO:227, and a VH CDR1 comprising SEQ ID NO:83, a VH CDR2 comprising SEQ ID NO:123, and a VH CDR3 comprising SEQ ID NO:163. In some aspects, the first antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150.

In some aspects, the first antigen binding site and the third antigen binding site are specific for a different OX40 antigens. In some aspects, the first antigen binding site comprises a VL CDR1 comprising SEQ ID NO:220, a VL CDR2 comprising SEQ ID NO:221, and a VL CDR3 comprising SEQ ID NO:203, and a VH CDR1 comprising SEQ ID NO:83, a VH CDR2 comprising SEQ ID NO:123, and a VH CDR3 comprising SEQ ID NO:163; and the third antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150. In some aspects, the third antigen binding site comprises a VL CDR1 comprising SEQ ID NO:220, a VL CDR2 comprising SEQ ID NO:221, and a VL CDR3 comprising SEQ ID NO:203, and a VH CDR1 comprising SEQ ID NO:83, a VH CDR2 comprising SEQ ID NO:123, and a VH CDR3 comprising SEQ ID NO:163; and the first antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150. In some aspects, the first antigen binding site comprises a VL CDR1 comprising SEQ ID NO:220, a VL CDR2 comprising SEQ ID NO:221, and a VL CDR3 comprising SEQ ID NO:227, and a VH CDR1 comprising SEQ ID NO:83, a VH CDR2 comprising SEQ ID NO:123, and a VH CDR3 comprising SEQ ID NO:163; and the third antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150. In some aspects, the third antigen binding site comprises a VL CDR1 comprising SEQ ID NO:220, a VL CDR2 comprising SEQ ID NO:221, and a VL CDR3 comprising SEQ ID NO:227, and a VH CDR1 comprising SEQ ID NO:83, a VH CDR2 comprising SEQ ID NO:123, and a VH CDR3 comprising SEQ ID NO:163; and the first antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150.

In some aspects, the OX40 antigen binding molecule comprises a fifth polypeptide chain, wherein (a) the first polypeptide chain further comprises a domain N and a domain O, wherein the domains are arranged, from N-terminus to C-terminus, in a N-O-A-B-D-E orientation, and wherein domain N has a variable region domain amino acid sequence, domain O has a constant region amino acid sequence; (b) the fifth polypeptide chain comprises a domain P and a domain Q, wherein the domains are arranged, from N-terminus to C-terminus, in a P-Q orientation, and wherein domain P has a variable region domain amino acid sequence and domain Q has a constant region amino acid sequence; and (c) the first and the fifth polypeptides are associated through an interaction between the N and the P domains and an interaction between the O and the Q domains to form the OX40 antigen binding molecule.

In some aspects, (a) the amino acid sequences of domain N and domain A are identical, the amino acid sequences of domain H is different from the sequence of domain N and domain A, the amino acid sequences of domain O and domain B are identical, the amino acid sequences of domain I is different from the sequence of domain O and domain B, the amino acid sequences of domain P and domain F are identical, the amino acid sequences of domain L is different from the sequence of domain P and domain F, the amino acid sequences of domain Q and domain G are identical, the amino acid sequences of domain M is different from the sequence of domain Q and domain G; and (b) wherein the interaction between the A domain and the F domain form a first antigen binding site specific for a first antigen, the interaction between the H domain and the L domain form a second antigen binding site specific for a second antigen, and the interaction between the N domain and the P domain form a third antigen binding site specific for the first antigen. In some aspects, the first antigen is a first epitope of the OX40 antigen. In some aspects, the second antigen is a second epitope of the OX40 antigen. In some aspects, the first epitope and the second epitope are non-overlapping epitopes.

In some aspects, (a) the amino acid sequences of domain N, domain A, and domain H are different, the amino acid sequences of domain O, domain B, and domain I are different, the amino acid sequences of domain P, domain F, and domain L are different, and the amino acid sequences of domain Q, domain G, and domain M are different; and (b) the interaction between the A domain and the F domain form a first antigen binding site specific for a first antigen, the interaction between the H domain and the L domain form a second antigen binding site specific for a second antigen, and the interaction between the N domain and the P domain form a third antigen binding site specific for a third antigen.

In some aspects, the OX40 antigen binding molecule comprises a sixth polypeptide chain, wherein: (a) the third polypeptide chain further comprises a domain R and a domain S, wherein the domains are arranged, from N-terminus to C-terminus, in a R-S-H-I-J-K orientation, and wherein domain R has a variable region amino acid sequence and domain S has a constant domain amino acid sequence; (b) the sixth polypeptide chain comprises: a domain T and a domain U, wherein the domains are arranged, from N-terminus to C-terminus, in a T-U orientation, and wherein domain T has a variable region amino acid sequence and domain U has a constant domain amino acid sequence; and (c) the third and the sixth polypeptides are associated through an interaction between the R and the T domains and an interaction between the S and the U domains to form the OX40 antigen binding molecule.

In some aspects, (a) the amino acid sequences of domain R and domain A are identical, the amino acid sequences of domain H is different from the sequence of domain R and domain A, the amino acid sequences of domain S and domain B are identical, the amino acid sequences of domain I is different from the sequence of domain S and domain B, the amino acid sequences of domain T and domain F are identical, the amino acid sequences of domain L is different from the sequence of domain T and domain F, the amino acid sequences of domain U and domain G are identical, the amino acid sequences of domain M is different from the sequence of domain U and domain G, and (b) the interaction between the A domain and the F domain form a first antigen binding site specific for a first antigen, the interaction between the H domain and the L domain form a second antigen binding site specific for a second antigen, and the interaction between the R domain and the T domain form a third antigen binding site specific for the first antigen. In some aspects, the first antigen is a first epitope of the OX40 antigen. In some aspects, the second antigen is a second epitope of the OX40 antigen. In some aspects, the first epitope and the second epitope are non-overlapping epitopes.

In some aspects, (a) the amino acid sequences of domain R and domain H are identical, the amino acid sequences of domain A is different from the sequence of domain R and domain H, the amino acid sequences of domain S and domain I are identical, the amino acid sequences of domain B is different from the sequence of domain S and domain I, the amino acid sequences of domain T and domain L are identical, the amino acid sequences of domain F is different from the sequence of domain T and domain L, the amino acid sequences of domain U and domain M are identical, the amino acid sequences of domain G is different from the sequence of domain U and domain M, and (b) the interaction between the A domain and the F domain form a first antigen binding site specific for a first antigen, the interaction between the H domain and the L domain form a second antigen binding site specific for a second antigen, and the interaction between the R domain and the T domain form a third antigen binding site specific for the second antigen. In some aspects, the second antigen is a first epitope of the OX40 antigen. In some aspects, the first antigen is a second epitope of the OX40 antigen. In some aspects, the first epitope and the second epitope are non-overlapping epitopes.

In some aspects, (a) the amino acid sequences of domain R, domain A, and domain H are different, the amino acid sequences of domain S, domain B, and domain I are different, the amino acid sequences of domain T, domain F, and domain L are different, and the amino acid sequences of domain U, domain G, and domain M are different; and (b) the interaction between the A domain and the F domain form a first antigen binding site specific for a first antigen, the interaction between the H domain and the L domain form a second antigen binding site specific for a second antigen, and the interaction between the R domain and the T domain form a third antigen binding site specific for a third antigen.

Also described herein are purified binding molecules comprising any of the multivalent antibody constructs or the OX40 antigen binding molecules described herein. In some aspects, the purified binding molecule is purified by a purification method comprising a CH1 affinity purification step. In some aspects, the purified binding molecule is purified by a single-step purification method.

In some aspects, the multivalent antibody constructs, the OX40 antigen binding molecules, or the purified binding molecules described herein comprise a biophysical property selected from the group consisting of high yield, high purity, homogeneity, stability, long-term stability, acid stability, thermostability, low antibody cross-interaction, low antibody self-interaction, low hydrophobic binding, and cyno crossreactivity. In some aspects, the multivalent antibody constructs, the OX40 antigen binding molecules, or the purified binding molecules described herein comprise the biophysical property of high yield. In some aspects, the multivalent antibody constructs, the OX40 antigen binding molecules, or the purified binding molecules described herein comprise the biophysical property of high purity. In some aspects, the multivalent antibody constructs, the OX40 antigen binding molecules, or the purified binding molecules described herein comprise the biophysical property of homogeneity. In some aspects, the multivalent antibody constructs, the OX40 antigen binding molecules, or the purified binding molecules described herein comprise the biophysical property of stability. In some aspects, the multivalent antibody constructs, the OX40 antigen binding molecules, or the purified binding molecules described herein comprise the biophysical property of long-term stability. In some aspects, the multivalent antibody constructs, the OX40 antigen binding molecules, or the purified binding molecules described herein comprise the biophysical property of acid stability. In some aspects, the multivalent antibody constructs, the OX40 antigen binding molecules, or the purified binding molecules described herein comprise the biophysical property of thermostability. In some aspects, the multivalent antibody constructs, the OX40 antigen binding molecules, or the purified binding molecules described herein comprise the biophysical property of low antibody cross-interaction. In some aspects, the multivalent antibody constructs, the OX40 antigen binding molecules, or the purified binding molecules described herein comprise the biophysical property of low antibody self-interaction. In some aspects, the multivalent antibody constructs, the OX40 antigen binding molecules, or the purified binding molecules described herein comprise the biophysical property of low hydrophobic binding. In some aspects, the multivalent antibody constructs, the OX40 antigen binding molecules, or the purified binding molecules described herein comprise the biophysical property of cyno crossreactivity.

Also described herein are pharmaceutical compositions comprising any of the multivalent antibody constructs, the OX40 antigen binding molecules, or the purified binding molecules described herein, and a pharmaceutically acceptable diluent.

Also described herein are methods of treating cancer, comprising administering a therapeutically effective amount of any of the pharmaceutical compositions described herein to a patient in need thereof.

Also described herein are isolated polynucleotides encoding an amino acid sequence comprising any of the multivalent antibody constructs, the OX40 antigen binding molecules, or the purified binding molecules described herein.

Also described herein are vectors comprising any of the isolated polynucleotides described herein.

Also described herein are host cells comprising any of the vectors described herein.

### 5. BRIEF DESCRIPTION OF THE DRAWINGS

FIG. 1 presents schematic architectures, with respective naming conventions, for various binding molecules (also called antibody constructs) described herein.

FIG. 2A-E present higher resolution schematics of polypeptide chains and their domains for the bivalent (1×1) antibody constructs described herein. FIG. 2A presents a higher resolution schematic of polypeptide chains and their domains, with respective naming conventions, for the bivalent (1×1) antibody constructs described herein. FIG. 2B presents a higher resolution schematic of polypeptide chains and their domains for the “BC1” bivalent (1×1) format. FIG. 2C presents a higher resolution schematic of polypeptide chains and their domains for the “BC6” bivalent (1×1) format. FIG. 2D presents a higher resolution schematic of polypeptide chains and their domains for the “BC28” bivalent (1×1) format. FIG. 2E presents a higher resolution schematic of polypeptide chains and their domains for the “BC44” bivalent (1×1) format.

FIG. 3A-C present higher resolution schematics of polypeptide chains and their domains for the trivalent (2×1) antibody constructs described herein. FIG. 3A presents a schematic of polypeptide chains and their domains, with respective naming conventions, for the trivalent (2×1) antibody constructs described herein. FIG. 3B presents a higher resolution schematic of polypeptide chains and their domains for the “BC1 (2×1)” trivalent (2×1) format. FIG. 3C presents a higher resolution schematic of polypeptide chains and their domains for the “TB111” trivalent (1×1) format.

FIG. 4A-C present higher resolution schematics of polypeptide chains and their domains for the trivalent (1×2) antibody constructs described herein. FIG. 4A presents a schematic of polypeptide chains and their domains, with respective naming conventions, for the trivalent (1×2) antibody constructs described herein. FIG. 4B illustrates features of an exemplary trivalent 1×2 construct “CTLA4-4×Nivo×CTLA4-4.” FIG. 4C illustrates features of an exemplary trivalent 1×2 trispecific construct, “BC28-1×1×1a.”

FIG. 4D-F present higher resolution schematics of polypeptide chains and their domains for the tetravalent (2×2) antibody constructs described herein. FIG. 4D presents a schematic of polypeptide chains and their domains, with respective naming conventions, for certain tetravalent 2×2 constructs described herein. FIG. 4E illustrates certain salient features of the exemplary tetravalent 2×2 construct, “BC22-2×2.” FIG. 4F illustrates certain salient features of another exemplary tetravalent 2×2 construct.

FIG. 5 illustrates schematically functional differences between two antibody-mediated strategies for receptor clustering. FIG. 5A illustrates clustering by crosslinking antibody agonists that require an independent crosslinking agent (“First Generation Agonists”). FIG. 5B illustrates clustering by multispecific/multivalent antibodies capable of driving receptor clustering without the use of independent crosslinking agent, such as those described herein.

FIG. 6 shows epitope binning data for 17 unique OX40 binders obtained from a single phage display screening campaign.

FIG. 7 shows the setup, in 96 well format, of 96 bispecific bivalent (1×1) B-Body constructs. Each construct has two anti-OX40 specificities. Numerical numbers represent unique OX40 binders.

FIG. 8 tabulates concentrations in mg/mL of the respective bivalent 1×1 B-Body constructs after one-step purification. The average concentration was 950+/−500 μg/mL.

FIG. 9 shows NFκB activation by the 96 bispecific bivalent 1×1 B-Body constructs. Black column: 6 nM bispecific bivalent (1×1) B-Body. Open column: 6 nM of the respective 1×1 B-Body with 20 nM goat-anti-human (GAH) antibody added as an independent crosslinking agent. Data are normalized, with activation by crosslinked 6 nM OX40L-Fc ligand set to 1.

FIG. 10 shows NFκB activation by 96 trivalent (2×1) anti-OX40 B-Body constructs. Black column: 6 nM trivalent (2×1) B-Body. Open column: 6 nM of the respective (2×1) B-Body with 20 nM goat-anti-human (GAH) antibody added as an independent crosslinking agent. Data are normalized, with activation by crosslinked 6 nM OX40L-Fc ligand set to 1.

FIG. 11 compares agonist activity of three clinical OX40 agonists to activation by crosslinked natural ligand (OX40L-FC+GAH), with FIG. 11A showing the activity of the mAbs in the absence of the independent crosslinking agent, GAH (goat anti-human Fc antibody), and FIG. 11B showing the activity of the mAbs in the presence of the independent crosslinking agent, GAH.

FIG. 12 compares the three anti-OX40 clinical mAbs in the absence of GAH crosslinking to a bispecific bivalent (1×1) construct from our first campaign, “10×9”, a monospecific trivalent construct from our first campaign, “2×2×2”, and crosslinked antigen. Both constructs are seen to possess activity comparable to the crosslinked natural ligand, OX40L-Fc, in the absence of an independent cross-linking agent, and to be far superior as agonists as compared to the three known clinical anti-OX40 mAbs.

FIG. 13 shows the results of a high throughput screen for greater than 900 combinations of B-body candidate OX40 agonists tested in the HEK 293-NFkb-GFP/Luc-OX40 covering a wide range of affinity, epitope, and antibody construct geometry combinations. Arrows indicate clinical OX40 candidates used as controls (arrows from left to right: Pogalizumab, Tavolixizumab, and GSK3174998), each demonstrating activity below the 100% agonism by the OX40L-Fc fusion protein.

FIG. 14 shows agonist activity of three bivalent OX40 agonists in the absence and presence (+GAH) of the goat-anti-human (GAH) antibody crosslinking agent, as well as agonist activity of the control, crosslinked natural ligand-Fc fusion (OX40L-Fc), in the absence and presence of GAH (OX40L-Fc+GAH).

FIG. 15 shows dose response curves for a subset of bispecific OX40 agonists using both bivalent and trivalent formats identified during the high throughput screen.

FIG. 16A illustrates OX40 and OX40L bound in trimer from a top view (left panel) and side view (right panel). The extracellular domain of OX40 consists of four cysteine rich domains (CRD) with boundaries for each CRD noted.

FIG. 16B-G shows binding of the indicated monospecific antibodies to different OX40 fragments having a series of truncations from the N-terminus (AA 2-214, AA 66-214, AA 108-214, and AA 127-214), with the specific epitope region determined listed next to each monospecific antibody or ligand. FIG. 16B shows binding of candidate “2×2” for the different OX40 truncations. FIG. 16C shows binding of candidate “8×8” for the different OX40 truncations. FIG. 16D shows binding of the OX40 ligand “OX40L” for the different OX40 truncations. FIG. 16E shows binding of clinical antibody “GSK3174998” for the different OX40 truncations. FIG. 16F shows binding of clinical antibody “Pogalizumab” for the different OX40 truncations. FIG. 16G shows binding of clinical antibody “Tavolixizumab” for the different OX40 truncations.

FIG. 17 shows simultaneous binding of OX40 by different combinations of candidate antigen binding sites.

FIG. 18 shows the result summary from testing non-overlapping epitope binding for all possible combinations of the panel of the 40 antigen binding sites identified in the screen.

FIG. 19 shows T cell activation by plate bound (“coated”) and soluble OX40 agonists OX40 2-2×8 and GSK3174998 (“clinical”). Left panel shows T cell proliferation and right panel shows IL-2 secretion.

FIG. 20 shows a summary of primary CD4+ naïve T cell stimulatory activity for different multispecific multivalent candidate OX40 agonists. The X-axis represents the IL2 secretion, while the Y-axis is the CD4+/CD45RA+/CD25− T cell proliferation stimulated by each candidate. The shaded circle provides a cutoff identifying those agonists considered the most potent.

FIG. 21 shows the kinetics of T cell activation monitored by microscopy and charted using cell size measurement using the IncuCyte system to track the growth and proliferation of T cell clusters.

FIG. 22 shows a non-reducing SDS-PAGE analysis of two-step purified candidate OX40 agonists and two clinical monoclonal antibodies.

FIG. 23A-C shows dose response curves for activation, as monitored by cytokine secretion, of T cells using OX40 candidates OX40:24-11×11 and OX40:24-24×11 in a soluble 2×1 format, as well as by soluble GSK3174998 “GSK”, plate-bound GSK3174998 “GSK-Coated), and cross-linked GSK3174998 (“GSK+GAH”). FIG. 23A shows activation as monitored by TNFα secretion. FIG. 23B shows activation as monitored by IL-2 secretion. FIG. 23C shows activation as monitored by IFNγ secretion.

FIG. 24A-C shows dose response curves for activation, as monitored by cytokine secretion, of T cells using OX40 candidates OX40:24-11×11, OX40:24-24×11, and OX40:24-24(WEE)×11 in a soluble 2×1 format, and OX40 candidates OX40:24×11 and OX40:11×24 in a soluble 1×1 B-body format, as well as by cross-linked GSK3174998 (“GSK+GAH”). FIG. 24A shows activation as monitored by TNFα secretion. FIG. 24B shows activation as monitored by IL-2 secretion. FIG. 24C shows activation as monitored by IFNγ secretion.

FIG. 25A-C shows dose response curves for activation, as monitored by cytokine secretion, of T cells at Day 3 using OX40 candidates OX40:24-11×11, OX40:24-24×11, OX40:24-24(WEE)×11, and OX40:24(WEE)-11×11 in a soluble 2×1 format. FIG. 25A shows activation as monitored by TNFα secretion. FIG. 25B shows activation as monitored by IL-2 secretion. FIG. 25C shows activation as monitored by IFNγ secretion.

FIG. 26A-C shows dose response curves for activation, as monitored by cytokine secretion, of T cells at Day 4 using OX40 candidates OX40:24-11×11, OX40:24-24×11, OX40:24-24(WEE)×11, and OX40:24(WEE)-11×11 in a soluble 2×1 format. FIG. 26A shows activation as monitored by TNFα secretion. FIG. 26B shows activation as monitored by IL-2 secretion. FIG. 26C shows activation as monitored by IFNγ secretion.

FIG. 27A-C shows dose response curves for activation of T cells at Day 5 using OX40 candidates OX40:24-11×11, OX40:24-24×11, OX40:24-24(WEE)×11, and OX40:24(WEE)-11×11 in a soluble 2×1 format monitored by cytokine secretion. FIG. 27A shows activation as monitored by TNFα secretion. FIG. 27B shows activation as monitored by IL-2 secretion. FIG. 27C shows activation as monitored by IFNγ secretion.

FIG. 28A-D shows dose response curves for kinetics of the activation of T cells using OX40 candidates OX40:24-11×11, OX40:24-24×11, OX40:24-24(WEE)×11, and OX40:24(WEE)-11×11 in a soluble 2×1 format monitored by proliferation. FIG. 28A shows proliferation across Days 1-5 using the candidate OX40:24-24×11. FIG. 28B shows proliferation across Days 1-5 using the candidate OX40:24-24(WEE)×11. FIG. 28C shows proliferation across Days 1-5 using the candidate OX40:24-11×11. FIG. 28B shows proliferation across Days 1-5 using the candidate OX40:24(WEE)-11×11.

FIG. 29 shows dose response curves for activation of T cells using OX40 candidates OX40:24-24×11 and OX40:24-24(WEE)×11 in a soluble 2×1 format, OX40 candidate OX40:24×11 in a soluble 1×1 B-body format, and soluble and cross-linked (“+GAH”) OX40 candidate OX40:24-11×11 as monitored by TNFα secretion, as well as activation by soluble and cross-linked GSK3174998 (“GSK+GAH”).

FIG. 30A-B shows activation, as monitored by cytokine secretion, of T cells using OX40 candidates OX40:24-11×11, OX40:24-24×11, OX40:24-24(WEE)×11, and OX40:24-24×38 in a soluble 2×1 format, OX40 candidate OX40:11×24 in a soluble 1×1 B-body format, OX40 candidates OX40:11 and OX40:24 in a native IgG format, a combination of both OX40 candidates OX40:11 and OX40:24 in a native IgG format, by soluble and cross-linked GSK3174998 (“GSK+GAH”), as well as an anti-CD3 antibody or untreated (“no anti-CD3”) conditions. FIG. 27A shows activation as monitored by TNFα secretion. FIG. 27B shows activation as monitored by IL-2 secretion.

FIG. 31 shows dose response curves for activation of T cells as monitored in an NFκB Luc2 OX40 Jurkat T cell stimulation assay of OX40 candidates OX40:24-11×11 and OX40:24-24×11 in a soluble 2×1 format, and OX40 candidates OX40:24×11 and OX40:11×24 in a soluble 1×1 B-body format, as well as by soluble (“GSK”) and cross-linked (“GSK+GAH”) GSK3174998.

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

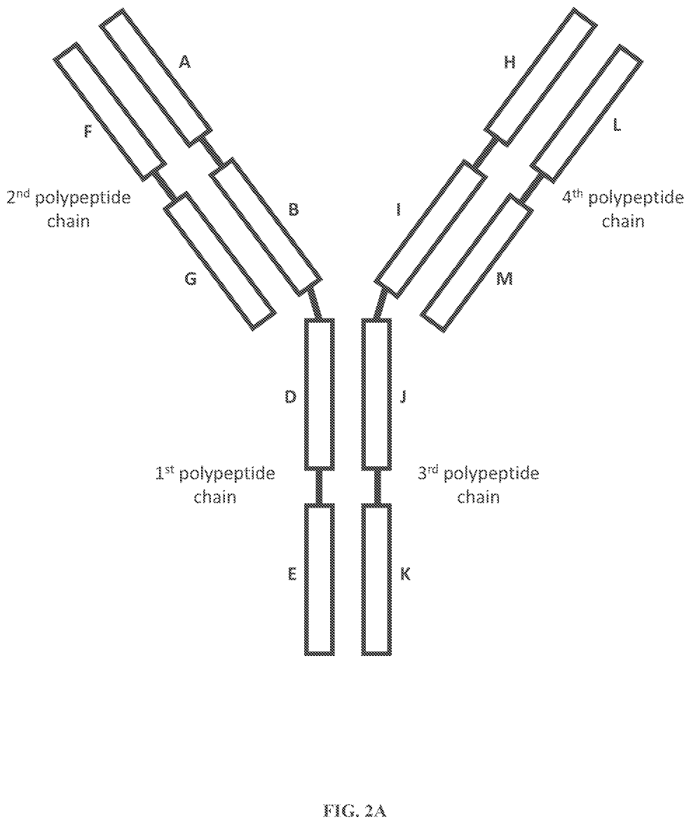

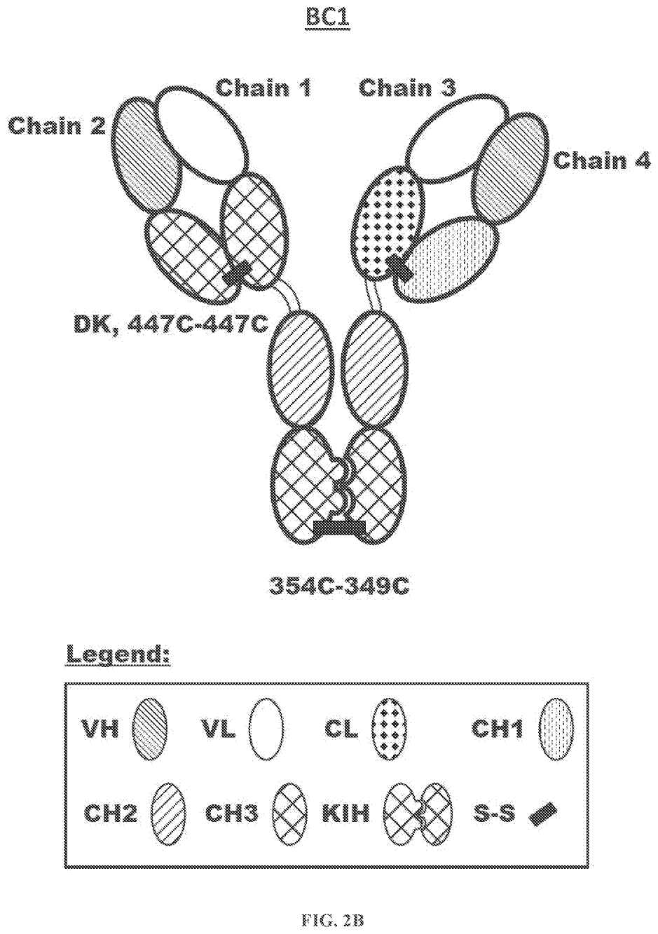

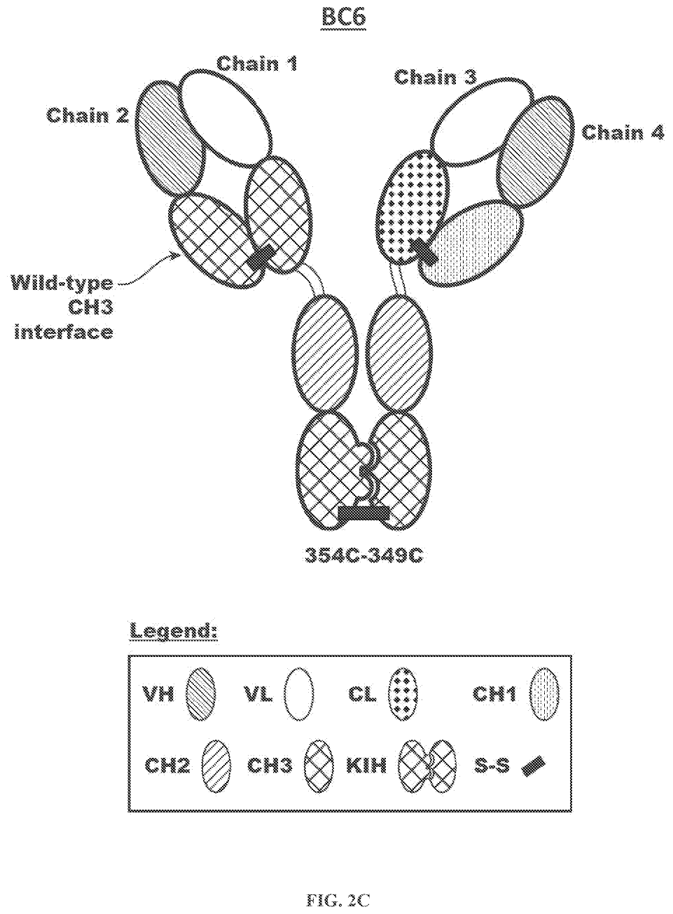

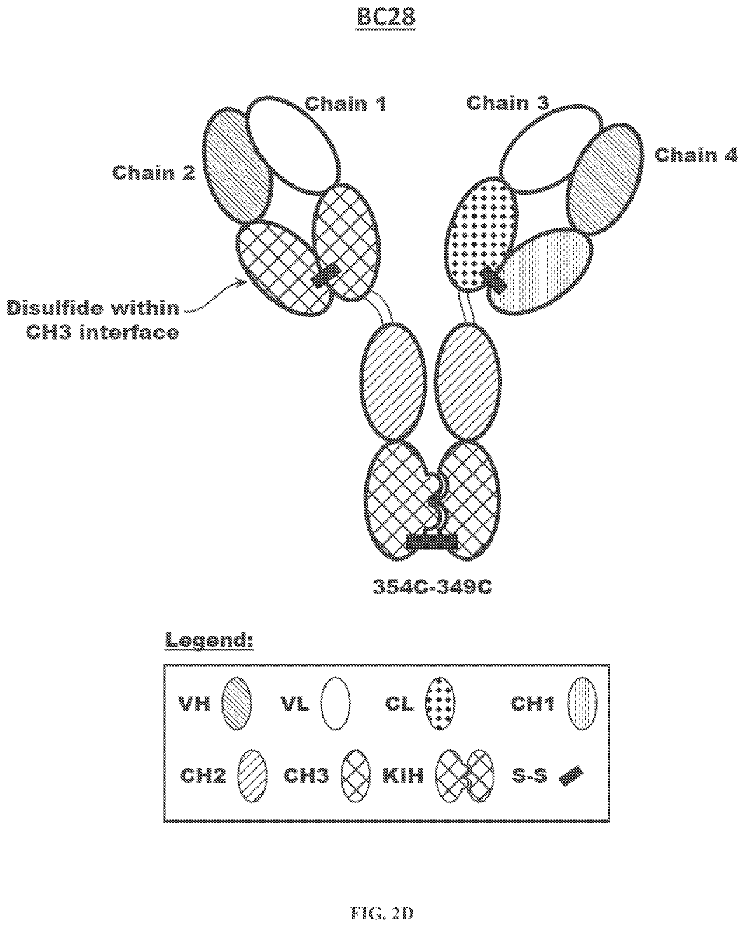

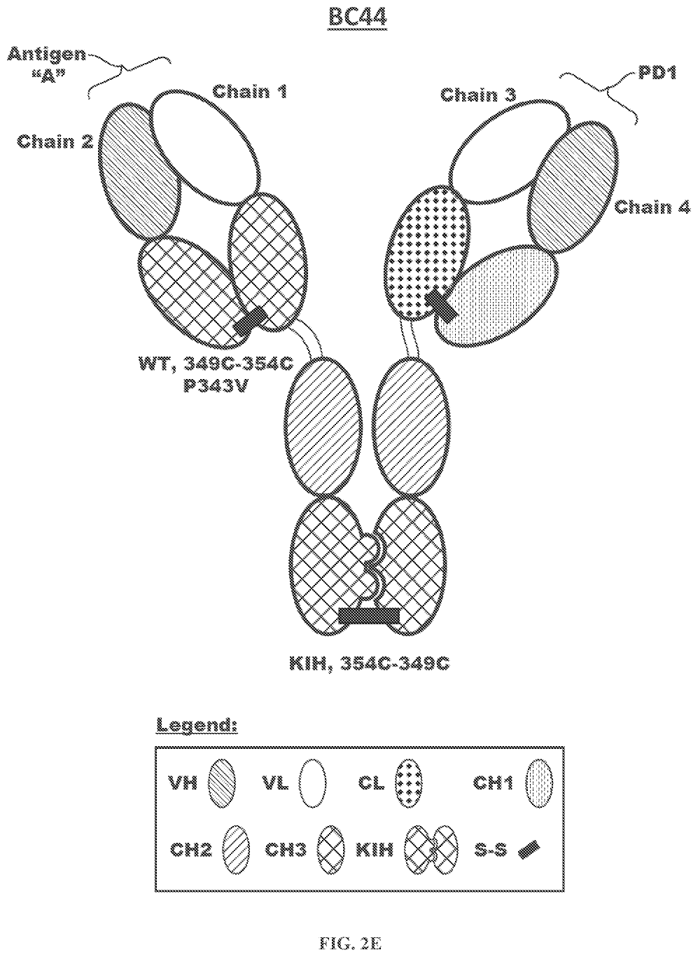

By “antigen binding site” is meant a region of a binding molecule, that specifically recognizes or binds to a given antigen or epitope. “B-Body,” as used herein and with reference to FIGS. 2A, 3A, 4A and 4D refers to binding molecules comprising the features of a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, and wherein domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; and (c) the first and the second polypeptides are associated through an interaction between the A and the F domains and an interaction between the B and the G domains to form the binding molecule. B-bodies are described in more detail in International Patent Application No. PCT/US2017/057268, herein incorporated by reference in its entirety.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis, arthritis, or cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

### 6.2. Other Interpretational Conventions

Unless otherwise specified, all references to sequences herein are to amino acid sequences.

Unless otherwise specified, antibody constant region residue numbering is according to the Eu index as described at

www.imgt.org/IMGTScientificChart/Numbering/Hu_IGHGnber.html#refs
(accessed Aug. 22, 2017) and in Edelman et al., Proc. Natl. Acad. USA, 63:78-85 (1969), which are hereby incorporated by reference in their entireties, and identifies the residue according to its location in an endogenous constant region sequence regardless of the residue's physical location within a chain of the binding molecules described herein. By “endogenous sequence” or “native sequence” is meant any sequence, including both nucleic acid and amino acid sequences, which originates from an organism, tissue, or cell and has not been artificially modified or mutated.

Polypeptide chain numbers (e.g., a “first” polypeptide chains, a “second” polypeptide chain. etc. or polypeptide “chain 1,” “chain 2,” etc.) are used herein as a unique identifier for specific polypeptide chains that form a binding molecule and is not intended to connote order or quantity of the different polypeptide chains within the binding molecule.

In this disclosure, “comprises,” “comprising,” “containing,” “having,” “includes,” “including,” and linguistic variants thereof have the meaning ascribed to them in U.S. patent law, permitting the presence of additional components beyond those explicitly recited.

Ranges provided herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or apparent from context, as used herein the term “or” is understood to be inclusive. Unless specifically stated or apparent from context, as used herein, the terms “a”, “an”, and “the” are understood to be singular or plural.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### 6.3. Overview of Experimental Results

We have developed multivalent antibody constructs that are readily expressed to high level in standard transient transfection systems with high fidelity pairing of cognate heavy chain pairs and cognate heavy and light chain pairs, and that can be purified in a single step to purity levels sufficient to allow in vitro assay. Following a standard library panning campaign to identify phage-displayed human Fabs that bind the TNFRSF member OX40, we used our novel multivalent constructs to assess the identified antigen binding sites for monovalent binding to OX40. See Example 1.

Because our constructs are suitable for high throughput expression and assay, we then recloned antigen binding sites having specificity for different OX40 epitopes into a wide variety of monospecific and bispecific bivalent and trivalent combinations. We expressed and tested these multivalent constructs in high throughput assays for OX40 agonist activity, both in the absence and presence of an agent that further cross-links the antibody construct. Our constructs demonstrated a wide range of agonist activity in the absence of independent crosslinking agent; some have agonist activity in the absence of cross-linker greater than that of crosslinked OX40 ligand. The best constructs exhibited agonist activity in the absence of independent crosslinking agent superior to that observed with three known mAb clinical candidates. A number of our constructs also demonstrated a wide range of increased activity upon further crosslinking.

### 6.4. Receptor-Clustering Multivalent Antibody Constructs

Accordingly, in a first aspect, multivalent antibody constructs are provided. The construct is capable of (i) binding a cell surface receptor target that requires clustering for agonist activity, and (ii) clustering the receptor target on the cell surface in the absence of an independent cross-linking agent. Each of the target receptor-binding antigen binding sites of the construct is contributed by antibody variable region binding domains.

In various embodiments, the multivalent construct is monospecific.

In various embodiments, the construct is multispecific.

In embodiments of the multivalent antibody construct that are multispecific, the construct comprises a first antigen binding site specific for a first epitope of the target receptor, and a second antigen binding site specific for a second antigenic target.

In some embodiments, the second antigenic target is a second epitope of the target receptor. In some aspects, the first epitope and the second epitope are non-overlapping epitopes. In some embodiments, the second antigenic target is an epitope of a second protein.

In particular embodiments, the second antigenic target is an epitope of a second protein, wherein the second protein is a second cell surface receptor. In certain embodiments, the target cell surface receptor and the second cell surface receptor are commonly expressed on the surface of at least some mammalian cells.

In typical embodiments, whether monospecific or multispecific, the target receptor is a TNF Receptor superfamily (TNFRSF) member.

In various embodiments, the TNFRSF member is TNFR1 (also known as CD120a and TNFRSF1A), TNFR2 (also known as CD120b and TNFRSF1B), TNFRSF3 (also known as LTβR), TNFRSF4 (also known as OX40 and CD134), TNFRSF5 (also known as CD40), TNFRSF6 (also known as FAS and CD95), TNFRSF6B (also known as DCR3), TNFRSF7 (also known as CD27), TNFRSF8 (also known as CD30), TNFRSF9 (also known as 4-1BB), TNFRSF10A (also known as TRAILR1, DR4, and CD26), TNFRSF10B (also known as TRAILR2, DR5, and CD262), TNFRSF10C (also known as TRAILR3, DCR1, CD263), TNFRSF10D (also known as TRAILR4, DCR2, and CD264), TNFRSF11A (also known as RANK and CD265), TNFRSF11B (also known as OPG), TNFRSF12A (also known as FN14, TWEAKR, and CD266), TNFRSF13B (also known as TACI and CD267), TNFRSF13C (also known as BAFFR, BR3, and CD268), TNFRSF14 (also known as HVEM and CD270), TNFRSF16 (also known as NGFR, p75NTR, and CD271), or TNFRSF17 (also known as BCMA and CD269), TNFRSF18 (also known as GITR and CD357), TNFRSF19 (also known as TROY, TAJ, and TRADE), TNFRSF21 (also known as CD358), TNFRSF25 (also known as Apo-3, TRAMP, LARD, or WS-1), EDA2R (also known as XEDAR).

In some embodiments, the target receptor is OX40 (TNFRSF4), CD40 (TNFRSF5), or 4-1BB (TNFRSF9). In particular embodiments, the target receptor is OX40. In particular embodiments, the target receptor is CD40. In certain embodiments, the target receptor is 4-1BB.

In typical embodiments, the target receptor is a human TNFRSF. In certain of these embodiments, the target receptor is human OX40, human CD40, or human 4-1BB. In particular embodiments, the target receptor is human OX40. In particular embodiments, the target receptor is human CD40. In particular embodiments, the target receptor is human 4-1BB.

In other embodiments, the target receptor is not a TNFRSF member. In certain embodiments, the target receptor is CD20. In a particular embodiment, the target receptor is human CD20.

In various embodiments, the presence of an independent cross-linking agent does not increase agonist activity above that achieved in the absence of the independent cross-linking agent. In certain embodiments, the presence of an independent cross-linking agent does not increase agonist activity above that achieved in the absence of the independent cross-linking agent when tested in vitro. In particular embodiments in which the independent cross-linking agent is cross-linked natural ligand of the target receptor, the presence of cross-linked ligand for the target receptor does not increase in vitro agonist activity above that achieved in the absence of the cross-linked ligand. In certain embodiments, the presence of an independent cross-linking agent does not increase agonist activity above that achieved in the absence of the independent cross-linking agent when the multivalent antibody construct is administered in vivo.

In various embodiments, the presence of an independent cross-linking agent increases agonist activity above that achieved in the absence of the independent cross-linking agent. In certain embodiments, the presence of an independent cross-linking agent increases agonist activity above that achieved in the absence of the independent cross-linking agent when tested in vitro. In particular embodiments in which the independent cross-linking agent is cross-linked natural ligand of the target receptor, the presence of cross-linked ligand for the target receptor increases in vitro agonist activity above that achieved in the absence of the cross-linked ligand. In certain embodiments, cross-linked target receptor ligand increases in vitro agonist activity 10%, 20%, 30%, 40%, 50%, 60%, 70%, 80%, 90%, 100%, 200%, 300% or even 400% above that achieved in the absence of the cross-linked ligand. In specific embodiments, cross-linked target receptor ligand increases in vitro agonist activity more than 2-fold above that achieved in the absence of the cross-linked ligand. In certain embodiments, cross-linked target receptor ligand increases in vitro agonist activity more than 3-fold above that achieved in the absence of the cross-linked ligand

In certain embodiments, the presence of an independent cross-linking agent increases agonist activity above that achieved in the absence of the independent cross-linking agent when the multivalent construct is administered in vivo.

### 6.4.1. Bivalent Constructs

In some embodiments, the multivalent construct is bivalent.

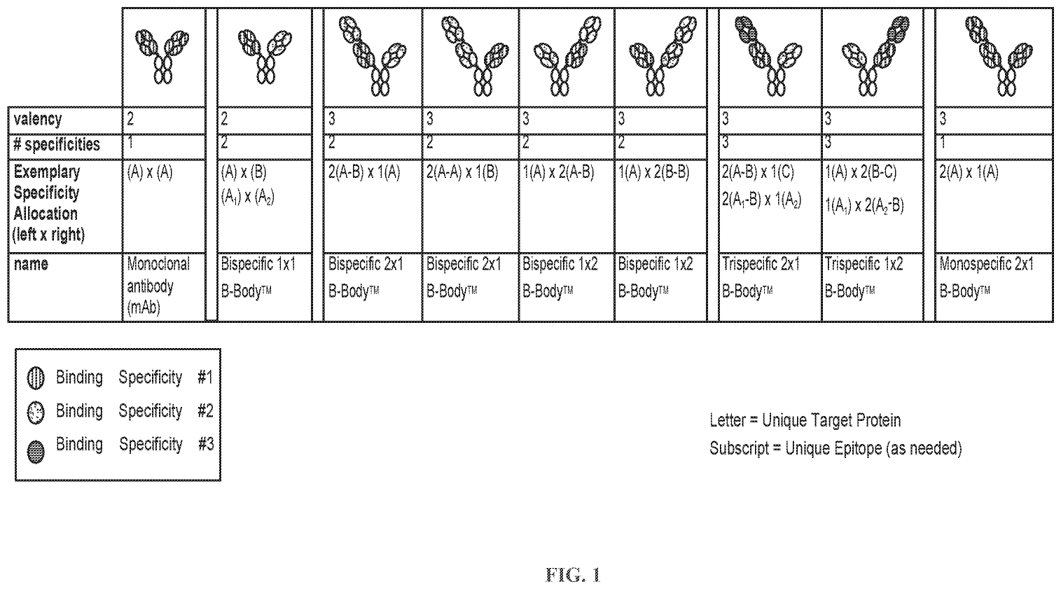

In various embodiments, the bivalent construct is a bivalent (1×1) construct. The basic architecture of bivalent (1×1) constructs is included among the architectures schematized in FIG. 1, and is specifically shown in greater detail in FIG. 2A.

With reference to FIG. 2A, in a first series of embodiments, the binding molecules comprise a first, a second, a third, and a fourth polypeptide chain, wherein: a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, wherein domain H has a variable region domain amino acid sequence, and wherein domain I, domain J, and domain K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence domain; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the bivalent binding molecule.

### 6.4.1.1. Specific Binding Molecule Domain Architectures

In some embodiments, the bivalent binding molecules comprise a native antibody architecture, wherein the binding molecule is structured as described in Section 6.4.1 wherein domains A and H comprise VH amino acid sequences, domains F and L comprise VL amino acid sequences, domains B and I comprise CH1, domains G and M comprise CL, domains D and J comprise CH2, and domains E and K comprise CH3.

In preferred embodiments, the binding molecule is a B-Body™. B-Body™ binding molecules are described in International Patent Application No. PCT/US2017/057268. In some embodiments, the binding molecule is structured as described in Section 6.4.1 wherein domains A and H comprise VL, domains B and G comprise CH3, domain I comprises CL or CH1, domain M comprises CH1 or CL, domains D and J comprise CH2, and domains E and K comprise CH3. In some embodiments, domain I comprises CL and domain M comprises CH1. In some embodiments, domain I comprises CH1 and domain M comprises CL.

In some embodiments, the binding molecule is a CrossMab™. CrossMab™ antibodies are described in U.S. Pat. Nos. 8,242,247; 9,266,967; and 8,227,577, U.S. Patent Application Pub. No. 20120237506, U.S. Patent Application Pub. No. US20090162359, WO2016016299, WO2015052230. In some embodiments, the binding molecule is a bivalent, bispecific antibody, comprising: a) the light chain and heavy chain of an antibody specifically binding to a first antigen; and b) the light chain and heavy chain of an antibody specifically binding to a second antigen, wherein constant domains CL and CH1 from the antibody specifically binding to a second antigen are replaced by each other. In some embodiments, the binding molecule is structured as described in Section 6.4.1 wherein A is VH, B is CH1, D is CH2, E is CH3, F is VL, G is CL, H is VL or VH, I is CL, J is CH2, K is CH3, L is VH or VL, and M is CH1.

In some embodiments, the binding molecule is an antibody having a general architecture described in U.S. Pat. No. 8,871,912 and WO2016087650. In some embodiments, the binding molecule is a domain-exchanged antibody comprising a light chain (LC) composed of VL-CH3, and a heavy chain (HC) comprising VH-CH3-CH2-CH3, wherein the VL-CH3 of the LC dimerizes with the VH-CH3 of the HC thereby forming a domain-exchanged LC/HC dimer comprising a CH3LC/CH3HC domain pair. In some embodiments, the binding molecule is structured as described in Section 6.4.1 wherein A is VH, B is CH3, D is CH2, E is CH3, F is VL, G is CH3, H is VH, I is CH1, J is CH2, K is CH3, L is VL, and M is CL.

In some embodiments, the binding molecule is as described in WO2017011342. In some embodiments, the binding molecule is structured as described in Section 6.4.1 wherein A is VH or VL, B is CH2 from IgM or IgE, D is CH2, E is CH3, F is VL or VH, G is CH2 from IgM or IgE, H is VH, I is CH1, J is CH2, K is CH3, L is VL, and M is CL.

In some embodiments, the binding molecule is as described in WO2006093794. In some embodiments, the binding molecule is structured as described in Section 6.4.1 wherein A is VH, B is CH1, D is CH2, E is CH3, F is VL, G is CL, H is VL, I is CL or CH1, J is CH2, K is CH3, L is VH, and M is CH1 or CL.

In various embodiments, the first and third polypeptide chains are identical in sequence to one another, and the second and fourth polypeptide are identical in sequence to one another. In these embodiments, association of the first and third polypeptide chains through interactions between domains E & K (see Section 6.4.1.16 below) form a bivalent monospecific antibody construct.

In other embodiments, the first and third polypeptide chains are non-identical in sequence to one another, and the second and fourth polypeptide are non-identical in sequence to one another. In these embodiments, association of the first and third polypeptide chains through interactions between domains E & K (see Section 6.4.1.16 below) is capable of forming a bivalent bispecific antibody construct.

### 6.4.1.2. Domain A (Variable Region)

In the bivalent (1×1) binding molecules described herein, domain A has a variable region domain amino acid sequence. Variable region domain amino acid sequences, as described herein, are variable region domain amino acid sequences of an antibody including VL and VH antibody domain sequences. VL and VH sequences are described in greater detail in Sections 6.4.1.2.1 and 6.4.1.2.2, respectively. In a preferred embodiment, domain A has a VL antibody domain sequence and domain F has a VH antibody domain sequence. In some embodiments, domain A has a VH antibody domain sequence and domain F has a VL antibody domain sequence.

### 6.4.1.2.1. VL Regions

The VL amino acid sequences in the binding molecules described herein are typically sequences of a native antibody light chain variable domain. In a typical arrangement in both natural antibodies and the antibody constructs described herein, a specific VL amino acid sequence associates with a specific VH amino acid sequence to form an antigen-binding site. In various embodiments, the VL amino acid sequences are mammalian sequences, including human sequences, synthesized sequences, or combinations of human, non-human mammalian, mammalian, and/or synthesized sequences, as described in further detail in Sections 6.4.1.2.3 and 6.4.1.2.4.

In particular embodiments, the VL amino acid sequences are human antibody light chain sequences. In certain embodiments, the VL amino acid sequences are lambda (λ) light chain variable domain sequences. In a preferred embodiment, the VL amino acid sequences are kappa (κ) light chain variable domain sequences.

In various embodiments, VL amino acid sequences are mutated sequences of naturally occurring (e.g., “native”) sequences.

In the bivalent (1×1) binding molecules described herein, the C-terminus of domain A is connected to the N-terminus of domain B. In certain embodiments, domain A has a VL amino acid sequence that is mutated at its C-terminus at the junction between domain A and domain B, as described in greater detail in Section 6.4.4.

### 6.4.1.2.2. VH Regions

The VH amino acid sequences in the binding molecules described herein are typically sequences of a native antibody heavy chain variable domain. In a typical antibody arrangement in both nature and in the binding molecules described herein, a specific VH amino acid sequence associates with a specific VL amino acid sequence to form an antigen-binding site. In various embodiments, VH amino acid sequences are mammalian sequences, including human sequences, synthesized sequences, or combinations of non-human mammalian, mammalian, and/or synthesized sequences, as described in further detail in Sections 6.4.1.2.3 and 6.4.1.2.4. In various embodiments, VH amino acid sequences are mutated sequences of naturally occurring (e.g., “native”) sequences.

### 6.4.1.2.3. Complementarity Determining Regions

VH and VL amino acid sequences may comprise highly variable sequences termed “complementarity determining regions” (CDRs), typically three CDRs (CDR1, CD2, and CDR3). In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences. In various embodiments, the CDRs are naturally occurring sequences that have been mutated to alter the binding affinity of the antigen-binding site for a particular antigen or epitope. In certain embodiments, the naturally occurring CDRs have been mutated in an in vivo host through affinity maturation and somatic hypermutation. In certain embodiments, the CDRs have been mutated in vitro through methods including, but not limited to, PCR-mutagenesis and chemical mutagenesis. In various embodiments, the CDRs are synthesized sequences including, but not limited to, CDRs obtained from random sequence CDR libraries and rationally designed CDR libraries.

### 6.4.1.2.4. Framework Regions and CDR Grafting

VH and VL amino acid sequences may comprise “framework region” (FR) sequences. FRs are generally conserved sequence regions that act as a scaffold for interspersed CDRs (see Section 6.4.1.2.3), typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, camel, donkey, goat, and human sequences. In a preferred embodiment, the FRs are human sequences. In various embodiments, the FRs are naturally occurring sequences. In particular embodiments, the FRs are human FR sequences. In various embodiments, the FRs are synthesized sequences including, but not limited, rationally designed sequences.

In a variety of embodiments, the FRs and the CDRs are both from the same naturally occurring variable domain sequence. In a variety of embodiments, the FRs and the CDRs are from different variable domain sequences, wherein the CDRs are grafted onto the FR scaffold with the CDRs providing specificity for a particular antigen. In certain embodiments, the grafted CDRs are all derived from the same naturally occurring variable domain sequence. In certain embodiments, the grafted CDRs are derived from different variable domain sequences. In certain embodiments, the grafted CDRs are synthesized sequences including, but not limited to, CDRs obtained from random sequence CDR libraries and rationally designed CDR libraries. In certain embodiments, the grafted CDRs and the FRs are from the same species. In certain embodiments, the grafted CDRs and the FRs are from different species. In a preferred grafted CDR embodiment, an antibody is “humanized”, wherein the grafted CDRs are non-human mammalian sequences including, but not limited to, mouse, rat, hamster, rabbit, camel, donkey, and goat sequences, and the FRs are human sequences. Humanized antibodies are discussed in more detail in U.S. Pat. No. 6,407,213, the entirety of which is hereby incorporated by reference for all it teaches. In various embodiments, portions or specific sequences of FRs from one species are used to replace portions or specific sequences of another species' FRs.

### 6.4.1.3. Domain B (Constant Region)

In the bivalent (1×1) binding molecules, domain B has a constant region domain sequence. Constant region domain amino acid sequences, as described herein, are typically sequences of a constant region domain of a native antibody.

In a variety of embodiments, the constant region sequences are mammalian sequences, including, but not limited to, mouse, rat, hamster, rabbit, camel, donkey, goat, and human sequences. In a preferred embodiment, the constant region sequences are human sequences. In certain embodiments, the constant region sequences are from an antibody light chain. In particular embodiments, the constant region sequences are from a lambda or kappa light chain. In certain embodiments, the constant region sequences are from an antibody heavy chain. In particular embodiments, the constant region sequences are an antibody heavy chain sequence that is an IgA1, IgA2, IgD, IgE, IgG1, IgG2, IgG3, IgG4, or IgM isotype. In a specific embodiment, the constant region sequences are from an IgG isotype. In a preferred embodiment, the constant region sequences are from an IgG1 isotype. In preferred specific embodiments, the constant region sequence is a CH3 sequence. CH3 sequences are described in greater detail in Section 6.4.1.3.1. In other preferred embodiments, the constant region sequence is an orthologous CH2 sequence. Orthologous CH2 sequences are described in greater detail in Section 6.4.1.3.2.

In some embodiments, domain B has a CH1 sequence. In some embodiments, domain B has a CH2 sequence from IgE. In some embodiments, domain B has a CH2 sequence from IgM.

In particular embodiments, the constant region sequence has been mutated to include one or more orthogonal mutations. In a preferred embodiment, domain B has a constant region sequence that is a CH3 sequence comprising knob-hole (synonymously, “knob-in-hole,” “KIH”) orthogonal mutations, as described in greater detail in Section 6.4.1.15.2, and either a S354C or a Y349C mutation that forms an engineered disulfide bridge with a CH3 domain containing an orthogonal mutation, as described in in greater detail in Section 6.4.1.15.1. In some preferred embodiments, the knob-hole orthogonal mutation is a T366W mutation.

### 6.4.1.3.1. CH3 Regions

CH3 amino acid sequences, as described herein, are typically sequences of the C-terminal domain of a native antibody heavy chain.

In a variety of embodiments, the CH3 sequences are mammalian sequences, including, but not limited to, mouse, rat, hamster, rabbit, camel, donkey, goat, and human sequences. In a preferred embodiment, the CH3 sequences are human sequences. In certain embodiments, the CH3 sequences are from an IgA1, IgA₂, IgD, IgE, IgM, IgG₁, IgG₂, IgG₃, IgG₄ isotype or C_{H4} sequences from an IgE or IgM isotype. In a specific embodiment, the CH3 sequences are from an IgG isotype. In a preferred embodiment, the CH3 sequences are from an IgG₁ isotype.

In certain embodiments, the CH3 sequences are endogenous sequences. In particular embodiments, the CH3 sequence is UniProt accession number P01857 amino acids 224-330. In various embodiments, a CH3 sequence is a segment of an endogenous CH3 sequence. In particular embodiments, a CH3 sequence has an endogenous CH3 sequence that lacks the N-terminal amino acids G224 and Q225. In particular embodiments, a CH3 sequence has an endogenous CH3 sequence that lacks the C-terminal amino acids P328, G329, and K330. In particular embodiments, a CH3 sequence has an endogenous CH3 sequence that lacks both the N-terminal amino acids G224 and Q225 and the C-terminal amino acids P328, G329, and K330. In preferred embodiments, a binding molecule has multiple domains that have CH3 sequences, wherein a CH3 sequence can refer to both a full endogenous CH3 sequence as well as a CH3 sequence that lacks N-terminal amino acids, C-terminal amino acids, or both.

In certain embodiments, the CH3 sequences are endogenous sequences that have one or more mutations. In particular embodiments, the mutations are one or more orthogonal mutations that are introduced into an endogenous CH3 sequence to guide specific pairing of specific CH3 sequences, as described in more detail in Sections 6.4.1.15.1-6.4.1.15.3.

In certain embodiments, the CH3 sequences are engineered to reduce immunogenicity of the antibody by replacing specific amino acids of one allotype with those of another allotype and referred to herein as isoallotype mutations, as described in more detail in Stickler et al. (Genes Immun. 2011 April; 12(3): 213-221), which is herein incorporated by reference for all that it teaches. In particular embodiments, specific amino acids of the G1m1 allotype are replaced. In a preferred embodiment, isoallotype mutations D356E and L358M are made in the CH3 sequence.

In a preferred embodiment, domain B has a human IgG1 CH3 amino acid sequence with the following mutational changes: P343V; Y349C; and a tripeptide insertion, 445P, 446G, 447K. In other preferred embodiments, domain B has a human IgG1 CH3 sequence with the following mutational changes: T366K; and a tripeptide insertion, 445K, 446S, 447C. In still other preferred embodiments, domain B has a human IgG1 CH3 sequence with the following mutational changes: Y349C and a tripeptide insertion, 445P, 446G, 447K.

In certain embodiments, domain B has a human IgG1 CH3 sequence with a 447C mutation incorporated into an otherwise endogenous CH3 sequence.

In the bivalent (1×1) binding molecules described herein, the N-terminus of domain B is connected to the C-terminus of domain A. In certain embodiments, domain B has a CH3 amino acid sequence that is mutated at its N-terminus at the junction between domain A and domain B, as described in greater detail in Section 6.4.4.1.

In the binding molecules, the C-terminus of domain B is connected to the N-terminus of domain D. In certain embodiments, domain B has a CH3 amino acid sequence that is extended at the C-terminus at the junction between domain B and domain D, as described in greater detail in Section 6.4.4.3.

### 6.4.1.3.2. Orthologous CH2 Regions

CH2 amino acid sequences, as described herein, are typically sequences of the third domain of a native antibody heavy chain, with reference from the N-terminus to C-terminus. CH2 amino acid sequences, in general, are discussed in more detail in Section 6.4.1.4. In a series of embodiments, a binding molecule has more than one paired set of CH2 domains that have CH2 sequences, wherein a first set has CH2 amino acid sequences from a first isotype and one or more orthologous sets of CH2 amino acid sequences from another isotype. The orthologous CH2 amino acid sequences, as described herein, are able to interact with CH2 amino acid sequences from a shared isotype, but not significantly interact with the CH2 amino acid sequences from another isotype present in the binding molecule. In particular embodiments, all sets of CH2 amino acid sequences are from the same species. In preferred embodiments, all sets of CH2 amino acid sequences are human CH2 amino acid sequences. In other embodiments, the sets of CH2 amino acid sequences are from different species. In particular embodiments, the first set of CH2 amino acid sequences is from the same isotype as the other non-CH2 domains in the binding molecule. In a specific embodiment, the first set has CH2 amino acid sequences from an IgG isotype and the one or more orthologous sets have CH2 amino acid sequences from an IgM or IgE isotype. In certain embodiments, one or more of the sets of CH2 amino acid sequences are endogenous CH2 sequences. In other embodiments, one or more of the sets of CH2 amino acid sequences are endogenous CH2 sequences that have one or more mutations. In particular embodiments, the one or more mutations are orthogonal knob-hole mutations, orthogonal charge-pair mutations, or orthogonal hydrophobic mutations. Orthologous CH2 amino acid sequences useful for the binding molecules are described in more detail in international PCT applications WO2017/011342 and WO2017/106462, herein incorporated by reference in their entirety.

### 6.4.1.4. Domain D (Constant Region)

In the bivalent (1×1) binding molecules described herein, domain D has a constant region amino acid sequence. Constant region amino acid sequences are described in more detail herein, for example in Section and 6.4.1.3.

In a preferred series of embodiments, domain D has a CH2 amino acid sequence. CH2 amino acid sequences, as described herein, are typically sequences of the third domain of a native antibody heavy chain, with reference from the N-terminus to C-terminus. In a variety of embodiments, the CH2 sequences are mammalian sequences, including but not limited to mouse, rat, hamster, rabbit, camel, donkey, goat, and human sequences. In a preferred embodiment, the CH2 sequences are human sequences. In certain embodiments, the CH2 sequences are from a IgA1, IgA₂, IgD, IgE, IgG₁, IgG₂, IgG₃, IgG₄, or IgM isotype. In a preferred embodiment, the CH2 sequences are from an IgG₁ isotype.

In certain embodiments, the CH2 sequences are endogenous sequences. In particular embodiments, the sequence is Uniprot accession number P01857 amino acids 111-223. In a preferred embodiment, the CH2 sequences have an N-terminal hinge region peptide that connects the N-terminal variable domain-constant domain segment to the CH2 domain, as discussed in more detail in Sections 6.4.4.3 and 6.4.4.4. In some embodiments, the CH2 sequence comprises one or more mutations that reduce effector function, as discussed in more detail in Section 6.6.4.

In the binding molecules, the N-terminus of domain D is connected to the C-terminus of domain B. In certain embodiments, domain B has a CH3 amino acid sequence that is extended at the C-terminus at the junction between domain D and domain B, as described in greater detail in Section 6.4.4.3.

In the binding molecules, the C-terminus of domain D is connected to the N-terminus of domain E. In particular embodiments, domain D is connected to the N-terminus of domain E that has a CH1 amino acid sequence or CL amino acid sequence, as described in greater detail in Section 6.4.4.5.

### 6.4.1.5. Domain E (Constant Region)

In the bivalent (1×1) binding molecules, domain E has a constant region domain amino acid sequence. Constant region amino acid sequences are described in greater detail in Section 6.4.1.3.

In certain embodiments, the constant region sequence is a CH3 sequence. CH3 sequences are described in greater detail in Section 6.4.1.3.1.

In particular embodiments, the constant region sequence has been mutated to include one or more orthogonal mutations. In a preferred embodiment, domain E has a constant region sequence that is a CH3 sequence comprising knob-hole (synonymously, “knob-in-hole,” “KIH”) orthogonal mutations, as described in greater detail in Section 6.4.1.15.2, and either a S354C or a Y349C mutation that forms an engineered disulfide bridge with a CH3 domain containing an orthogonal mutation, as described in in greater detail in Section 6.4.1.15.1. In some preferred embodiments, the knob-hole orthogonal mutation is a T366W mutation.

In certain embodiments, the constant region domain sequence is a CH1 sequence. CH1 sequences are described in greater detail in Section 6.4.1.9.1. In certain embodiments, the N-terminus of the CH1 domain is connected to the C-terminus of a CH2 domain, as described in greater detail in Section 6.4.4.5.

In certain embodiments, the constant region sequence is a CL sequence. CL sequences are described in greater detail in Section 6.4.1.9.2. In certain embodiments, the N-terminus of the CL domain is connected to the C-terminus of a CH2 domain, as described in greater detail in Section 6.4.4.5.

### 6.4.1.6. Domain F (Variable Region)

In the bivalent (1×1) binding molecules, domain F has a variable region domain amino acid sequence. Variable region domain amino acid sequences, as discussed in greater detail in Section 6.4.1.2, are variable region domain amino acid sequences of an antibody including VL and VH antibody domain sequences. VL and VH sequences are described in greater detail in Sections 6.4.1.2.1 and 6.4.1.2.2, respectively. In a preferred embodiment, domain F has a VH antibody domain sequence. In some embodiments, domain F has a VL antibody domain sequence.

### 6.4.1.7. Domain G (Constant Region)

In the binding molecules, domain G has a constant region amino acid sequence. Constant region amino acid sequences are described in greater detail in Section 6.4.1.3.

In preferred embodiments, domain G has a CH3 amino acid sequence. CH3 sequences are described in greater detail in Section 6.4.1.3.1. In other preferred embodiments, the constant region sequence is an orthologous CH2 sequence. Orthologous CH2 sequences are described in greater detail in Section 6.4.1.3.2.

In certain preferred embodiments, domain G has a human IgG1 CH3 sequence with the following mutational changes: S354C; and a tripeptide insertion, 445P, 446G, 447K. In some preferred embodiments, domain G has a human IgG1 CH3 sequence with the following mutational changes: S354C; and 445P, 446G, 447K tripeptide insertion. In some preferred embodiments, domain G has a human IgG1 CH3 sequence with the following changes: L351D, and a tripeptide insertion of 445G, 446E, 447C.

### 6.4.1.8. Domain H (Variable Region)

In the binding molecules, domain H has a variable region domain amino acid sequence. Variable region domain amino acid sequences, discussed in greater detail in Section 6.4.1.2, are variable region domain amino acid sequences of an antibody including VL and VH antibody domain sequences. VL and VH sequences are described in greater detail in Sections 6.4.1.2.1 and 6.4.1.2.2, respectively. In a preferred embodiment, domain H has a VL antibody domain sequence. In some embodiments, domain H has a VH antibody domain sequence.

### 6.4.1.9. Domain I (Constant Region)

In the binding molecules, domain I has a constant region domain amino acid sequence. Constant region amino acid sequences are described in greater detail in Section 6.4.1.3. In a series of preferred embodiments of the binding molecules, domain I has a CL amino acid sequence. In another series of embodiments, domain I has a CH1 amino acid sequence. CH1 and CL amino acid sequences are described in further detail in Sections 6.4.1.9.1 and 6.4.1.9.2, respectively.

### 6.4.1.9.1. CH1 Domains

CH1 amino acid sequences, as described herein, are typically sequences of the second domain of a native antibody heavy chain, with reference from the N-terminus to C-terminus. In certain embodiments, the CH1 sequences are endogenous sequences. In a variety of embodiments, the CH1 sequences are mammalian sequences, including, but not limited to mouse, rat, hamster, rabbit, camel, donkey, goat, and human sequences. In a preferred embodiment, the CH1 sequences are human sequences. In certain embodiments, the CH1 sequences are from an IgA₁, IgA₂, IgD, IgE, IgG₁, IgG₂, IgG₃, IgG₄, or IgM isotype. In a preferred embodiment, the CH1 sequences are from an IgG₁ isotype. In preferred embodiments, the CH1 sequence is Uniprot accession number P01857 amino acids 1-98.

### 6.4.1.9.2. CL Domains

CL amino acid sequences, as described herein, are typically sequences of the second domain of a native antibody light chain, with reference from the N-terminus to C-terminus. In certain embodiments, the CL sequences are endogenous sequences. In a variety of embodiments, the CL sequences are mammalian sequences, including, but not limited to mouse, rat, hamster, rabbit, camel, donkey, goat, and human sequences. In a preferred embodiment, CL sequences are human sequences.

In certain embodiments, the CL amino acid sequences are lambda (λ) light chain constant domain sequences. In particular embodiments, the CL amino acid sequences are human lambda light chain constant domain sequences. In preferred embodiments, the lambda (λ) light chain sequence is UniProt accession number P0CG04.

In certain embodiments, the CL amino acid sequences are kappa (κ) light chain constant domain sequences. In a preferred embodiment, the CL amino acid sequences are human kappa (κ) light chain constant domain sequences. In a preferred embodiment, the kappa light chain sequence is UniProt accession number P01834.

### 6.4.1.10. Domain J (Constant Region)

In the bivalent (1×1) binding molecules described herein, domain J has a constant region amino acid sequence. Constant region amino acid sequences are described in more detail herein, for example in Section 6.4.1.3. In a preferred series of embodiments, domain J has a CH2 amino acid sequence. CH2 amino acid sequences are described in greater detail in Section 6.4.1.4. In a preferred embodiment, the CH2 amino acid sequence has an N-terminal hinge region that connects domain J to domain I, as described in greater detail in Section 6.4.4.4.

In the binding molecules, the C-terminus of domain J is connected to the N-terminus of domain K. In particular embodiments, domain J is connected to the N-terminus of domain K that has a CH1 amino acid sequence or CL amino acid sequence, as described in greater detail in Section 6.4.4.5.

### 6.4.1.11. Domain K (Constant Region)

In the binding molecules, domain K has a constant region domain amino acid sequence. Constant region domain amino acid sequences are described in greater detail in Section 6.4.1.3. In certain embodiments, the constant region sequence is a CH3 sequence. CH3 sequences are described in greater detail in Section 6.4.1.3.1. In a preferred embodiment, domain K has a constant region sequence that is a CH3 sequence comprising knob-hole orthogonal mutations, as described in greater detail in Section 6.4.1.15.2, isoallotype mutations, as described in more detail in 6.4.1.3.1., and either a S354C or a Y349C mutation that forms an engineered disulfide bridge with a CH3 domain containing an orthogonal mutation, as described in in greater detail in Section 6.4.1.15.1. In some preferred embodiments, the knob-hole orthogonal mutations combined with isoallotype mutations are the following mutational changes: D356E, L358M, T366S, L368A, and Y407V.

In certain embodiments, the constant region domain sequence is a CH1 sequence. In certain embodiments, the N-terminus of the CH1 domain is connected to the C-terminus of a CH2 domain, as described in greater detail in Section 6.4.4.5. In certain embodiments, the constant region sequence is a CL sequence. In certain embodiments, the N-terminus of the CL domain is connected to the C-terminus of a CH2 domain, as described in greater detail in Section 6.4.4.5. CH1 and CL amino acid sequences are described in further detail in Sections 6.4.1.9.1 and 6.4.1.9.2, respectively.

### 6.4.1.12. Domain L (Variable Region)

In the binding molecules, domain L has a variable region domain amino acid sequence. Variable region domain amino acid sequences, discussed in greater detail in Section 6.4.1.2, are variable region domain amino acid sequences of an antibody including VL and VH antibody domain sequences. VL and VH sequences are described in greater detail in Sections 6.4.1.2.1 and 6.4.1.2.2, respectively. In a preferred embodiment, domain L has a VH antibody domain sequence. In some embodiments, domain L has a VL antibody domain sequence.

### 6.4.1.13. Domain M (Constant Region)

In the binding molecules, domain M has a constant region domain amino acid sequence. Constant region amino acid sequences are described in greater detail in Section 6.4.1.3. In a series of preferred embodiments of the binding molecules, domain I has a CH1 amino acid sequence and domain M has a CL amino acid sequence. In another series of preferred embodiments, domain I has a CL amino acid sequence and domain M has a CH1 amino acid sequence. CH1 and CL amino acid sequences are described in further detail in Sections 6.4.1.9.1 and 6.4.1.9.2, respectively.

### 6.4.1.14. Pairing of Domains A & F

In the binding molecules, a domain A VL or VH amino acid sequence and a cognate domain F VH or VL amino acid sequence are associated and form an antigen binding site (ABS). The A:F antigen binding site (ABS) is capable of specifically binding an epitope of an antigen. Antigen binding by an ABS is described in greater detail in Section 6.4.1.14.1.

In a variety of multivalent embodiments, the ABS formed by domains A and F (A:F) is identical in sequence to one or more other ABSs within the binding molecule and therefore has the same recognition specificity as the one or more other sequence-identical ABSs within the binding molecule.

In a variety of multivalent embodiments, the A:F ABS is non-identical in sequence to one or more other ABSs within the binding molecule. In certain embodiments, the A:F ABS has a recognition specificity different from that of one or more other sequence-non-identical ABSs in the binding molecule. In particular embodiments, the A:F ABS recognizes a different antigen from that recognized by at least one other sequence-non-identical ABS in the binding molecule. In particular embodiments, the A:F ABS recognizes a different epitope of an antigen that is also recognized by at least one other sequence-non-identical ABS in the binding molecule. In these embodiments, the ABS formed by domains A and F recognizes an epitope of antigen, wherein one or more other ABSs within the binding molecule recognizes the same antigen but not the same epitope.

### 6.4.1.14.1. Binding of Antigen by ABS

An ABS, and the binding molecule comprising such ABS, is said to “recognize” the epitope (or more generally, the antigen) to which the ABS specifically binds, and the epitope (or more generally, the antigen) is said to be the “recognition specificity” or “binding specificity” of the ABS.

The ABS is said to bind to its specific antigen or epitope with a particular affinity.

As described herein, “affinity” refers to the strength of interaction of non-covalent intermolecular forces between one molecule and another. The affinity, i.e. the strength of the interaction, can be expressed as a dissociation equilibrium constant (KD), wherein a lower KD value refers to a stronger interaction between molecules. KD values of antibody constructs are measured by methods well known in the art including, but not limited to, bio-layer interferometry (e.g. Octet/FORTEBIO®), surface plasmon resonance (SPR) technology (e.g. Biacore®), and cell binding assays. For purposes herein, affinities are dissociation equilibrium constants measured by bio-layer interferometry using Octet/FORTEBIO®.

“Specific binding,” as used herein, refers to an affinity between an ABS and its cognate antigen or epitope in which the KD value is below 10-6M, 10-7M, 10-8M, 10-9M, or 10-10M.

The number of ABSs in a binding molecule as described herein defines the “valency” of the binding molecule. As schematized in FIG. 1, a binding molecule having a single ABS is “monovalent”. A binding molecule having a plurality of ABSs is said to be “multivalent”. A multivalent binding molecule having two ABSs is “bivalent.” A multivalent binding molecule having three ABSs is “trivalent.” A multivalent binding molecule having four ABSs is “tetravalent.”

In various multivalent embodiments, all of the plurality of ABSs have the same recognition specificity. As schematized in FIG. 1, such a binding molecule is a “monospecific” “multivalent” binding construct. In other multivalent embodiments, at least two of the plurality of ABSs have different recognition specificities. Such binding molecules are multivalent and “multispecific”. In multivalent embodiments in which the ABSs collectively have two recognition specificities, the binding molecule is “bispecific.” In multivalent embodiments in which the ABSs collectively have three recognition specificities, the binding molecule is “trispecific.”

In multivalent embodiments in which the ABSs collectively have a plurality of recognition specificities for different epitopes present on the same antigen, the binding molecule is “multiparatopic.” Multivalent embodiments in which the ABSs collectively recognize two epitopes on the same antigen are “biparatopic.”

In various multivalent embodiments, multivalency of the binding molecule improves the avidity of the binding molecule for a specific target. As described herein, “avidity” refers to the overall strength of interaction between two or more molecules, e.g. a multivalent binding molecule for a specific target, wherein the avidity is the cumulative strength of interaction provided by the affinities of multiple ABSs. Avidity can be measured by the same methods as those used to determine affinity, as described above. In certain embodiments, the avidity of a binding molecule for a specific target is such that the interaction is a specific binding interaction, wherein the avidity between two molecules has a KD value below 10⁻⁶M, 10⁻⁷M, 10⁻⁸M, 10⁻⁹M, or 10⁻¹⁰M. In certain embodiments, the avidity of a binding molecule for a specific target has a KD value such that the interaction is a specific binding interaction, wherein the one or more affinities of individual ABSs do not have has a KD value that qualifies as specifically binding their respective antigens or epitopes on their own. In certain embodiments, the avidity is the cumulative strength of interaction provided by the affinities of multiple ABSs for separate antigens on a shared specific target or complex, such as separate antigens found on an individual cell. In certain embodiments, the avidity is the cumulative strength of interaction provided by the affinities of multiple ABSs for separate epitopes on a shared individual antigen.

### 6.4.1.15. Pairing of Domains B & G

In the binding molecules described herein, a domain B constant region amino acid sequence and a domain G constant region amino acid sequence are associated. Constant region domain amino acid sequences are described in greater detail in Section 6.4.1.3.

In a series of preferred embodiments, domain B and domain G have CH3 amino acid sequences. CH3 sequences are described in greater detail in Section 6.4.1.3.1. The sequence may be a CH3 sequence from human IgG1.

In various embodiments, the amino acid sequences of the B and the G domains are identical. In certain of these embodiments, the sequence is an endogenous CH3 sequence.

In a variety of embodiments, the amino acid sequences of the B and the G domains are different, and separately comprise respectively orthogonal modifications in an endogenous CH3 sequence, wherein the B domain interacts with the G domain, and wherein neither the B domain nor the G domain significantly interacts with a CH3 domain lacking the orthogonal modification.

“Orthogonal modifications” or synonymously “orthogonal mutations” as described herein are one or more engineered mutations in an amino acid sequence of an antibody domain that alter the affinity of binding of a first domain having orthogonal modification for a second domain having a complementary orthogonal modification, as compared to binding of the first and second domains in the absence of the orthogonal modifications. In some embodiments, the orthogonal modifications decrease the affinity of binding of the first domain having the orthogonal modification for the second domain having the complementary orthogonal modification, as compared to binding of the first and second domains in the absence of the orthogonal modifications. In preferred embodiments, the orthogonal modifications increase the affinity of binding of the first domain having the orthogonal modification for the second domain having the complementary orthogonal modification, as compared to binding of the first and second domains in the absence of the orthogonal modifications. In certain preferred embodiments, the orthogonal modifications decrease the affinity of a domain having the orthogonal modifications for a domain lacking the complementary orthogonal modifications.

In certain embodiments, orthogonal modifications are mutations in an endogenous antibody domain sequence. In a variety of embodiments, orthogonal modifications are modifications of the N-terminus or C-terminus of an endogenous antibody domain sequence including, but not limited to, amino acid additions or deletions. In particular embodiments, orthogonal modifications include, but are not limited to, engineered disulfide bridges, knob-in-hole mutations, and charge-pair mutations, as described in greater detail in Sections 6.4.1.15.1-6.4.1.15.3. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations, as described in greater detail in Section 6.4.1.3.1.

### 6.4.1.15.1. Orthogonal Engineered Disulfide Bridges in CH3

In a variety of embodiments, the orthogonal modifications comprise mutations that generate engineered disulfide bridges between a first and a second domain. As described herein, “engineered disulfide bridges” are mutations that provide non-endogenous cysteine amino acids in two or more domains such that a non-native disulfide bond forms when the two or more domains associate. Engineered disulfide bridges are described in greater detail in Merchant et al. (Nature Biotech (1998) 16:677-681), the entirety of which is hereby incorporated by reference for all it teaches. In certain embodiments, engineered disulfide bridges improve orthogonal association between specific domains. In a particular embodiment, the mutations that generate engineered disulfide bridges are a K392C mutation in one of a first or second CH3 domains, and a D399C in the other CH3 domain. In a preferred embodiment, the mutations that generate engineered disulfide bridges are a S354C mutation in one of a first or second CH3 domains, and a Y349C in the other CH3 domain. In another preferred embodiment, the mutations that generate engineered disulfide bridges are a 447C mutation in both the first and second CH3 domains that are provided by extension of the C-terminus of a CH3 domain incorporating a KSC tripeptide sequence.

### 6.4.1.15.2. Orthogonal Knob-Hole Mutations

In a variety of embodiments, orthogonal modifications comprise knob-hole (synonymously, knob-in-hole) mutations. As described herein, knob-hole mutations are mutations that change the steric features of a first domain's surface such that the first domain will preferentially associate with a second domain having complementary steric mutations relative to association with domains without the complementary steric mutations. Knob-hole mutations are described in greater detail in U.S. Pat. Nos. 5,821,333 and 8,216,805, each of which is incorporated herein in its entirety. In various embodiments, knob-hole mutations are combined with engineered disulfide bridges, as described in greater detail in Merchant et al. (Nature Biotech (1998) 16:677-681)), incorporated herein by reference in its entirety. In various embodiments, knob-hole mutations, isoallotype mutations, and engineered disulfide mutations are combined.

In certain embodiments, the knob-in-hole mutations are a T366Y mutation in a first domain, and a Y407T mutation in a second domain. In certain embodiments, the knob-in-hole mutations are a F405A in a first domain, and a T394W in a second domain. In certain embodiments, the knob-in-hole mutations are a T366Y mutation and a F405A in a first domain, and a T394W and a Y407T in a second domain. In certain embodiments, the knob-in-hole mutations are a T366W mutation in a first domain, and a Y407A in a second domain. In certain embodiments, the combined knob-in-hole mutations and engineered disulfide mutations are a S354C and T366W mutations in a first domain, and a Y349C, T366S, L368A, and a Y407V mutation in a second domain. In a preferred embodiment, the combined knob-in-hole mutations, isoallotype mutations, and engineered disulfide mutations are a S354C and T366W mutations in a first domain, and a Y349C, D356E, L358M, T366S, L368A, and a Y407V mutation in a second domain.

### 6.4.1.15.3. Orthogonal Charge-Pair Mutations

In a variety of embodiments, orthogonal modifications are charge-pair mutations. As described herein, “charge-pair mutations” are mutations that affect the charge of an amino acid in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562, 9,248,182, and 9,358,286, each of which is incorporated by reference herein for all they teach. In certain embodiments, charge-pair mutations improve stability between specific domains. In a preferred embodiment, the charge-pair mutations are a T366K mutation in a first domain, and a L351D mutation in the other domain.

### 6.4.1.16. Pairing of Domains E & K

In various embodiments, the E domain has a CH3 amino acid sequence.

In various embodiments, the K domain has a CH3 amino acid sequence.

In a variety of embodiments, the amino acid sequences of the E and K domains are identical, wherein the sequence is an endogenous CH3 sequence. CH3 sequences are described in Section 6.4.1.3.1. In some embodiments, the CH3 sequences of domains E and K are IgG-CH3 sequences.

In a variety of embodiments, the sequences of the E and K domains are different. In a variety of embodiments, the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification. In certain embodiments, the orthogonal modifications include, but are not limited to, engineered disulfide bridges, knob-in-hole mutations, and charge-pair mutations, as described in greater detail in sections 6.4.1.15.1-6.4.1.15.3. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations, as described in greater detail in Section 6.4.1.3.1.

In a variety of embodiments, the amino acid sequences of the E domain and the K domain are endogenous sequences of two different antibody domains, the domains selected to have a specific interaction that promotes the specific association between the first and the third polypeptides. In various embodiments, the two different amino acid sequences are a CH1 sequence and a CL sequence. CH1 sequences and CL sequences are described in greater detail in Sections 6.4.1.9.1 and 6.4.1.9.2, respectively. Use of CH1 and CL sequences at the C-terminus of a heavy chain to promote specific heavy chain association is described in U.S. Pat. No. 8,242,247, the entirety of which is hereby incorporated by reference for all it teaches. In certain embodiments, the CH1 sequence and the CL sequences are both endogenous sequences. In certain embodiments, the CH1 sequence and the CL sequences separately comprise respectively orthogonal modifications in endogenous CH1 and CL sequences. In particular embodiments, the orthogonal modifications in endogenous CH1 and CL sequences are an engineered disulfide bridge selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In a preferred embodiment, the engineered cysteines are at position 128 of the CH1 sequence and position 118 of the CL Kappa sequence, as numbered by the Eu index.

### 6.4.1.17. Pairing of Domains I & M and Pairing of Domains H & L

In a variety of embodiments, domain I has a CL sequence and domain M has a CH1 sequence. In a variety of embodiments, domain H has a VL sequence and domain L has a VH sequence. In a preferred embodiment, domain H has a VL amino acid sequence, domain I has a CL amino acid sequence, domain L has a VH amino acid sequence, and domain M has a CH1 amino acid sequence. In another preferred embodiment, domain H has a VL amino acid sequence, domain I has a CL amino acid sequence, domain L has a VH amino acid sequence, domain M has a CH1 amino acid sequence, and domain K has a CH3 amino acid sequence.

In a variety of embodiments, the amino acid sequences of the I domain and the M domain separately comprise respectively orthogonal modifications in an endogenous sequence, wherein the I domain interacts with the M domain, and wherein neither the I domain nor the M domain significantly interacts with a domain lacking the orthogonal modification. In a series of embodiments, the orthogonal mutations in the I domain are in a CL sequence and the orthogonal mutations in the M domain are in CH1 sequence. Orthogonal mutations are described in more detail in Sections 6.4.1.15.1-6.4.1.15.3. In a variety of embodiments, the orthogonal mutations in the CL sequence and the CH1 sequence are charge-pair mutations. In specific embodiments the charge-pair mutations are a F118S, F118A or F118V mutation in the CL sequence with a corresponding A141L in the CH1 sequence, or a T129R mutation in the CL sequence with a corresponding K147D in the CH1 sequence, as numbered by the Eu index and described in greater detail in Bonisch et al. (Protein Engineering, Design & Selection, 2017, pp. 1-12), herein incorporated by reference for all that it teaches. In a series of preferred embodiments the charge-pair mutations are a N138K mutation in the CL sequence with a corresponding G166D in the CH1 sequence, or a N138D mutation in the CL sequence with a corresponding G166K in the CH1 sequence, as numbered by the Eu index.

In a variety of embodiments, the orthogonal mutations in the CL sequence and the CH1 sequence generate an engineered disulfide bridge. In a series of preferred embodiments, the mutations that provide non-endogenous cysteine amino acids are a F118C mutation in the CL sequence with a corresponding A141C in the CH1 sequence, or a F118C mutation in the CL sequence with a corresponding L128C in the CH1 sequence, or a S162C mutations in the CL sequence with a corresponding P171C mutation in the CH1 sequence, as numbered by the Eu index.

In a variety of embodiments, the amino acid sequences of the H domain and the L domain separately comprise respectively orthogonal modifications in an endogenous sequence, wherein the H domain interacts with the L domain, and wherein neither the H domain nor the L domain significantly interacts with a domain lacking the orthogonal modification. In a series of embodiments, the orthogonal mutations in the H domain are in a VL sequence and the orthogonal mutations in the L domain are in VH sequence. In specific embodiments, the orthogonal mutations are charge-pair mutations at the VH/VL interface. In preferred embodiments, the charge-pair mutations at the VH/VL interface are a Q39E in VH with a corresponding Q38K in VL, or a Q39K in VH with a corresponding Q38E in VL, as described in greater detail in Igawa et al. (Protein Eng. Des. Sel., 2010, vol. 23, 667-677), herein incorporated by reference for all it teaches.

In certain embodiments, the interaction between the A domain and the F domain form a first antigen binding site specific for a first antigen, and the interaction between the H domain and the L domain form a second antigen binding site specific for a second antigen. In certain embodiments, the interaction between the A domain and the F domain form a first antigen binding site specific for a first antigen, and the interaction between the H domain and the L domain form a second antigen binding site specific for the first antigen.

### 6.4.1.18. Bivalent Specificity

In various embodiments, the bivalent construct is monospecific. In these embodiments, the bivalent construct comprises two copies of a first antigen binding site specific for a first epitope of the target receptor.

In various embodiments, the bivalent construct is bispecific. In these embodiments, the construct comprises a first antigen binding site specific for a first epitope of the target receptor, and a second antigen binding site specific for a second antigenic target. In some bispecific embodiments, the second antigenic target is a second epitope of the target receptor. In some aspects, the first epitope and the second epitope are non-overlapping epitopes. In some embodiments, the second antigenic target is an epitope of a second protein.

In some bispecific bivalent embodiments, the antigen binding site specific for a first epitope of the target receptor is an A:F antigen binding site. In some embodiments, the antigen binding site specific for a first epitope of the target receptor is an H:L antigen binding site.

### 6.4.2. Trivalent Constructs

In another series of embodiments, the binding molecules have three antigen binding sites and are therefore termed “trivalent.”

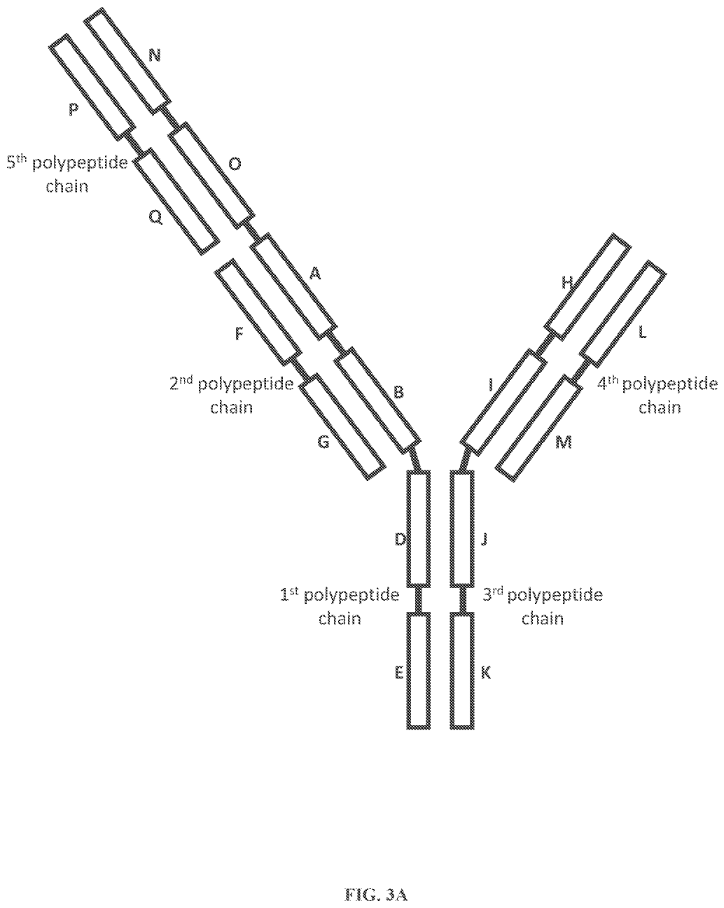

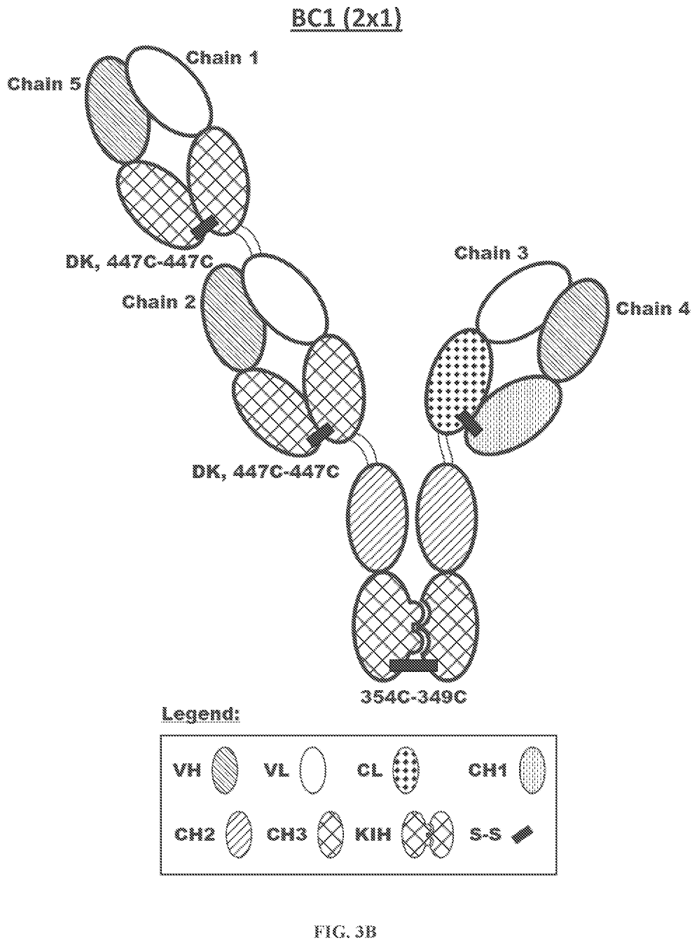

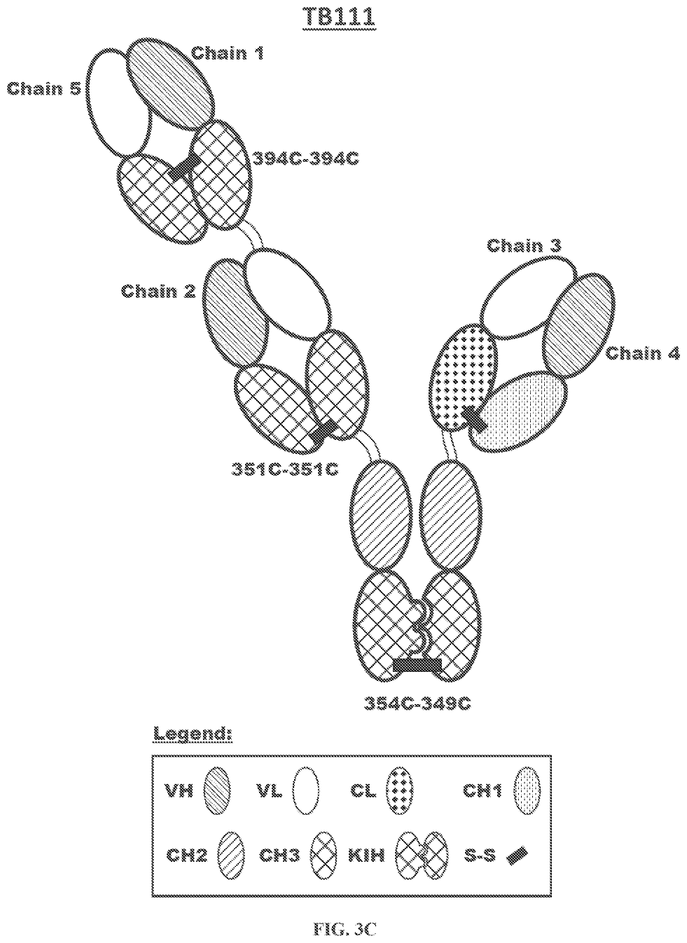

With reference to FIG. 3A, in various trivalent embodiments the binding molecules further comprise a fifth polypeptide chain, wherein (a) the first polypeptide chain further comprises a domain N and a domain O, wherein the domains are arranged, from N-terminus to C-terminus, in a N-O-A-B-D-E orientation, and wherein domain N has a variable region amino acid sequence, domain O has a constant region amino acid sequence; (b) the binding molecule further comprises a fifth polypeptide chain, comprising: a domain P and a domain Q, wherein the domains are arranged, from N-terminus to C-terminus, in a P-Q orientation, and wherein domain P has a variable region amino acid sequence and domain Q has a constant region amino acid sequence; and (c) the first and the fifth polypeptides are associated through an interaction between the N and the P domains and an interaction between the O and the Q domains to form the binding molecule. As schematized in FIG. 1, these trivalent embodiments are termed “2×1” trivalent constructs.

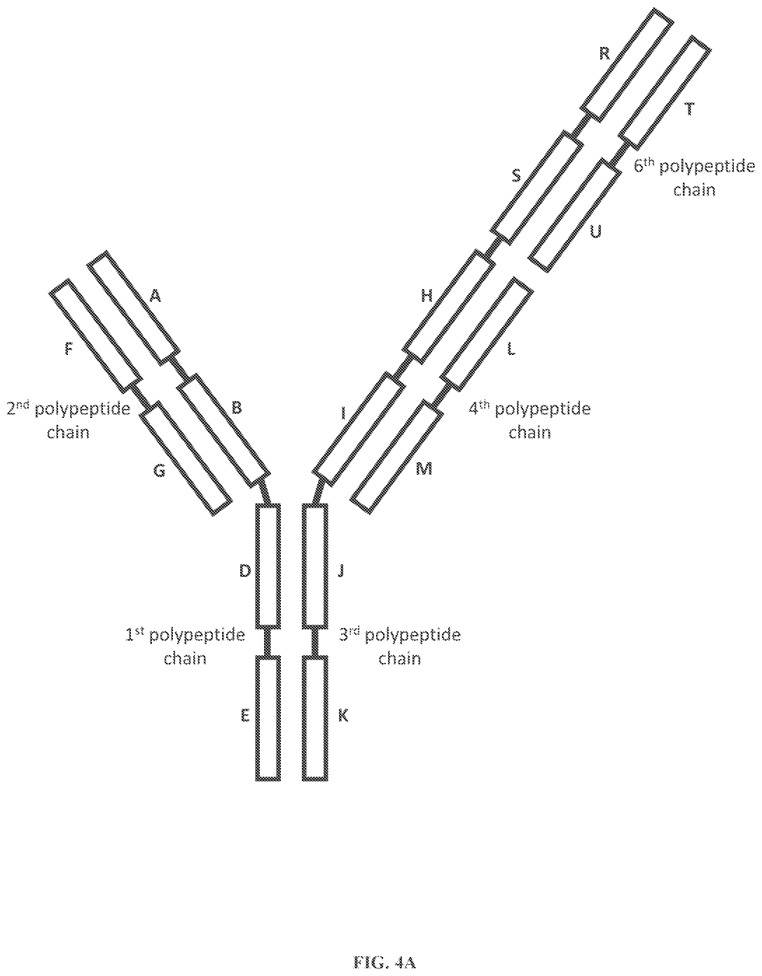

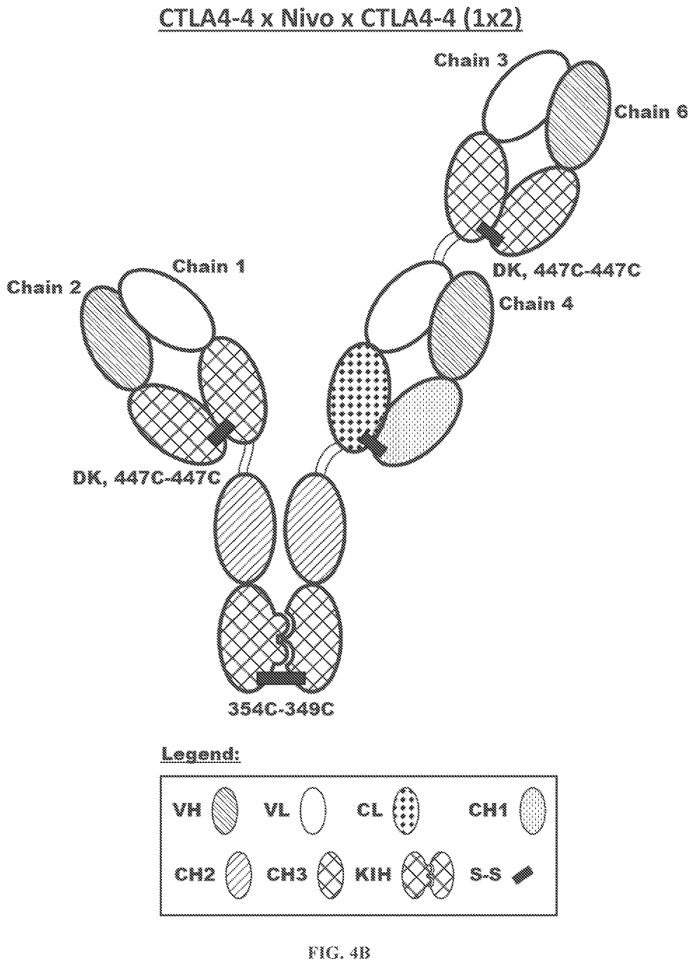

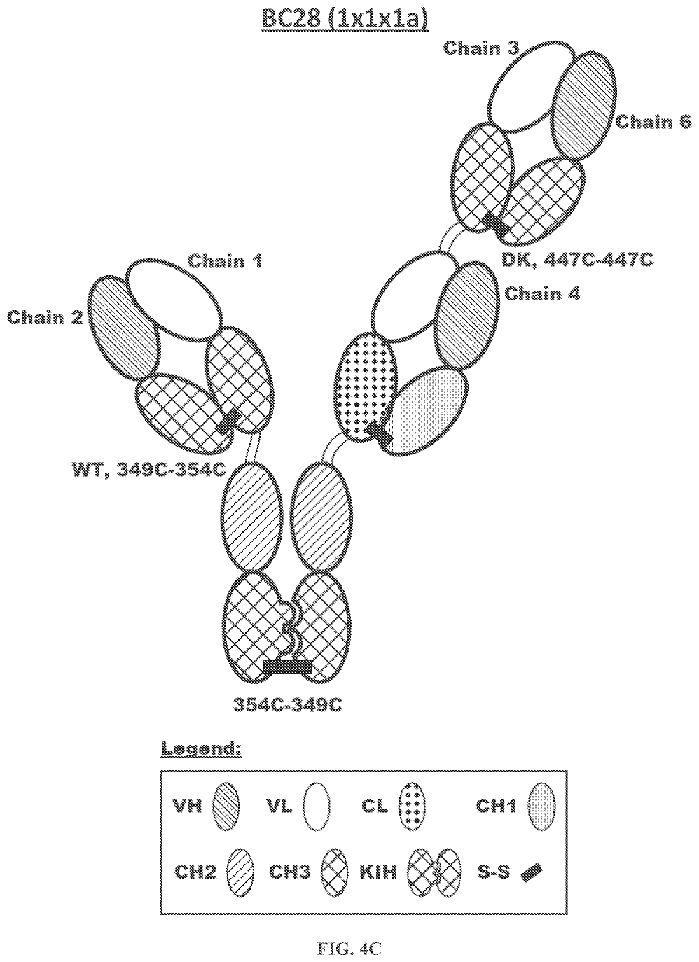

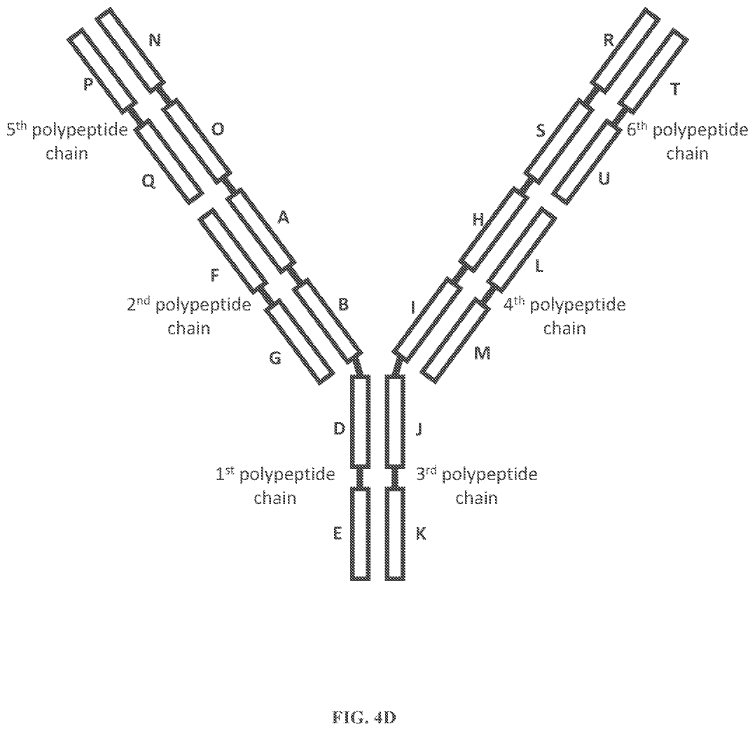

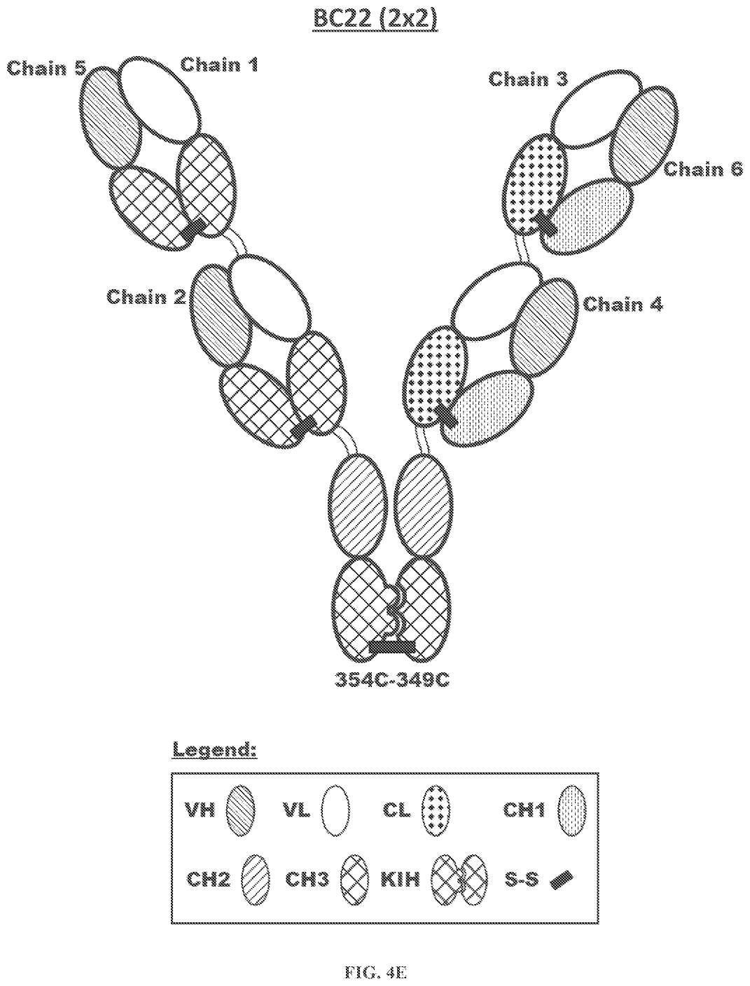

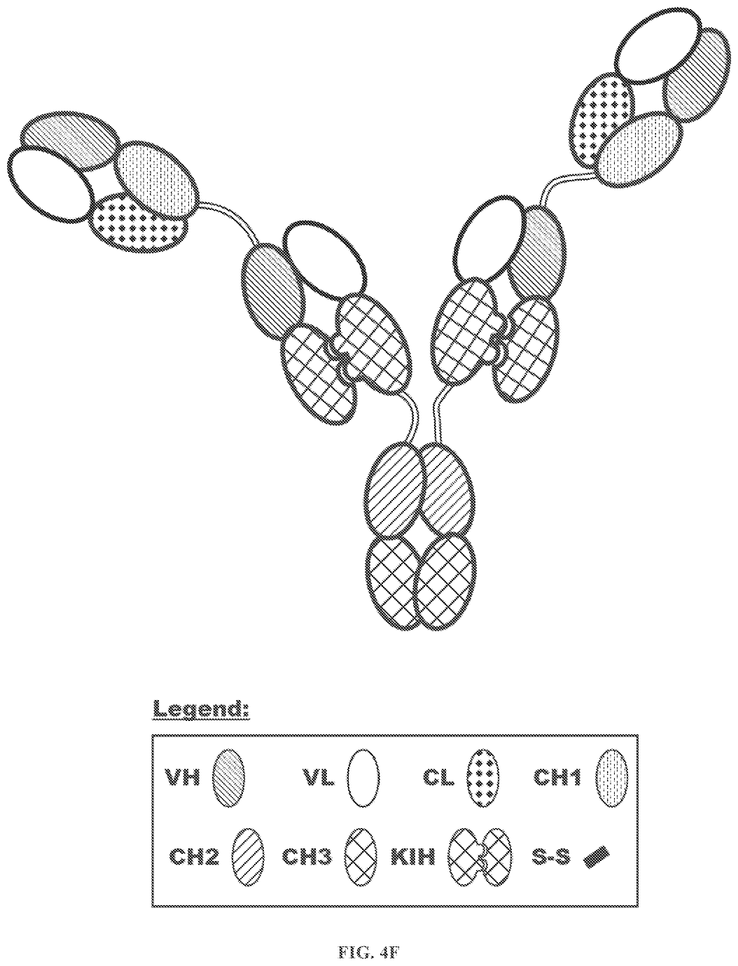

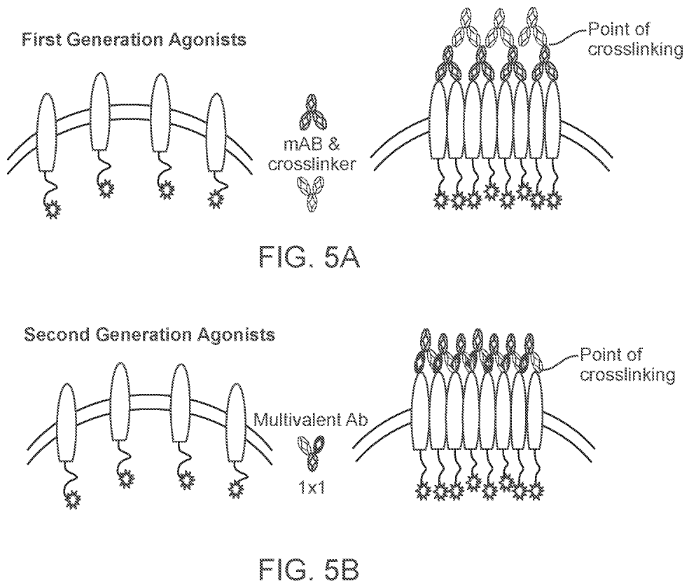

With reference to FIG. 4A, in a further series of trivalent embodiments, the binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has a variable region amino acid sequence and domain S has a constant domain amino acid sequence; (b) the binding molecule further comprises a sixth polypeptide chain, comprising: a domain T and a domain U, wherein the domains are arranged, from N-terminus to C-terminus, in a T-U orientation, and wherein domain T has a variable region amino acid sequence and domain U has a constant domain amino acid sequence; and (c) the third and the sixth polypeptides are associated through an interaction between the R and the T domains and an interaction between the S and the U domains to form the binding molecule. As schematized in FIG. 1, these trivalent embodiments are termed “1×2” trivalent constructs.

In a variety of embodiments, the domain O is connected to domain A through a peptide linker. In a variety of embodiments, the domain S is connected to domain H through a peptide linker. In a preferred embodiment, the peptide linker connecting either domain O to domain A or connecting domain S to domain H is a 6 amino acid GSGSGS peptide sequence, as described in more detail in Section 6.4.4.6.

### 6.4.2.1. Trivalent 2×1 Bispecific Constructs [2(A-A)×1(B)]

With reference to FIG. 3A, in a variety of embodiments the amino acid sequences of domain N and domain A are identical, the amino acid sequences of domain H is different from domains N and A; the amino acid sequences of domain O and domain B are identical, the amino acid sequences of domain I is different from domains O and B; the amino acid sequences of domain P and domain F are identical, the amino acid sequences of domain L is different from domains P and F; the amino acid sequences of domain Q and domain G are identical, the amino acid sequences of domain M is different from domains Q and G; and the interaction between the A domain and the F domain form a first antigen binding site specific for a first antigenic epitope, the interaction between the H domain and the L domain form a second antigen binding site specific for a second antigenic epitope, and the domain N and domain P form a third antigen binding site specific for the first antigenic epitope. These trivalent constructs are bispecific, with the A:F antigen binding site identical to the N:P antigen binding site, and the H:L binding site being specific for a second antigenic epitope.

In various embodiments, the construct contains one copy of the antigen binding site (ABS) specific for a first epitope of the target receptor. In some embodiments, the antigen binding site specific for a first epitope of the target receptor is an A:F antigen binding site. In some embodiments, the antigen binding site specific for a first epitope of the target receptor is an N:P antigen binding site. In some embodiments, the antigen binding site specific for a first epitope of the target receptor is an H:L antigen binding site. In various embodiments, the construct contains two copies of the antigen binding site specific for a first epitope of the target receptor. In some embodiments, a first antigen binding site specific for a first epitope of the target receptor is an A:F antigen binding site. In some embodiments, a first antigen binding site specific for a first epitope of the target receptor is an N:P antigen binding site. In some embodiments, a first antigen binding site specific for a first epitope of the target receptor is an H:L antigen binding site. In certain embodiments, a first antigen binding site specific for a first epitope of the target receptor is an A:F antigen binding site and a second antigen binding site specific for a first epitope of the target receptor is an N:P antigen binding site.

In various embodiments, the second antigenic epitope is a second epitope of the target receptor. In some aspects, the first epitope and the second epitope are non-overlapping epitopes. In other embodiments, the second antigenic epitope is an epitope of a second protein. In particular embodiments, the second protein is a second cell surface receptor.

### 6.4.2.2. Trivalent 2×1 Bispecific Constructs [2(A-B)×1(A)]

With reference to FIG. 3A, in a variety of embodiments the amino acid sequences of domain N and domain H are identical, the amino acid sequences of domain A is different from domains N and H, the amino acid sequences of domain O and domain I are identical, the amino acid sequences of domain B is different from domains O and I, the amino acid sequences of domain P and domain L are identical, the amino acid sequences of domain F is different from domains P and L, the amino acid sequences of domain Q and domain M are identical, the amino acid sequences of domain G is different from domains Q and M; and the interaction between the A domain and the F domain form a first antigen binding site specific for a first antigenic epitope, the interaction between the H domain and the L domain form a second antigen binding site specific for a second antigenic epitope, and the domain N and domain P form a third antigen binding site specific for the second antigenic epitope.

In various embodiments, the construct contains one copy of the antigen binding site (ABS) specific for a first epitope of the target receptor. In some embodiments, the antigen binding site specific for a first epitope of the target receptor is an A:F antigen binding site. In some embodiments, the antigen binding site specific for a first epitope of the target receptor is an N:P antigen binding site. In some embodiments, the antigen binding site specific for a first epitope of the target receptor is an H:L antigen binding site.

In various embodiments, the construct contains two copies of the antigen binding site specific for a first epitope of the target receptor. In some embodiments, a first antigen binding site specific for a first epitope of the target receptor is an A:F antigen binding site. In some embodiments, a first antigen binding site specific for a first epitope of the target receptor is an N:P antigen binding site. In some embodiments, a first antigen binding site specific for a first epitope of the target receptor is an H:L antigen binding site. In certain embodiments, a first antigen binding site specific for a first epitope of the target receptor is an N:P antigen binding site and a second antigen binding site specific for a first epitope of the target receptor is an H:L antigen binding site.

In various embodiments, the second antigenic epitope is a second epitope of the target receptor. In some aspects, the first epitope and the second epitope are non-overlapping epitopes. In other embodiments, the second antigenic epitope is an epitope of a second protein. In particular embodiments, the second protein is a second cell surface receptor.

### 6.4.2.3. Trivalent 2×1 Bispecific Constructs [2(B-A)×1(B)]

With reference to FIG. 3A, in a variety of embodiments the amino acid sequences of domain A and domain H are identical, the amino acid sequences of domain N is different from domains A and H, the amino acid sequences of domain B and domain I are identical, the amino acid sequences of domain O is different from domains B and I, the amino acid sequences of domain F and domain L are identical, the amino acid sequences of domain P is different from domains F and L, the amino acid sequences of domain G and domain M are identical, the amino acid sequences of domain Q is different from domains G and M; and the interaction between the A domain and the F domain form a first antigen binding site specific for a first antigenic epitope, the interaction between the H domain and the L domain form a second antigen binding site specific for a second antigenic epitope, and the domain N and domain P form a third antigen binding site specific for the second antigenic epitope.

In various embodiments, the construct contains one copy of the antigen binding site (ABS) specific for a first epitope of the target receptor. In some embodiments, the antigen binding site specific for a first epitope of the target receptor is an A:F antigen binding site. In some embodiments, the antigen binding site specific for a first epitope of the target receptor is an N:P antigen binding site. In some embodiments, the antigen binding site specific for a first epitope of the target receptor is an H:L antigen binding site.

In various embodiments, the construct contains two copies of the antigen binding site specific for a first epitope of the target receptor. In some embodiments, a first antigen binding site specific for a first epitope of the target receptor is an A:F antigen binding site. In some embodiments, a first antigen binding site specific for a first epitope of the target receptor is an N:P antigen binding site. In some embodiments, a first antigen binding site specific for a first epitope of the target receptor is an H:L antigen binding site. In certain embodiments, a first antigen binding site specific for a first epitope of the target receptor is an N:P antigen binding site and a second antigen binding site specific for a first epitope of the target receptor is an H:L antigen binding site.

In various embodiments, the second antigenic epitope is a second epitope of the target receptor. In some aspects, the first epitope and the second epitope are non-overlapping epitopes. In other embodiments, the second antigenic epitope is an epitope of a second protein. In particular embodiments, the second protein is a second cell surface receptor.

### 6.4.2.4. Trivalent 2×1 Trispecific Constructs [2(A-B)×1(C)]

With reference to FIG. 3A, in a variety of embodiments, the amino acid sequences of domain N, domain A, and domain H are different, the amino acid sequences of domain O, domain B, and domain I are different, the amino acid sequences of domain P, domain F, and domain L are different, and the amino acid sequences of domain Q, domain G, and domain M are different; and the interaction between the A domain and the F domain form a first antigen binding site specific for a first antigenic epitope, the interaction between the H domain and the L domain form a second antigen binding site specific for a second antigenic epitope, and the domain N and domain P form a third antigen binding site specific for a third antigenic epitope. These are trispecific trivalent (2×1) constructs.

In certain embodiments, domain O has a constant region sequence that is a CL from a kappa light chain and domain Q has a constant region sequence that is a CH1 from an IgG1 isotype, as discussed in more detail in Sections 6.4.1.9.2 and 6.4.1.9.1, respectively. In a preferred embodiment, domain O and domain Q have CH3 sequences such that they specifically associate with each other, as discussed in more detail in Section 6.4.1.15.

In various embodiments, the antigen binding site specific for a first epitope of the target receptor is an A:F antigen binding site. In some embodiments, the antigen binding site specific for a first epitope of the target receptor is an N:P antigen binding site. In some embodiments, the antigen binding site specific for a first epitope of the target receptor is an H:L antigen binding site.

In some embodiments, the second antigenic target is a second epitope of the target receptor. In some aspects, the first epitope and the second epitope are non-overlapping epitopes. In some embodiments, the second antigenic target is a first epitope of a second protein. In some embodiments, the third antigenic target is a third epitope of the target receptor. In some embodiments, the third antigenic target is a second epitope of a second protein. In some aspects, the first epitope of the second protein and the second epitope of the second protein are non-overlapping epitopes. In some embodiments, the third antigenic target is a first epitope of a third protein. In some embodiments, the second protein or third protein is a second cell surface receptor or third cell surface receptor.

### 6.4.2.5. Trivalent 2×1 Monospecific Constructs

With reference to FIG. 3A, in a variety of embodiments the amino acid sequences of domain N, domain A, and domain H are identical, the amino acid sequences of domain O and domain B are identical; the amino acid sequences of domain P, domain F, and domain L are identical; and the amino acid sequences of domain Q and domain G are identical; and the interaction between the A domain and the F domain form a first antigen binding site specific for a first antigenic epitope, the interaction between the H domain and the L domain form a second antigen binding site specific for the first antigenic epitope, and the domain N and domain P form a third antigen binding site specific for the first antigenic epitope. These trivalent constructs are monospecific; all three antigen binding sites are specific for a first epitope of the target receptor.

With reference to FIG. 3A, in another series of embodiments, the amino acid sequences of domain N, domain A, and domain H are identical, the amino acid sequences of domain O, domain B, and domain I are identical, the amino acid sequences of domain P, domain F, and domain L are identical, and the amino acid sequences of domain Q, domain G, and domain M are identical; and the interaction between the A domain and the F domain form a first antigen binding site specific for a first antigenic epitope, the interaction between the H domain and the L domain form a second antigen binding site specific for the first antigenic epitope, and the domain N and domain P form a third antigen binding site specific for the first antigenic epitope. These trivalent constructs are monospecific; all three antigen binding sites are specific for a first epitope of the target receptor.

### 6.4.2.6. Trivalent 1×2 Bispecific Constructs [1(A)×2(B-A)]

With reference to FIG. 4A, in a variety of embodiments, the amino acid sequences of domain R and domain A are identical, the amino acid sequences of domain H is different from domain R and A, the amino acid sequences of domain S and domain B are identical, the amino acid sequences of domain I is different from domain S and B, the amino acid sequences of domain T and domain F are identical, the amino acid sequences of domain L is different from domain T and F, the amino acid sequences of domain U and domain G are identical, the amino acid sequences of domain M is different from domain U and G and the interaction between the A domain and the F domain form a first antigen binding site specific for a first antigenic epitope, the interaction between the H domain and the L domain form a second antigen binding site specific for a second antigenic epitope, and the domain R and domain T form a third antigen binding site specific for the first antigenic epitope.

In various embodiments, the trivalent construct contains one copy of the antigen binding site (ABS) specific for a first epitope of the target receptor. In some embodiments, the antigen binding site specific for a first epitope of the target receptor is an A:F antigen binding site. In some embodiments, the antigen binding site specific for a first epitope of the target receptor is an R:T antigen binding site. In some embodiments, the antigen binding site specific for a first epitope of the target receptor is an H:L antigen binding site.

In various embodiments, the bispecific trivalent construct contains two copies of the antigen binding site specific for a first epitope of the target receptor. In some embodiments, a first antigen binding site specific for a first epitope of the target receptor is an A:F antigen binding site. In some embodiments, a first antigen binding site specific for a first epitope of the target receptor is an R:T antigen binding site. In some embodiments, a first antigen binding site specific for a first epitope of the target receptor is an H:L antigen binding site.

In some embodiments, a first antigen binding site specific for a first epitope of the target receptor is an A:F antigen binding site and a second antigen binding site specific for a first epitope of the target receptor is an R:T antigen binding site.

In various embodiments, the second antigenic target is a second epitope of the target receptor. In some aspects, the first epitope and the second epitope are non-overlapping epitopes. In certain embodiments, the second antigenic target is an epitope of a second protein. In particular embodiments, the second protein is a second cell surface receptor.

### 6.4.2.7. Trivalent 1×2 Bispecific Constructs [1(A)×2(B-B)]

With reference to FIG. 4A, in a variety of embodiments, the amino acid sequences of domain R and domain H are identical, the amino acid sequences of domain A is different from domain R and H, the amino acid sequences of domain S and domain I are identical, the amino acid sequences of domain B is different from domain S and I, the amino acid sequences of domain T and domain L are identical, the amino acid sequences of domain F is different from domain T and L, the amino acid sequences of domain U and domain M are identical, the amino acid sequences of domain G is different from domain U and M and the interaction between the A domain and the F domain form a first antigen binding site specific for a first antigenic epitope, the interaction between the H domain and the L domain form a second antigen binding site specific for a second antigenic epitope, and the domain R and domain T form a third antigen binding site specific for the second antigenic epitope.

In various embodiments, the trivalent construct contains one copy of the antigen binding site (ABS) specific for a first epitope of the target receptor. In some embodiments, the antigen binding site specific for a first epitope of the target receptor is an A:F antigen binding site. In some embodiments, the antigen binding site specific for a first epitope of the target receptor is an R:T antigen binding site. In some embodiments, the antigen binding site specific for a first epitope of the target receptor is an H:L antigen binding site.

In various embodiments, the bispecific trivalent construct contains two copies of the antigen binding site specific for a first epitope of the target receptor. In some embodiments, a first antigen binding site specific for a first epitope of the target receptor is an A:F antigen binding site. In some embodiments, a first antigen binding site specific for a first epitope of the target receptor is an R:T antigen binding site. In some embodiments, a first antigen binding site specific for a first epitope of the target receptor is an H:L antigen binding site.

In some embodiments, a first antigen binding site specific for a first epitope of the target receptor is an R:T antigen binding site and a second antigen binding site specific for a first epitope of the target receptor is an H:L antigen binding site.

In various embodiments, the second antigenic target is a second epitope of the target receptor. In some aspects, the first epitope and the second epitope are non-overlapping epitopes. In certain embodiments, the second antigenic target is an epitope of a second protein. In particular embodiments, the second protein is a second cell surface receptor.

### 6.4.2.8. Trivalent 1×2 Trispecific Constructs [1(A)×2(B-C)]

With reference to FIG. 4A, in a variety of embodiments, the amino acid sequences of domain R, domain A, and domain H are different, the amino acid sequences of domain S, domain B, and domain I are different, the amino acid sequences of domain T, domain F, and domain L are different, and the amino acid sequences of domain U, domain G, and domain M are different; and the interaction between the A domain and the F domain form a first antigen binding site specific for a first antigenic epitope, the interaction between the H domain and the L domain form a second antigen binding site specific for a second antigenic epitope, and the domain R and domain T form a third antigen binding site specific for a third antigenic epitope. These are trispecific trivalent (1×2) constructs.

In particular embodiments, domain S has a constant region sequence that is a CL from a kappa light chain and domain U has a constant region sequence that is a CH1 from an IgG1 isotype, as discussed in more detail in Sections 6.4.1.9.2 and 6.4.1.9.1, respectively. In a preferred embodiment, domain S and domain U have CH3 sequences such that they specifically associate with each other, as discussed in more detail in Section 6.4.1.15.

In various embodiments, the antigen binding site specific for a first epitope of the target receptor is an A:F antigen binding site. In various embodiments, the antigen binding site specific for a first epitope of the target receptor is an R:T antigen binding site. In various embodiments, the antigen binding site specific for a first epitope of the target receptor is an H:L antigen binding site.

In some embodiments, the second antigenic target is a second epitope of the target receptor. In some aspects, the first epitope and the second epitope are non-overlapping epitopes. In some embodiments, the second antigenic target is a first epitope of a second protein. In some embodiments, the third antigenic target is a third epitope of the target receptor. In some embodiments, the third antigenic target is a second epitope of a second protein. In some aspects, the first epitope of the second protein and the second epitope of the second protein are non-overlapping epitopes. In some embodiments, the third antigenic target is a first epitope of a third protein.

In a variety of embodiments, the second protein or third protein is a second or third cell surface receptor.

### 6.4.2.1. Trivalent 1×2 Monospecific Constructs

With reference to FIG. 4A, in a variety of embodiments, the amino acid sequences of domain R, domain A, and domain H are identical, the amino acid sequences of domain S and domain B are identical, the amino acid sequences of domain T, domain F, and domain L are identical, and the amino acid sequences of domain U and domain G are identical; and the interaction between the A domain and the F domain form a first antigen binding site specific for a first antigenic epitope, the interaction between the H domain and the L domain form a second antigen binding site specific for the first antigenic epitope, and the domain R and domain T form a third antigen binding site specific for the first antigenic epitope. These trivalent constructs are monospecific; all three antigen binding sites are specific for a first epitope of the target receptor.

### 6.4.3. Tetravalent 2×2 Binding Molecules

In a variety of embodiments, the binding molecules have 4 antigen binding sites and are therefore termed “tetravalent.”

With reference to FIG. 4D, in a further series of embodiments, the binding molecules further comprise a fifth and a sixth polypeptide chain, wherein (a) the first polypeptide chain further comprises a domain N and a domain O, wherein the domains are arranged, from N-terminus to C-terminus, in a N-O-A-B-D-E orientation; (b) the third polypeptide chain further comprises a domain R and a domain S, wherein the domains are arranged, from N-terminus to C-terminus, in a R-S-H-I-J-K orientation; (c) the binding molecule further comprises a fifth and a sixth polypeptide chain, wherein the fifth polypeptide chain comprises a domain P and a domain Q, wherein the domains are arranged, from N-terminus to C-terminus, in a P-Q orientation, and the sixth polypeptide chain comprises a domain T and a domain U, wherein the domains are arranged, from N-terminus to C-terminus, in a T-U orientation; and (d) the first and the fifth polypeptides are associated through an interaction between the N and the P domains and an interaction between the O and the Q domains, and the third and the sixth polypeptides are associated through an interaction between the R and the T domains and an interaction between the S and the U domains to form the binding molecule.

In a variety of embodiments, the domain O is connected to domain A through a peptide linker and the domain S is connected to domain H through a peptide linker. In a preferred embodiment, the peptide linker connecting domain O to domain A and connecting domain S to domain H is a 6 amino acid GSGSGS peptide sequence, as described in more detail in Section 6.4.4.6.

### 6.4.3.1. Tetravalent 2×2 Bispecific Constructs

With reference to FIG. 4D, in a series of tetravalent 2×2 bispecific binding molecules, the amino acid sequences of domain N and domain A are identical, the amino acid sequences of domain H and domain R are identical, the amino acid sequences of domain O and domain B are identical, the amino acid sequences of domain I and domain S are identical, the amino acid sequences of domain P and domain F are identical, the amino acid sequences of domain L and domain T are identical, the amino acid sequences of domain Q and domain G are identical, the amino acid sequences of domain M and domain U are identical; and wherein the interaction between the A domain and the F domain form a first antigen binding site specific for a first antigen, the domain N and domain P form a second antigen binding site specific for the first antigen, the interaction between the H domain and the L domain form a third antigen binding site specific for a second antigen, and the interaction between the R domain and the T domain form a fourth antigen binding site specific for the second antigen.

With reference to FIG. 4D, in another series of tetravalent 2×2 bispecific binding molecules, the amino acid sequences of domain H and domain A are identical, the amino acid sequences of domain N and domain R are identical, the amino acid sequences of domain I and domain B are identical, the amino acid sequences of domain O and domain S are identical, the amino acid sequences of domain L and domain F are identical, the amino acid sequences of domain P and domain T are identical, the amino acid sequences of domain M and domain G are identical, the amino acid sequences of domain Q and domain U are identical; and wherein the interaction between the A domain and the F domain form a first antigen binding site specific for a first antigen, the domain N and domain P form a second antigen binding site specific for a second antigen, the interaction between the H domain and the L domain form a third antigen binding site specific for the first antigen, and the interaction between the R domain and the T domain form a fourth antigen binding site specific for the second antigen.

In a particular embodiment, FIG. 4E shows the overall architecture of a 2×2 tetravalent bispecific construct “BC22-2×2”. The 2×2 tetravalent bispecific represents a “BC1” scaffold, as described in greater detail in Section 6.4.5.1, with duplications of each variable domain-constant domain segment.

### 6.4.3.2. Tetravalent 2×2 Monospecific Constructs

With reference to FIG. 4D, in a variety of embodiments, the amino acid sequences of domain N, domain A, domain H and domain R are identical, the amino acid sequences of domain O and domain B are identical, the amino acid sequences of domain I and domain S are identical, the amino acid sequences of domain P, domain F, domain L, and domain T are identical, the amino acid sequences of domain Q and domain G are identical, the amino acid sequences of domain M and domain U are identical; and wherein the interaction between the A domain and the F domain form a first antigen binding site specific for a first antigen, the domain N and domain P form a second antigen binding site specific for the first antigen, the interaction between the H domain and the L domain form a third antigen binding site specific for the first antigen, and the interaction between the R domain and the T domain form a fourth antigen binding site specific for the first antigen.

With reference to FIG. 4D, in another series of tetravalent 2×2 monospecific embodiments, the amino acid sequences of domain N, domain A, domain H and domain R are identical, the amino acid sequences of domain I and domain B are identical, the amino acid sequences of domain O and domain S are identical, the amino acid sequences of domain P, domain F, domain L, and domain T are identical, the amino acid sequences of domain M and domain G are identical, the amino acid sequences of domain Q and domain U are identical; and wherein the interaction between the A domain and the F domain form a first antigen binding site specific for a first antigen, the domain N and domain P form a second antigen binding site specific for the first antigen, the interaction between the H domain and the L domain form a third antigen binding site specific for the first antigen, and the interaction between the R domain and the T domain form a fourth antigen binding site specific for the first antigen.

### 6.4.4. Domain Junctions

### 6.4.4.1. Junctions Connecting VL and CH3 Domains

In a variety of embodiments, the amino acid sequence that forms a junction between the C-terminus of a VL domain and the N-terminus of a CH3 domain is an engineered sequence. In certain embodiments, one or more amino acids are deleted or added in the C-terminus of the VL domain. In certain embodiments, the junction connecting the C-terminus of a VL domain and the N-terminus of a CH3 domain is one of the sequences described in Table 1 below. In particular embodiments, A111 is deleted in the C-terminus of the VL domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In certain embodiments, one or more amino acids are deleted or added to both the C-terminus of the VL domain and the N-terminus of the CH3 domain. In particular embodiments, A111 is deleted in the C-terminus of the VL domain and P343 is deleted in the N-terminus of the CH3 domain. In a preferred embodiment, A111 and V110 are deleted in the C-terminus of the VL domain. In another preferred embodiment, A111 and V110 are deleted in the C-terminus of the VL domain and the N-terminus of the CH3 domain has a P343V mutation.

TABLE 1
Variants of Variable Domain/Constant Domain
Junctions for 1^{st} Polypeptide Chain

| | VL | | | | | | CH3 | | | | |
|---|---|---|---|---|---|---|---|---|---|---|---|
| Variant | 106 | 107 | 108 | 109 | 110 | 111 | 343 | 344 | 345 | 346 | Sequence |
| BC1 | I | K | R | T | | | P | R | E | P | IKRTPREP |
| | | | | | | | | | | | (SEQ ID |
| | | | | | | | | | | | NO: 36) |
| BC13 | I | K | R | T | | | P | R | E | P | IKRTPREP |
| | | | | | | | | | | | (SEQ ID |
| | | | | | | | | | | | NO: 36) |
| BC14 | I | K | R | T | | | P | R | E | P | IKRTPREP |
| | | | | | | | | | | | (SEQ ID |
| | | | | | | | | | | | NO: 36) |
| BC15 | I | K | R | T | V | | | R | E | P | IKRTVREP |
| | | | | | | | | | | | (SEQ ID |
| | | | | | | | | | | | NO: 37) |
| BC16 | I | K | R | T | | | | R | E | P | IKRTREP |
| | | | | | | | | | | | (SEQ ID |
| | | | | | | | | | | | NO: 38) |
| BC17 | I | K | R | T | V | | P | R | E | P | IKRTVPREP |
| | | | | | | | | | | | (SEQ ID |
| | | | | | | | | | | | NO: 39) |
| BC24 | I | K | R | T | | | P | R | E | P | IKRTPREP |
| | | | | | | | | | | | (SEQ ID |
| | | | | | | | | | | | NO: 36) |
| BC25 | I | K | R | T | | | P | R | E | P | IKRTPREP |
| | | | | | | | | | | | (SEQ ID |
| | | | | | | | | | | | NO: 36) |
| BC26 | I | K | R | T | V | A | | | E | P | IKRTVAEP |
| | | | | | | | | | | | (SEQ ID |
| | | | | | | | | | | | NO: 40) |
| BC27 | I | K | R | T | V | A | P | R | E | P | IKRTVAPREP |
| | | | | | | | | | | | (SEQ ID |
| | | | | | | | | | | | NO: 41) |
| BC44 | I | K | R | T | | | V | R | E | P | IKRTVREP |
| | | | | | | | | | | | (SEQ ID |
| | | | | | | | | | | | NO: 37) |
| BC45 | I | K | R | T | | | P | R | E | P | IKRTPREP |
| | | | | | | | | | | | (SEQ ID |
| | | | | | | | | | | | NO: 36) |
| BC5 | I | K | R | T | | | P | R | E | P | IKRTPREP |
| | | | | | | | | | | | (SEQ ID |
| | | | | | | | | | | | NO: 36) |
| BC6 | I | K | R | T | | | P | R | E | P | IKRTPREP |
| | | | | | | | | | | | (SEQ ID |
| | | | | | | | | | | | NO: 36) |
| BC28 | I | K | R | T | | | P | R | E | P | IKRTPREP |
| | | | | | | | | | | | (SEQ ID |
| | | | | | | | | | | | NO: 36) |
| BC30 | I | K | R | T | | | P | R | E | P | IKRTPREP |
| | | | | | | | | | | | (SEQ ID |
| | | | | | | | | | | | NO: 36) |

### 6.4.4.2. Junctions Connecting VH and CH3 Domains

In a variety of embodiments, the amino acid sequence that forms a junction between the C-terminus of a VH domain and the N-terminus of a CH3 domain is an engineered sequence. In certain embodiments, one or more amino acids are deleted or added in the C-terminus of the VH domain. In certain embodiments, the junction connecting the C-terminus of a VH domain and the N-terminus of the CH3 domain is one of the sequences described in Table 2 below. In particular embodiments, K117 and G118 are deleted in the C-terminus of the VH domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In particular embodiments, P343, R344, and E345 are deleted in the N-terminus of the CH3 domain. In certain embodiments, one or more amino acids are deleted or added to both the C-terminus of the VH domain and the N-terminus of the CH3 domain. In a preferred embodiment, T116, K117, and G118 are deleted in the C-terminus of the VH domain.

TABLE 2
Variants of Variable Domain/Constant Domain Junctions for 2^{nd} Polypeptide Chain

| | VH | | | | | | | CH3 | | | | |
|---|---|---|---|---|---|---|---|---|---|---|---|---|
| Variant | 112 | 113 | 114 | 115 | 116 | 117 | 118 | 343 | 344 | 345 | 346 | Sequence |
| BC1 | S | S | A | S | | | | P | R | E | P | SSASPREP |
| | | | | | | | | | | | | (SEQ ID NO: 42) |
| BC13 | S | S | A | S | T | | | | R | E | P | SSASTREP |
| | | | | | | | | | | | | (SEQ ID NO: 43) |
| BC14 | S | S | A | S | T | | | P | R | E | P | SSASTPREP |
| | | | | | | | | | | | | (SEQ ID NO: 44) |
| BC15 | S | S | A | S | | | | P | R | E | P | SSASPREP |
| | | | | | | | | | | | | (SEQ ID NO: 42) |
| BC16 | S | S | A | S | | | | P | R | E | P | SSASPREP |
| | | | | | | | | | | | | (SEQ ID NO: 42) |
| BC17 | S | S | A | S | | | | P | R | E | P | SSASPREP |
| | | | | | | | | | | | | (SEQ ID NO: 42) |
| BC24 | S | S | A | S | T | K | G | | | E | P | SSASTKGEP |
| | | | | | | | | | | | | (SEQ ID NO: 45) |
| BC25 | S | S | A | S | T | K | G | | R | E | P | SSASTKGREP |
| | | | | | | | | | | | | (SEQ ID NO: 46) |
| BC26 | S | S | A | S | | | | P | R | E | P | SSASPREP |
| | | | | | | | | | | | | (SEQ ID NO: 42) |
| BC27 | S | S | A | S | | | | P | R | E | P | SSASPREP |
| | | | | | | | | | | | | (SEQ ID NO: 42) |
| BC44 | S | S | A | S | | | | P | R | E | P | SSASPREP |
| | | | | | | | | | | | | (SEQ ID NO: 42) |
| BC45 | S | S | A | S | | | | P | R | E | P | SSASPREP |
| | | | | | | | | | | | | (SEQ ID NO: 42) |
| BC5 | S | S | A | S | | | | P | R | E | P | SSASPREP |
| | | | | | | | | | | | | (SEQ ID NO: 42) |
| BC6 | S | S | A | S | | | | P | R | E | P | SSASPREP |
| | | | | | | | | | | | | (SEQ ID NO: 42) |
| BC28 | S | S | A | S | | | | P | R | E | P | SSASPREP |
| | | | | | | | | | | | | (SEQ ID NO: 42) |
| BC30 | S | S | A | S | | | | P | R | E | P | SSASPREP |
| | | | | | | | | | | | | (SEQ ID NO: 42) |

### 6.4.4.3. Junctions Connecting Constant Region Domain C-Terminus to CH2 N-Terminus (Hinge)

In the binding molecules described herein, the N-terminus of the CH2 domain has a “hinge” region amino acid sequence. As used herein, hinge regions are sequences of an antibody heavy chain that link the N-terminal variable domain-constant domain segment of an antibody (e.g., the segment corresponding to domain A connected to domain B) and a CH2 domain of an antibody. In addition, the hinge region typically provides both flexibility between the N-terminal variable domain-constant domain segment and CH2 domain, as well as amino acid sequence motifs that form disulfide bridges between heavy chains (e.g. the first and the third polypeptide chains). As used herein, the hinge region amino acid sequence is SEQ ID NO:18.

In embodiments wherein the constant region domain is a CH3 amino acid sequence, the CH3 amino acid sequence is extended at the C-terminus at the junction between the C-terminus of the CH3 domain and the N-terminus of a CH2 domain. In certain embodiments, a CH3 amino acid sequence is extended at the C-terminus at the junction between the C-terminus of the CH3 domain and a hinge region, which in turn is connected to the N-terminus of a CH2 domain. In a preferred embodiment, the CH3 amino acid sequence is extended by inserting a PGK tripeptide sequence followed by the DKTHT motif of an IgG1 hinge region.

In a particular embodiment, the extension at the C-terminus of the CH3 domain incorporates amino acid sequences that can form a disulfide bond with orthogonal C-terminal extension of another CH3 domain. In a preferred embodiment, the extension at the C-terminus of the CH3 domain incorporates a KSC tripeptide sequence that is followed by the DKTHT motif of an IgG1 hinge region that forms a disulfide bond with orthogonal C-terminal extension of another CH3 domain that incorporates a GEC motif of a kappa light chain.

### 6.4.4.4. Junctions Connecting CL C-Terminus and CH2 N-Terminus (Hinge)

In a variety of embodiments, a CL amino acid sequence is connected through its C-terminus to a hinge region, which in turn is connected to the N-terminus of a CH2 domain. Hinge region sequences are described in greater detail in Section 6.4.4. In a preferred embodiment, the hinge region amino acid sequence is SEQ ID NO:18.

### 6.4.4.5. Junctions Connecting CH2 C-Terminus to Constant Region Domain

In a variety of embodiments, a CH2 amino acid sequence is connected through its C-terminus to the N-terminus of a constant region domain. Constant regions are described in more detail in Section 6.4.1.5. In a preferred embodiment, the CH2 sequence is connected to a CH3 sequence via its endogenous sequence. In other embodiments, the CH2 sequence is connected to a CH1 or CL sequence. Examples discussing connecting a CH2 sequence to a CH1 or CL sequence are described in more detail in U.S. Pat. No. 8,242,247, which is hereby incorporated in its entirety.

### 6.4.4.6. Junctions Connecting Domain O to Domain A or Domain S to Domain H on Trivalent Molecules

In a variety of embodiments, heavy chains of antibodies (e.g. the first and third polypeptide chains) are extended at their N-terminus to include additional domains that provide additional ABSs. With reference to FIG. 3A, FIG. 4A, in certain embodiments, the C-terminus of the constant region domain amino acid sequence of a domain O and/or a domain S is connected to the N-terminus of the variable region domain amino acid sequence of a domain A and/or a domain H, respectively. In some preferred embodiments, the constant region domain is a CH3 amino acid sequence and the variable region domain is a VL amino acid sequence. In some preferred embodiments, the constant region domain is a CL amino acid sequence and the variable region domain is a VL amino acid sequence. In certain embodiments, the constant region domain is connected to the variable region domain through a peptide linker. In a preferred embodiment, the peptide linker is a 6 amino acid GSGSGS peptide sequence.

In a variety of embodiments, light chains of antibodies (e.g. the second and fourth polypeptide chains) are extended at their N-terminus to include additional variable domain-constant domain segments of an antibody. In certain embodiments, the constant region domain is a CH1 amino acid sequence and the variable region domain is a VH amino acid sequence.

### 6.4.5. Exemplary Bivalent Binding Molecules

### 6.4.5.1. “BC1” Bivalent (1×1) Format

With reference to FIG. 2A and illustrated in FIG. 2B, in a series of embodiments the bivalent binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a T366K mutation and a C-terminal extension incorporating a KSC tripeptide sequence that is followed by the DKTHT motif of an IgG1 hinge region, domain D has a human IgG1 CH2 amino acid sequence, and domain E has human IgG1 CH3 amino acid with a S354C and T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a L351D mutation and a C-terminal extension incorporating a GEC amino acid disulfide motif; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule.

In particular embodiments, domain A and domain F form a first antigen binding site specific for a first antigen, domain H and domain L form a second antigen binding site specific for a second antigen.

In particular embodiments, domain A and domain F form a first antigen binding site specific for a first antigen, domain H and domain L form a second antigen binding site specific for the first antigen.

In preferred embodiments, the first polypeptide chain has a scaffold sequence SEQ ID NO:23, the second polypeptide chain has a scaffold sequence SEQ ID NO:24, the third polypeptide chain has a scaffold sequence SEQ ID NO:25, and the fourth polypeptide chain has a scaffold sequence SEQ ID NO:26, as described in more details in Section 6.10.2.1.

### 6.4.5.2. “BC6” Bivalent (1×1) Format

With reference to FIG. 2A and illustrated in FIG. 2C, in a series of embodiments the bivalent binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a C-terminal extension incorporating a KSC tripeptide sequence that is followed by the DKTHT motif of an IgG1 hinge region, domain D has a human IgG1 CH2 amino acid sequence, and domain E has human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a C-terminal extension incorporating a GEC amino acid disulfide motif; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule.

In particular embodiments, domain A and domain F form a first antigen binding site specific for a first antigen, domain H and domain L form a second antigen binding site specific for a second antigen.

In particular embodiments, domain A and domain F form a first antigen binding site specific for a first antigen, domain H and domain L form a second antigen binding site specific for the first antigen.

### 6.4.5.3. “BC28” Bivalent (1×1) Format

With reference to FIG. 2A and illustrated in FIG. 2D, in a series of embodiments the bivalent (1×1) binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation and a C-terminal extension incorporating a PGK tripeptide sequence that is followed by the DKTHT motif of an IgG1 hinge region, domain D has a human IgG1 CH2 amino acid sequence, and domain E has a human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule.

In particular embodiments, domain A and domain F form a first antigen binding site specific for a first antigen, domain H and domain L form a second antigen binding site specific for a second antigen.

In particular embodiments, domain A and domain F form a first antigen binding site specific for a first antigen, domain H and domain L form a second antigen binding site specific for the first antigen.

### 6.4.5.4. “BC44” Bivalent (1×1) Format

With reference to FIG. 2A and illustrated in FIG. 2E, in a series of embodiments the bivalent (1×1) binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation, a P343V mutation, and a C-terminal extension incorporating a PGK tripeptide sequence that is followed by the DKTHT motif of an IgG1 hinge region, domain D has a human IgG1 CH2 amino acid sequence, and domain E has human IgG1 CH3 amino acid with a S354C mutation and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, T366S, L368A, and a Y407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the binding molecule.

In particular embodiments, domain A and domain F form a first antigen binding site specific for a first antigen, domain H and domain L form a second antigen binding site specific for a second antigen.

In particular embodiments, domain A and domain F form a first antigen binding site specific for a first antigen, domain H and domain L form a second antigen binding site specific for the first antigen.

### 6.4.6. Exemplary Trivalent Binding Molecules

### 6.4.6.1. Trivalent 2×1 Bispecific B-Body “BC1-2×1”

With reference to Section 6.4.5.1 and FIG. 3A, in a series of embodiments, FIG. 3B illustrates the salient features of a trivalent 2×1 bispecific B-Body binding molecule further comprising a fifth polypeptide chain and as described below:

1st polypeptide chain
Domain N=VL
Domain O=CH3 (T366K, 447C)
Domain A=VL
Domain B=CH3 (T366K, 447C)
Domain D=CH2
Domain E=CH3 (Knob, 354C)

5th polypeptide chain (=“BC1” chain 2)
Domain P=VH
Domain Q=CH3 (L351D, 447C)

2nd polypeptide chain (=“BC1” chain 2)
Domain F=VH
Domain G=CH3 (L351D, 447C)

3rd polypeptide chain (=“BC1” chain 3)
Domain H=VL
Domain I=CL (Kappa)
Domain J=CH2
Domain K=CH3 (Hole, 349C)

4th polypeptide chain (=“BC1” chain 4)
Domain L=VH
Domain M=CH1

### 6.4.6.2. Trivalent 1×2 Bispecific B-Body “BC28-1×2”

With reference to Section 6.4.5.3. and FIG. 4A, in a series of embodiments, the binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has the first VL amino acid sequence and domain S has a human IgG1 CH3 amino acid sequence with a Y349C mutation and a C-terminal extension incorporating a PGK tripeptide sequence that is followed by GSGSGS linker peptide connecting domain S to domain H; (b) the binding molecule further comprises a sixth polypeptide chain, comprising: a domain T and a domain U, wherein the domains are arranged, from N-terminus to C-terminus, in a T-U orientation, and wherein domain T has the first VH amino acid sequence and domain U has a human IgG1 CH3 amino acid sequence with a S354C mutation and a C-terminal extension incorporating a PGK tripeptide sequence; (c) the third and the sixth polypeptides are associated through an interaction between the R and the T domains and an interaction between the S and the U domains to form the binding molecule, and (d) domain R and domain T form a third antigen binding site specific for the first antigen.

### 6.5. Other Binding Molecule Platforms

The various antibody platforms described above are not limiting. The antigen binding sites described herein, including specific CDR subsets, can be formatted into any binding molecule platform including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

### 6.6. Further Modifications

In a further series of embodiments, the binding molecules described herein have additional modifications.

### 6.6.1. Antibody-Drug Conjugates

In various embodiments, the binding molecule is conjugated to a therapeutic agent (i.e. drug) to form a binding molecule-drug conjugate. Therapeutic agents include, but are not limited to, chemotherapeutic agents, imaging agents (e.g. radioisotopes), immune modulators (e.g. cytokines, chemokines, or checkpoint inhibitors), and toxins (e.g. cytotoxic agents). In certain embodiments, the therapeutic agents are attached to the binding molecule through a linker peptide, as discussed in more detail in Section 6.6.3.

Methods of preparing antibody-drug conjugates (ADCs) that can be adapted to conjugate drugs to the binding molecules disclosed herein are described, e.g., in U.S. Pat. No. 8,624,003 (pot method), U.S. Pat. No. 8,163,888 (one-step), U.S. Pat. No. 5,208,020 (two-step method), U.S. Pat. Nos. 8,337,856, 5,773,001, 7,829,531, 5,208,020, 7,745,394, WO 2017/136623, WO 2017/015502, WO 2017/015496, WO 2017/015495, WO 2004/010957, WO 2005/077090, WO 2005/082023, WO 2006/065533, WO 2007/030642, WO 2007/103288, WO 2013/173337, WO 2015/057699, WO 2015/095755, WO 2015/123679, WO 2015/157286, WO 2017/165851, WO 2009/073445, WO 2010/068759, WO 2010/138719, WO 2012/171020, WO 2014/008375, WO 2014/093394, WO 2014/093640, WO 2014/160360, WO 2015/054659, WO 2015/195925, WO 2017/160754, Storz (MAbs. 2015 November-December; 7(6): 989-1009), Lambert et al. (Adv Ther, 2017 34: 1015), Diamantis et al. (British Journal of Cancer, 2016, 114, 362-367), Carrico et al. (Nat Chem Biol, 2007. 3: 321-2), We et al. (Proc Natl Acad Sci USA, 2009. 106: 3000-5), Rabuka et al. (Curr Opin Chem Biol., 2011 14: 790-6), Hudak et al. (Angew Chem Int Ed Engl., 2012: 4161-5), Rabuka et al. (Nat Protoc., 2012 7:1052-67), Agarwal et al. (Proc Natl Acad Sci USA., 2013, 110: 46-51), Agarwal et al. (Bioconjugate Chem., 2013, 24: 846-851), Barfield et al. (Drug Dev. and D., 2014, 14:34-41), Drake et al. (Bioconjugate Chem., 2014, 25:1331-41), Liang et al. (J Am Chem Soc., 2014, 136:10850-3), Drake et al. (Curr Opin Chem Biol., 2015, 28:174-80), and York et al. (BMC Biotechnology, 2016, 16(1):23), each of which is hereby incorporated by reference in its entirety for all that it teaches.

### 6.6.2. Additional Binding Moieties

In various embodiments, the binding molecule has modifications that comprise one or more additional binding moieties. In certain embodiments the binding moieties are antibody fragments or antibody formats including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In particular embodiments, the one or more additional binding moieties are attached to the C-terminus of the first or third polypeptide chain. In particular embodiments, the one or more additional binding moieties are attached to the C-terminus of both the first and third polypeptide chain. In particular embodiments, the one or more additional binding moieties are attached to the C-terminus of both the first and third polypeptide chains. In certain embodiments, individual portions of the one or more additional binding moieties are separately attached to the C-terminus of the first and third polypeptide chains such that the portions form the functional binding moiety.

In particular embodiments, the one or more additional binding moieties are attached to the N-terminus of any of the polypeptide chains (e.g. the first, second, third, fourth, fifth, or sixth polypeptide chains). In certain embodiments, individual portions of the additional binding moieties are separately attached to the N-terminus of different polypeptide chains such that the portions form the functional binding moiety.

In certain embodiments, the one or more additional binding moieties are specific for a different antigen or epitope of the ABSs within the binding molecule. In certain embodiments, the one or more additional binding moieties are specific for the same antigen or epitope of the ABSs within the binding molecule. In certain embodiments, wherein the modification is two or more additional binding moieties, the additional binding moieties are specific for the same antigen or epitope. In certain embodiments, wherein the modification is two or more additional binding moieties, the additional binding moieties are specific for different antigens or epitopes.

In certain embodiments, the one or more additional binding moieties are attached to the binding molecule using in vitro methods including, but not limited to, reactive chemistry and affinity tagging systems, as discussed in more detail in Section 6.6.3. In certain embodiments, the one or more additional binding moieties are attached to the binding molecule through Fc-mediated binding (e.g. Protein A/G). In certain embodiments, the one or more additional binding moieties are attached to the binding molecule using recombinant DNA techniques, such as encoding the nucleotide sequence of the fusion product between the binding molecule and the additional binding moieties on the same expression vector (e.g. plasmid).

### 6.6.3. Functional/Reactive Groups

In various embodiments, the binding molecule has modifications that comprise functional groups or chemically reactive groups that can be used in downstream processes, such as linking to additional moieties (e.g. drug conjugates and additional binding moieties, as discussed in more detail in Sections 6.6.1. and 6.6.2.) and downstream purification processes.

In certain embodiments, the modifications are chemically reactive groups including, but not limited to, reactive thiols (e.g. maleimide based reactive groups), reactive amines (e.g. N-hydroxysuccinimide based reactive groups), “click chemistry” groups (e.g. reactive alkyne groups), and aldehydes bearing formylglycine (FGly). In certain embodiments, the modifications are functional groups including, but not limited to, affinity peptide sequences (e.g. HA, HIS, FLAG, GST, MBP, and Strep systems etc.). In certain embodiments, the functional groups or chemically reactive groups have a cleavable peptide sequence. In particular embodiments, the cleavable peptide is cleaved by means including, but not limited to, photocleavage, chemical cleavage, protease cleavage, reducing conditions, and pH conditions. In particular embodiments, protease cleavage is carried out by intracellular proteases. In particular embodiments, protease cleavage is carried out by extracellular or membrane associated proteases. ADC therapies adopting protease cleavage are described in more detail in Choi et al. (Theranostics, 2012; 2(2): 156-178.), the entirety of which is hereby incorporated by reference for all it teaches.

### 6.6.4. Reduced Effector Function

In certain embodiments, the binding molecule has one or more engineered mutations in an amino acid sequence of an antibody domain that reduce the effector functions naturally associated with antibody binding. Effector functions include, but are not limited to, cellular functions that result from an Fc receptor binding to an Fc portion of an antibody, such as antibody-dependent cellular cytotoxicity (ADCC, also referred to as antibody-dependent cell-mediated cytotoxicity), complement fixation (e.g. C1q binding), antibody dependent cellular-mediated phagocytosis (ADCP), and opsonization. Engineered mutations that reduce the effector functions are described in more detail in U.S. Pub. No. 2017/0137530, Armour, et al. (Eur. J. Immunol. 29(8) (1999) 2613-2624), Shields, et al. (J. Biol. Chem. 276(9) (2001) 6591-6604), and Oganesyan, et al. (Acta Cristallographica D64 (2008) 700-704), each herein incorporated by reference in its entirety.

In specific embodiments, the binding molecule has one or more engineered mutations in an amino acid sequence of an antibody domain that reduce binding of an Fc portion of the binding molecule by FcR receptors. In some embodiments, the FcR receptors are FcRγ receptors. In particular embodiments, the FcR receptors are FcγRIIa and/or FcγRIIIA receptors.

### 6.7. Pharmaceutical Compositions

In another aspect, pharmaceutical compositions are provided that comprise a binding molecule as described herein and a pharmaceutically acceptable carrier or diluent. In typical embodiments, the pharmaceutical composition is sterile.

In various embodiments, the pharmaceutical composition comprises the binding molecule at a concentration of 0.1 mg/ml-100 mg/ml. In specific embodiments, the pharmaceutical composition comprises the binding molecule at a concentration of 0.5 mg/ml, 1 mg/ml, 1.5 mg/ml, 2 mg/ml, 2.5 mg/ml, 5 mg/ml, 7.5 mg/ml, or 10 mg/ml. In some embodiments, the pharmaceutical composition comprises the binding molecule at a concentration of more than 10 mg/ml. In certain embodiments, the binding molecule is present at a concentration of 20 mg/ml, 25 mg/ml, 30 mg/ml, 35 mg/ml, 40 mg/ml, 45 mg/ml, or even 50 mg/ml or higher. In particular embodiments, the binding molecule is present at a concentration of more than 50 mg/ml.

In various embodiments, the pharmaceutical compositions are described in more detail in U.S. Pat. Nos. 8,961,964, 8,945,865, 8,420,081, 6,685,940, 6,171,586, 8,821,865, 9,216,219, U.S. application Ser. No. 10/813,483, WO 2014/066468, WO 2011/104381, and WO 2016/180941, each of which is incorporated herein in its entirety.

### 6.8. Methods of Manufacturing

The binding molecules described herein can readily be manufactured by expression using standard cell free translation, transient transfection, and stable transfection approaches currently used for antibody manufacture. In specific embodiments, Expi293 cells (ThermoFisher) can be used for production of the binding molecules using protocols and reagents from ThermoFisher, such as ExpiFectamine, or other reagents known to those skilled in the art, such as polyethylenimine as described in detail in Fang et al. (Biological Procedures Online, 2017, 19:11), herein incorporated by reference for all it teaches.

As further described in the Examples below, the expressed proteins can be readily purified using a CH1 affinity resin, such as the CaptureSelect CH1 resin and provided protocol from ThermoFisher. Further purification can be effected using ion exchange chromatography as is routinely used in the art.

### 6.9. Methods of Treatment

In another aspect, methods of treatment are provided. The methods comprise administering a therapeutically effective amount of the pharmaceutical compositions described herein to a patient in need thereof.

In typical embodiments, the target receptor is a member of the TNFRSF, and the disease to be treated is cancer. In certain embodiments, the target receptor is OX40 (TNFRSF4), CD40 (TNFRSF5), or 4-1BB (TNFRSF9), and the pharmaceutical construct is administered in an amount that is therapeutically effective in treating cancer.

### 6.10. Examples

The following examples are provided by way of illustration, not limitation.

### 6.10.1. Methods

Non-limiting, illustrative methods for the purification of the various antigen-binding proteins and their use in various assays are described in more detail below.

### 6.10.1.1. Expi293 Expression

The various antigen-binding proteins tested were expressed using the Expi293 transient transfection system according to manufacturer's instructions. Briefly, four plasmids coding for four individual chains were mixed at 1:1:1:1 mass ratio, unless otherwise stated, and transfected with ExpiFectamine 293 transfection kit to Expi 293 cells. Cells were cultured at 37° C. with 8% CO2, 100% humidity and shaking at 125 rpm. Transfected cells were fed once after 16-18 hours of transfections. The cells were harvested at day 5 by centrifugation at 2000 g for 10 munities. The supernatant was collected for affinity chromatography purification.

### 6.10.1.2. Protein A and Anti-CH1 Purification

Cleared supernatants containing the various antigen-binding proteins were separated using either a Protein A (ProtA) resin or an anti-CH1 resin on an AKTA Purifier FPLC. In examples where a head-to-head comparison was performed, supernatants containing the various antigen-binding proteins were split into two equal samples. For ProtA purification, a 1 mL Protein A column (GE Healthcare) was equilibrated with PBS (5 mM sodium potassium phosphate pH 7.4, 150 mM sodium chloride). The sample was loaded onto the column at 5 ml/min. The sample was eluted using 0.1 M acetic acid pH 4.0. The elution was monitored by absorbance at 280 nm and the elution peaks were pooled for analysis. For anti-CH1 purification, a 1 mL CaptureSelect™ XL column (ThermoFisher) was equilibrated with PBS. The sample was loaded onto the column at 5 ml/min. The sample was eluted using 0.1 M acetic acid pH 4.0. The elution was monitored by absorbance at 280 nm and the elution peaks were pooled for analysis.

### 6.10.1.3. SDS-Page Analysis

Samples containing the various separated antigen-binding proteins were analyzed by reducing and non-reducing SDS-PAGE for the presence of complete product, incomplete product, and overall purity. 2 μg of each sample was added to 15 μL SDS loading buffer. Reducing samples were incubated in the presence of 10 mM reducing agent at 75° C. for 10 minutes. Non-reducing samples were incubated at 55° C. for 5 minutes without reducing agent. The reducing and non-reducing samples were loaded into a 4-15% gradient TGX gel (BioRad) with running buffer and run for 30 minutes at 250 volts. Upon completion of the run, the gel was washed with DI water and stained using GelCode Blue Safe Protein Stain (ThermoFisher). The gels were destained with DI water prior to analysis. Densitometry analysis of scanned images of the destained gels was performed using standard image analysis software to calculate the relative abundance of bands in each sample.

### 6.10.1.4. IEX Chromatography

Samples containing the various separated antigen-binding proteins were analyzed by cation exchange chromatography for the ratio of complete product to incomplete product and impurities. Cleared supernatants were analyzed with a 5-ml MonoS column (GE Lifesciences) on an AKTA Purifier FPLC. The MonoS column was equilibrated with buffer A 10 mM MES pH 6.0. The samples were loaded onto the column at 2 ml/min. The sample was eluted using a 0-30% gradient with buffer B (10 mM MES pH 6.0, 1 M sodium chloride) over 6 CV. The elution was monitored by absorbance at 280 nm and the purity of the samples was calculated by peak integration to identify the abundance of the monomer peak and contaminants peaks. The monomer peak and contaminant peaks were separately pooled for analysis by SDS-PAGE as described above.

### 6.10.1.5. Analytical SEC Chromatography

Samples containing the various separated antigen-binding proteins were analyzed by analytical size exclusion chromatography for the ratio of monomer to high molecular weight product and impurities. Cleared supernatants were analyzed with an industry standard TSK G3000SW×1 column (Tosoh Bioscience) on an Agilent 1100 HPLC. The TSK column was equilibrated with PBS. 25 μL of each sample at 1 mg/mL was loaded onto the column at 1 ml/min. The sample was eluted using an isocratic flow of PBS for 1.5 CV. The elution was monitored by absorbance at 280 nm and the elution peaks were analyzed by peak integration.

### 6.10.1.6. Mass Spec

Samples containing the various separated antigen-binding proteins were analyzed by mass spectrometry to confirm the correct species by molecular weight. All analysis was performed by a third-party research organization. Briefly, samples were treated with a cocktail of enzymes to remove glycosylation. Samples were both tested in the reduced format to specifically identify each chain by molecular weight. Samples were all tested under non-reducing conditions to identify the molecular weights of all complexes in the samples. Mass spec analysis was used to identify the number of unique products based on molecular weight.

### 6.10.1.7. NFκB Luc2 OX40 Jurkat T Cell Stimulation Assay

The NFκB Luc2 OX40 Jurkat T cell Stimulation Assay (Promega, Cat #CS197704, CS197707) was performed according to manufacturer's instructions. Briefly, the Thaw-and-Use Jurkat/OX40 cells were thawed at 37 deg C. and diluted in assay buffer as recommended. The Thaw-and-Use cells were dispensed into 96-well plates (50 uL/well) and incubated overnight in a CO2 incubator at 37 deg C. The next day, serial dilutions of the OX40 ligand are made as a standard control at 3× of the final concentration. 25 ul of the standards as well as test samples at 3× final concentration are added to the well and incubated in a CO2 incubator at 37 deg C. for 5 hours. Upon completion of the incubation period, 75 uL of the Bio-Glo reagent is added to each well and incubated at ambient room temperature for 5-10 minutes. The signal from each well is then read using a standard plate reader with glow-type luminescence.

### 6.10.1.8. T Cell Stimulation Assay

T-cell stimulation was measured using multiple assays to follow the cytokine production as well as impact of T cell proliferation. Measurement of T cell activation through measurement of cytokine (IL-2, TNFα, and IFNγ) production was performed using the Cytokine Screen Opteia ELISA Kit (BD Cat #555212, 555190, & 555142) according to the manufacturer's instructions. Briefly, 96-well ELISA plates were coated with the specific capture antibody overnight using 100 uL/well according to instructions. The ELISA plates were blocked with 150 uL RPMI per well. Serial dilutions of the cytokine standards were prepared in RPMI+10% HI FBS to cover the indicated ranges (IL-2: 500-7.8 pg/mL, TNFα and IFNγ: 300-4.6 pg/mL). Supernantants of samples from the treated T cells as well as standards were added to the plate and incubated for 2 hours at room temperature. The wells were washed four times with 150 uL 0.5% PBST. The detection antibodies were added at 1:250 dilution in PBS and incubated for 1 hour at room temperature. The wells were washed four times with 150 uL 0.5% PBST. 100 uL/well of the TMB substrate was added and incubated until color change observed at which point 50 uL of 0.1M HCl was added to stop the reaction. The final signal was measured by reading the absorbance on plate reader at 450 nm.

To monitor T cell proliferation, 96 well plates were coated overnight with anti-CD3 antibody in PBS. Some wells were additionally coated with anti-OX40 antibodies in PBS. The next day, excess liquid was removed from the plate by flicking and naïve CD4+ T cells were plated in RPMI+10% FBS. Wells that were not coated overnight with anti-OX40 antibodies, received anti-OX40 antibodies in the media at the listed concentrations. Plates were incubated at 37° C./5% CO2 for 3-5 days. Following the incubation, the PrestoBlue assay (Thermo Fisher A13261) was carried out according to manufacturer's directions. Briefly, PrestoBlue reagent was added directly to the wells at 1/10^{th} the volume of media within the wells. The plates were then incubated at 37° C./5% CO2 for 10 min-overnight. The fluorescence of each well was determined using a Safire plate reader (Tecan) with an excitation wavelength of 560 nm and an emission wavelength of 590 nm.

### 6.10.1.9. IncuCyte System

Real-time activation of T cells was monitored using the IncuCyte system (Sartorius). The kinetics of T cell activation were monitored for 3 to 6 days by microscopy in a controlled growth environment. T cell activation is charted using cell size measurement to track the growth and proliferation of T cell clusters.

### 6.10.2. Example 1: Bivalent Anti-OX40 Agonist Antibodies

### 6.10.2.1. Human Anti-OX40 Antibody Discovery by Phage Display

Phage display of human Fab libraries was carried out using standard protocols. Biotinylated extracellular domain of human OX40 protein was purchased from Acro Biosystems. Phage clones were screened for the ability to bind human OX40 by phage ELISA using standard protocols. Briefly, Fab-formatted phage libraries were constructed using expression vectors capable of replication and expression in phage (also referred to as a phagemid). Both the heavy chain and the light chain were encoded for in the same expression vector, where the heavy chain was fused to a truncated variant of the phage coat protein pIII. The light chain and heavy chain are expressed as a separate polypeptides, and the light chain and heavy chain-pIII fusion assemble in the bacterial periplasm, where the redox potential enables disulfide bond formation, to form the antibody containing the candidate ABS

The library was created using sequences derived from a specific human heavy chain variable domain (VH3-23) and a specific human light chain variable domain (Vk-1). Light chain variable domains within the screened library were generated with diversity introduced into the VL CDR3 (L3) and where the light chain VL CDR1 (L1) and CDR2 (L2) remained the human germline sequence. For the screened library, all three CDRs of the VH domain were diversified to match the positional amino acid frequency by CDR length found in the human antibody repertoire. The phage display heavy chain (SEQ ID NO:19) and light chain (SEQ ID NO:20) scaffolds used in the library are listed below, where a lower case “x” represents CDR amino acids that were varied to create the library, and bold italic represents the CDR sequences that were constant.

Diversity was created through Kunkel mutagenesis using primers to introduce diversity into VL CDR3 and VH CDR1 (H1), CDR2 (H2) and CDR3 (H3) to mimic the diversity found in the natural antibody repertoire, as described in more detail in Kunkel, T A (PNAS Jan. 1, 1985. 82 (2) 488-492), herein incorporated by reference in its entirety. Briefly, single-stranded DNA were prepared from isolated phage using standard procedures and Kunkel mutagenesis carried out. Chemically synthesized DNA was then electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to ˜5×10¹² phage from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD₆₀₀˜0.6. After 20 minutes, infected cells were sub-cultured in 25 mL 2×YT+Ampicillin and M13K07 helper phage and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with 100 nM bead-immobilized antigen using standard procedures. In total, 3-4 rounds of phage panning were performed to enrich in phage displaying Fabs specific for the target antigen. Target-specific enrichment was confirmed using polyclonal and monoclonal phage ELISA. DNA sequencing was used to determine isolated Fab clones containing a candidate ABS.

To measure binding affinity in discovery campaigns, the VL and VH domains were formatted into a bivalent monospecific native human full-length IgG1 architecture and immobilized to a biosensor on an Octet (Pall ForteBio) biolayer interferometer. Soluble antigens were then added to the system and binding measured.

For experiments performed using the B-Body format, VL variable regions of individual clones were formatted into Domain A and/or H, and VH region into Domain F and/or L of a bivalent 1×1 B-Body “BC1” scaffold shown below and with reference to FIG. 2B.

In a first discovery campaign, the VL and VH domains were formatted only into Domain H and L, respectively, and the constructs each contained the same A:F antigen binding site with a known expression profile for an unrelated target. The sequence of the common first polypeptide and common second polypeptide chain are provided, respectively, in SEQ ID NO:1 and SEQ ID NO:2. In subsequent discovery campaigns, the VL and VH domains were formatted into a bivalent monospecific native IgG architecture.

“BC1” Scaffold:
1^{st} polypeptide chain (SEQ ID NO:23)
   Domain A=Antigen 1 B-Body Domain A/H Scaffold (SEQ ID NO:21)
   Domain B=CH3 (T366K; 445K, 446S, 447C tripeptide insertion)
   Domain D=CH2
   Domain E=CH3 (T366W, S354C)
2^{nd} polypeptide chain (SEQ ID NO:24):
   Domain F=Antigen 1 B-Body Domain F/L Scaffold (SEQ ID NO:22)
   Domain G=CH3 (L351D; 445G, 446E, 447C tripeptide insertion)
3^{rd} polypeptide chain (SEQ ID NO:25):
   Domain H=Antigen 2 B-Body Domain A/H Scaffold (SEQ ID NO:21)
   Domain I=CL (Kappa)
   Domain J=CH2
   Domain K=CH3 (Y349C, D356E, L358M, T366S, L368A, Y407V)
4^{th} polypeptide chain (SEQ ID NO:26):
   Domain L=Anitgen 2 B-Body Domain F/L Scaffold (SEQ ID NO:22)
   Domain M=CH1.

For BC1 2×1 formats (see FIGS. 3A and 3B), the variable domains were formatted into the 2(A-A)×1(B) format described in Section 6.4.2.1, where the 1st polypeptide scaffold chain is SEQ ID NO:27. The other BC1 2×1 chains are identical to the BC1 chains, with the 5th chain identical to the 2nd chain. For candidates using the variable domains formatted into the 2(B-A)×1(B) format as described below, e.g., the OX40:24-11×11 described in Section 6.10.13, and see Section 6.4.2.3.

### 6.10.2.2. Monospecific Bivalent Construct Expression, Purification and Characterization

In the first discovery campaign, the B-Body plasmids coding for OX40 antigen binding sites (ABS) in Domains H and L (H:L) and a common antigen binding site with a known expression profile in domains A and F (A:F) were transfected into cells using the Expi 293 expression system at 1.5 mL scale and the antibody constructs expressed in 96-well deep well block following standard protocols.

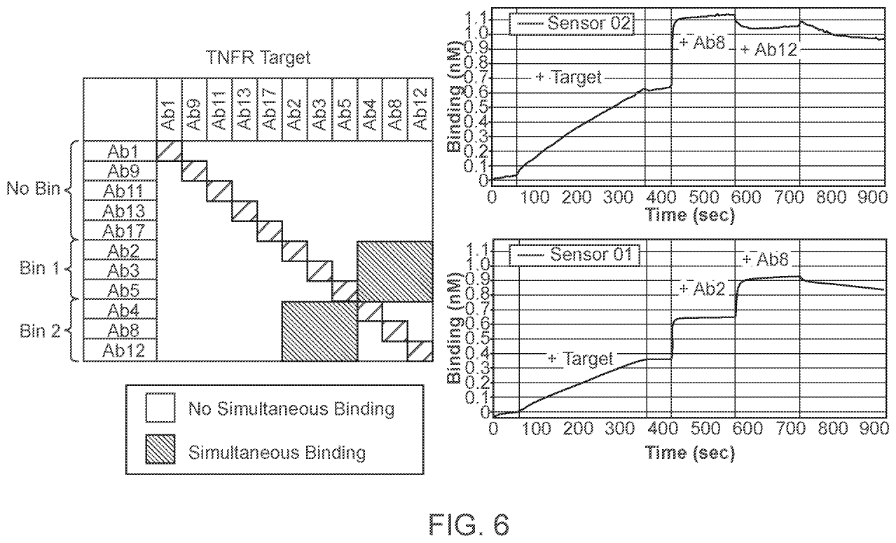

The B-Body protein was purified in 96-well format using CaptureSelect CH1 affinity resin (ThermoFisher) and average yield was ˜50 μg B-Body/mL culture. The bispecific 1×1 B-Body proteins—each containing one OX40 antigen binding site—were evaluated for overall yield, protein purity, affinity for OX40, and cell binding. As shown in FIG. 6, the clones were tested for cross-competition and sorted into epitope bins. Affinity was determined using biolayer interferometry (BLI, Octet/FORTEBIO®). The first discovery campaign identified 17 clones that can be expressed in the Expi 293 system, bind to human OX40 on the cell surface with monovalent affinity in the range of 1-100 nM, and do not exhibit non-specific binding.

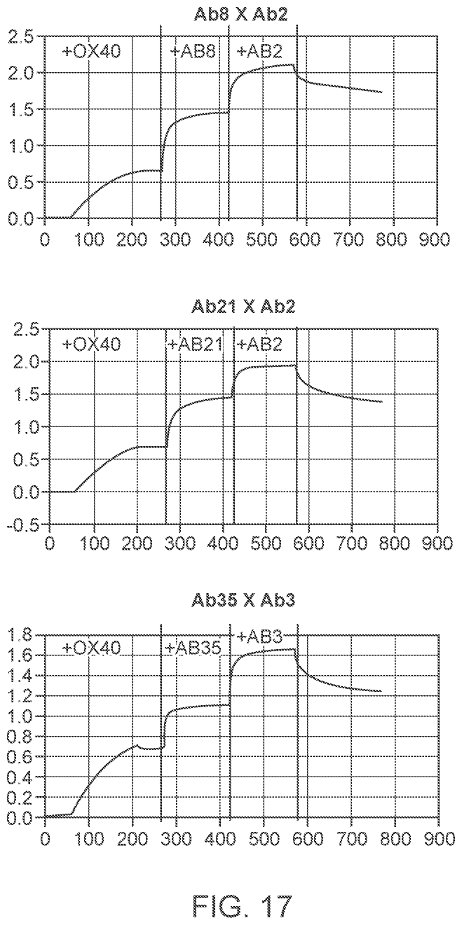

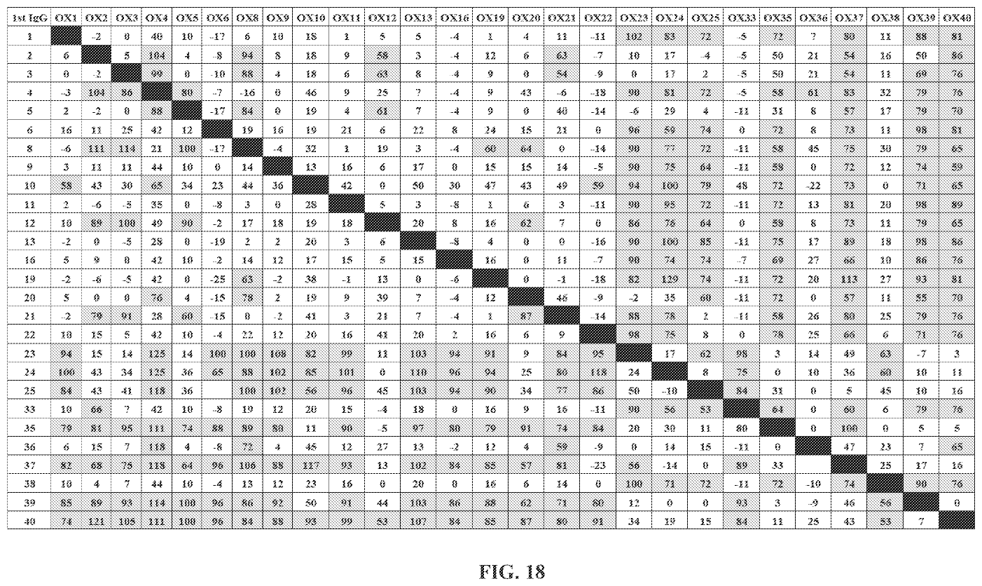

In subsequent discovery campaigns, the VL and VH domains were formatted into a bivalent monospecific native IgG architecture. As shown in FIG. 17 and FIG. 18, clones were tested for cross-competition and sorted into epitope bins. Affinity was determined using biolayer interferometry (BLI, Octet/FORTEBIO®).

In total, the antibody discovery campaigns identified 40 OX40 clones. Table 3 lists the VH CDR1/2/3 sequences identified. Table 4 lists the VL CDR3 sequences identified, and the constant CDR1 and CDR2 sequences used in the screen.

TABLE 3
Candidate hOX40 VH Antigen Binding Sites

| ABS | CDR1 | SEQ ID NO | CDR2 | SEQ ID NO | CDR3 | SEQ ID NO |
|---|---|---|---|---|---|---|
| 01 | SSYY | 60 | GIHPYSILTR | 100 | GYYYVADHVF | 140 |
| 02 | DGYY | 61 | AIESSSGYTY | 101 | AYYTGM | 141 |
| 03 | SDYY | 62 | AITSTGGSTY | 102 | GDYTGM | 142 |
| 04 | DGYY | 63 | YIHPYGGYTR | 103 | TRYDTGM | 143 |
| 05 | SVYY | 64 | AIDPGSSYTY | 104 | SSYTAM | 144 |
| 06 | TDYH | 65 | GISSYTGQTD | 105 | GISGGF | 145 |
| 07 | TSYY | 66 | LIDPDSSITD | 106 | MDTIVL | 146 |
| 08 | PTYY | 67 | YIYSGGGSTR | 107 | TDASSAL | 147 |
| 09 | WSYY | 68 | AITPYDGYTY | 108 | GSVYTGM | 148 |
| 10 | SSYY | 69 | YIGSQGGFTD | 109 | QGYGYAL | 149 |
| 11 | SSYV | 70 | YIFPYGGTTY | 110 | GYYYVSDRVM | 150 |
| 12 | TRYY | 71 | YIAPQGRSTH | 111 | SLYYGGM | 151 |
| 13 | SSYI | 72 | YIFPYSGETY | 112 | GAYYYTDLVF | 152 |
| 14 | SWYP | 73 | WIYPISGYTD | 113 | QGFVVGGAF | 153 |
| 15 | FSYY | 74 | WISPYGKRTH | 114 | RYGRFGYRSYVAM | 154 |
| 16 | SSYY | 75 | AIRPYGSDTS | 115 | GYYYSWDYPPWVF | 155 |
| 17 | STYY | 76 | QIDPTDWWTD | 116 | GYSPVFDIVIEFGL | 156 |
| 18 | SRYP | 77 | SIYPWGGYTY | 117 | ESGPGAM | 157 |
| 19 | PSYL | 78 | YIHPYSGYTD | 118 | GFYGGSDLVL | 158 |
| 20 | SDYY | 79 | AIEPSDGYTY | 119 | GDYPGM | 159 |
| 21 | SSYY | 80 | YISPYGSYTK | 120 | SDYYGAL | 160 |
| 22 | DDYY | 81 | TXSSSHAYTY | 121 | ESVYPMGAM | 161 |
| 23 | TSYI | 82 | LIASYDSYTD | 122 | SYDGVGHYLYGLGGF | 162 |
| 24 | LSYY | 83 | YIDPYSGGTD | 123 | VGLSFYAQEPVL | 163 |
| 25 | SSYL | 84 | YIDPWDDGTQ | 124 | SWGQYYDYYDVF | 164 |
| 26 | SSYY | 85 | VISPYAGSTK | 125 | GFGYYAEFDSAL | 165 |
| 27 | STYY | 86 | AISPYHGDTS | 126 | VEGIGM | 166 |
| 28 | STYY | 87 | GIYSSGGYTF | 127 | TYRYYGM | 167 |
| 29 | SDYW | 88 | YITPYGDETD | 128 | IIQLLGM | 168 |
| 30 | SSYV | 89 | FIHPLSDSTG | 129 | GYYYSSDYVM | 169 |
| 31 | SFYY | 90 | YIQSEGSVTY | 130 | FDAYVAM | 170 |
| 32 | ASYH | 91 | WISPSGSTTR | 131 | VYHGTGL | 171 |
| 33 | SHYY | 92 | VIDPQADRTD | 132 | DYMYFVM | 172 |
| 34 | SRYF | 93 | WIYSYGSTTG | 133 | RSQYSVM | 173 |
| 35 | SSYA | 94 | WIDSGDGDTF | 134 | SLGYYYYGHGVF | 174 |
| 36 | SSYT | 95 | YIDSKGGYTS | 135 | SWYDTGHFGYDAVF | 175 |
| 37 | SDYI | 96 | YISSYGGYTS | 136 | AAYPFYDYDPAF | 176 |
| 38 | SSYY | 97 | SIYSDTDYTY | 137 | EGFVYPSSTAL | 177 |
| 39 | ASYE | 98 | AIDPYDGETY | 138 | DFSSYYGLAMGF | 178 |
| 40 | YSYF | 99 | AIDSYSGDTY | 139 | GYGDAYYFYEYGAM | 179 |
| 24 | LSYY | 83 | YIDPYSGGTD | 123 | VGLSFYAQEPVL | 163 |
| WEE | | | | | | |

TABLE 4
Candidate hOX40 VL Antigen Binding Sites

| | | SEQ | | SEQ | | SEQ |
|---|---|---|---|---|---|---|
| ABS | CDR1 | ID NO | CDR2 | ID NO | CDR3 | ID NO |
| 01 | RASQSVSSAVA | 220 | SASSLYS | 221 | FQDSPV | 180 |
| 02 | RASQSVSSAVA | 220 | SASSLYS | 221 | YIYGPL | 181 |
| 03 | RASQSVSSAVA | 220 | SASSLYS | 221 | YIYSPA | 182 |
| 04 | RASQSVSSAVA | 220 | SASSLYS | 221 | WYSDPE | 183 |
| 05 | RASQSVSSAVA | 220 | SASSLYS | 221 | YIYDPS | 184 |
| 06 | RASQSVSSAVA | 220 | SASSLYS | 221 | YARPPR | 185 |
| 07 | RASQSVSSAVA | 220 | SASSLYS | 221 | YYFWPW | 186 |
| 08 | RASQSVSSAVA | 220 | SASSLYS | 221 | YVSSPE | 187 |
| 09 | RASQSVSSAVA | 220 | SASSLYS | 221 | YDYSPA | 188 |
| 10 | RASQSVSSAVA | 220 | SASSLYS | 221 | VDSTPV | 189 |
| 11 | RASQSVSSAVA | 220 | SASSLYS | 221 | YTSHPG | 190 |
| 12 | RASQSVSSAVA | 220 | SASSLYS | 221 | FYSSPE | 191 |
| 13 | RASQSVSSAVA | 220 | SASSLYS | 221 | YSSSPV | 192 |
| 14 | RASQSVSSAVA | 220 | SASSLYS | 221 | WSAKLY | 193 |
| 15 | RASQSVSSAVA | 220 | SASSLYS | 221 | YTSSPY | 194 |
| 16 | RASQSVSSAVA | 220 | SASSLYS | 221 | ADYSLT | 195 |
| 17 | RASQSVSSAVA | 220 | SASSLYS | 221 | ASWGLT | 196 |
| 18 | RASQSVSSAVA | 220 | SASSLYS | 221 | YERIPY | 197 |
| 19 | RASQSVSSAVA | 220 | SASSLYS | 221 | YYGSLY | 198 |
| 20 | RASQSVSSAVA | 220 | SASSLYS | 221 | VTYTPL | 199 |
| 21 | RASQSVSSAVA | 220 | SASSLYS | 221 | YYTSPE | 200 |
| 22 | RASQSVSSAVA | 220 | SASSLYS | 221 | LSSWPL | 201 |
| 23 | RASQSVSSAVA | 220 | SASSLYS | 221 | SDSSPW | 202 |
| 24 | RASQSVSSAVA | 220 | SASSLYS | 221 | WDDSPY | 203 |
| 25 | RASQSVSSAVA | 220 | SASSLYS | 221 | LFSHPY | 204 |
| 26 | RASQSVSSAVA | 220 | SASSLYS | 221 | WYSTPY | 205 |
| 27 | RASQSVSSAVA | 220 | SASSLYS | 221 | AYGDLR | 206 |
| 28 | RASQSVSSAVA | 220 | SASSLYS | 221 | GYSDPQ | 207 |
| 29 | RASQSVSSAVA | 220 | SASSLYS | 221 | GSSSPL | 208 |
| 30 | RASQSVSSAVA | 220 | SASSLYS | 221 | YSDWPY | 209 |
| 31 | RASQSVSSAVA | 220 | SASSLYS | 221 | HSSSLE | 210 |
| 32 | RASQSVSSAVA | 220 | SASSLYS | 221 | VDTSLG | 211 |
| 33 | RASQSVSSAVA | 220 | SASSLYS | 221 | ADTQPL | 212 |
| 34 | RASQSVSSAVA | 220 | SASSLYS | 221 | WSSSPE | 213 |
| 35 | RASQSVSSAVA | 220 | SASSLYS | 221 | YHTSLH | 214 |
| 36 | RASQSVSSAVA | 220 | SASSLYS | 221 | YYGGLP | 215 |
| 37 | RASQSVSSAVA | 220 | SASSLYS | 221 | SYSSPY | 216 |
| 38 | RASQSVSSAVA | 220 | SASSLYS | 221 | YYSEPV | 217 |
| 39 | RASQSVSSAVA | 220 | SASSLYS | 221 | VHSYPS | 218 |
| 40 | RASQSVSSAVA | 220 | SASSLYS | 221 | WTRSLT | 219 |
| 24 | RASQSVSSAVA | 220 | SASSLYS | 221 | WEESPY | 227 |
| WEE | | | | | | |

### 6.10.2.3. Bispecific Bivalent Expression, Purification, and Characterization

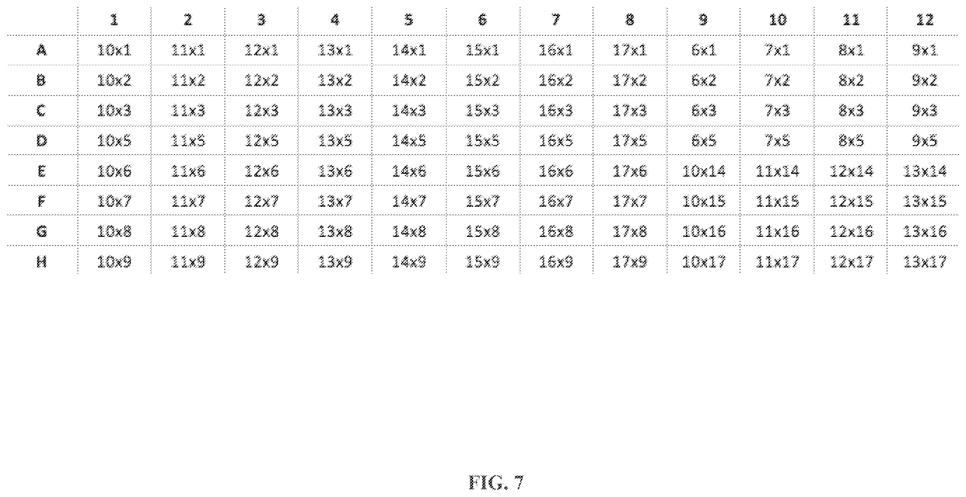

The OX40 antigen binding site of each of the initial 17 clones we identified was then recloned into a bivalent (1×1) B-Body construct, as either antigen binding site A:F or H:L. 96 unique bispecific bivalent 1×1 B-Body proteins were constructed, each construct having two OX40 antigen binding specificities. FIG. 7 shows the setup of bivalent 1×1 B-Body expression in 96-well format. Numerical numbers represent unique OX40 binders.

Each construct was expressed and purified. The purity was normally >85% as estimated by SDS PAGE. The concentration of purified antibodies was ˜1 mg/mL on average after one-step affinity purification using CH1 affinity resin and neutralization. The proteins were directly used for activation assay at 1 μg/mL after ˜1000× dilution in DMEM media.

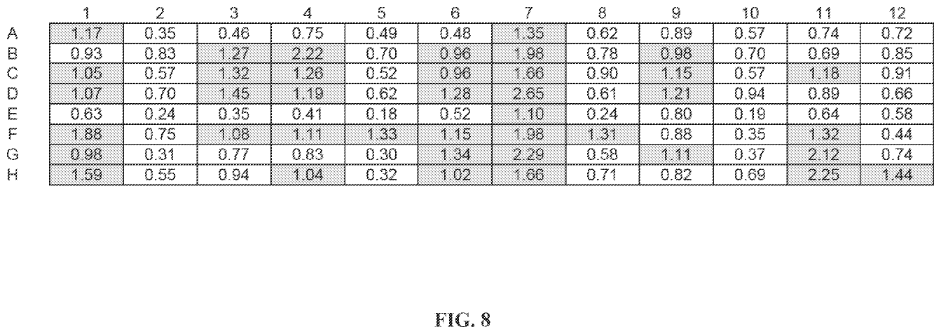

FIG. 8 tabulates concentrations (in mg/mL) of the respective bivalent 1×1 B-Body constructs after one-step purification. The average concentration was 950+/−500 μg/mL.

### 6.10.2.4. Assay Cell Line Generation

Luminescent-based reporter cell lines were generated to assay the NFκb pathway activation by OX40. In brief, a plasmid coding the full-length human OX40 under a CMV promotor with hygromycin resistance was transfected into NFκB/293/GFP-Luc (catalog number: TR860A-1, SystemBio) cells. Selection was performed with 200 μg/mL Hygromycin B for three weeks. The pool was detached, labeled with anti-human OX40-phycoerythrin antibody, and sorted for PE positive and GFP negative cells by FACS. The ˜106 collected cells were expanded for two weeks under DMEM+200 μg/mL Hygromycin B and sorted again for GFP negative. The 2nd sorted pool was annotated as NFκb/293/GFP-Luc-OX40 to assay NFkb activation.

### 6.10.2.5. NFκB Activation by OX40 Agonist

For high throughput screening, activation assays were prepared in half-area 96-well plates containing 5×10⁴NFkb/293/GFP-Luc-OX40 cells, 6 nM B-Body antibodies, with or without 20 nM Goat-anti-human (GAH) antibody. After a 6 hr incubation at 37° C., an equal volume of One-step BPS Luminescence Kit mix was added and the luminescence was measured. The luminescence intensity is proportional to agonist activity through OX40. An activation assay with an antibody titration (0.01-100 nM) was performed with candidates showing top potency from high throughput single point activation.

### 6.10.2.6. Results

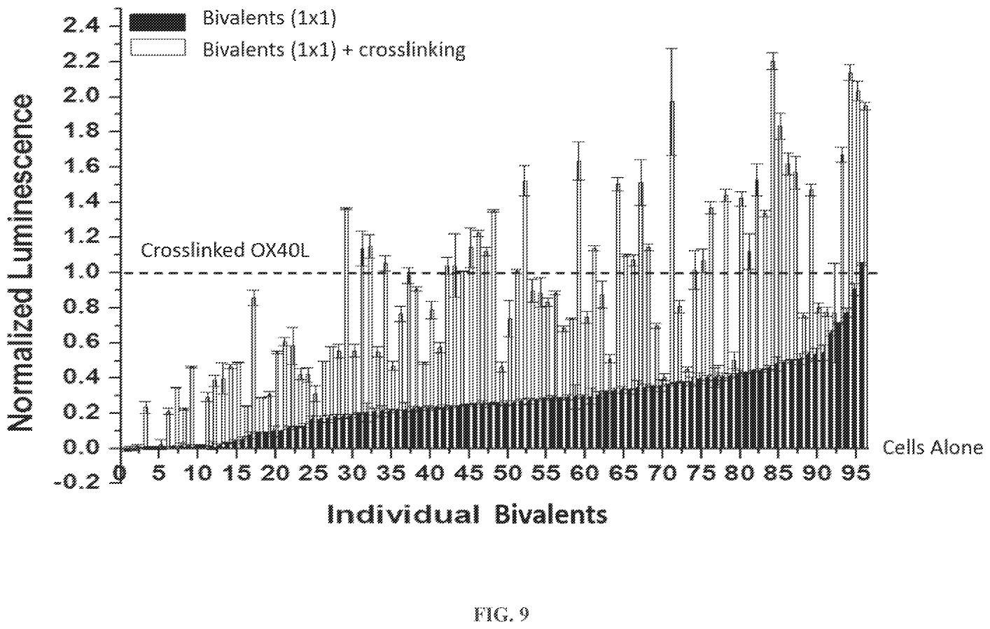

NFκb activation assay of the bivalent 1×1 B-Body constructs was set up at 1 μg/mL with NFκb/293/GFP-Luc-OX40 cells. The luminescence intensity was measured after 6 hrs of stimulation. The intensity was normalized by reference to blank cells without antibody (Luminescence=0) and cells exposed to OX40 ligand Fc fusion protein (“OX40L-Fc”) crosslinked using a goat anti-human Fc antibody (“OX40L-Fc+GAH”) (luminescence=1). The data are shown in FIG. 9. Black column: 6 nM bivalent 1×1 B-Body. Open column: 6 nM of the respective 1×1 B-Body with 20 nM goat-anti-human (GAH) antibody added as an independent crosslinking agent.

An initial screen tested approximately 150 bispecific bivalent 1×1 B-Body constructs and approximately 30 monospecific bivalent 1×1 B-Body constructs. As we hypothesized, in the absence of an independent crosslinking agent, the bivalent 1×1 B-Body constructs displayed a diverse range of agonist activity from 0 to 1. Some matched the potency of the cross-linked ligand (see FIG. 9).

We also observed a wide range of sensitivity to additional crosslinking of the bivalent 1×1 B-Body construct by an independent crosslinking agent, goat anti-human Fc antibody (GAH), from no additional enhancement to 3-fold additional enhancement.

“OX-10×9” demonstrated the strongest agonist activity for the bispecific bivalent (1×1) candidates tested in this panel, exhibiting comparable agonist activity to cross-linked OX40L, with its activity enhanced an additional 2-fold with additional GAH crosslinking. The sequences of the polypeptide chains are provided as SEQ ID NO:3 (chain 1), SEQ ID NO:4 (chain 2), SEQ ID NO:5 (chain 3), and SEQ ID NO:6 (chain 4).

### 6.10.3. Example 2: Trivalent Anti-OX40 Agonist Candidates

We also cloned the variable regions of the initial 17 OX40 agonist candidates we identified into antigen binding sites in the trivalent 2×1 B-Body format (see FIG. 3) and trivalent 1×2 format (see FIG. 4). By selectively pairing variable regions, we created and screened in the range of 100 OX40 bispecific trivalent 2×1 B-Body constructs.

The trivalent B-Body constructs were expressed at 1.5 mL scale in 96-well deep well blocks and purified with CH1 affinity resin.

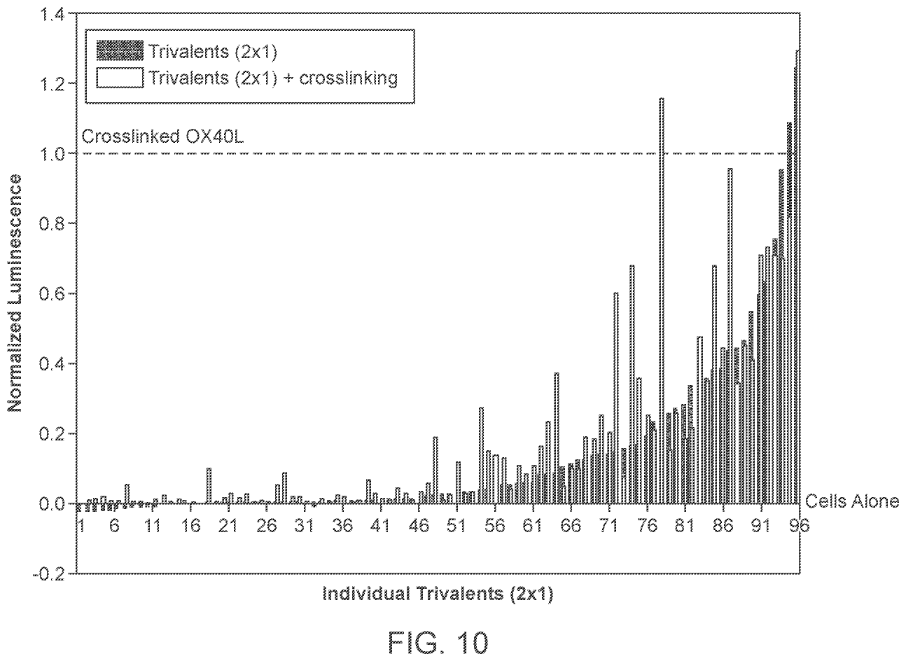

High throughput screening was performed for OX40 agonist trivalent constructs essentially as described above for bivalent constructs. Data are shown in FIG. 10.

Monospecific trivalent 2×1 B-Body, “OX40:2-2×2”, stood out as a potent OX40 agonist in the panel tested. The A:F, N:P, and H:L antigen binding sites are identical in this monospecific trivalent construct. The sequence of the polypeptide chains are provided as SEQ ID NO:7 (first polypeptide chain), SEQ ID NO:8 (second polypeptide chain), SEQ ID NO:9 (third polypeptide chain), SEQ ID NO:10 (fourth polypeptide chain), SEQ ID NO:11 (fifth polypeptide chain). The sequence of chain 2 and chain 5 are identical.

### 6.10.4. Example 3: Comparison to Existing Anti-OX40 Agonist Monoclonal Antibody Clinical Constructs

To understand better the therapeutic potential of our top agonists, we used published sequence to prepare three known clinical anti-OX40 monoclonal antibodies, Tavolixizumab (heavy chain, SEQ ID NO:14; light chain, SEQ ID NO:15), Pagolizumab (heavy chain, SEQ ID NO:12; light chain, SEQ ID NO:13) and GSK3174998 (heavy chain, SEQ ID NO:16; light chain, SEQ ID NO:17) and included them as benchmarks in our activation assays.

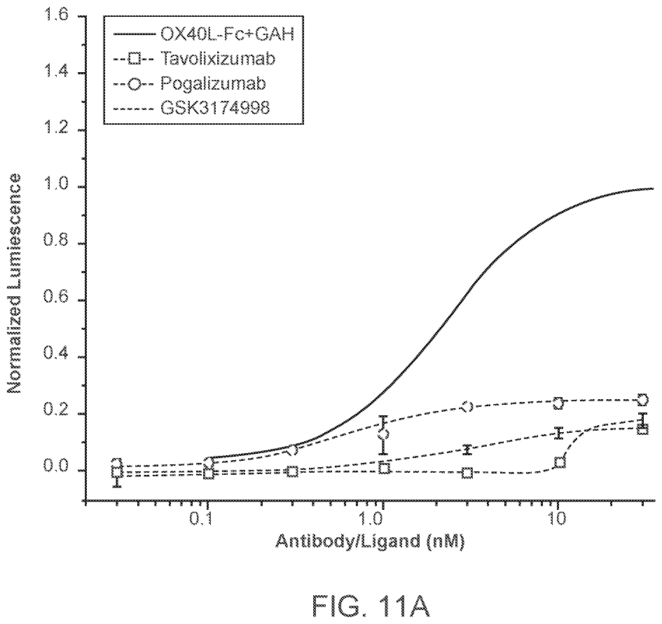

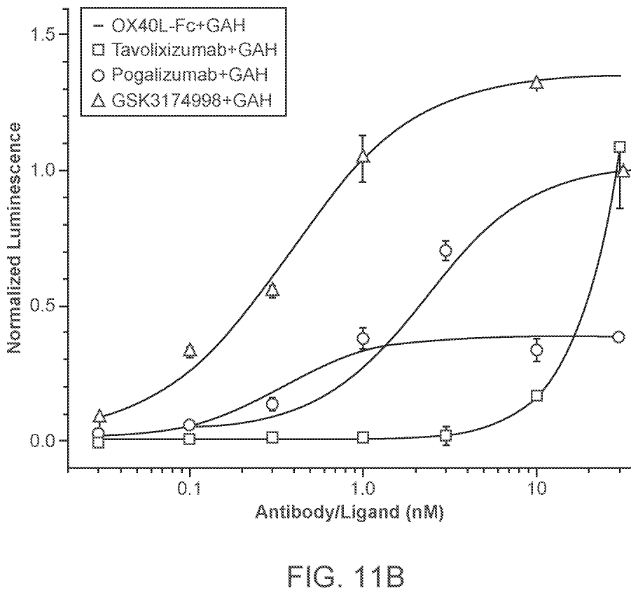

FIG. 11 shows agonist activity of three clinical OX40 agonists, in comparison to crosslinked natural ligand (OX40L-Fc+GAH). FIG. 11A shows the activity of the mAbs in the absence of the independent crosslinking agent, GAH. FIG. 11B shows the activity of the mAbs in the presence of the independent crosslinking agent, GAH.

As expected, the agonist activities of the three clinical mAbs demonstrated minimal activity by themselves, but were comparable to the natural ligand, OX40L, in presence of cross-linking. As shown in FIGS. 11A and 11B, the activation assay was performed in the range of 0.01-30 nM mAb. At a fixed concentration such as 6 nM, the efficacy of OX40 agonist largely depends on the amount of crosslinker. Therefore, experimental conditions below 10 nM of mAb, such as 6 NM, were considered reliable for interpretation of observed efficacies.

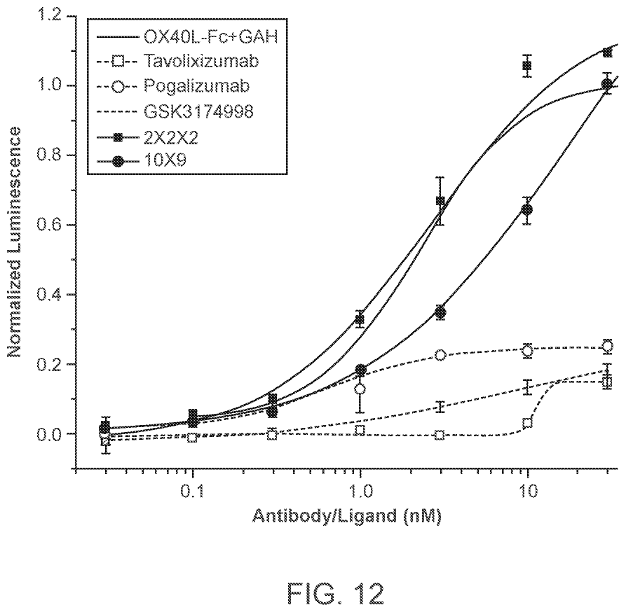

As discussed above, our two strongest OX40 agonists from this first discovery campaign were the bispecific bivalent (1×1) construct “10×9” and monospecific trivalent (2×1) construct “2×2×2”. FIG. 12 compares the three anti-OX40 clinical mAbs in the absence of GAH crosslinking (black dashed lines) to our top bivalent construct “10×9” (blue solid line), top trivalent construct “2×2×2” (red solid line), and crosslinked antigen (black solid line). Both of our constructs were seen to possess activity comparable to the crosslinked natural ligand, OX40L, in the absence of an independent cross-linking agent, and demonstrated increased agonism as compared to the three known clinical anti-OX40 mAbs.

### 6.10.5. Example 4: Expanded High Throughput Agonist Discovery

An expanded screen, performed essentially as described above, increased the number of identified candidate B-body OX40 agonists from 17 to 40. Briefly, B-Body candidate agonists were transiently expressed and purified using the one-step CH1 purification scheme. Candidates were added to HEK 293-NFkb-GFP/Luc-OX40 in soluble form without additional cross-linker or immobilization, and luminescence was read as agonistic activity from NFkB activation through OX40. The natural OX40 ligand Fc fusion protein (“OX40L-Fc”) was used to establish 100% agonism. Three clinical anti-OX40 monoclonal antibodies were also tested (arrows from left to right: Pogalizumab, Tavolixizumab, and GSK3174998)

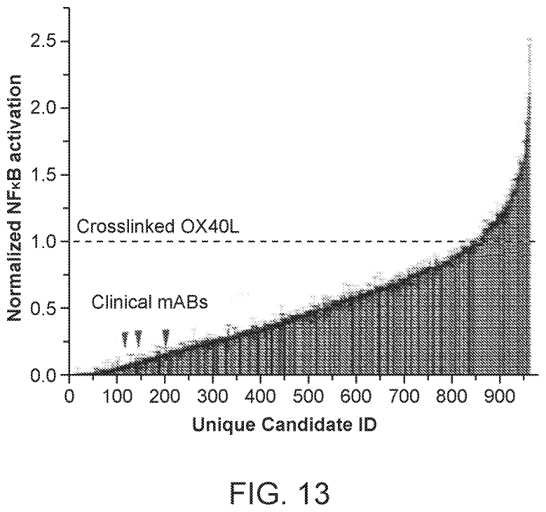

As shown in FIG. 13, greater than 900 combinations of affinity, epitope, and geometries (either the 1×1 bivalent or 2×1 trivalent B-Body platform constructs) were screened and resulted in a wide range of activities in the reporter cell assay. The three clinical OX40 antibodies demonstrated minimal activity in the absence of an additional cross-linker consistent with published results.

### 6.10.6. Example 5: Candidate Agonist Activity Demonstrates a Range of Responses

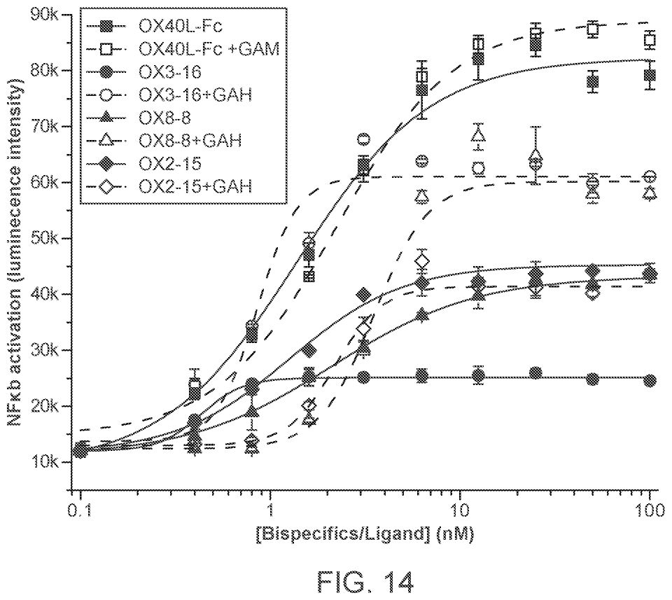

FIG. 14 shows agonist activity of three bivalent OX40 agonists in the absence and presence (+GAH) of the goat-anti-human (GAH) antibody crosslinking agent, as well as agonist activity of the control, crosslinked natural ligand-Fc fusion (OX40L-Fc), in the absence and presence of goat-anti-mouse (GAM) antibody crosslinking agent (OX40L-Fc+GAM).

The three bivalent OX40 agonists tested displayed a large variation in EC₅₀, maximum efficacy and sensitivity to cross-linking. The difference in the dose response curves highlight potential differences in the mechanism of agonism for each. Thus, bivalent OX40 agonists with varying agonist characteristics can be identified, and can be classified based on properties additional to simple affinity. The improved characterization of each OX40 agonist may identify potential beneficial properties that can be exploited in a clinical setting.

### 6.10.7. Example 6: Dose Response Curves for Bispecific Agonist Candidates

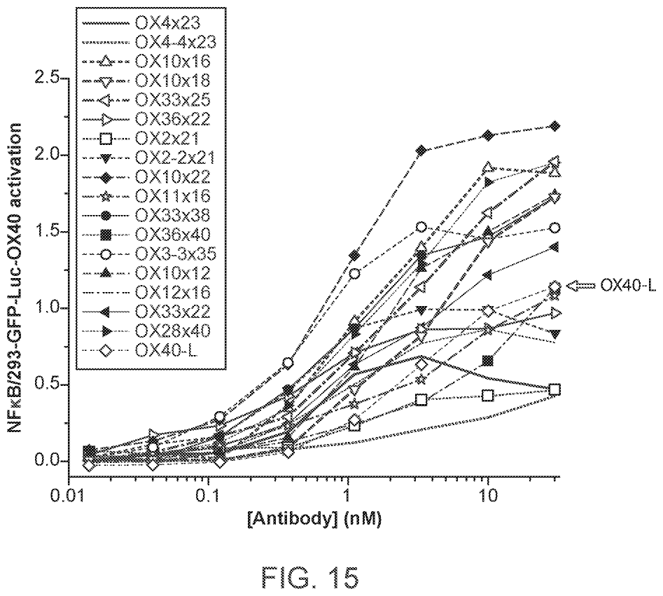

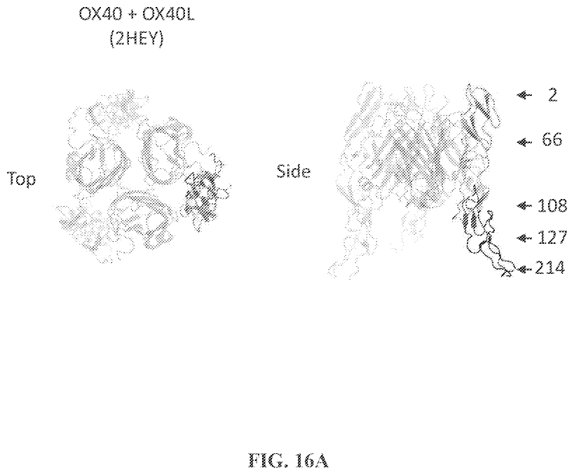

FIG. 15 shows dose response curves for a subset of bispecific OX40 agonists identified during the high throughput screen. Agonist activity was tested using NFκB activation to identify potent agonists. Candidates were expressed and purified by one-step CH1 affinity chromatography. Dose response experiments were performed using the HEK 293-NFkb-GFP/Luc-OX40 reporter assay in a range of 0.03 nM to 30 nM. Multiple bispecific OX40 agonists were identified that are more potent than cross-linked OX40 ligand-Fc fusion (OX40L-Fc).

### 6.10.8. Example 7: Epitope Mapping of OX40 Antigen Binding Sequences

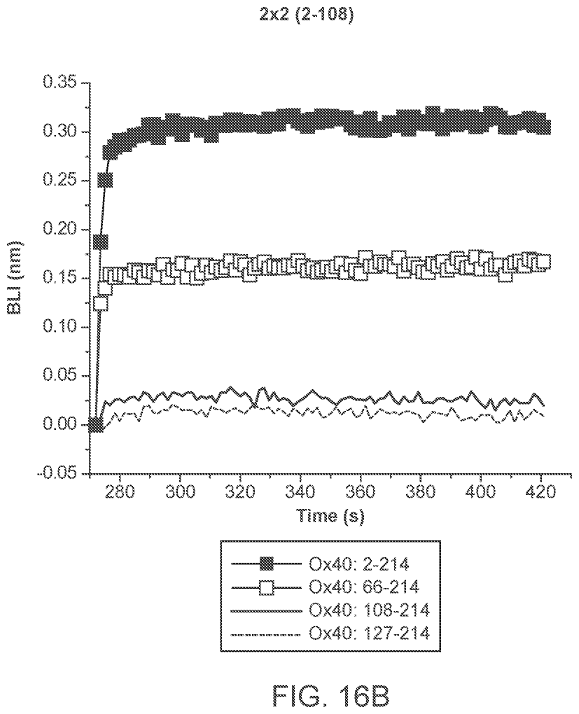

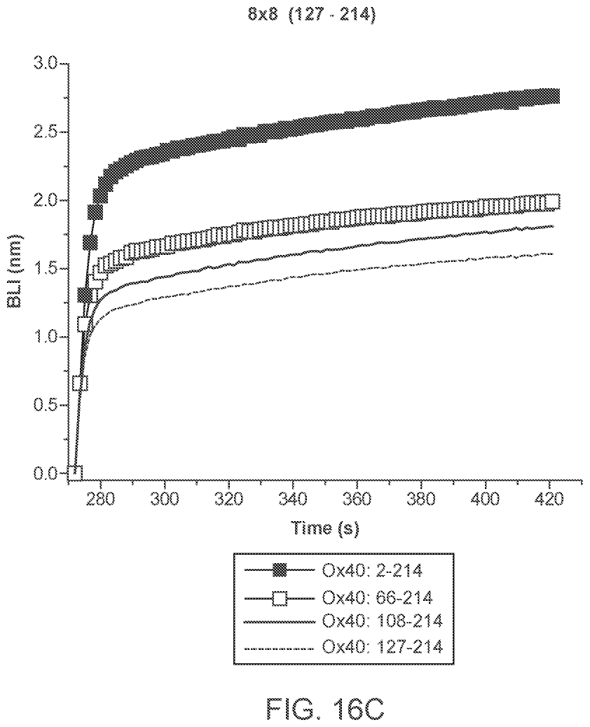

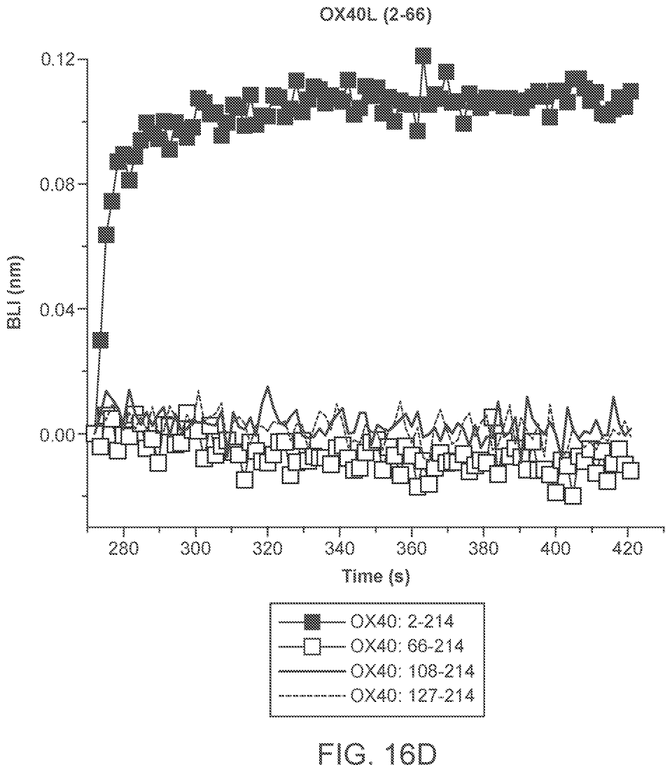

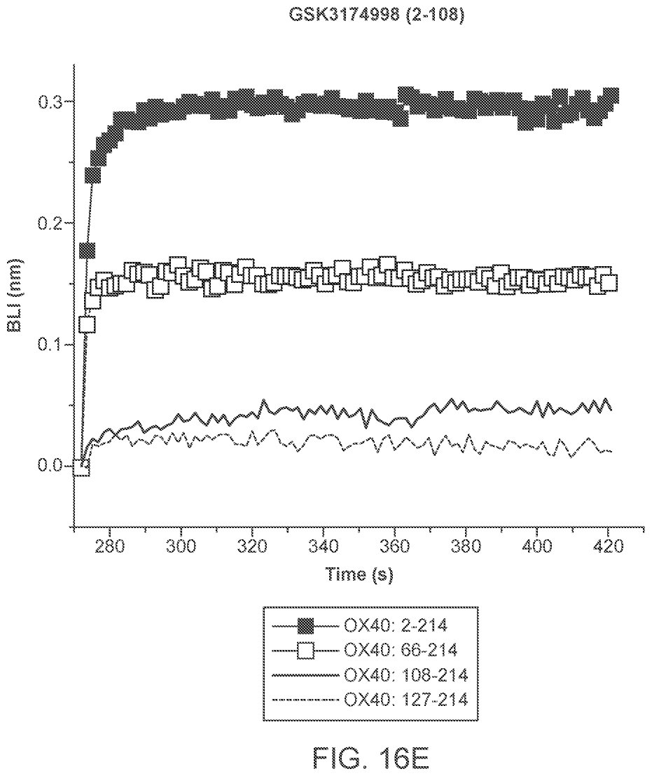

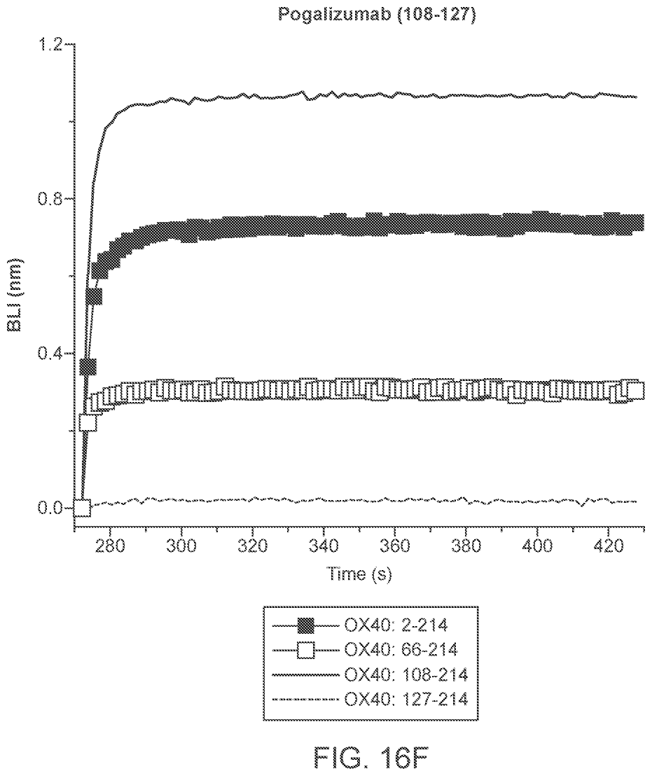

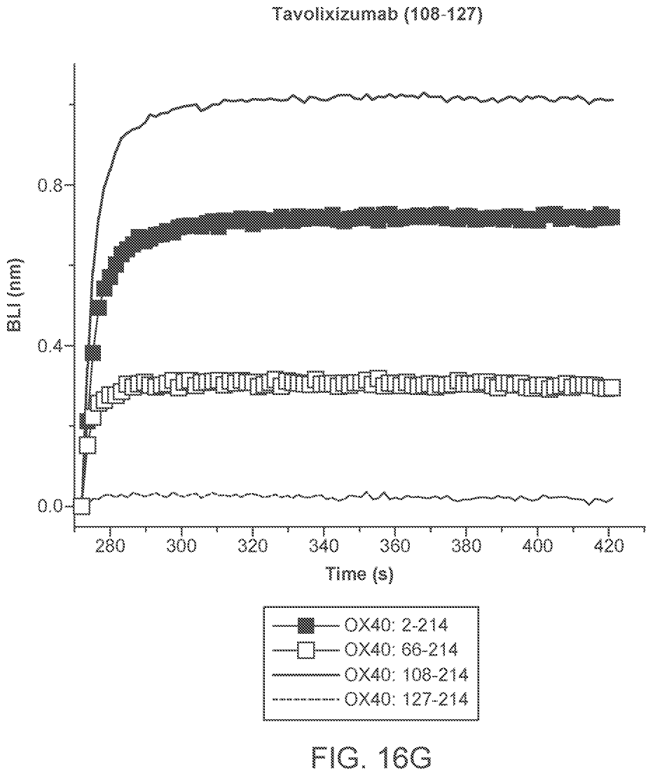

Two candidates with non-overlapping OX40 antigen binding sites (OX40:2 and OX40:8) identified in the screen as well as OX40L-Fc and clinical OX40 antibodies were investigated further to determine the specific OX40 epitope bound by each. FIG. 16A illustrates OX40 and OX40L bound in trimer from a top view (left panel) and side view (right panel). The extracellular domain of OX40 consists of four cysteine rich domains (CRD) with boundaries for each CRD noted. FIG. 16B shows binding for the different monospecific antibodies to different OX40 fragments having a series of truncations from the N-terminus (AA 2-214, AA 66-214, AA 108-214, and AA 127-214). The OX40 fragments were prepared as Fc fusion proteins, and also contained a signal peptide, an Avi-tag, a TEV cleavage site, and a HIS tag for purification. The full length or truncated OX40-Fc fusion proteins were immobilized onto BLI sensor and the different monospecific antibodies included the clinical antibodies GSK3174998, Pogalizumab, and Tavolixizumab, as well as monospecific bivalent BC1 formatted candidates with either the OX40 antigen binding site OX40:2 (“2×2”) or the OX40 antigen binding site OX40:8 (“8×8”).

OX40L demonstrated binding only to immobilized full length fragment (OX40:2-214), indicating that OX40L only bound the first CRD (amino acids 2-66). The OX40:2 antigen binding site and the clinical antibody GSK3174998 demonstrated binding to the full length fragment (OX40:2-214) and partial binding to the first truncation (OX40:66-214), indicating that both bound the first and second CRD (amino acids 2-108). The other two clinical antibodies, Pogalizumab and Tavolixizumab, demonstrated the strongest binding to the fragment OX40:108-214, while binding was no longer present in the OX40:127-214 truncation, indicating that both bound the third CRD (amino acids 108-127). The OX40:8 antigen binding site demonstrated binding to all tested truncations of OX40, indicating binding to the fourth CRD (amino acids 127-214). Thus, our OX40 screen identified antigen binding sites that bind epitopes that did not overlap (OX40:2 binding an epitope within amino acids 2-108 and OX40:8 binding an epitope within amino acids 127-214), as well as an antigen binding site that binds an epitope different from that bound by the tested clinical monoclonal antibodies (OX40:8 binding an epitope within amino acids 127-214).

### 6.10.9. Example 8: Measuring Binding of Non-Overlapping Epitopes

Candidate OX40 antigen binding sites identified in the screen were tested in combination for simultaneous binding to OX40. As shown in FIG. 17, 100 nM biotinylated OX40 was immobilized through streptavidin to a BLI sensor (“+OX40”). After baseline equilibration, 100 nM of a first candidate antigen binding site formatted in a native monospecific IgG antibody conformation (top panel OX40:8; middle panel OX40:21; bottom panel OX40:35) was added as indicated, with each demonstrating binding to OX40. Next, 100 nM of a second candidate antigen binding site also in a native monospecific IgG antibody conformation (top panel OX40:2; middle panel OX40:2; bottom panel OX40:3) was added together with 100 nM of the first antibody, as indicated. In the three combinations tested, additional binding by the second antibody was demonstrated indicating ability of both antibodies to simultaneously bind OX40. Thus, the antigen binding site combinations bound non-overlapping epitopes.

FIG. 18 summarizes the results of the binding experiments for the panel of the 40 antigen binding sites in all possible combinations (i.e., a 40×40 matrix). The level of the BLI response is based on the mass of the antibodies bound. The expected BLI response level was predicted for complete binding by both a first and second OX40 candidate agonist. Thus, the percentage of expected binding when both candidates are added was calculated and used to determine if both are binding at the same time. Molecules with a higher percentage of expected binding indicated simultaneous binding of OX40 by both candidates, suggestive of non-overlapping epitopes. The top row identifies the first antibody added to the sensor, and the first column identifies the second antibody. Shaded squares identify binding site combinations that demonstrated simultaneous binding to non-overlapping epitopes.

### 6.10.10. Example 9: T Cell Activation by Non-Crosslinked OX40 Agonist Candidates

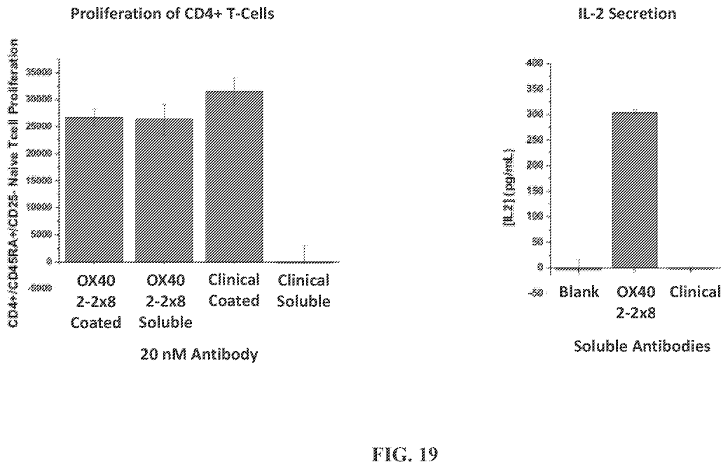

Candidate OX40 agonists were screened in CD4+/CD45RA+/CD25 naive T cell assays. Soluble candidates were directly applied to the primary cell assay and a clinical mAB GSK3174998 was applied in both soluble and plate-coated forms as controls. The T cell proliferation was assayed by PrestoBlue and IL-2 secretion was quantified by ELISA. As shown in FIG. 19, GSK3173998 only stimulated T cell proliferation when bound to a plate, while no proliferation was detected when soluble GSK3173998 was added (left panel). In contrast, the bispecific trivalent B-body OX40:2-2×8 stimulated similar levels of T cell proliferation regardless of being soluble or plate bound, suggesting receptor clustering activity in the absence of a crosslinking agent. Measuring IL-2 secretion also demonstrated activity of soluble OX40:2-2×8 but not soluble GSK3173998.

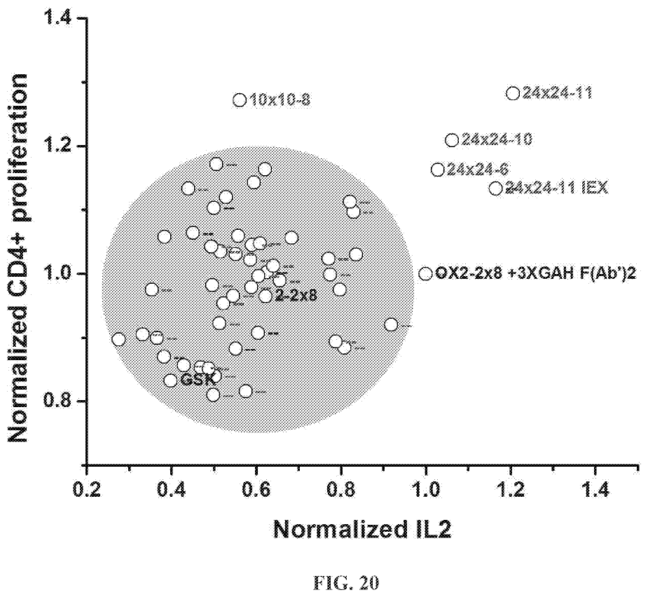

FIG. 20 shows a summary of T cell stimulatory activity for different multi specific multivalent candidate OX40 agonists. The X-axis represents the IL2 secretion, while the Y-axis is the CD4+/CD45RA+/CD25− T cell proliferation for each candidate. The shaded circle provides a cutoff for those agonists considered potent, with those lying outside of the circle considered potent. All of the agonists lying outside the circle were bispecific trivalent B-bodies in a 2×1 format and had the highest potency.

Candidates of interest identified in the screen are:

OX40:24-24×11
Chain 1: VL (OX40:24)-CH3 (BC1)-GS linker-VL (OX40:24)-CH3 (BC1)-CH2-CH3 (Knob, 354C) [SEQ ID NO:47]
Chain 2: VH (OX40:24)-CH3 (BC1) [SEQ ID NO:48]
Chain 3: VL (OX40:11)-CL-CH2-CH3 (Hole, 349C) [SEQ ID NO:49]
Chain 4: VH (OX40:11)-CH1 [SEQ ID NO:50]
Chain 5: equivalent to chain 2

OX40: 24-24×10
Chain 1: VL (OX40:24)-CH3 (BC1)-GS linker-VL (OX40:24)-CH3 (BC1)-CH2-CH3 (Knob, 354C) [SEQ ID NO:47]
Chain 2: VH (OX40:24)-CH3 (BC1) [SEQ ID NO:48]
Chain 3: VL (OX40:10)-CL-CH2-CH3 (Hole, 349C) [SEQ ID NO:51]
Chain 4: VH (OX40:10)-CH1 [SEQ ID NO:52]
Chain 5: equivalent to chain 2

OX40: 24-24×6
Chain 1: VL (OX40:24)-CH3 (BC1)-GS linker-VL (OX40:24)-CH3 (BC1)-CH2-CH3 (Knob, 354C) [SEQ ID NO:47]
Chain 2: VH (OX40:24)-CH3 (BC1) [SEQ ID NO:48]
Chain 3: VL (OX40:6)-CL-CH2-CH3 (Hole, 349C) [SEQ ID NO:53]
Chain 4: VH (OX40:6)-CH1 [SEQ ID NO:54]
Chain 5: equivalent to chain 2

OX40: 24-24×4
Chain 1: VL (OX40:24)-CH3 (BC1)-GS linker-VL (OX40:24)-CH3 (BC1)-CH2-CH3 (Knob, 354C) [SEQ ID NO:47]
Chain 2: VH (OX40:24)-CH3 (BC1) [SEQ ID NO:48]
Chain 3: VL (OX40:4)-CL-CH2-CH3 (Hole, 349C) [SEQ ID NO:55]
Chain 4: VH (OX40:4)-CH1 [SEQ ID NO:56]
Chain 5: equivalent to chain 2

### 6.10.11. Example 10: Real-Time Quantification T Cell Activation

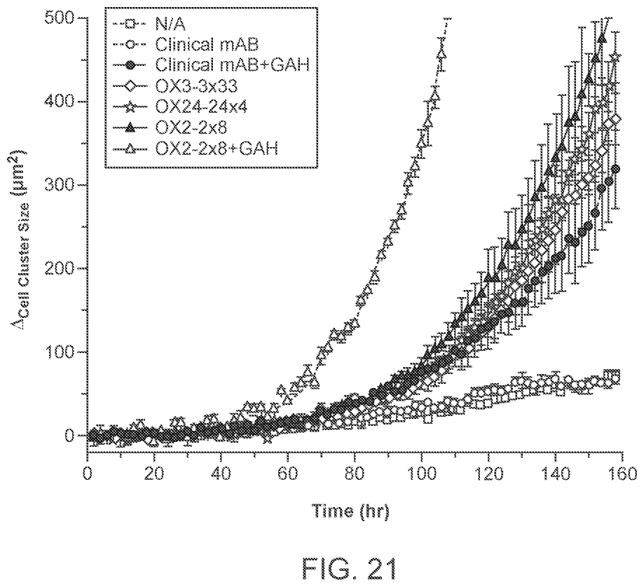

Real-time activation of T cells was monitored using the Incucyte system (Sartorius). The kinetics of T cell activation were monitored by microscopy and charted using cell size measurement to track the growth and proliferation of T cell clusters. As shown in FIG. 21, the clinical monoclonal antibody GSK3174998 (“Clinical mAB”) required cross-linking to stimulate significant T cell proliferation. In contrast, three separate bispecific trivalent B-body stimulated T cell proliferation in the absence of additional cross-linking agents and with kinetics faster than cross-linked GSK3174998. In addition, cross-linking of bispecific trivalent B-body OX40:2-2×8 increased the kinetics of T cell proliferation even further. Thus, the screens described above identified multivalent multispecific agonists capable of OX40 receptor clustering activity in primary cells.

### 6.10.12. Example 11: Two Step Purification of OX40 Agonist Candidates

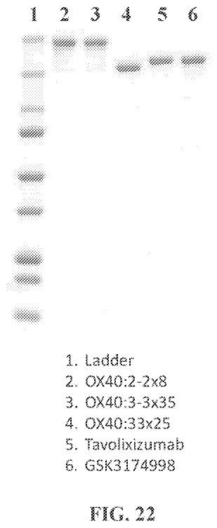

Candidate OX40 agonists and clinical monoclonal antibodies were purified using a two-step purification process. OX40:2-2×8, OX40:3-3×25, and OX40:33×25 were purified by CH1 and anion exchange chromatography, while the clinical antibodies were purified by Protein A and anion exchange chromatography. FIG. 22 shows a non-reducing SDS-PAGE analysis of the two-step purified antibodies demonstrating a high level of purity.

### 6.10.13. Example 12: T Cell Activation by OX40 Agonist Candidates

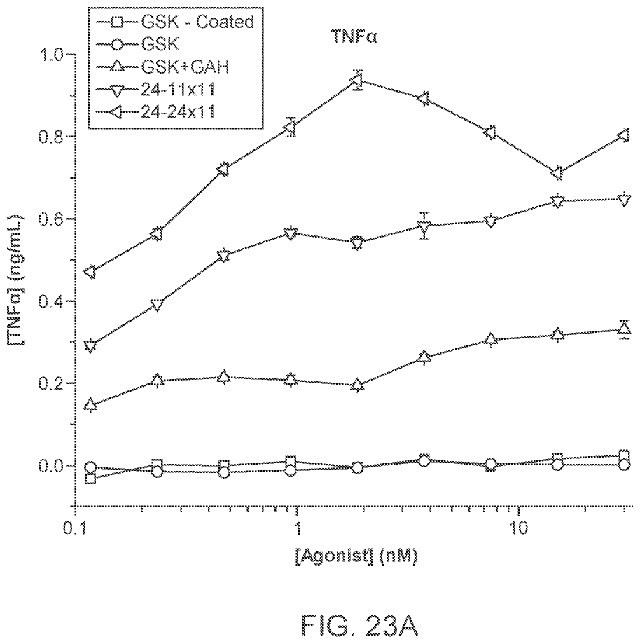

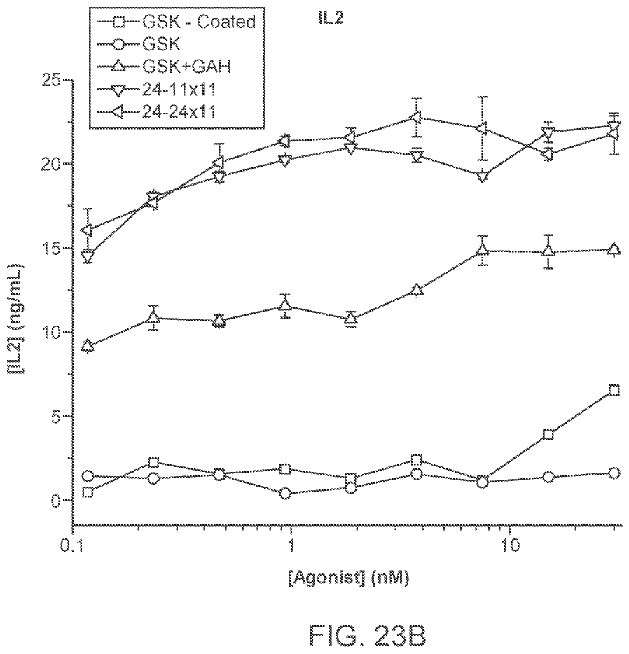

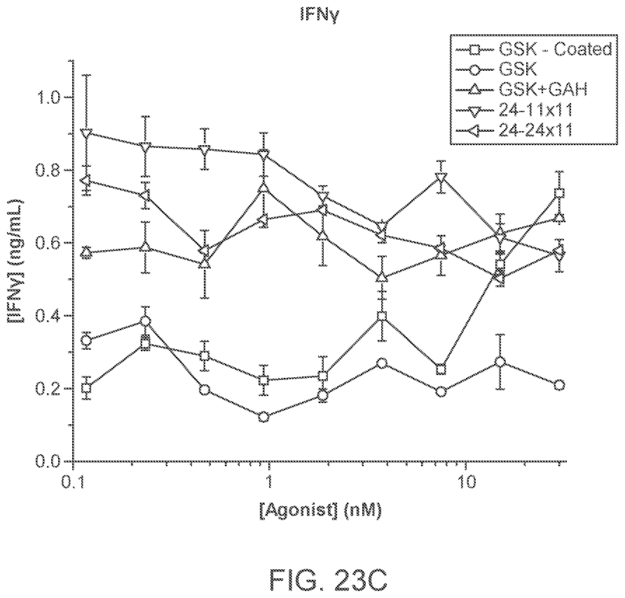

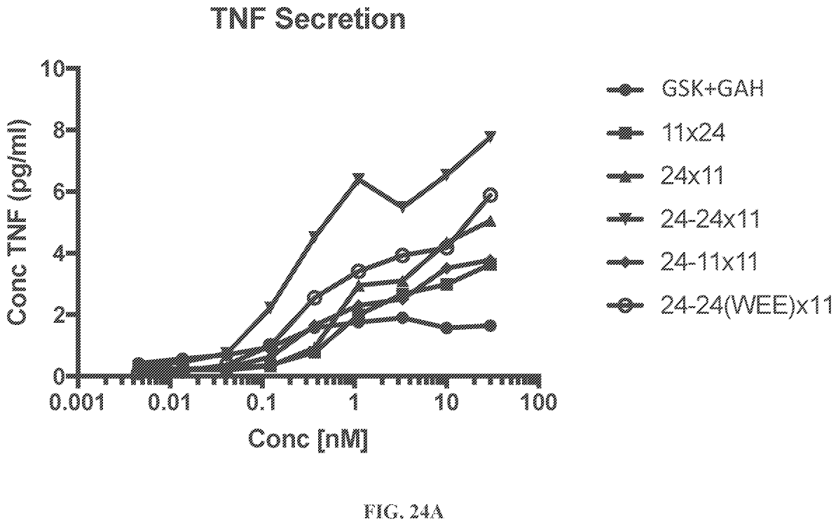

Activation of T cells using OX40 agonist candidates in a soluble 2×1 format was monitored by cytokine secretion (see FIG. 23-27). The OX40:24-24×11 is described above, and OX40:24-11×11 is described below.

OX40: 24-11×11
Chain 1: VL (OX40:24)-CH3 (BC1)-GS linker-VL (OX40:11)-CL-CH2-CH3 (Knob, 354C) [SEQ ID NO:57]
Chain 2: VH (OX40:11)-CH1 [see SEQ ID NO:50]
Chain 3: VL (OX40:11)-CL-CH2-CH3 (Hole, 349C) [SEQ ID NO:49]
Chain 4: VH (OX40:11)-CH1 [see SEQ ID NO:50]
Chain 5: VH (OX40:24)-CH3 (BC1) [SEQ ID NO:48]

As shown in FIG. 23, OX40:24-11×11 and OX40:24-24×11 both stimulated T cells greater than cross-linked GSK3174998 (“GSK+GAH”) as measured by TNFα and IL-2 secretion (FIG. 23A and FIG. 23B, respectively), and comparable activity to cross-linked GSK3174998 by IFNγ secretion (FIG. 23C). Notably, both OX40:24-11×11 and OX40:24-24×11 demonstrated activity at the lowest antibody concentration tested suggesting the constructs were active in the soluble format, while soluble GSK3174998 did not result in detectable activation and plate-bound (“coated”) GSK3174998 demonstrated activity only at higher antibody concentrations.

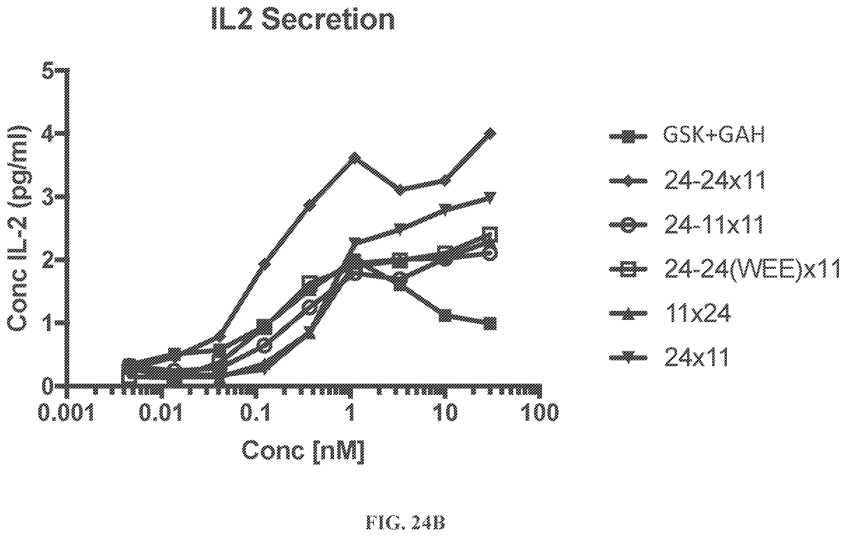

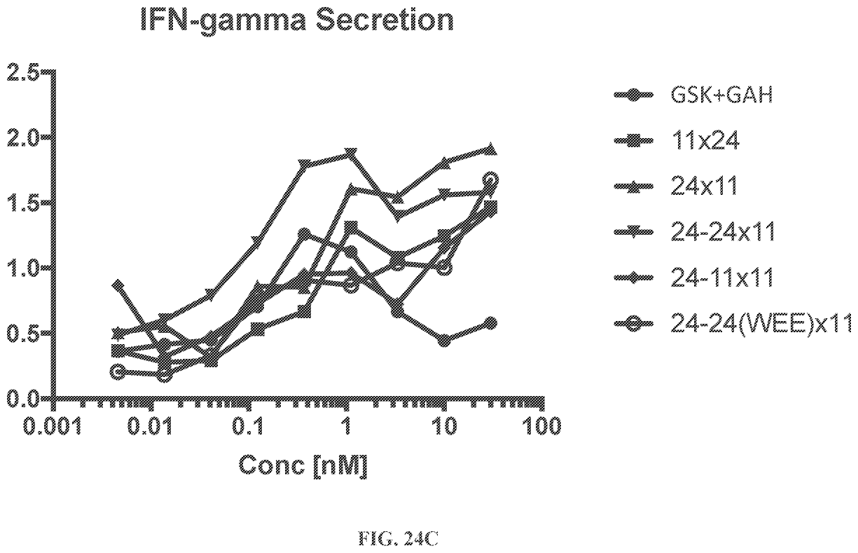

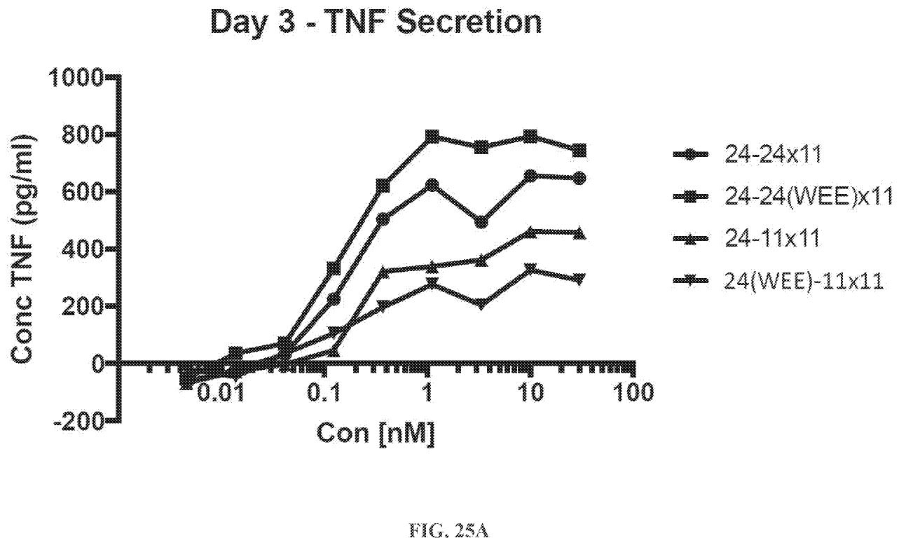

As shown in FIG. 24, OX40:24-11×11 and OX40:24-24×11 both demonstrated activity in the sub-nanomolar range as measured by TNF, IL-2, and IFNγ secretion (FIG. 24A-C, respectively).

Also shown in FIG. 24 is a modified OX40:24-24×11 construct, termed OX40:24-24(WEE)×11, with two aspartic acids in each OX40 antigen binding site modified to glutamic acid residues to remove a potential proteolytic cleavage site in Chain 1 (see SEQ ID NO:59, all other chains equivalent). OX40:24-24(WEE)×11 demonstrated agonist activity greater than the crosslinked GSK3174998 control, though slightly less than the unmodified version in this assay.

A modified version of OX40:24-11×11, termed “OX40:24(WEE)-11×11,” was also constructed (see SEQ ID NO:58 for Chain 1, all other chains equivalent).

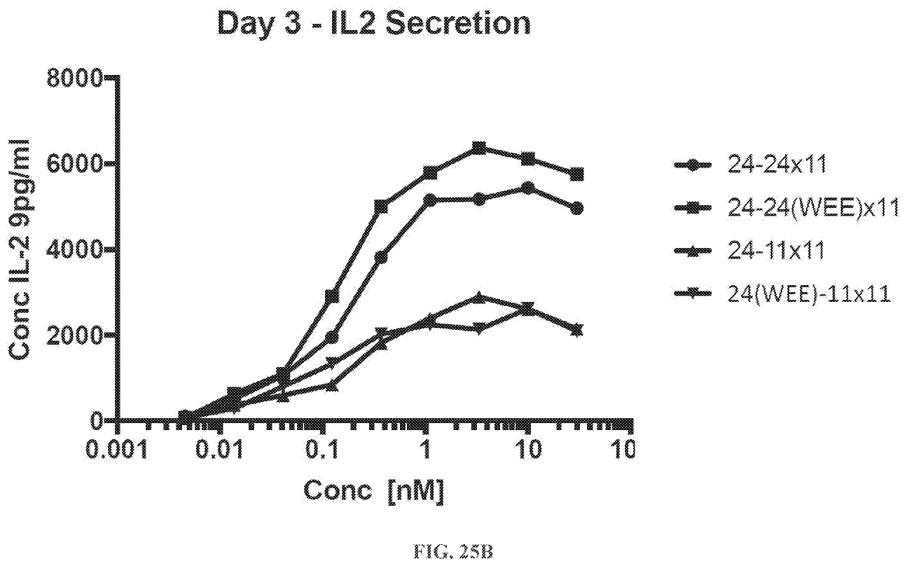

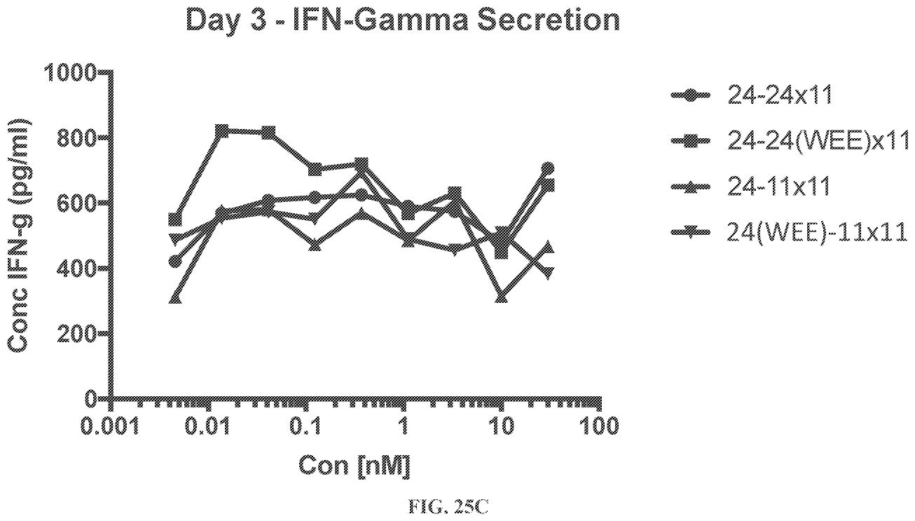

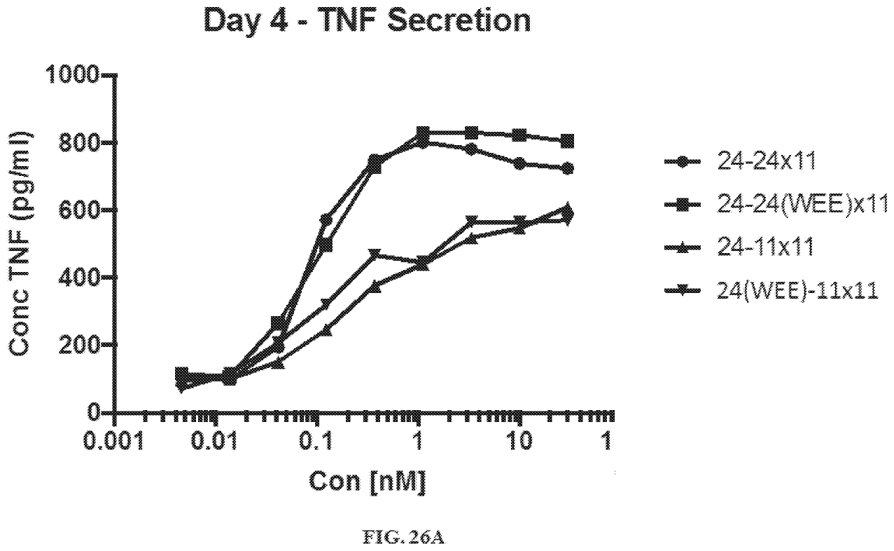

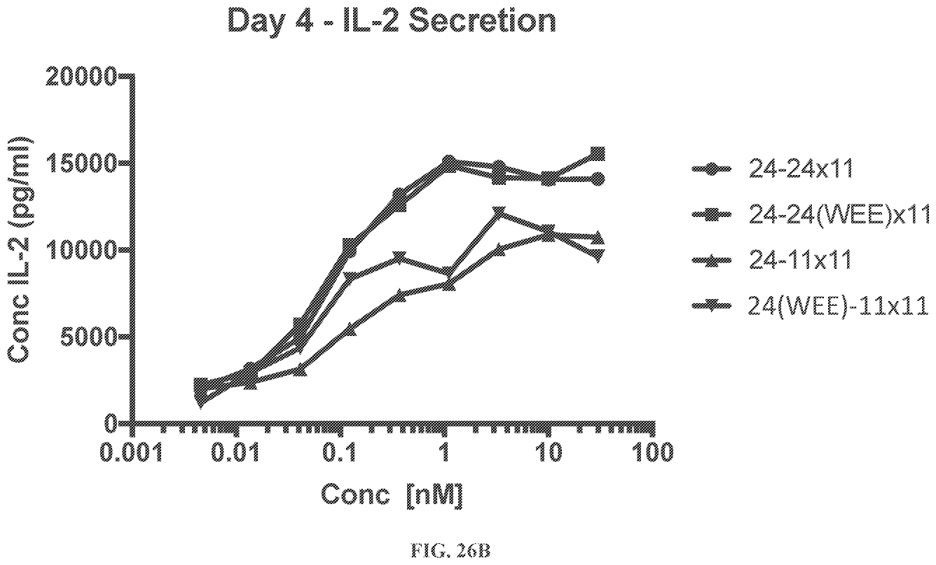

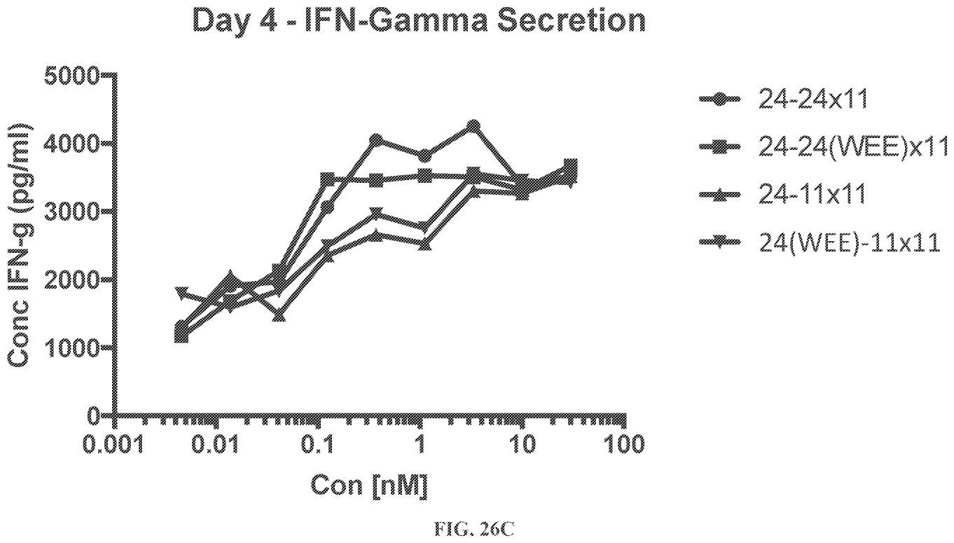

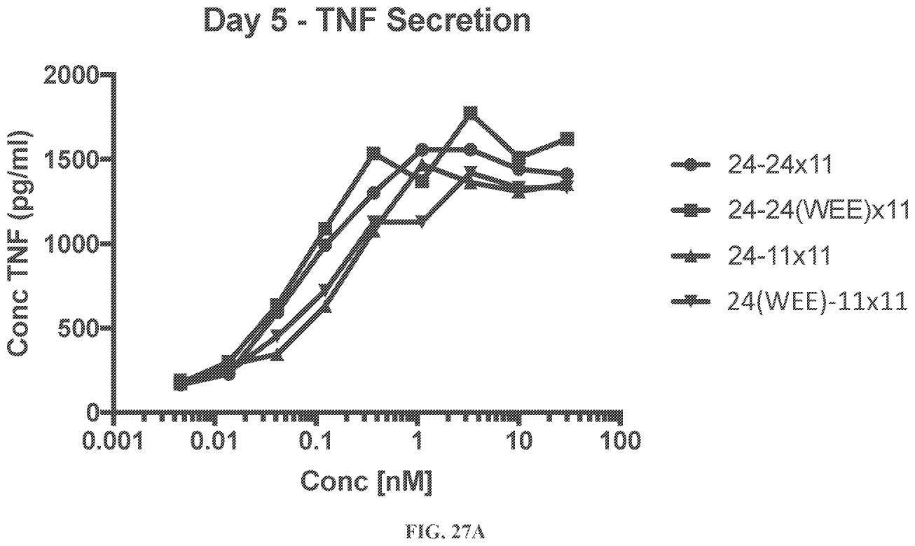

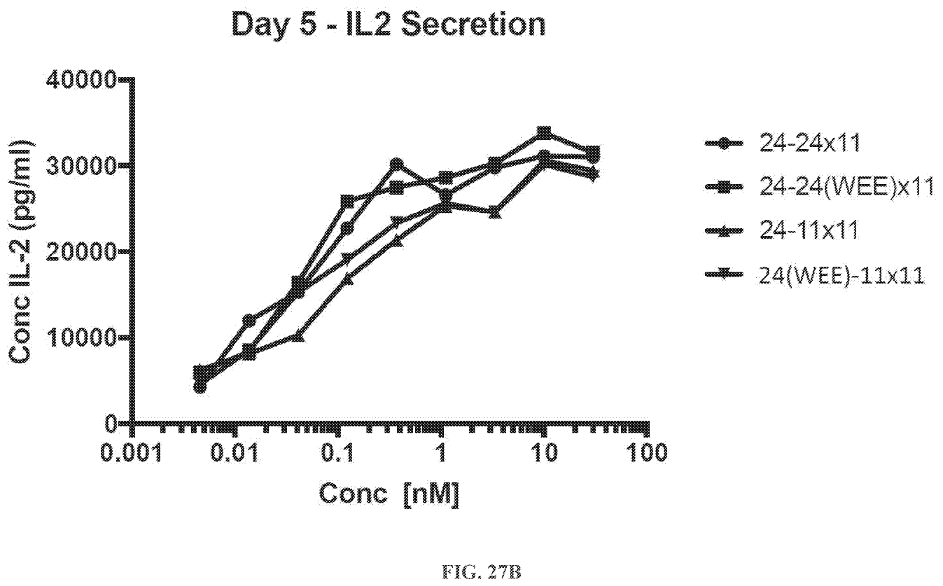

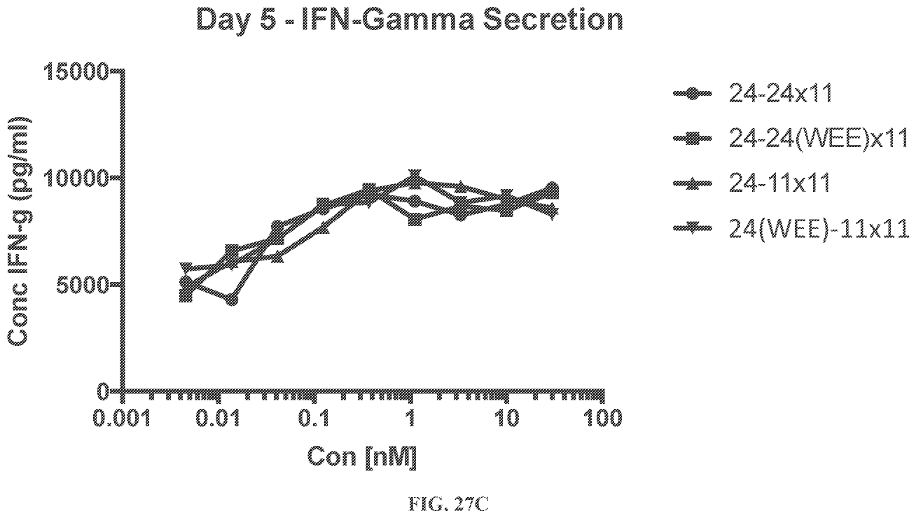

The kinetics of cytokine secretion were also tested. As shown in FIGS. 25-27, both OX40:24-24×11 and OX40:24-24(WEE)×11 demonstrated greater activity than OX40:24-11×11 and OX40:24(WEE)-11×11 as measured by TNFα and IL-2 secretion at Day 3 (FIG. 25A and FIG. 25B, respectively). No dose dependent response for IFNγ secretion was seen for any of the constructs at Day 3 (FIG. 25C). The difference between OX40:24-24×11 and OX40:24-11×11 based constructs for TNFα and IL-2 secretion was reduced at Day 4 (FIG. 26A and FIG. 26B, respectively), and even further reduced to almost negligible differences at Day 5 (FIG. 27A and FIG. 27B, respectively). Dose dependent response for IFNγ secretion was seen at Days 4 and 5 (FIG. 26C and FIG. 27C, respectively), but with minimal difference between constructs. Thus, trivalent bispecific formats using different combinations of ABS binding sites were demonstrated to have different agonistic kinetics for cytokine secretion.

### 6.10.14. Example 13: T Cell Proliferation by OX40 Agonist Candidates

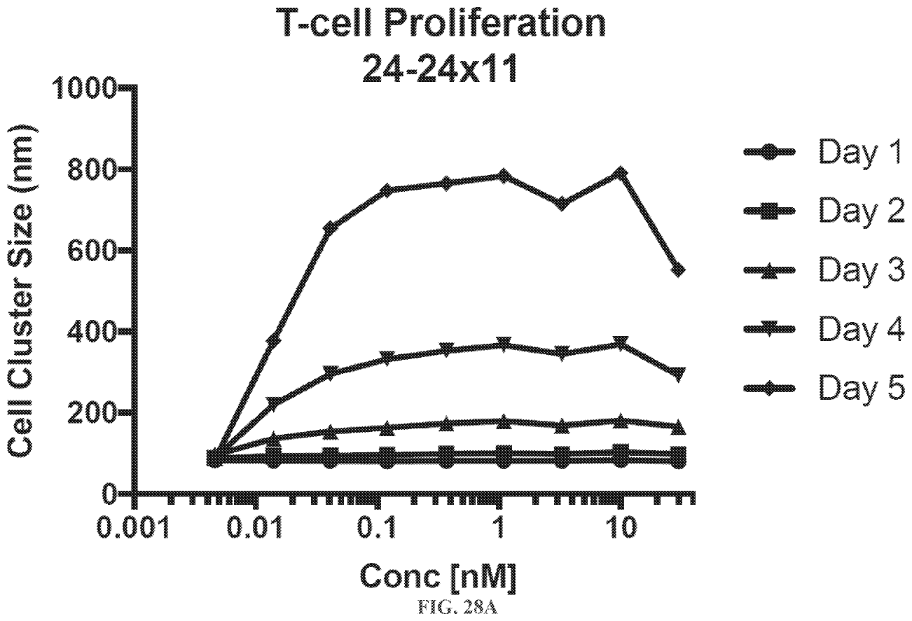

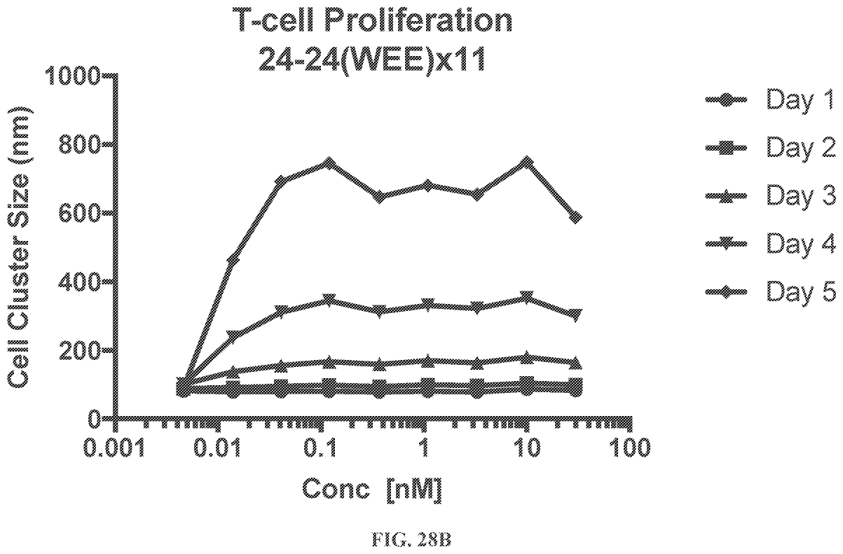

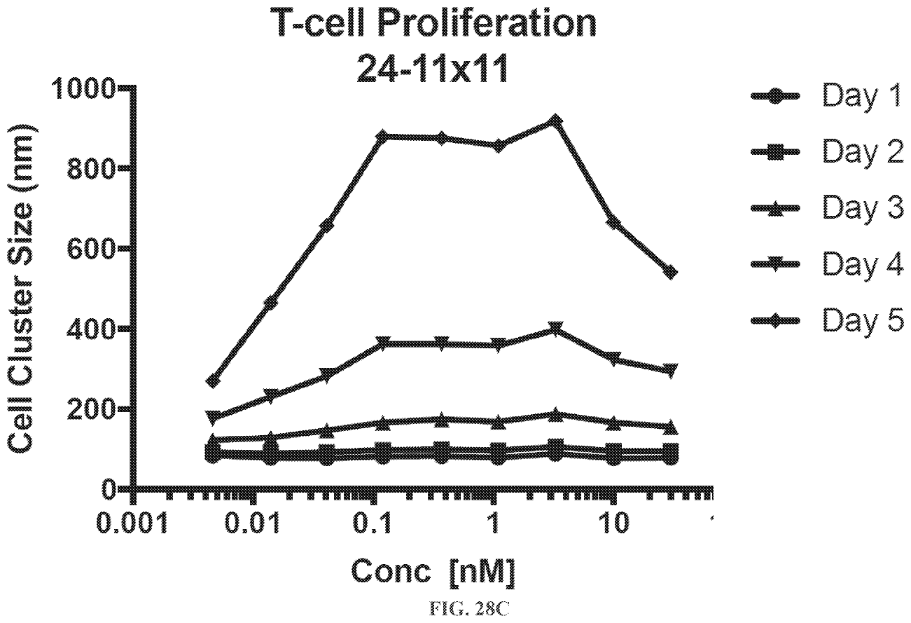

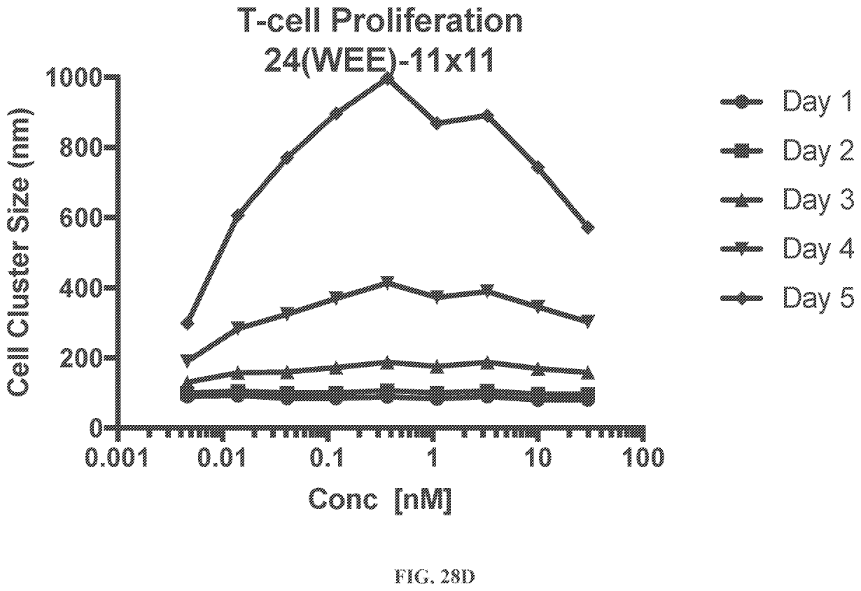

Activation of T cells using OX40 agonist candidates in a soluble 2×1 format was also monitored by proliferation. As shown in FIG. 28, candidates OX40:24-24×11 (FIG. 28A), OX40:24-24(WEE)×11 (FIG. 28B), OX40:24-11×11 (FIG. 28C), and OX40:24(WEE)-11×11 (FIG. 28D) all proliferated with similar kinetics. Notably, proliferation for all the constructs was attenuated at higher antibody concentrations. However, the concentration of antibody that resulted in attenuated proliferation varied between constructs, with peak proliferation having resulted at 10 nM for OX40:24-24×11 based constructs at concentrations below 10 nM for OX40:24-11×11 based constructs. Thus, trivalent bispecific formats using different combinations of ABS binding sites were demonstrated to have different T cell proliferation agonism activities.

### 6.10.15. Example 14: T Cell Activation by Different Valency Formats

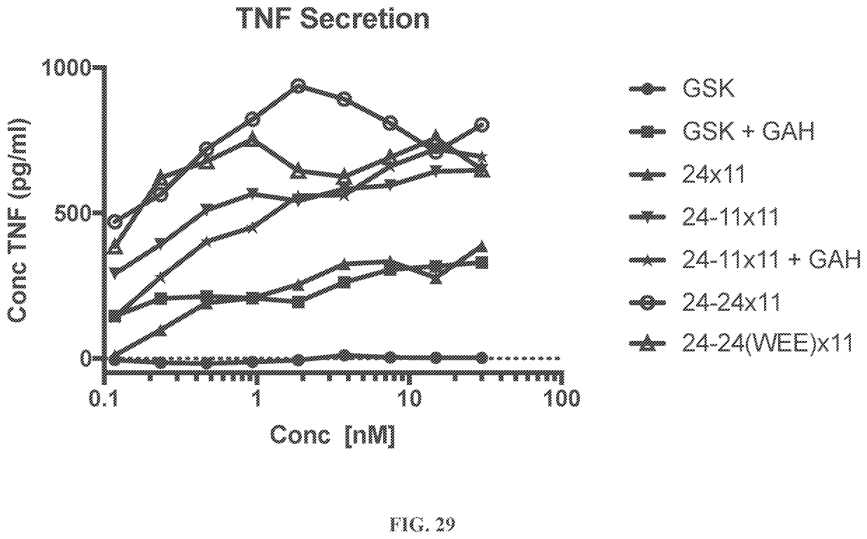

Various B-body constructs with different valencies using ABS OX40:24 and OX40:11 were tested for agonist activity. As shown in FIG. 24, bispecific bivalent 1×1 B-body candidates OX40:24×11 (Chain 1 SEQ ID NO:222, Chains 2-4 SEQ ID NO:48-50) and OX40:11×24 (Chains 1-4 see SEQ ID NO:223-226) resulted in cytokine production as measured by TNF, IL-2, and IFNγ secretion (FIG. 24A-C, respectively). As shown in FIG. 29, the bispecific bivalent 1×1 candidate OX40:24×11 demonstrated activity greater than soluble and plate-bound (“coated”) GSK3174998, and activity comparable to cross-linked GSK3174998 by TNFα secretion.

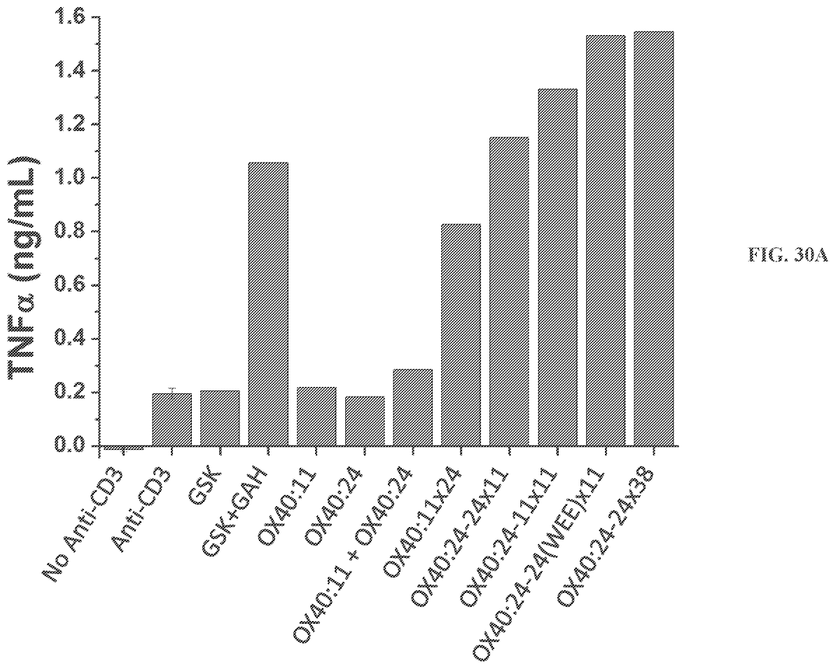

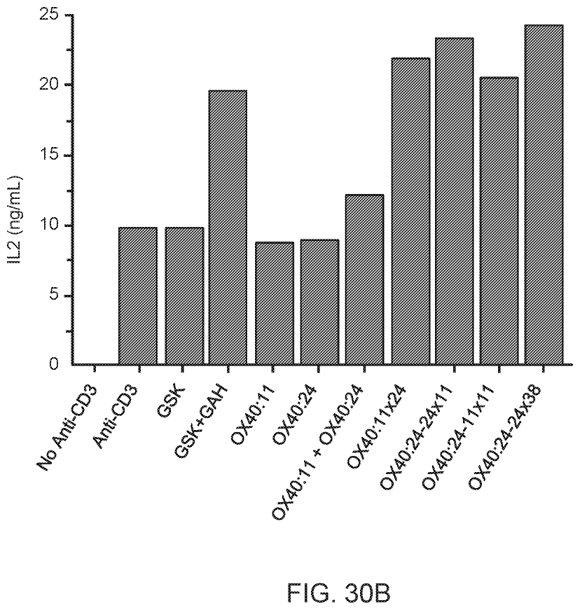

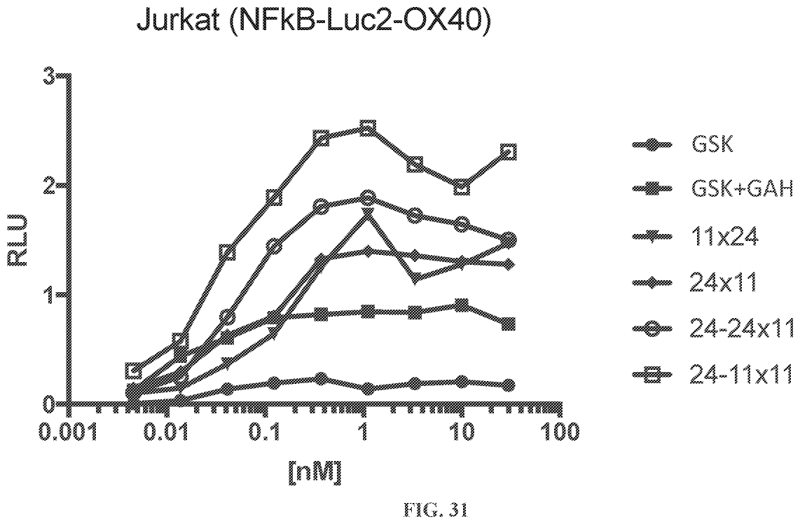

Monospecific bivalent 1×1 candidates OX40:24 and OX40:11 formatted in a native IgG architecture were compared alone and in combination against a bispecific bivalent 1×1 candidate (OX40:11×24) and various bispecific trivalent 2×1 candidates. As shown in FIG. 30, native IgG formats of OX40:11 and OX40:24 candidates demonstrated minimal activity comparable to soluble anti-CD3 and soluble GSK3174998 as measured by TNFα and IL-2 secretion (FIG. 30A and FIG. 30B, respectively). A combination of both OX40:11 and OX40:24 candidates (“OX40:11+OX40:24”) resulted in detectable activity, though significantly below the bispecific bivalent 1×1 candidate and bispecific trivalent 2×1 candidates. Thus, bispecific formats (both bivalent and trivalent) resulted in significant agonist activity, while monospecific formats of combinations of monospecific native IgG formats did not.

Notably, the bispecific trivalent 2×1 candidates demonstrated increased agonist activity compared to the bispecific bivalent 1×1 candidate as measured by TNFα secretion (FIG. 30A).

### 6.10.16. Example 15: T Cell Activation by Cross-Linking

Various B-body constructs using ABS OX40:24 and OX40:11 were tested for agonist activity in combination with cross-linking. As shown in FIG. 29, the clinical antibody GSK3174998 required addition of a soluble cross-linker (“GSK+GAH”) to demonstrate activity as compared to GSK3174998 in the absence of cross-linking (“GSK”) as measured by TNFα secretion. In contrast, cross-linking the OX40:24-11×11 candidate (“24-11×11+GAH) did not increase activity, and potentially reduced activity. Thus, the optimal format for the bispecific trivalent 2×1 candidate was in a soluble form in the absence of additional cross-linker.

### 6.10.17. Example 16: Jurkat T Cell Activation by Candidates

Various B-body constructs using ABS OX40:24 and OX40:11 were tested for agonist activity in an NFκB Luc2 OX40 Jurkat T cell stimulation assay. As shown in FIG. 32, bispecific trivalent OX40:24-24×11 and OX40:24-11×11 candidates demonstrated the greatest activity as measured by luciferase, with OX40:24-11×11 detectably above OX40:24-24×11. Bispecific bivalent OX40:24×11 and OX40:11×24 candidates also demonstrated activity, with both above the cross-linked clinical antibody GSK3174998. Thus a range of agonist activity was seen across B-body formats, with bispecific trivalent candidates having increased potency in comparison to bispecific bivalent candidates.

### 6.10.18. Example 17: Biophysical Properties of OX40 Agonist Candidates

Bispecific trivalent OX40 agonist candidates OX40:24-24×11 and OX40:24-11×11 were assessed for biophysical properties, such as those relevant for production of antibodies in a clinical setting or on an industrial scale. As shown in Table 5, the candidates OX40:24-24×11 and OX40:24-11×11 demonstrated biophysical properties useful in manufacturing for clinical and industrial settings. Properties were assessed using standard assays. Examples of biophysical properties and methods to assess the same are described in more detail in Jain et al. (Proc Natl Acad Sci USA. 2017 Jan. 31; 114(5):944-949.), herein incorporated by reference for all it teaches. Properties assessed were yield, purity, homogeneity, stability, long-term stability, acid stability, thermostability, low antibody cross-interaction, low antibody self-interaction, low hydrophobic binding, and cyno crossreactivity.

TABLE 5
Manufacturing Properties of OX40 Agonist Candidates

| Assay | OX40:24-24x11 | OX40:24-11x11 |
|---|---|---|
| Primary Cell Activity | +++ | +++ |
| Yield/Purity | + | +++ |
| Homogeneity | +++ | +++ |
| Accelerated Stability | +++ | +++ |
| Acid Stability | ++ | ++ |
| Stability at 25 mg/ml | +++ | +++ |
| Thermostability | +++ | +++ |
| Analytical HPLC | ++ | ++ |
| (HIC, SMAC, & CIC) | | |
| Cyno Crossreactivity | +++ | +++ |

### 6.10.19. Discussion

OX40 mAbs require cross-linking to generate observable agonistic activities, as we demonstrated in our cellular assay with Tavolixizumab, Pagolizumab and GSK3174998. The clinical trials of these known clinical-stage antibodies therefore rely on the Fc receptor engagement to effect agonist activity, which may contribute to the low response rate that has been observed so far. The only clinical trial with significant efficacy (12/30 with tumor shrinkage) was conducted with a mouse anti-human OX40 antibody, and all responders were demonstrated to have significant amount of mouse-anti-human-antibody (MAHA), which likely act as cross-linkers, increasing in vivo efficacy of the OX40 antibody.

The B-Body platform, described in detail in U.S. patent application Ser. No. 15/787,640, filed Oct. 18, 2017, incorporated herein by reference, provides superior orthogonality, dramatically decreased incomplete pairing, and increased yield to ˜100 μg antibody construct/mL cell culture in various valency formats. Together with standardized cloning protocols, high throughput protein expression, and single-step purification, the B-Body platform allowed us to perform high throughput cellular assay screening for multivalent agonist antibodies.

Using this system, we successfully demonstrated that multivalent antibodies can be potent OX40 agonists by themselves, in the absence of an independent crosslinking agent, such as cellular FcγR or MAHA. Our antibody constructs were capable of clustering a TNFR superfamily member on the cell membrane through multivalent binding to the extracellular domain of TNFR. Our best performing candidates were superior to 3 known mAb clinical candidates, and offer a solution to the current challenge of low efficacy of human OX40 agonists in clinical trials.

This strategy is generally applicable to all TNFR superfamily members, and to certain other receptors that analogously require clustering for agonist activity. For example, this approach should be effective in producing agonists of CD20; it has been shown the efficacy of the anti-CD20 monoclonal antibody, rituximab, is largely due to CD20 cross-linking. Although standard bivalent monospecific mAbs can cluster receptors in principle, their potential is far less than that of our multivalent, multispecific, antibody constructs. We believe the combination of the high throughput discovery power of our B-Body platform and the strategy of using “multivalent agonist by clustering receptors” can open the door to treatment of new disease indications.

### 7. SEQUENCES

>Screening B-Body Chain 1 [SEQ ID NO: 1]
DIQMTQSPSSLSASVGDRVTITCRASQSVSSAVAWYQQKPGKAPKLLIYSASSLYSGVPSRFSGSRSGTDFTLTISSLQPEDF
ATYYCQQRDSYLWTFGQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVYTLPPCRDELTKNQVSLWCLVKGFYPSDIAVEWESNGQPENNYKTTPPVLDSDGSFFLYSKLTVDKSRWQQGN
VFSCSVMHEALHNHYTQKSLSLSPGK
>Screening B-Body Chain 2 [SEQ ID NO: 2]
EISEVQLVESGGGLVQPGGSLRLSCAASGFTFSTYYIHWVRQAPGKGLEWVAVIYPYTGFTYYADSVKGRFTISADTSKNTAY
LQMNSLRAEDTAVYYCARGEYTVLDYWGQGTLVTVSSASPREPQVYTDPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSGEC
>OX-10x9 Chain 1 [SEQ ID NO: 3]
DIQMTQSPSSLSASVGDRVTITCRASQSVSSAVAWYQQKPGKAPKLLIYSASSLYSGVPSRFSGSRSGTDFTLTISSLQPEDF
ATYYCQQVDSTPVTFGQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVYTLPPCRDELTKNQVSLWCLVKGFYPSDIAVEWESNGQPENNYKTTPPVLDSDGSFFLYSKLTVDKSRWQQGN
VFSCSVMHEALHNHYTQKSLSLSPGK
>OX-10x9 Chain 2 [SEQ ID NO: 4]
EVQLVESGGGLVQPGGSLRLSCAASGFTFSSYYIHWVRQAPGKGLEWVAYIGSQGGFTDYADSVKGRFTISADTSKNTAYLQM
NSLRAEDTAVYYCARQGYGYALDYWGQGTLVTVSSASPREPQVYTDPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSGEC
>OX-10-9 Chain 3[SEQ ID NO: 5]
DIQMTQSPSSLSASVGDRVTITCRASQSVSSAVAWYQQKPGKAPKLLIYSASSLYSGVPSRFSGSRSGTDFTLTISSLQPEDF
ATYYCQQYDYSPATFGQGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVCTLPPSREEMTKNQVSLSCAVKGFYPSDIAVEWESNGQPENNYKTTPPVLDSDGSFFLVSKLTVDKSRWQQGN
VFSCSVMHEALHNHYTQKSLSLSPGK
>OX-10-9 Chain 4 [SEQ ID NO: 6]
EVQLVESGGGLVQPGGSLRLSCAASGFTFWSYYIHWVRQAPGKGLEWVAAITPYDGYTYYADSVKGRFTISADTSKNTAYLQM
NSLRAEDTAVYYCARGSVYTGMDYWGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPPKSC
>OX-2-2x2 Chain 1 [SEQ ID NO: 7]
DIQMTQSPSSLSASVGDRVTITCRASQSVSSAVAWYQQKPGKAPKLLIYSASSLYSGVPSRFSGSRSGTDFTLTISSLQPEDF
ATYYCQQYIYGPLTFGQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCGGGGSGGGGSDIQMTQSPSSLSASVGDRVTITCRA
SQSVSSAVAWYQQKPGKAPKLLIYSASSLYSGVPSRFSGSRSGTDFTLTISSLQPEDFATYYCQQYIYGPLTFGQGTKVEIKR
TPREPQVYTLPPSRDELTKNQVSLKCLVKGFYPSDIAVEWESNGQPENNYKTTPPVLDSDGSFFLYSKLTVDKSRWQQGNVFS
CSVMHEALHNHYTQKSLSLSKSCDKTHTCPPCPAPELLGGPSVFLEPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTLPPCRDELTKNQV
SLWCLVKGFYPSDIAVEWESNGQPENNYKTTPPVLDSDGSFFLYSKLTVDKSRWQQGNVFSCSVMHEALHNHYTQKSLSLSPG
K
>OX-2-2x2 Chain 2 [SEQ ID NO: 8]
EVQLVESGGGLVQPGGSLRLSCAASGFTEDGYYTHWVRQAPGKGLEWVAAIESSSGYTYYADSVKGRFTISADTSKNTAYLQM
NSLRAEDTAVYYCARAYYTGMDYWGQGTLVTVSSASPREPQVYTDPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSGEC
>OX-2-2x2 Chain 3 [SEQ ID NO: 9]
DIQMTQSPSSLSASVGDRVTITCRASQSVSSAVAWYQQKPGKAPKLLIYSASSLYSGVPSRFSGSRSGTDFTLTISSLQPEDF
ATYYCQQYIYGPLTFGQGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECDKTHTCPPCPAPELLGGPSVFLEPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVCTLPPSREEMTKNQVSLSCAVKGFYPSDIAVEWESNGQPENNYKTTPPVLDSDGSFFLVSKLTVDKSRWQQGN
VFSCSVMHEALHNHYTQKSLSLSPGK
>OX-2-2x2 Chain 4 [SEQ ID NO: 10]
EVQLVESGGGLVQPGGSLRLSCAASGFTEDGYYTHWVRQAPGKGLEWVAAIESSSGYTYYADSVKGRFTISADTSKNTAYLQM
NSLRAEDTAVYYCARAYYTGMDYWGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
>OX-2-2x2 Chain 5 [SEQ ID NO: 11]
EVQLVESGGGLVQPGGSLRLSCAASGFTFDGYYIHWVRQAPGKGLEWVAAIESSSGYTYYADSVKGRFTISADTSKNTAYLQM
NSLRAEDTAVYYCARAYYTGMDYWGQGTLVTVSSASPREPQVYTDPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSGEC
>Pogalizumab (IgG1-kappa) heavy chain [SEQ ID NO: 12]
EVQLVQSGAEVKKPGASVKVSCKASGYTFTDSYMSWVRQAPGQGLEWIGDMYPDNGDSSYNQKFRERVTITRDTSTSTAYLEL
SSLRSEDTAVYYCVLAPRWYFSVWGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
>Pogalizumab (IgG1-kappa) light chain [SEQ ID NO: 13]
DIQMTQSPSSLSASVGDRVTITCRASQDISNYLNWYQQKPGKAPKLLIYYTSRLRSGVPSRFSGSGSGTDFTLTISSLQPEDF
ATYYCQQGHTLPPTFGQGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
>Tavolixizumab (IgG1-kappa) heavy chain [SEQ ID NO: 14]
QVQLQESGPGLVKPSQTLSLTCAVYGGSFSSGYWNWIRKHPGKGLEYIGYISYNGITYHNPSLKSRITINRDTSKNQYSLQLN
SVTPEDTAVYYCARYKYDYDGGHAMDYWGQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
>Tavolixizumab (IgG1-kappa) Light chain [SEQ ID NO: 15]
DIQMTQSPSSLSASVGDRVTITCRASQDISNYLNWYQQKPGKAPKLLIYYTSKLHSGVPSRFSGSGSGTDYTLTISSLQPEDF
ATYYCQQGSALPWTFGQGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
>GSK3174998 Heavy chain [SEQ ID NO: 16]
EVKLEESGGGLVQPGGSMKLSCAASGFTFSDAWMDWVRQSPEKGLEWVAEIRSKANNHATYYAESVNGRFTISRDDSKSSVYL
QMNSLRAEDTGIYYCTWGEVFYFDYWGQGTTLTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
>GSK3174998 Light chain [SEQ ID NO: 17]
DIQMTQSPSSLSASLGGKVTITCKSSQDINKYIAWYQHKPGKGPRLLIHYTSTLQPGIPSRFSGSGSGRDYSFSISNLEPEDI
ATYYCLQYDNLLTFGAGTKLELKRTVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
>Hinge [SEQ ID NO: 18
DKTHTCPPCP
>Phage display heavy chain (SEQ ID NO: 19):
EVQLVESGGGLVQPGGSLRLSCAASGFTExxxx [symbol] WVRQAPGKGLEWVAxxxxxxxxxxx [symbol] RFTISADTSKNTAYLQ
MNSLRAEDTAVYYCARxxxxxxxxxxxxx [symbol] WGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPSVF
LEPPKPKDTLMISRTPEVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGEYPSDIAVEWESNGQPENNYKTTPPVLDSDGSFFLY
SKLTVDKSRWQQGNVFSCSVMHEALHNHYTQKSLSLSPGK
>Phage display light chain (SEQ ID NO: 20):
DIQMTQSPSSLSASVGDRVTITC [symbol] VAWYQQKPGKAPKLLIY [symbol] [symbol] GVPSRFSGSRSGTDFTLTISSLQPEDF
ATYYC [symbol] xxxxxx [symbol] GQGTKVEIKRTVAAPSVFIFPPSDSQLKSGTASVVCLLNNFYPREAKVQWKVDNALQSGNSQESVTEQ
DSKDSTYSLSSTLTLSKADYEKHKVYACEVTHQGLSSPVTKSFNRGEC
>B-Body Domain A/H SCaffold (SEQ ID NO: 21):
DIQMTQSPSSLSASVGDRVTITC [symbol] VAWYQQKPGKAPKLLIY [symbol] [symbol] GVPSRFSGSRSGTDFTLTISSLQPEDF
ATYYC [symbol] xxxxxx [symbol] GQGTKVEIKRT
>B-Body Domain F/LSCaffold (SEQ ID NO: 22):
EVQLVESGGGLVQPGGSLRLSCAASGFTExxxx [symbol] WVRQAPGKGLEWVAxxxxxxxxxxx [symbol] RFTISADTSKNTAYLQ
MNSLRAEDTAVYYCARxxxxxxxxxxxxx [symbol] WGQGTLVTVSSAS
>BC1 Chain 1 SCaffold [SEQ ID NO: 23]
DIQMTQSPSSLSASVGDRVTITC [symbol] VAWYQQKPGKAPKLLIY [symbol] [symbol] GVPSRFSGSRSGTDFTLTISSLQPEDF
ATYYC [symbol] xxxxxx [symbol] GQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCDKTHTCPPCPAPELLGGPSVFLEPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVYTLPPCRDELTKNQVSLWCLVKGEYPSDIAVEWESNGQPENNYKTTPPVLDSDGSFFLYSKLTVDKSRWQQGN
VFSCSVMHEALHNHYTQKSLSLSPGK
> BC! Chain 2 SCaffold [SEQ ID NO: 24]
EVQLVESGGGLVQPGGSLRLSCAASGFTExxxx [symbol] WVRQAPGKGLEWVAxxxxxxxxxxx [symbol] RFTISADTSKNTAYLQ
MNSLRAEDTAVYYCARxxxxxxxxxxxxx [symbol] WGQGTLVTVSSASPREPQVYTDPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLSGEC
> BC! Chain 3 SCaffold [SEQ ID NO: 25]
DIQMTQSPSSLSASVGDRVTITC [symbol] VAWYQQKPGKAPKLLIY [symbol] [symbol] GVPSRFSGSRSGTDFTLTISSLQPEDF
ATYYC [symbol] xxxxxx [symbol] GQGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECDKTHTCPPCPAPELLGGPSVFLEPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVCTLPPSREEMTKNQVSLSCAVKGFYPSDIAVEWESNGQPENNYKTTPPVLDSDGSFFLVSKLTVDKSRWQQGN
VFSCSVMHEALHNHYTQKSLSLSPGK
> BC1 Chain 4 SCaffold [SEQ ID NO: 26]
EVQLVESGGGLVQPGGSLRLSCAASGFTExxxx [symbol] WVRQAPGKGLEWVAxxxxxxxxxxx [symbol] RFTISADTSKNTAYLQ
MNSLRAEDTAVYYCARxxxxxxxxxxxxx [symbol] WGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPPKSC
>BC1 2x1 Chain 1 SCaffold [SEQ ID NO: 27]
DIQMTQSPSSLSASVGDRVTITC [symbol] VAWYQQKPGKAPKLLIY [symbol] [symbol] GVPSRFSGSRSGTDFTLTISSLQPEDF
ATYYC [symbol] xxxxxx [symbol] GQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCGGGGSGGGGSDIQMTQSPSSLSASVGDRVTITC [symbol]
VAWYQQKPGKAPKLLIYSASSLYSGVPSRFSGSRSGTDFTLTISSLQPEDFATYYC [symbol] xxxxxx [symbol] GQGTKVEI
KRTPREPQVYTLPPSRDELTKNQVSLKCLVKGFYPSDIAVEWESNGQPENNYKTTPPVLDSDGSFFLYSKLTVDKSRWQQG
NVFSCSVMHEALHNHYTQKSLSLSKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQPREPQVYTLPPCR
DELTKNQVSLWCLVKGFYPSDIAVEWESNGQPENNYKTTPPVLDSDGSFFLYSKLTVDKSRWQQGNVFSCSVMHEALHNHY
TQKSLSLSPGK
>OX40: 2-214-Fc (SEQ ID NO: 28)
MGWSLILLFLVAVATRVLSCVGARRLGRGPCAALLLLGLGLSTVTGLHCVGDTYPSNDRCCHECRPGNGMVSRCSRSQNTVCR
PCGPGFYNDVVSSKPCKPCTWCNLRSGSERKQLCTATQDTVCRCRAGTQPLDSYKPGVDCAPCPPGHFSPGDNQACKPWTNCT
LAGKHTLQPASNSSDAICEDRDPPATQPQETQGPPARPITVQPTEAWPRTSQGPSTRPVEVPGGRASSGLNDIFEAQKIEWHE
GTENLYFQS

[symbol]

[symbol]

[symbol]

[symbol]

[symbol]

[symbol]

[symbol]

[symbol]

[symbol]

[symbol]

[symbol]

[symbol]

[symbol]

[symbol]

GSSHHHHHHHH
WT Fc = bold italic
Signal peptide = MGWSLILLFLVAVATRVLS (SEQ ID NO: 29)
Avi Tag = GLNDIFEAQKIEWHE (SEQ ID NO: 30)
TEVcleavage site = ENLYFQ (SEQ ID NO: 31)
His Tag = HEIHHHHHH (SEQ ID NO: 32)
>OX40: 66-214-Fc (SEQ ID NO: 33):
MGWSLILLFLVAVATRVLSPCGPGFYNDVVSSKPCKPCTWCNLRSGSERKQLCTATQDTVCRCRAGTQPLDSYKPGVDCAPCP
PGHFSPGDNQACKPWTNCTLAGKHTLQPASNSSDAICEDRDPPATQPQETQGPPARPITVQPTEAWPRTSQGPSTRPVEVPGG
RASSGLNDIFEAQKIEWHEGTENLYFQSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNVFSCSVMHEALHNHYTQKSL
SLSPGKGSSHHHHHHHH
>OX40: 108-214-Fc (SEQ ID NO: 34):
MGWSLILLFLVAVATRVLSRCRAGTQPLDSYKPGVDCAPCPPGHFSPGDNQACKPWTNCTLAGKHTLQPASNSSDAICEDRDP
PATQPQETQGPPARPITVQPTEAWPRTSQGPSTRPVEVPGGRASSGLNDIFEAQKIEWHEGTENLYFQSDKTHTCPPCPAPEL
LGGPSVFLFPPKPKDTLMISRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGKGSSHHHHHHHH
>OX40: 127-214-Fc (SEQ ID NO: 35):
MGWSLILLFLVAVATRVLSAPCPPGHFSPGDNQACKPWTNCTLAGKHTLQPASNSSDAICEDRDPPATQPQETQGPPARPITV
QPTEAWPRTSQGPSTRPVEVPGGRASSGLNDIFEAQKIEWHEGTENLYFQSDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGKGSSHHHHHHHH
>OX40: 24-24x11 Chain 1 [SEQ ID NO: 47]
DIQMTQSPSSLSASVGDRVTITCRASQSVSSAVAWYQQKPGKAPKLLIYSASSLYSGVPSRFSGSRSGTDFTLTISSLQPEDF
ATYYCQQWDDSPYTFGQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCGGGGSGGGGSDIQMTQSPSSLSASVGDRVTITCRA
SQSVSSAVAWYQQKPGKAPKLLIYSASSLYSGVPSRFSGSRSGTDFTLTISSLQPEDFATYYCQQWDDSPYTFGQGTKVEIKR
TPREPQVYTLPPSRDELTKNQVSLKCLVKGFYPSDIAVEWESNGQPENNYKTTPPVLDSDGSFFLYSKLTVDKSRWQQGNVFS
CSVMHEALHNHYTQKSLSLSKSCDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTLPPCRDELTKNQV
SLWCLVKGFYPSDIAVEWESNGQPENNYKTTPPVLDSDGSFFLYSKLTVDKSRWQQGNVFSCSVMHEALHNHYTQKSLSLSPG
K
>OX40: 24-24x11 Chain 2 [SEQ ID NO: 48]
EVQLVESGGGLVQPGGSLRLSCAASGFTFLSYYIHWVRQAPGKGLEWVAYIDPYSGGTDYADSVKGRFTISADTSKNTAYLQM
NSLRAEDTAVYYCARVGLSFYAQEPVLDYWGQGTLVTVSSASPREPQVYTDPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSGEC
>OX40: 24-24x11 Chain 3 [SEQ ID NO: 49]
DIQMTQSPSSLSASVGDRVTITCRASQSVSSAVAWYQQKPGKAPKLLIYSASSLYSGVPSRFSGSRSGTDFTLTISSLQPEDF
ATYYCQQYTSHPGTFGQGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVCTLPPSREEMTKNQVSLSCAVKGFYPSDIAVEWESNGQPENNYKTTPPVLDSDGSFFLVSKLTVDKSRWQQGN
VFSCSVMHEALHNHYTQKSLSLSPGK
>OX40:24-24x11 Chain 4 [SEQ ID NO: 50]
EVQLVESGGGLVQPGGSLRLSCAASGFTFSSYVIHWVRQAPGKGLEWVAYIFPYGGTTYYADSVKGRFTISADTSKNTAYLQM
NSLRAEDTAVYYCARGYYYVSDRVMDYWGQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEPPKSC
>OX40: 24-24x11 Chain 5 [see SEQ ID NO: 48]
>OX40: 24-24x10 Chain 1 [see SEQ ID NO: 47]
>OX-24-24x10 Chain 2 [see SEQ ID NO: 48]
>OX-24-24x10 Chain 3 [SEQ ID NO: 51]
DIQMTQSPSSLSASVGDRVTITCRASQSVSSAVAWYQQKPGKAPKLLIYSASSLYSGVPSRFSGSRSGTDFTLTISSLQPEDF
ATYYCQQVDSTPVTFGQGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVCTLPPSREEMTKNQVSLSCAVKGFYPSDIAVEWESNGQPENNYKTTPPVLDSDGSFFLVSKLTVDKSRWQQGN
VFSCSVMHEALHNHYTQKSLSLSPGK
>OX-24-24x10 Chain 4 [SEQ ID NO: 52]
EVQLVESGGGLVQPGGSLRLSCAASGFTFSSYYIHWVRQAPGKGLEWVAYIGSQGGFTDYADSVKGRFTISADTSKNTAYLQM
NSLRAEDTAVYYCARQGYGYALDYWGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPPKSC
>OX-24-24x10 Chain 5 [see SEQ ID NO: 48]
>OX-24-24x6 Chain 1 [see SEQ ID NO: 47]
>OX-24-24x6 Chain 2 [see SEQ ID NO: 48]
>OX-24-24x6 Chain 3 [SEQ ID NO: 53]
DIQMTQSPSSLSASVGDRVTITCRASQSVSSAVAWYQQKPGKAPKLLIYSASSLYSGVPSRFSGSRSGTDFTLTISSLQPEDF
ATYYCQQYARPPRTFGQGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVCTLPPSREEMTKNQVSLSCAVKGFYPSDIAVEWESNGQPENNYKTTPPVLDSDGSFFLVSKLTVDKSRWQQGN
VFSCSVMHEALHNHYTQKSLSLSPGK
>OX-24-24x6 Chain 4 [SEQ ID NO: 54]
EVQLVESGGGLVQPGGSLRLSCAASGFTFTDYHIHWVRQAPGKGLEWVAGISSYTGQTDYADSVKGRFTISADTSKNTAYLQM
NSLRAEDTAVYYCARGISGGFGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPPKSC
>OX-24-24x6 Chain 5 [see SEQ ID NO: 48]
>OX-24-24x4 Chain 1 [see SEQ ID NO: 47]
>OX-24-24x4 Chain 2 [see SEQ ID NO: 48]
>OX-24-24x4 Chain 3 [SEQ ID NO: 55]
DIQMTQSPSSLSASVGDRVTITCRASQSVSSAVAWYQQKPGKAPKLLIYSASSLYSGVPSRFSGSRSGTDFTLTISSLQPEDF
ATYYCQQWYSDPETFGQGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECDKTHTCPPCPAPELLGGPSVFLEPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVCTLPPSREEMTKNQVSLSCAVKGFYPSDIAVEWESNGQPENNYKTTPPVLDSDGSFFLVSKLTVDKSRWQQGN
VFSCSVMHEALHNHYTQKSLSLSPGK
>OX-24-24x4 Chain 4 [SEQ ID NO: 56]
EVQLVESGGGLVQPGGSLRLSCAASGFTEDGYYTHWVRQAPGKGLEWVAYIHPYGGYTRYADSVKGRFTISADTSKNTAYLQM
NSLRAEDTAVYYCARTRYDTGMDYWGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPPKSC
>OX-24-24x4 Chain 5 [see SEQ ID NO: 48]
>OX40: 24-11x11 Chain 1 [SEQ ID NO: 57]
DIQMTQSPSSLSASVGDRVTITCRASQSVSSAVAWYQQKPGKAPKLLIYSASSLYSGVPSRFSGSRSGTDFTLTISSLQPEDF
ATYYCQQWDDSPYTFGQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCDGSGSGSGSGSDIQMTQSPSSLSASVGDRVTITCR
ASQSVSSAVAWYQQKPGKAPKLLIYSASSLYSGVPSRFSGSRSGTDFTLTISSLQPEDFATYYCQQYTSHPGTFGQGTKVEIK
RTVAAPSVFIFPPSDEQLKSGTASVVCLLNNFYPREAKVQWKVDNALQSGNSQESVTEQDSKDSTYSLSSTLTLSKADYEKHK
VYACEVTHQGLSSPVTKSFNRGECKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTLPPCRDELTKNQV
SLWCLVKGFYPSDIAVEWESNGQPENNYKTTPPVLDSDGSFFLYSKLTVDKSRWQQGNVFSCSVMHEALHNHYTQKSLSLSPG
K
>OX40: 24-11x11 Chain 2 [see SEQ ID NO: 50]
>OX40: 24-11x11 Chain 3 [see SEQ ID NO: 49]
>OX40: 24-11x11 Chain 4 [see SEQ ID NO: 50]
>OX40: 24-11x11 Chain 5 [see SEQ ID NO: 48]
>OX40: 24 (WEE)-11x11 Chain 1 [SEQ ID NO: 58]
DIQMTQSPSSLSASVGDRVTITCRASQSVSSAVAWYQQKPGKAPKLLIYSASSLYSGVPSRFSGSRSGTDFTLTISSLQPEDF
ATYYCQQWEESPYTFGQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCDGSGSGSGSGSDIQMTQSPSSLSASVGDRVTITCR
ASQSVSSAVAWYQQKPGKAPKLLIYSASSLYSGVPSRFSGSRSGTDFTLTISSLQPEDFATYYCQQYTSHPGTFGQGTKVEIK
RTVAAPSVFIFPPSDEQLKSGTASVVCLLNNFYPREAKVQWKVDNALQSGNSQESVTEQDSKDSTYSLSSTLTLSKADYEKHK
VYACEVTHQGLSSPVTKSFNRGECKTHTCPPCPAPELLGGPSVFLEPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTLPPCRDELTKNQV
SLWCLVKGFYPSDIAVEWESNGQPENNYKTTPPVLDSDGSFFLYSKLTVDKSRWQQGNVFSCSVMHEALHNHYTQKSLSLSPG
K
>OX40: 24-24(WEE)x11 Chain 1 [SEQ ID NO: 59]
DIQMTQSPSSLSASVGDRVTITCRASQSVSSAVAWYQQKPGKAPKLLIYSASSLYSGVPSRFSGSRSGTDFTLTISSLQPEDF
ATYYCQQWEESPYTFGQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCGGGGSGGGGSDIQMTQSPSSLSASVGDRVTITCRA
SQSVSSAVAWYQQKPGKAPKLLIYSASSLYSGVPSRFSGSRSGTDFTLTISSLQPEDFATYYCQQWEESPYTFGQGTKVEIKR
TPREPQVYTLPPSRDELTKNQVSLKCLVKGFYPSDIAVEWESNGQPENNYKTTPPVLDSDGSFFLYSKLTVDKSRWQQGNVFS
CSVMHEALHNHYTQKSLSLSKSCDKTHTCPPCPAPELLGGPSVFLEPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTLPPCRDELTKNQV
SLWCLVKGFYPSDIAVEWESNGQPENNYKTTPPVLDSDGSFFLYSKLTVDKSRWQQGNVFSCSVMHEALHNHYTQKSLSLSPG
K
>OX40: 24x11 Chain 1 [SEQ ID NO: 222]
DIQMTQSPSSLSASVGDRVTITCRASQSVSSAVAWYQQKPGKAPKLLIYSASSLYSGVPSRFSGSRSGTDFTLTISSLQPEDF
ATYYCQQWDDSPYTFGQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCDKTHTCPPCPAPELLGGPSVFLEPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVYTLPPCRDELTKNQVSLWCLVKGFYPSDIAVEWESNGQPENNYKTTPPVLDSDGSFFLYSKLTVDKSRWQQGN
VFSCSVMHEALHNHYTQKSLSLSPGK
>OX40: 24x11 Chain 2 [see SEQ ID NO: 48]
>OX40: 24x11 Chain 3 [see SEQ ID NO: 49]
>OX40: 24x11 Chain 4 [see SEQ ID NO: 50]
>OX40: 11x24 Chain 1 [SEQ ID NO: 223]
DIQMTQSPSSLSASVGDRVTITCRASQSVSSAVAWYQQKPGKAPKLLIYSASSLYSGVPSRFSGSRSGTDFTLTISSLQPEDF
ATYYCQQYTSHPGTFGQGTKVEIKRTPREPQVYTLPPSRDELTKNQVSLKCLVKGFYPSDIAVEWESNGQPENNYKTTPPVLD
SDGSFFLYSKLTVDKSRWQQGNVFSCSVMHEALHNHYTQKSLSLSKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVYTLPPCRDELTKNQVSLWCLVKGFYPSDIAVEWESNGQPENNYKTTPPVLDSDGSFFLYSKLTVDKSRWQQGN
VFSCSVMHEALHNHYTQKSLSLSPGK
>OX40: 11x24 Chain 2 [SEQ ID NO: 224]
EVQLVESGGGLVQPGGSLRLSCAASGFTFSSYVIHWVRQAPGKGLEWVAYIFPYGGTTYYADSVKGRFTISADTSKNTAYLQM
NSLRAEDTAVYYCARGYYYVSDRVMDYWGQGTLVTVSSASPREPQVYTDPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSGEC
>OX40: 11x24 Chain 3 [SEQ ID NO: 225]
DIQMTQSPSSLSASVGDRVTITCRASQSVSSAVAWYQQKPGKAPKLLIYSASSLYSGVPSRFSGSRSGTDFTLTISSLQPEDF
ATYYCQQWDDSPYTFGQGTKVEIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISK
AKGQPREPQVCTLPPSREEMTKNQVSLSCAVKGFYPSDIAVEWESNGQPENNYKTTPPVLDSDGSFFLVSKLTVDKSRWQQGN
VFSCSVMHEALHNHYTQKSLSLSPGK
>OX40: 11x24 Chain 4 [SEQ ID NO: 226]
EVQLVESGGGLVQPGGSLRLSCAASGFTFLSYYIHWVRQAPGKGLEWVAYIDPYSGGTDYADSVKGRFTISADTSKNTAYLQM
NSLRAEDTAVYYCARVGLSFYAQEPVLDYWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKVEPPKSC

### 8. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 9. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A multivalent antibody construct,
wherein the construct is capable of (i) binding a cell surface receptor target that requires clustering for agonist activity, and (ii) clustering the receptor target on the cell surface in the absence of an independent cross-linking agent or one or more Fc mutations that drive hexamer formation, and
wherein each of the target receptor-binding antigen binding sites of the construct is contributed by antibody variable region binding domains.

2. The multivalent construct of claim 1, wherein the construct is monospecific.

3. The multivalent construct of claim 1, wherein the construct is multispecific.

4. The multi specific multivalent construct of claim 3, wherein the construct comprises a first antigen binding site specific for a first epitope of the target receptor, and a second antigen binding site specific for a second antigenic target.

5. The multispecific multivalent construct of claim 4, wherein the second antigenic target is a second epitope of the target receptor, optionally wherein the first epitope and the second epitope are non-overlapping epitopes.

6. The multispecific multivalent construct of claim 4, wherein the second antigenic target is an epitope of a second protein.

7. The multispecific multivalent construct of claim 6, wherein the second protein is a second cell surface receptor.

8. The multispecific multivalent construct of claim 7, wherein the target cell surface receptor and the second cell surface receptor are commonly expressed on the surface of at least some mammalian cells.

9. The multivalent construct of any one of claims 1-8, wherein the target receptor is a TNF Receptor superfamily (TNFRSF) member.

10. The multivalent construct of claim 9, wherein the target receptor is OX40 (TNFRSF4), CD40 (TNFRSF5), or 4-1BB (TNFRSF9).

11. The multivalent construct of claim 9 or claim 10, wherein the target receptor is a human TNFRSF.

12. The multivalent construct of claim 11, wherein the target receptor is human OX40, human CD40, or human 4-1BB.

13. The multivalent construct of claim 12, wherein the target receptor is human OX40.

14. The multivalent construct of any of claims 1-13, wherein the construct is bivalent.

15. The multivalent construct of claim 14, wherein the bivalent construct is a bivalent (1×1) construct.

16. The bivalent construct of claim 15, wherein the construct is monospecific.

17. The bivalent construct of claim 15, wherein the construct is bispecific.

18. The bivalent bispecific construct of claim 17, wherein the second antigenic target is a second epitope of the target receptor, optionally wherein the first epitope and the second epitope are non-overlapping epitopes.

19. The bivalent bispecific construct of claim 17, wherein the second antigenic target is an epitope of a second protein.

20. The bivalent bispecific construct of claim 19, wherein the second protein is a second cell surface receptor.

21. The bispecific bivalent construct of any one of claims 17-20, wherein the antigen binding site specific for a first epitope of the target receptor is an A:F antigen binding site.

22. The bispecific bivalent construct of any one of claims 17-20, wherein the antigen binding site specific for a first epitope of the target receptor is an H:L antigen binding site.

23. The multivalent construct of any of claims 1-13, wherein the construct is trivalent.

24. The trivalent construct of claim 23, wherein the construct is a trivalent (2×1) construct.

25. The trivalent construct of claim 24, wherein the construct is monospecific.

26. The trivalent construct of claim 24, wherein the construct is bispecific.

27. The bispecific trivalent construct of claim 26, wherein the construct contains one copy of the antigen binding site (ABS) specific for a first epitope of the target receptor.

28. The bispecific trivalent construct of claim 27, wherein the antigen binding site specific for a first epitope of the target receptor is an A:F antigen binding site.

29. The bispecific trivalent construct of claim 27, wherein the antigen binding site specific for a first epitope of the target receptor is an N:P antigen binding site.

30. The bispecific trivalent construct of claim 27, wherein the antigen binding site specific for a first epitope of the target receptor is an H:L antigen binding site.

31. The bispecific trivalent construct of claim 26, wherein the construct contains two copies of the antigen binding site specific for a first epitope of the target receptor.

32. The bispecific trivalent construct of claim 31, wherein a first antigen binding site specific for a first epitope of the target receptor is an A:F antigen binding site.

33. The bispecific trivalent construct of claim 31, wherein a first antigen binding site specific for a first epitope of the target receptor is an N:P antigen binding site.

34. The bispecific trivalent construct of claim 31, wherein a first antigen binding site specific for a first epitope of the target receptor is an H:L antigen binding site.

35. The bispecific trivalent construct of claim 31, wherein a first antigen binding site specific for a first epitope of the target receptor is an A:F antigen binding site and a second antigen binding site specific for a first epitope of the target receptor is an N:P antigen binding site.

36. The bispecific trivalent construct of claim 31, wherein a first antigen binding site specific for a first epitope of the target receptor is an A:F antigen binding site and a second antigen binding site specific for a first epitope of the target receptor is an H:L antigen binding site.

37. The bispecific trivalent construct of claim 31, wherein a first antigen binding site specific for a first epitope of the target receptor is an N:P antigen binding site and a second antigen binding site specific for a first epitope of the target receptor is an H:L antigen binding site.

38. The bispecific trivalent construct of any one of claims 26-37, wherein the second antigenic target is a second epitope of the target receptor, optionally wherein the first epitope and the second epitope are non-overlapping epitopes.

39. The bispecific trivalent construct of any one of claims 26-37, herein the second antigenic target is an epitope of a second protein.

40. The bispecific trivalent construct of claim 39, wherein the second protein is a second cell surface receptor.

41. The trivalent construct of claim 24, wherein the constructs trispecific.

42. The trispecific trivalent construct of claim 41, wherein the antigen binding site specific for a first epitope of the target receptor is an A:F antigen binding site.

43. The tri specific trivalent construct of claim 41, wherein the antigen binding site specific for a first epitope of the target receptor is an N:P antigen binding site.

44. The trispecific trivalent construct of claim 41, wherein the antigen binding site specific for a first epitope of the target receptor is an H:L antigen binding site.

45. The trispecific trivalent construct of any one of claims 41-44, wherein the second antigenic target is a second epitope of the target receptor, optionally wherein the first epitope and the second epitope are non-overlapping epitopes.

46. The bispecific trivalent construct of any one of claims 41-44, wherein the second antigenic target is a first epitope of a second protein.

47. The trispecific trivalent construct of any one of claims 41-46, wherein the third antigenic target is a third epitope of the target receptor.

48. The trispecific trivalent construct of any one of claims 41-46, wherein the third antigenic target is a second epitope of a second protein, optionally wherein the first epitope of the second protein and the second epitope of the second protein are non-overlapping epitopes.

49. The trispecific trivalent construct of any one of claims 41-46, wherein the third antigenic target is a first epitope of a third protein.

50. The trispecific trivalent construct of any one of claim 46, 48, or 49, wherein the second protein or third protein is a second or third cell surface receptor.

51. The trivalent construct of claim 23, wherein the constructs a trivalent (1×2) construct.

52. The trivalent construct of claim 51, wherein the construct is monospecific.

53. The trivalent construct of claim 51, wherein the construct is bispecific.

54. The bispecific trivalent construct of claim 53, wherein the construct contains one copy of the antigen binding site (ABS) specific for a first epitope of the target receptor.

55. The bispecific trivalent construct of claim 54, wherein the antigen binding site specific for a first epitope of the target receptor is an A:F antigen binding site.

56. The bispecific trivalent construct of claim 54, wherein the antigen binding site specific for a first epitope of the target receptor is an R:T antigen binding site.

57. The bispecific trivalent construct of claim 54, wherein the antigen binding site specific for a first epitope of the target receptor is an H:L antigen binding site.

58. The bispecific trivalent construct of claim 53, wherein the construct contains two copies of the antigen binding site specific for a first epitope of the target receptor.

59. The bispecific trivalent construct of claim 58, wherein a first antigen binding site specific for a first epitope of the target receptor is an A:F antigen binding site.

60. The bispecific trivalent construct of claim 58, wherein a first antigen binding site specific for a first epitope of the target receptor is an R:T antigen binding site.

61. The bispecific trivalent construct of claim 58, wherein a first antigen binding site specific for a first epitope of the target receptor is an H:L antigen binding site.

62. The bispecific trivalent construct of claim 58, wherein a first antigen binding site specific for a first epitope of the target receptor is an A:F antigen binding site and a second antigen binding site specific for a first epitope of the target receptor is an R:T antigen binding site.

63. The bispecific trivalent construct of claim 58, wherein a first antigen binding site specific for a first epitope of the target receptor is an A:F antigen binding site and a second antigen binding site specific for a first epitope of the target receptor is an H:L antigen binding site.

64. The bispecific trivalent construct of claim 58, wherein a first antigen binding site specific for a first epitope of the target receptor is an R:T antigen binding site and a second antigen binding site specific for a first epitope of the target receptor is an H:L antigen binding site.

65. The bispecific trivalent construct of any one of claims 53-64, wherein the second antigenic target is a second epitope of the target receptor, optionally wherein the first epitope and the second epitope are non-overlapping epitopes.

66. The bispecific trivalent construct of any one of claims 53-64, wherein the second antigenic target is an epitope of a second protein.

67. The bispecific trivalent construct of claim 66, wherein the second protein is a second cell surface receptor.

68. The trivalent construct of claim 51, wherein the construct s trispecific.

69. The tri specific trivalent construct of claim 68, wherein the antigen binding site specific for a first epitope of the target receptor is an A:F antigen binding site.

70. The trispecific trivalent construct of claim 68, wherein the antigen binding site specific for a first epitope of the target receptor is an R antigen binding site.

71. The trispecific trivalent construct of claim 68, wherein the antigen binding site specific for a first epitope of the target receptor is an H:L antigen binding site.

72. The trispecific trivalent construct of any one of claims 68-71, wherein the second antigenic target is a second epitope of the target receptor, optionally wherein the first epitope and the second epitope are non-overlapping epitopes.

73. The bispecific trivalent construct of any one of claims 68-71, wherein the second antigenic target is a first epitope of a second protein.

74. The trispecific trivalent construct of any one of claims 68-73, wherein the third antigenic target is a third epitope of the target receptor.

75. The trispecific trivalent construct of any one of claims 68-73, wherein the third antigenic target is a second epitope of a second protein, optionally wherein the first epitope of the second protein and the second epitope of the second protein are non-overlapping epitopes.

76. The trispecific trivalent construct of any one of claims 68-73, wherein the third antigenic target is a first epitope of a third protein.

77. The tri specific trivalent construct of any one of claim 73, 75 or 76 wherein the second protein or third protein is a second or third cell surface receptor.

78. The multivalent antibody construct of any one of claims 1-77, wherein the presence of an independent cross-linking agent does not increase agonist activity in vitro above that achieved in the absence of the independent cross-linking agent.

79. The multivalent antibody construct of any one of claims 1-77, wherein the presence of an independent cross-linking agent increases agonist activity in vitro above that achieved in the absence of the independent cross-linking agent.

80. The multivalent antibody construct of claim 79, wherein the presence of an independent cross-linking agent increases agonist in vitro activity 50% above activity observed in the absence of the independent cross-linking agent.

81. The bivalent (1×1) antibody constructs of any one of claims 15-22, wherein the construct comprises:
a first, second, third, and fourth polypeptide chain, wherein:
(a) the first polypeptide chain comprises a domain A, a domain B, a domain D, and a domain E,
wherein the domains are arranged, from N-terminus to C-terminus, in a A-B-D-E orientation, and
domain A has a VL amino acid sequence, domain B has a CH3 amino acid sequence, domain D has a CH2 amino acid sequence, and domain E has a constant region domain amino acid sequence;
(b) the second polypeptide chain comprises a domain F and a domain G,
wherein the domains are arranged, from N-terminus to C-terminus, in a F-G orientation, and
wherein domain F has a amino acid sequence and domain G has a CH3 amino acid sequence;
(c) the third polypeptide chain comprises a domain H, a domain I, a domain J, and a domain K,
wherein the domains are arranged, from N-terminus to C-terminus, in a H-I-J-K orientation, and
wherein domain H has a variable region domain amino acid sequence, domain I has a constant region domain amino acid sequence, domain J has a CH2 amino acid sequence, and K has a constant region domain amino acid sequence;
(d) the fourth polypeptide chain comprises a domain L and a domain M,
wherein the domains are arranged, from N-terminus to C-terminus, in a L-M orientation, and
wherein domain L has a variable region domain amino acid sequence and domain M has a constant region domain amino acid sequence;
(e) the first and the second polypeptides are associated through an interaction between the A and the F domains and an interaction between the B and the G domains;
(f) the third and the fourth polypeptides are associated through an interaction between the H and the I, domains and an interaction between the I and the M domains; and
(g) the first and the third polypeptides are associated through an interaction between the D and the J domains and an interaction between the E and the K domains to form the binding molecule.

82. The construct of claim 81, wherein the amino acid sequences of the B and the G domains are identical, wherein the sequence is an endogenous CH3 sequence.

83. The construct of claim 81, wherein the amino acid sequences of the B and the G domains are different and separately comprise respectively orthogonal modifications in an endogenous CH3 sequence, wherein the B domain interacts with the G domain, and wherein neither the B domain nor the G domain interacts with a CH3 domain lacking the orthogonal modification.

84. The binding molecule of claim 83, wherein the orthogonal modifications comprise mutations that generate engineered disulfide bridges between domain B and G.

85. The binding molecule of claim 84, wherein the mutations that generate engineered disulfide bridges are a S354C mutation in one of the B domain and G domain, and a 349C in the other domain.

86. The binding molecule of any one of claims 83-85, wherein the orthogonal modifications comprise knob-in-hole mutations.

87. The binding molecule of claim 86, wherein the knob-in hole mutations are a T366W mutation in one of the B domain and G domain, and a T366S, L368A, and a Y407V mutation in the other domain.

88. The binding molecule of any one of claims 83-87, wherein the orthogonal modifications comprise charge-pair mutations.

89. The binding molecule of claim 88, wherein the charge-pair mutations are a T366K mutation in one of the B domain and G domain, and a L351D mutation in the other domain.

90. The binding molecule of any one of claims 80-89, wherein the domain E has a CH3 amino acid sequence.

91. The binding molecule of any one of claims 81-90, wherein the amino acid sequences of the E and K domains are identical, wherein the sequence is an endogenous CH3 sequence.

92. The binding molecule of any one of claims 81-90, wherein the amino acid sequences of the E and K domains are different.

93. The binding molecule of claim 92, wherein the different sequences separately comprise respectively orthogonal modifications in an endogenous CH3 sequence, wherein the E domain interacts with the K domain, and wherein neither the 1 domain nor the K domain interacts with a CH3 domain lacking the orthogonal modification.

94. The binding molecule of claim 93, wherein the orthogonal modifications comprise mutations that generate engineered disulfide bridges between domain E and K.

95. The binding molecule of claim 94, wherein the mutations that generate engineered disulfide bridges are a S354C mutation in one of the E domain and K domain, and a 349C in the other domain.

96. The binding molecule of any one of claims 93-95, wherein the orthogonal modifications in the E and K domains comprise knob-in-hole mutations.

97. The binding molecule of claim 96, wherein the knob-in hole mutations are a T366W mutation in one of the E domain or K domain and a T366S, L368A, and a Y407V mutation in the other domain.

98. The binding molecule of any one of claims 93-97, wherein the orthogonal modifications comprise charge-pair mutations.

99. The binding molecule of claim 98, wherein the charge-pair mutations are a T366K mutation in one of the E domain or K domain and a corresponding L351D mutation in the other domain.

100. The binding molecule of claim 92, wherein the amino acid sequences of the E domain and the K domain are endogenous sequences of two different antibody domains, the domains selected to have a specific interaction that promotes the specific association between the first and the third polypeptides.

101. The binding molecule of claim 100, wherein the two different amino acid sequences are a CH1 sequence and a CL sequence.

102. The binding molecule of any one of claims 81-101, wherein domain I has a CL sequence and domain M has a CH1 sequence.

103. The binding molecule of any one of claims 81-102, wherein domain H has a VL sequence and domain L has a VH sequence.

104. The binding molecule of any one of claims 81-103, wherein:
domain H has a VL amino acid sequence;
domain I has a CL amino acid sequence;
domain K has a CH3 amino acid sequence;
domain L has a VH amino acid sequence;
and domain M has a CH1 amino acid sequence.

105. The construct of any one of claims 81-104, further comprising:
a sixth polypeptide chain,
wherein:
(a) the third polypeptide chain further comprises a domain R and a domain S, wherein the domains are arranged, from N-terminus to C-terminus, in a R-S-H-I-J-K orientation, and
wherein domain R has a VL amino acid sequence and domain S has a constant domain amino acid sequence;
(b) the binding molecule further comprises a sixth polypeptide chain, comprising:
a domain T and a domain U,
wherein the domains are arranged, from N-terminus to C-terminus, in a T-U orientation, and
wherein domain T has a VH amino acid sequence and domain U has a constant domain amino acid sequence; and
(c) the third and the sixth polypeptides are associated through an interaction between the R and the T domains and an interaction between the S and the U domains to form the binding molecule.

106. The construct of claim 105, wherein
(a) the amino acid sequences of domain R and domain A are identical,
the amino acid sequences of domain H is different from domain R and A,
the amino acid sequences of domain S and domain B are identical,
the amino acid sequences of domain I is different from domain S and B,
the amino acid sequences of domain T and domain F are identical,
the amino acid sequences of domain L is different from domain T and F,
the amino acid sequences of domain U and domain G are identical,
the amino acid sequences of domain M is different from domain U and G and
(b) the interaction between the A domain and the F domain form a first antigen binding site specific for a first antigen,
the interaction between the H domain and the L domain form a second antigen binding site specific for a second antigen, and
the domain R and domain T form a third antigen binding site specific for the first antigen.

107. The binding molecule of claim 105, wherein
(a) the amino acid sequences of domain R and domain H are identical,
the amino acid sequences of domain A is different from domain R and H,
the amino acid sequences of domain S and domain I are identical,
the amino acid sequences of domain B is different from domain S and I,
the amino acid sequences of domain T and domain L are identical,
the amino acid sequences of domain F is different from domain T and L,
the amino acid sequences of domain U and domain M are identical,
the amino acid sequences of domain G is different from domain U and M and
(b) the interaction between the A domain and the F domain form a first antigen binding site specific for a first antigen,
the interaction between the H domain and the L domain form a second antigen binding site specific for a second antigen, and
the domain R and domain T form a third antigen binding site specific for the second antigen.

108. The binding molecule of claim 105, wherein
(a) the amino acid sequences of domain R, domain A, and domain H are different,
the amino acid sequences of domain S, domain B, and domain I are different,
the amino acid sequences of domain T, domain F, and domain L are different, and
the amino acid sequences of domain U, domain G, and domain M are different; and
(b) the interaction between the A domain and the F domain form a first antigen binding site specific for a first antigen,
the interaction between the H domain and the L domain form a second antigen binding site specific for a second antigen, and
the domain R and domain T form a third antigen binding site specific for a third antigen.

109. The construct of any one of claims 81-104, further comprising:
a fifth polypeptide chain, wherein:
(a) the first polypeptide chain further comprises a domain N and a domain O, wherein the domains are arranged, from N-terminus to C-terminus, in a N-O-A-B-D-E orientation, and
wherein domain N has a VL amino acid sequence, domain O has a CH3 amino acid sequence;
(b) the binding molecule further comprises a fifth polypeptide chain, comprising:
a domain P and a domain Q, wherein the domains are arranged, from N-terminus to C-terminus, in a P-Q orientation, and
wherein domain P has a VH amino acid sequence and domain Q has a CH3 amino acid sequence; and
(c) the first and the fifth polypeptides are associated through an interaction between the N and the P domains and an interaction between the O and the Q domains to form the binding molecule.

110. The binding molecule of claim 109, wherein:
(a) the amino acid sequences of domain N and domain A are identical,
the amino acid sequences of domain H is different from domains N and A,
the amino acid sequences of domain O and domain B are identical,
the amino acid sequences of domain I is different from domains O and B,
the amino acid sequences of domain P and domain F are identical,
the amino acid sequences of domain L is different from domains P and F,
the amino acid sequences of domain Q and domain G are identical,
the amino acid sequences of domain M is different from domains Q and G; and
(b) wherein the interaction between the A domain and the F domain form a first antigen binding site specific for a first antigen, the interaction between the H domain and the L domain form a second antigen binding site specific for a second antigen, and the domain N and domain P form a third antigen binding site specific for the first antigen.

111. The binding molecule of claim 109, wherein:
(a) the amino acid sequences of domain N, domain A, and domain H are different,
the amino acid sequences of domain O, domain B, and domain I are different,
the amino acid sequences of domain P, domain F, and domain L are different, and
the amino acid sequences of domain Q, domain G, and domain M are different; and
(b) the interaction between the A domain and the F domain form a first antigen binding site specific for a first antigen,
the interaction between the H domain and the L domain form a second antigen binding site specific for a second antigen, and
the domain N and domain P form a third antigen binding site specific for a third antigen.

112. The binding molecule of any of the above claims 1-111, wherein the sequence that links the A domain and the B domain is IKRTPREP or IKRTVREP.

113. The binding molecule of any of the above claims 1-112, wherein the sequence that links the F domain and the G domain is SSASPREP.

114. The binding molecule of any of the above claims 1-113, wherein at least one CH3 amino acid sequence has a C-terminal tripeptide insertion linking the CH3 amino acid sequence to a hinge amino acid sequence, wherein the tripeptide insertion is selected from the group consisting of PGK, KSC, and GEC.

115. The binding molecule of any of the above claims 1-114, wherein the sequences are human sequences.

116. The binding molecule of any of the above claims 1-115, wherein at least one CH3 amino acid sequence is an IgG sequence.

117. The binding molecule of claim 116, wherein the IgG sequences are IgG1 sequences.

118. The binding molecule of any of the above claims 1-117, wherein at least one CH3 amino acid sequence has one or more isoallotype mutations.

119. The binding molecule of claim 118, wherein the isoallotype mutations are D356E and L358M.

120. The binding molecule of any of the above claims, wherein the CL amino acid sequence is a Cₖₐₚₚₐ sequence.

121. An OX40 binding molecule, the OX40 antigen binding molecule comprising:
a first antigen binding site specific for an OX40 antigen, wherein the first antigen binding site comprises:
A) a CDR1, a CDR2, and a CDR3 amino acid sequences of a specific light chain variable region (VL), wherein the CDR1, CDR2, and CDR3 VL sequences are selected from Table 4 corresponding to a specific OX40 antigen binding site (ABS); and
B) a CDR1, a CDR2, and a CDR3 amino acid sequences of a specific heavy chain variable region (VH), wherein the CDR1, CDR2, and CDR3 VH sequences are selected from Table 3 corresponding to the specific OX40 ABS.

122. The OX40 antigen binding molecule of claim 121, wherein the first antigen binding site is specific for a first epitope of the OX40 antigen.

123. The OX40 antigen binding molecule of any of claims 121-122, wherein the OX40 antigen comprises an OX40 domain selected from the group consisting of: OX40 amino acids 2-214, OX40 amino acids 66-214, OX40 amino acids 108-214, and OX40 amino acids 127-214.

124. The OX40 antigen binding molecule of any of claims 121-123, wherein the OX40 antigen comprises a human OX40 antigen.

125. The OX40 antigen binding molecule of any of claims 121-124, wherein the first antigen binding site comprises a VL CDR1 comprising SEQ ID NO:220, a VL CDR2 comprising SEQ ID NO:221, and a VL CDR3 comprising SEQ ID NO:203, and a VH CDR1 comprising SEQ ID NO:83, a VH CDR2 comprising SEQ ID NO:123, and a VH CDR3 comprising SEQ ID NO:163.

126. The OX40 antigen binding molecule of any of claims 121-124, wherein the first antigen binding site comprises a VL CDR1 comprising SEQ ID NO:220, a VL CDR2 comprising SEQ ID NO:221, and a VL CDR3 comprising SEQ ID NO:227, and a VH CDR1 comprising SEQ ID NO:83, a VH CDR2 comprising SEQ ID NO:123, and a VH CDR3 comprising SEQ ID NO:163.

127. The OX40 antigen binding molecule of any of claims 121-124, wherein the first antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150.

128. The OX40 antigen binding molecule of any of claims 121-124124, wherein the OX40 antigen binding molecule further comprises a second antigen binding site.

129. The OX40 antigen binding molecule of claim 128, wherein the second antigen binding site is specific for the OX40 antigen.

130. The OX40 antigen binding molecule of claim 129, wherein the second antigen binding site is specific for the first epitope of the OX40 antigen.

131. The OX40 antigen binding molecule of claim 129, wherein the second antigen binding site is specific for a second epitope of the OX40 antigen.

132. The OX40 antigen binding molecule of claim 131, wherein the first epitope and the second epitope are non-overlapping epitopes.

133. The OX40 antigen binding molecule of claim 131, wherein the first antigen binding site comprises a VL CDR1 comprising SEQ ID NO:220, a VL CDR2 comprising SEQ NO:221, and a VL CDR3 comprising SEQ ID NO:203, and a VH CDR1 comprising SEQ ID NO:83, a VH CDR2 comprising SEQ ID NO:123, and a VH CDR3 comprising SEQ ID NO:163; and
the second antigen binding site comprises a VL CDR1 comprising SEQ ID NO:220, a VL CDR2 comprising SEQ NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150.

134. The OX40 antigen binding molecule of claim 131, wherein the first antigen binding site comprises a VL CDR1 comprising SEQ ID NO:220, a VL CDR2 comprising SEQ ID NO:221, and a VL CDR3 comprising SEQ ID NO:227, and a VH CDR1 comprising SEQ ID NO:83, a VH CDR2 comprising SEQ ID NO:123, and a VH CDR3 comprising SEQ NO:163; and
the second antigen binding site comprises a VL CDR1 comprising SEQ ID NO:220, a VL CDR2 comprising SEQ ID NO:221, and a VL CDR3 comprising SEQ NO:190, and a VH CDR1 comprising SEQ ID NO:70, a VH CDR2 comprising SEQ ID NO:110, and a VH CDR3 comprising SEQ NO:150.

135. The OX40 antigen binding molecule of claim 128, wherein the second antigen binding site is specific for a second antigen different from the OX40 antigen.

136. The OX40 antigen binding molecule of claim 135, wherein the second antigen is a second cell surface receptor.

137. The OX40 antigen binding molecule of any of claims 121-136, wherein the OX40 antigen binding molecule comprises an antibody format selected from the group consisting of: full-length antibodies, Fab fragments, Fvs, scFvs, tandem scFvs, Diabodies, scDiabodies, DARTs, tandAbs, and minibodies.

138. The OX40 antigen binding molecule of any of claims 121-136, wherein the OX40 antigen binding molecule comprises:
a first and a second polypeptide chain, wherein:
(a) the first polypeptide chain comprises a domain A, a domain B, a domain D, and a domain E,
wherein the domains are arranged, from N-terminus to C-terminus, in a A-B-D-E orientation,
wherein domain A has a variable region domain amino acid sequence, and
wherein domain B, domain D, and domain E have a constant region domain amino acid sequence;
(b) the second polypeptide chain comprises a domain F and a domain G, wherein the domains are arranged, from N-terminus to C-terminus, in a F-G orientation, and
wherein domain F has a variable region domain amino acid sequence and domain G has a constant region domain amino acid sequence
c) the first and the second polypeptides are associated through an interaction between the A and the F domain and an interaction between the B domain and the G domain to form the OX40 antigen binding molecule, and wherein the interaction between the A domain and the F domain form a first antigen binding site.

139. The OX40 antigen binding molecule of claim 138, wherein the OX40 antigen binding molecule further comprises:
a third and a fourth polypeptide chain, wherein:
(a) the third polypeptide chain comprises a domain H, a domain I, a domain J, and a domain K,
wherein the domains are arranged, from N-terminus to C-terminus, in a H-I-J-K orientation, and
wherein domain H has a variable region domain amino acid sequence, and domains I, J, and K have a constant region domain amino acid sequence;
(b) the fourth polypeptide chain comprises a domain L and a domain M, wherein the domains are arranged; from N-terminus to C-terminus, in a L-M orientation, and
wherein domain L has a variable region domain amino acid sequence and domain M has a constant region amino acid sequence;
(c) the third and the fourth polypeptides are associated through an interaction between the H and the L domains and an interaction between the I and the M domains; and
(d) the first and the third polypeptides are associated through an interaction between the D domain and the J domain and an interaction between the E domain and the K domain to form the OX40 antigen binding molecule, and wherein the interaction between the H domain and the L domain form a second antigen binding site.

140. The OX40 antigen binding molecule of claim 138 or 139, wherein the first antigen binding site is specific for the OX40 antigen.

141. The OX40 antigen binding molecule of claim 140, wherein the second antigen binding site is specific for the OX40 antigen.

142. The OX40 antigen binding molecule of claim 141, wherein the first antigen binding site is specific for a first epitope of the OX40 antigen and the second antigen binding site is specific for a second epitope of the OX40 antigen.

143. The OX40 antigen binding molecule of claim 142, wherein the first epitope and the second epitope are non-overlapping epitopes.

144. The OX40 antigen binding molecule of any one of claims 138-143; wherein domain B and domain G have a CH3 amino acid sequence.

145. The OX40 antigen binding molecule of claim 144, wherein the amino acid sequences of the B domain and the G domain are identical, wherein the sequence is an endogenous CH3 sequence.

146. The OX40 antigen binding molecule of claim 144, wherein the amino acid sequences of the B domain and the G domain are different and separately comprise respectively orthogonal modifications in an endogenous CH3 sequence, wherein the B domain interacts with the G domain, and wherein neither the B domain nor the G domain significantly interacts with a CH3 domain lacking the orthogonal modification.

147. The OX40 antigen binding molecule of claim 146, wherein the orthogonal modifications of the B domain and the G domain comprise mutations that generate engineered disulfide bridges between the B domain and the G domain.

148. The OX40 antigen binding molecule of claim 147, wherein the mutations of the B domain and the G domain that generate engineered disulfide bridges are a S354C mutation in one of the B domain and G domain, and a 349C in the other domain.

149. The OX40 antigen binding molecule of any one of claims 146-148, wherein the orthogonal modifications of the B domain and the G domain comprise knob-in-hole mutations.

150. The OX40 antigen binding molecule of claim 149, wherein the knob-in hole mutations of the B domain and the G domain are a T366W mutation in one of the B domain and G domain, and a T366S, L368A, and a Y407V mutation in the other domain.

151. The OX40 antigen binding molecule of any one of claims 146-150, wherein the orthogonal modifications of the B domain and the G domain comprise charge-pair mutations.

152. The OX40 antigen binding molecule of claim 151, wherein the charge-pair mutations of the B domain and the G domain are a T366K mutation in one of the B domain and G domain, and a L351D mutation in the other domain.

153. The OX40 antigen binding molecule of any one of claims 138-152, wherein domain B and domain G have an IgM CH2 amino acid sequence or an IgE CH2 amino acid sequence.

154. The OX40 antigen binding molecule of claim 153, wherein the IgM CH2 amino acid sequence or the IgE CH2 amino acid sequence comprise orthogonal modifications.

155. The OX40 antigen binding molecule of any one of claims 139-154, wherein domain I has a CL sequence and domain M has a CH1 sequence.

156. The OX40 antigen binding molecule of any one of claims 139-154, wherein domain has a CH1 sequence and domain M has a CL sequence.

157. The OX40 antigen binding molecule of claim 155 or 156, wherein the CH1 sequence and the CL sequence each comprise one or more orthogonal modifications, wherein a domain having the CH1 sequence does not significantly interact with a domain having a CL sequence lacking the orthogonal modification.

158. The OX40 antigen binding molecule of claim 157, wherein the orthogonal modifications in the CH1 sequence and the CL sequence comprise mutations that generate engineered disulfide bridges between the at least one CH1 domain and a CL domain, the mutations selected from the group consisting of: an engineered cysteine at position 138 of the CH1 sequence and position 116 of the CL sequence; an engineered cysteine at position 128 of the CH1 sequence and position 119 of the CL sequence, and an engineered cysteine at position 129 of the CH1 sequence and position 210 of the CL sequence.

159. The OX40 antigen binding molecule of claim 157, wherein the orthogonal modifications in the CH1 sequence and the CL sequence comprise mutations that generate engineered disulfide bridges between the at least one CH1 domain and a CL domain, wherein the mutations comprise and engineered cysteines at position 128 of the CH1 sequence and position 118 of a CL Kappa sequence.

160. The OX40 antigen binding molecule of claim 157, wherein the orthogonal modifications in the CH1 sequence and the CL sequence comprise mutations that generate engineered disulfide bridges between the at least one CH1 domain and a CL domain, the mutations selected from the group consisting of: a F118C mutation in the CL sequence with a corresponding A141C in the CH1 sequence; a F118C mutation in the CL sequence with a corresponding L128C in the CH1 sequence; and a S162C mutations in the CL sequence with a corresponding P171C mutation in the CH1 sequence.

161. The OX40 antigen binding molecule of any of claims 157-160, wherein the orthogonal modifications in the CH1 sequence and the CL sequence comprise charge-pair mutations between the at least one CH1 domain and a CL domain, the charge-pair mutations selected from the group consisting of: a F118S mutation in the CL sequence with a corresponding A141L in the CH1 sequence; a F118A mutation in the CL sequence with a corresponding A141L in the CH1 sequence; a F118V mutation in the CL sequence with a corresponding A141L in the CH1 sequence; and a T129R mutation in the CL sequence with a corresponding K147D in the CH1 sequence.

162. The OX40 antigen binding molecule of any of claims 158-160, wherein the orthogonal modifications in the CH1 sequence and the CL sequence comprise charge-pair mutations between the at least one CH1 domain and a CL domain, the charge-pair mutations selected from the group consisting of: a N138K mutation in the CL sequence with a corresponding G166D in the CH1 sequence, and a N138D mutation in the CL sequence with a corresponding G166K in the CH1 sequence.

163. The OX40 antigen binding molecule of any of claims 138-162, wherein domain A has a VL amino acid sequence and domain F has a VH amino acid sequence.

164. The OX40 antigen binding molecule of any of claims 138-162, wherein domain A has a VH amino acid sequence and domain F has a VL amino acid sequence.

165. The OX40 antigen binding molecule of any of claims 139-164, wherein domain H has a VL amino acid sequence and domain L has a VH amino acid sequence.

166. The OX40 antigen binding molecule of any of claims 139-164, wherein domain H has a VH amino acid sequence and domain L has a VL amino acid sequence.

167. The OX40 antigen binding molecule of any of claims 139-166, wherein domain D and domain J have a CH2 amino acid sequence.

168. The OX40 antigen binding molecule of any one of claims 138-167, wherein the E domain has a CH3 amino acid sequence.

169. The OX40 antigen binding molecule of any one of claims 139-168, wherein the amino acid sequences of the E domain and the K domain are identical, wherein the sequence is an endogenous CH3 sequence.

170. The OX40 antigen binding molecule of any one of claims 139-169, wherein the amino acid sequences of the E domain and the K domain are different.

171. The OX40 antigen binding molecule of claim 170, wherein the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification.

172. The OX40 antigen binding molecule of claim 171, wherein the orthogonal modifications comprise mutations that generate engineered disulfide bridges between the E domain and the K domain.

173. The OX40 antigen binding molecule of claim 172, wherein the mutations that generate engineered disulfide bridges are a S354C mutation in one of the E domain and the K domain, and a 349C in the other domain.

174. The OX40 antigen binding molecule of any one of claims 170-173, wherein the orthogonal modifications in the E domain and the K domain comprise knob-in-hole mutations.

175. The OX40 antigen binding molecule of claim 174, wherein the knob-in hole mutations are a T366W mutation in one of the E domain or the K domain and a T366S, L368A, and a Y407V mutation in the other domain.

176. The OX40 antigen binding molecule of any one of claims 170-175, wherein the orthogonal modifications in the E domain and the K domain comprise charge-pair mutations.

177. The OX40 antigen binding molecule of claim 176, wherein the charge-pair mutations are a T366K mutation in one of the E domain or the K domain and a corresponding L351D mutation in the other domain.

178. The OX40 antigen binding molecule of claim 169, wherein the amino acid sequences of the E domain and the K domain are endogenous sequences of two different antibody domains, the domains selected to have a specific interaction that promotes the specific association between the first and the third polypeptides.

179. The OX40 antigen binding molecule of claim 178, wherein the two different amino acid sequences are a CH1 sequence and a CL sequence.

180. The OX40 antigen binding molecule of any one of claims 121-179, wherein the OX40 antigen binding molecule further comprises a third antigen binding site.

181. The OX40 antigen binding molecule of claim 180, wherein the third antigen binding site is specific for an OX40 antigen.

182. The OX40 antigen binding molecule of claim 181, wherein the first antigen binding site and the third antigen binding site are specific for the same OX40 antigen.

183. The OX40 antigen binding molecule of claim 182, wherein the first antigen binding site comprises a VL CDR1 comprising SEQ ID NO:220, a VL CDR2 comprising SEQ ID NO:221, and a VL CDR3 comprising SEQ ID NO:203, and a VH CDR1 comprising SEQ ID NO:83, a VH CDR2 comprising SEQ ID NO:123, and a VH CDR3 comprising SEQ NO:163.

184. The OX40 antigen binding molecule of claim 182, wherein the first antigen binding site comprises a VL CDR1 comprising SEQ ID NO:220, a VL CDR2 comprising SEQ ID NO:221, and a VL CDR3 comprising SEQ ID NO:227, and a VH CDR1 comprising SEQ ID NO:83, a VH CDR2 comprising SEQ ID NO:123, and a VH CDR3 comprising SEQ ID NO:163.

185. The OX40 antigen binding molecule of claim 182, wherein the first antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a CDR2 comprising SEQ ID NO:110, and a VH CDR3 comprising SEQ ID NO:150.

186. The OX40 antigen binding molecule of claim 181, wherein the first antigen binding site and the third antigen binding site are specific for a different OX40 antigens.

187. The OX40 antigen binding molecule of claim 186, wherein the first antigen binding site comprises a VL CDR1 comprising SEQ ID NO:220, a VL CDR2 comprising SEQ ID NO:221, and a VL CDR3 comprising SEQ ID NO:203, and a VH CDR1 comprising SEQ ID NO:83, a VH CDR2 comprising SEQ ID NO:123, and a VH CDR3 comprising SEQ ID NO:163; and
the third antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ NO:70, a VH CDR2 comprising SEQ ID NO:110, and a VH CDR3 comprising SEQ ID NO:150.

188. The OX40 antigen binding molecule of claim 186, wherein the third antigen binding site comprises a VL CDR1 comprising SEQ ID NO:220, a VL CDR2 comprising SEQ ID NO:221, and a VL CDR3 comprising SEQ ID NO:203, and a VH CDR1 comprising SEQ ID NO:83, a VH CDR2 comprising SEQ ID NO:123; and a VH CDR3 comprising SEQ ID NO:163; and
the first antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150.

189. The OX40 antigen binding molecule of claim 186, wherein the first antigen binding site comprises a VL CDR1 comprising SEQ ID NO:220, a VL CDR2 comprising SEQ ID NO:221, and a VL CDR3 comprising SEQ ID NO:227, and a VH CDR1 comprising SEQ ID NO:83, a VH CDR2 comprising SEQ ID NO:123, and a VH CDR3 comprising SEQ ID NO:163; and
the third antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150.

190. The OX40 antigen binding molecule of claim 186, wherein the third antigen binding site comprises a VL CDR1 comprising SEQ ID NO:220, a VL CDR2 comprising SEQ ID NO:221, and a VL CDR3 comprising SEQ ID NO:227, and a VH CDR1 comprising SEQ NO:83, a VH CDR2 comprising SEQ ID NO:123, and a VH CDR3 comprising SEQ ID NO:163; and
the first antigen binding site comprises a VL CDR1 comprising SEQ ID NO:220, a VL CDR2 comprising SEQ ID NO:221, and a VL CDR3 comprising SEQ ID NO:190, and a VH CDR1 comprising SEQ ID NO:70, a VH CDR2 comprising SEQ ID NO:110, and a VH CDR3 comprising SEQ ID NO:150.

191. The OX40 antigen binding molecule of any one of claims 180-190, wherein the OX40 antigen binding molecule comprises a fifth polypeptide chain, wherein
(a) the first polypeptide chain further comprises a domain N and a domain O, wherein the domains are arranged, from N-terminus to C-terminus, in a N-O-A-B-D-E orientation, and
wherein domain N has a variable region domain amino acid sequence, domain O has a constant region amino acid sequence;
(b) the fifth polypeptide chain comprises
a domain P and a domain Q, wherein the domains are arranged, from N-terminus to C-terminus, in a P-Q orientation, and
wherein domain P has a variable region domain amino acid sequence and domain Q has a constant region amino acid sequence; and
(c) the first and the fifth polypeptides are associated through an interaction between the N and the P domains and an interaction between the O and the Q domains to form the OX40 antigen binding molecule.

192. The OX40 antigen binding molecule of claim 191, wherein:
(a) the amino acid sequences of domain N and domain A are identical,
the amino acid sequences of domain H is different from the sequence of domain N and domain A,
the amino acid sequences of domain O and domain B are identical,
the amino acid sequences of domain I is different from the sequence of domain O and domain B,
the amino acid sequences of domain P and domain F are identical,
the amino acid sequences of domain L is different from the sequence of domain P and domain F,
the amino acid sequences of domain Q and domain G are identical,
the amino acid sequences of domain M is different from the sequence of domain Q and domain G; and
(b) wherein the interaction between the A domain and the F domain form a first antigen binding site specific for a first antigen, the interaction between the H domain and the L domain form a second antigen binding site specific for a second antigen, and the interaction between the N domain and the P domain form a third antigen binding site specific for the first antigen.

193. The OX40 antigen binding molecule of claim 192, wherein the first antigen is a first epitope of the OX40 antigen.

194. The OX40 antigen binding molecule of claim 193, wherein the second antigen is a second epitope of the OX40 antigen.

195. The OX40 antigen binding molecule of claim 194, wherein the first epitope and the second epitope are non-overlapping epitopes.

196. The OX40 antigen binding molecule of claim 191, wherein:
(a) the amino acid sequences of domain N, domain A, and domain H are different,
the amino acid sequences of domain O, domain B, and domain I are different,
the amino acid sequences of domain P, domain F, and domain L are different, and
the amino acid sequences of domain Q, domain G, and domain M are different; and
(b) the interaction between the A domain and the F domain form a first antigen binding site specific for a first antigen,
the interaction between the H domain and the L domain form a second antigen binding site specific for a second antigen, and
the interaction between the N domain and the P domain form a third antigen binding site specific for a third antigen.

197. The OX40 antigen binding molecule of any one of claims 180-186, wherein the OX40 antigen binding molecule comprises a sixth polypeptide chain, wherein:
(a) the third polypeptide chain further comprises a domain R and a domain S,
wherein the domains are arranged, from N-terminus to C-terminus, in a R-S-H-I-J-K orientation, and
wherein domain R has a variable region amino acid sequence and domain S has a constant domain amino acid sequence;
(b) the sixth polypeptide chain comprises:
a domain T and a domain U,
wherein the domains are arranged, from N-terminus to C-terminus, in a T-U orientation, and
wherein domain T has a variable region amino acid sequence and domain U has a constant domain amino acid sequence; and
(c) the third and the sixth polypeptides are associated through an interaction between the Rand the T domains and an interaction between the S and the U domains to form the OX40 antigen binding molecule.

198. The OX40 antigen binding molecule of claim 197, wherein:
(a) the amino acid sequences of domain R and domain A are identical,
the amino acid sequences of domain H is different from the sequence of domain R and domain A,
the amino acid sequences of domain S and domain B are identical,
the amino acid sequences of domain I is different from the sequence of domain S and domain B,
the amino acid sequences of domain and domain F are identical,
the amino acid sequences of domain L is different from the sequence of domain T and domain F,
the amino acid sequences of domain U and domain G are identical,
the amino acid sequences of domain M is different from the sequence of domain U and domain G, and
(b) the interaction between the A domain and the F domain form a first antigen binding site specific for a first antigen,
the interaction between the H domain and the L domain form a second antigen binding site specific for a second antigen, and
the interaction between the R domain and the T domain form a third antigen binding site specific for the first antigen.

199. The OX40 antigen binding molecule of claim 198, wherein the first antigen is a first epitope of the OX40 antigen.

200. The OX40 antigen binding molecule of claim 199, wherein the second antigen is a second epitope of the OX40 antigen.

201. The OX40 antigen binding molecule of claim 200, wherein the first epitope and the second epitope are non-overlapping epitopes.

202. The OX40 antigen binding molecule of claim 197, wherein:
(a) the amino acid sequences of domain R and domain H are identical,
the amino acid sequences of domain A is different from the sequence of domain R and domain H,
the amino acid sequences of domain S and domain I are identical,
the amino acid sequences of domain B is different from the sequence of domain S and domain I,
the amino acid sequences of domain T and domain L are identical,
the amino acid sequences of domain F is different from the sequence of domain T and domain L,
the amino acid sequences of domain U and domain M are identical,
the amino acid sequences of domain G is different from the sequence of domain U and domain M, and
(b) the interaction between the A domain and the F domain form a first antigen binding site specific for a first antigen,
the interaction between the H domain and the L domain form a second antigen binding site specific for a second antigen, and
the interaction between the R domain and the T domain form a third antigen binding site specific for the second antigen.

203. The OX40 antigen binding molecule of claim 202, wherein the second antigen is a first epitope of the OX40 antigen.

204. The OX40 antigen binding molecule of claim 203, wherein the first antigen is a second epitope of the OX40 antigen.

205. The OX40 antigen binding molecule of claim 204, wherein the first epitope and the second epitope are non-overlapping epitopes.

206. The OX40 antigen binding molecule of claim 197, wherein:
(a) the amino acid sequences of domain R, domain A, and domain H are different,
the amino acid sequences of domain S, domain B, and domain I are different,
the amino acid sequences of domain T, domain F, and domain L are different, and
the amino acid sequences of domain U, domain G, and domain M are different; and
(b) the interaction between the A domain and the F domain form a first antigen binding site specific for a first antigen,
the interaction between the H domain and the I, domain form a second antigen binding site specific for a second antigen, and
the interaction between the R domain and the T domain form a third antigen binding site specific for a third antigen.

207. A purified binding molecule comprising the multivalent antibody construct of any one of claims 1-120 or the OX40 antigen binding molecule of any one of claims 121-206.

208. The purified binding molecule of claim 207, wherein the purified binding molecule is purified by a purification method comprising a CH1 affinity purification step.

209. The purified binding molecule of claim 207 or 208, wherein the purified binding molecule is purified by a single-step purification method.

210. The multivalent antibody construct of any one of claims 1-120, the OX40 antigen binding molecule of any one of claims 121-206, or the purified binding molecule of any one of claims 207-209, wherein the multivalent antibody construct, the OX40 antigen binding molecule, or the purified binding molecule comprises a biophysical property selected from the group consisting of high yield, high purity, homogeneity, stability, long-term stability, acid stability, thermostability, low antibody cross-interaction, low antibody self-interaction, low hydrophobic binding, and cyno crossreactivity.

211. A pharmaceutical composition comprising the multivalent antibody construct of any one of claims 1-120, the OX40 antigen binding molecule of any one of claims 121-206, or the purified binding molecule of any one of claims 207-209, and a pharmaceutically acceptable diluent.

212. A method of treating cancer, comprising administering a therapeutically effective amount of the pharmaceutical composition of claim 211 to a patient in need thereof.

213. An isolated polynucleotide encoding an amino acid sequence comprising the multivalent antibody construct of any one of claims 1-120 or the OX40 antigen binding molecule of any one of claims 121-206.

214. A vector comprising the isolated polynucleotide of claim 213.

215. A host cell comprising the vector of claim 214.
